(19) United States
(12) Patent Application Publication
(10) Pub. No.: US 2010/0105664 A1
(43) Pub. Date: Apr. 29, 2010

(54) ORGANIC COMPOUNDS

(76) Inventors: Richard Heng, Hegenheim (FR); Guido Koch, Bettingen (CH); Achim Schlapbach, Lorrach (DE); Max Peter Seiler, Riehen (CH)

Correspondence Address: Fanelli Strain & Haag PLLC, 1455 Pennsylvania Ave., N.W., suite 400, Washington, DC, 20004, US

(21) Appl. No.: 12/442,264

(22) PCT Filed: Sep. 19, 2007
(86) PCT No.: PCT/EP07/08153

§ 371 Date: Mar. 20, 2009

(30) Foreign Application Priority Data
Sep. 21, 2006 (EP) 06121036.5

(51) Int. Cl. A61K 31/4439; C07D 401/14; C07D 413/14; A61K 31/5377; A61K 31/551; A61P 29/00

(52) U.S. Cl. 514/218; 546/279.1; 544/124; 540/575; 514/343; 514/235.5

(57) ABSTRACT

A compound of formula (I) or a pharmaceutically acceptable salt or prodrug ester thereof:

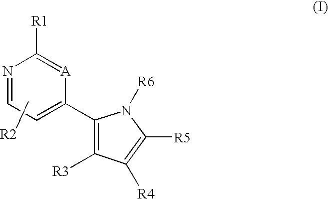

wherein the groups R1-R6 and A are as defined in the specification.

The present invention relates to novel heteroaromatic compounds as inhibitors of protein kinases, in particular mitogen-activated protein kinase-activated protein kinase-2 (MK2 or MAPKAP kinase-2) and 3-phosphoinositide-dependent protein kinase-1 (PDK-1).

Accordingly the present invention provides a compound of formula (I) or a pharmaceutically acceptable salt or a pharmaceutically-acceptable and -cleavable ester, or acid addition salt thereof

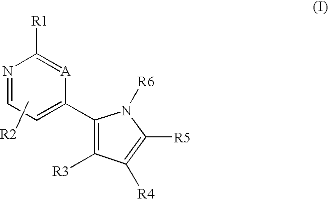

wherein A is CH or N;

R1 is selected from aryl, heteroaryl, aryl-C₂-C₆ alkenyl, heteroaryl-C₂-C₆ alkenyl, C₃-C₇ cycloalkyl, C₃-C₇ cycloalkyl-C₂-C₆ alkenyl, aryl-C₂-C₆ alkynyl, heteroaryl-C₂-C₆ alkynyl, C₃-C₇ cycloalkyl-C₂-C₆ alkynyl, heterocycloalkyl, heterocycloalkyl C₂-C₆ alkenyl, heterocycloalkyl C₂-C₆ alkynyl, amino, C₁-C₆ alkylamino, arylamino, heteroarylamino, aryloxy, heteroaryloxy, the group R1 being optionally substituted by halo, C₁-C₆ alkyl, cyano, heterocycloalkyl, heterocycloalkyl-C₁-C₆ alkyl, carbamoyl, carboxyl, carbonyl, carbonylamino, heterocycloalkylcarbonyl, amino, C₁-C₆ alkylamino, sulfonyl, C₁-C₆ alkylcarbonylamino, hydroxy, C₁-C₆ alkoxy, C₁-C₆ cycloalkyloxy, aryloxy, heteroaryloxy,

each of which may be optionally substituted by C₁-C₆ alkyl, cycloalkyl, heterocycloalkyl, C₁-C₆ alkoxy, amino, cyano, halo, carboxyl, carboxyalkyl, carbamoyl, hydroxyl;

R2 is selected from H, aryl, heteroaryl, and aryl-C₂-C₆ alkenyl,

the group R2 being optionally substituted by halo, C₁-C₆ alkyl, cyano, heterocycloalkyl, heterocycloalkyl-C₁-C₆ alkyl, carbamoyl, carbonyl, carbonylamino, heterocycloalkylcarbonyl, amino, C₁-C₆ alkylamino, sulfonyl, C₁-C₆ alkylcarbonylamino, hydroxy, C₁-C₆ alkoxy, C₁-C₆ cycloalkyloxy, aryloxy, heteroaryloxy,

each of which may be optionally substituted by C₁-C₆ alkyl, cycloalkyl, heterocycloalkyl, C₁-C₆ alkoxy, amino, cyano, halo, carboxyl, carboxyalkyl, carbamoyl, hydroxyl;

R3 is H, C₁-C₆ alkyl, cyano, amino, halo, CF₃ or CHF₂;

R4 is selected from cyano, carbamoyl, sulfamoyl, amidino, N-hydroxy amidino (i.e. —C(═NH)—NOH), carboxyl, carboxylic ester;

R5 is selected from optionally substituted C₁-C₆ alkyl, heterocycloalkyl or amino,

the optional substituents on R5 being selected from C₁-C₆ alkyl, halo, cyano, hydroxyl, amino, sulfonyl, aryl, carbonyl, C₁-C₆ alkylcarbonyl, C₁-C₆ alkyloxycarbonyl, C₁-C₆ alkyloxy, each of which substituents may be optionally substituted by C₁-C₆ alkyl, C₁-C₆ alkyloxy, hydroxyl, sulfonyl, aryl, halo, cyano, amino, alkylamino, dialkylamino;

R6 is H or C₁-C₆ alkyl or sulfonyl,

the group R6 being optionally substituted by C₁-C₆ alkyl, hydroxy, halo, cyano, amino, alkylamino, dialkylamino.

Preferably, R1 is selected from aryl, heteroaryl, and aryl-C₂-C₆ alkenyl, and R2 is selected from H, aryl, heteroaryl, and aryl-C₂-C₆ alkenyl, the groups R1 and R2 being optionally substituted by halo, C₁-C₆ alkyl, heterocycloalkyl, heterocycloalkyl-C₁-C₆ alkyl, carbamoyl, carbonyl, carboxyl, alkoxy, heterocycloalkylcarbonyl, amino, C₁-C₆ alkylamino, sulfonyl, C₁-C₆ alkylcarbonylamino,

each of which in turn may be optionally substituted by C₁-C₆ alkyl, C₁-C₆ alkoxy, amino.

In an alternative preferred embodiment, R2 is H. Preferably, R1 is aryl, heteroaryl, aryl-C₂-C₆ alkenyl, amino or arylamino, R1 being optionally substituted by halo, C₁-C₆ alkyl, heterocycloalkyl, heterocycloalkyl-C₁-C₆ alkyl, carbamoyl, carbonyl, carboxyl, alkoxy, heterocycloalkylcarbonyl, amino, C₁-C₆ alkylamino, sulfonyl, C₁-C₆ alkylcarbonylamino, each of which in turn may be optionally substituted by C₁-C₆ alkyl, C₁-C₆ alkoxy, amino.

More preferably, R1 is optionally substituted aryl, heteroaryl or aryl-C₂-C₆ alkenyl, the optional substituents being as previously defined.

Yet more preferably, R1 is optionally substituted aryl-C₂-C₆ alkenyl. More preferably, R1 is optionally substituted aryl-ethylenyl, most preferably optionally substituted styryl.

Alternatively preferably, R1 is optionally substituted aryl or heteroaryl. A preferred aryl group is phenyl. A preferred heteroaryl group is pyridine, pyrimidine or a fused bicyclic heteroaryl group.

R3 is preferably H or C₁-C₆ alkyl, more preferably R3 is H.

R4 is preferably cyano or carbamoyl.

R5 is preferably optionally substituted substituted C₁-C₆ alkyl or heterocycloalkyl, more preferably optionally substituted heterocycloalkyl.

R6 is preferably H.

A is preferably CH.

A second aspect of the invention provides a compound of formula (II) or a pharmaceutically acceptable salt or a pharmaceutically-acceptable and -cleavable ester, or acid addition salt thereof:

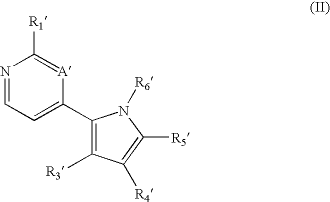

wherein

A′ is CH or N;

R₁′ is selected from optionally substituted aryl, heteroaryl, aryl-C₂-C₆ alkenyl, heteroaryl-C₂-C₆ alkenyl, C₃-C₇ cycloalkyl, C₃-C₇ cycloalkyl-C₂-C₆ alkenyl, aryl-C₂-C₆ alkynyl, heteroaryl-C₂-C₆ alkynyl, C₃-C₇ cycloalkyl-C₂-C₆ alkynyl, heterocycloalkyl, heterocycloalkyl C₂-C₆ alkenyl or heterocycloalkyl C₂-C₆ alkynyl, amino, C₁-C₆ alkylamino, arylamino, heteroarylamino, aryloxy, heteroaryloxy,

the optional substituents on R1′ being selected from halo, C₁-C₆ alkyl, cyano, heterocycloalkyl, heterocycloalkyl-C₁-C₆ alkyl, carbamoyl, carbonyl, heterocycloalkylcarbonyl, amino, C₁-C₆ alkylamino, sulfonyl, C₁-C₆ alkylcarbonylamino, hydroxy, C₁-C₆ alkoxy, C₁-C₆ cycloalkyloxy, aryloxy, heteroaryloxy,

each of which may be optionally substituted by C₁-C₆ alkyl, cycloalkyl, heterocycloalkyl, C₁-C₆ alkoxy, amino, cyano, halo, carboxyl, carboxyalkyl, carbamoyl, hydroxyl,

R₃′ H, halo or C₁-C₆ alkyl;

R₄′ is cyano, carbamoyl, amidino or N-hydroxy-amidino (—C(═NH)—NOH);

R₅′ is selected from optionally substituted C₁-C₆ alkyl, heterocycloalkyl, the optional substituents on R5 being selected from C₁-C₆ alkyl, halo, cyano, hydroxyl, amino, sulfonyl, aryl, carbonyl, C₁-C₆ alkylcarbonyl, C₁-C₆ alkyloxycarbonyl, C₁-C₆ alkyloxy, each of which substituents may be optionally substituted by C₁-C₆ alkyl, C₁-C₆ alkyloxy, hydroxyl, sulfonyl, aryl, halo, cyano, amino, alkylamino, dialkylamino;

R₆′ is H or C₁-C₆ alkyl, sulfonyl, optionally substituted by C₁-C₆ alkyl, hydroxy, halo, amino, alkylamino, dialkylamino.

Preferably A′ is CH.

R₁′ is preferably selected from optionally substituted aryl, heteroaryl, aryl-C₂-C₆ alkenyl.

R₃′ is preferably H.

R₄′ is preferably cyano. Alternatively preferably, R₄′ is carbamoyl, more preferably —CONH₂. Alternatively preferably, R₄′ is —C(═NH)—NOH.

For the avoidance of doubt, the terms listed below are to be understood to have the following meaning throughout the present description and claims:

The term “lower”, when referring to organic radicals or compounds means a compound or radical with may be branched or unbranched with up to and including 7 carbon atoms.

An alkyl group may be branched, unbranched or cyclic and contains 1 to 7 carbon atoms, preferably 1 to 4 carbon atoms. Lower alkyl represents, for example: methyl, ethyl, propyl, butyl, isopropyl, isobutyl, tertiary butyl or 2,2-dimethylpropyl.

An alkoxy group may be branched or unbranched and contains 1 to 7 carbon atoms, preferably 1 to 6 carbon atoms. Lower alkoxy represents, for example: methoxy, ethoxy, propoxy, butoxy, isopropoxy, isobutoxy or tertiary butoxy. Alkoxy includes cycloalkyloxy and cycloalkyl—lower alkyloxy.

An alkene, alkenyl or alkenoxy group is branched or unbranched and contains 2 to 7 carbon atoms, preferably 1 to 4 carbon atoms and contains at least one carbon-carbon double bond. Alkene, alkenyl or alkenyloxy represents for example vinyl, prop-1-enyl, allyl, butenyl, isopropenyl or isobutenyl and the oxy equivalents thereof.

An alkyne or alkynyl group is branched or unbranched and contains 2 to 7 carbon atoms, preferably 1 to 4 carbon atoms and contains at least one carbon-carbon triple bond. Alkyne or alkynyl or alkenyloxy represents for example ethynyl or propynyl.

In the present application, oxygen containing substituents, e.g. alkoxy, alkenyloxy, alkynyloxy, carbonyl, etc. encompass their sulphur containing homologues, e.g. thioalkyl, alkyl-thioalkyl, thioalkenyl, alkenyl-thioalkyl, thioalkynyl, thiocarbonyl, sulphone, sulphoxide etc.

Halo or halogen represents chloro, fluoro, bromo or iodo.

Aryl represents carbocyclic aryl or biaryl.

Carbocyclic aryl is an aromatic cyclic hydrocarbon containing from 6 to 18 ring atoms. It can be monocyclic, bicyclic or tricyclic, for example naphthyl, phenyl, or phenyl mono-, di- or trisubstituted by one, two or three substituents.

Heterocyclic aryl or heteroaryl is an aromatic monocyclic or bicyclic hydrocarbon containing from 5 to 18 ring atoms one or more of which are heteroatoms selected from O, N or S. Preferably there are one to three heteroatoms. Heterocyclic aryl represents, for example: tetrazolyl, imidazolyl, oxadiazolyl, pyridyl, indolyl, quinoxalinyl, quinolinyl, isoquinolinyl, benzothienyl, benzofuranyl, benzthiophenyl, benzopyranyl, benzothiopyranyl, furanyl, pyrrolyl, thiazolyl, oxazolyl, isoxazolyl, triazolyl, pyrazolyl, thienyl, benzimidazolyl, benzthiazolyl, benzoxazolyl, Heterocyclic aryl also includes such substituted radicals.

Cycloalkyl represents a cyclic hydrocarbon containing from 3 to 12 ring atoms preferably from 3 to 6 ring atoms. Cycloalkyl represents, for example: cyclopropyl, cyclobutyl, cyclopentyl, or cyclohexyl. The cycloalkyl may optionally be substituted.

Heterocycloalkyl represents a mono-, di- or tricyclic hydrocarbon which may be saturated or unsaturated and which contains one or more, preferably one to three heteroatoms selected from O, N or S. Preferably it contains between three and 18 ring atoms, more preferably between 3 and 8 ring atoms. The term heterocycloalkyl is intended also to include bridged heterocycloalkyl groups such as 3-hydroxy-8-aza-bicyclo[3.2.1]oct-8-yl or 2,6-diaza-tricyclo[3.3.1.1*3,7*]dec-1-yl.

Pharmaceutically acceptable salts include acid addition salts with conventional acids, for example mineral acids, e.g. hydrochloric acid, sulfuric or phosphoric acid, or organic acids, for example aliphatic or aromatic carboxylic or sulfonic acids, e.g. acetic, trifluoroacetic, propionic, succinic, glycolic, lactic, malic, tartaric, citric, ascorbic, maleic, fumaric, hydroxylmaleic, pyruvic, pamoic, methanesulfonic, toluenesulfonic, naphthalenesulfonic, sulfanilic or cyclohexylsulfamic acid; also amino acids, such as arginine and lysine. For compounds of the invention having acidic groups, for example a free carboxy group, pharmaceutically acceptable salts also represent metal or ammonium salts, such as alkali metal or alkaline earth metal salts, e.g. sodium, potassium, magnesium or calcium salts, as well as ammonium salts, which are formed with ammonia or suitable organic amines.

The agents of the invention which comprise free hydroxyl groups may also exist in the form of pharmaceutically acceptable, physiologically cleavable esters, and as such are included within the scope of the invention. Such pharmaceutically acceptable esters are preferably prodrug ester derivatives, such being convertible by solvolysis or cleavage under physiological conditions to the corresponding agents of the invention which comprise free hydroxyl groups. Suitable pharmaceutically acceptable prodrug esters are those derived from a carboxylic acid, a carbonic acid monoester or a carbamic acid, advantageously esters derived from an optionally substituted lower alkanoic acid or an arylcarboxylic acid.

Preferred compounds of formula (I) are:

5′-{2-[(E)-2-(4-Fluoro-phenyl)-vinyl]-pyridin-4-yl}-2,3,4,5-tetrahydro-1′H-[1,2′]bipyrrolyl-3′carbonitrile,

5′-{2-[(E)-2-(4-Fluoro-phenyl)-vinyl]-pyridin-4-yl}-2,3,4,5-tetrahydro-1′H-[1,2′]bipyrrolyl-3′carboxylic acid amide,

5-{2-[(E)-2-(4-Fluoro-phenyl)-vinyl]-pyridin-4-yl}-2-morpholin-4-yl-1H-pyrrole-3-carbonitrile,

5-{2-[(E)-2-(4-Fluoro-phenyl)-vinyl]-pyridin-4-yl}-2-morpholin-4-yl-1H-pyrrole-3-carboxylic acid amide,

2-(2-Amino-ethylamino)-5-[2-((E)-styryl)-pyridin-4-yl]-1H-pyrrole-3-carbonitrile,

2-(3-Hydroxy-propylamino)-5-[2-((E)-styryl)-pyridin-4-yl]-1H-pyrrole-3-carbonitrile,

2-(2-Hydroxy-ethylamino)-5-[2-((E)-styryl)-pyridin-4-yl]-1H-pyrrole-3-carbonitrile,

5-{2-[(E)-2-(4-Fluoro-phenyl)-vinyl]-pyridin-4-yl}-2-piperazin-1-yl-1H-pyrrole-3-carbonitrile trifluoroacetate,

5-{2-[(E)-2-(4-Fluoro-phenyl)-vinyl]-pyridin-4-yl)-2-piperazin-1-yl-1H-pyrrole-3-carboxylic acid amide,

2-Piperazin-1-yl-5-(2-quinolin-3-yl-pyridin-4-yl)-1H-pyrrole-3-carbonitrile trifluoroacetate,

2-Piperazin-1-yl-5-(2-quinolin-3-yl-pyridin-4-yl)-1H-pyrrole-3-carboxylic acid amide,

5-(2-Benzofuran-2-yl-pyridin-4-yl)-2-piperazin-1-yl-1H-pyrrole-3-carbonitrile trifluoroacetate,

2-Piperazin-1-yl-5-[2-((E)-styryl)-pyridin-4-yl]-1H-pyrrole-3-carbonitrile,

2-Piperazin-1-yl-5-[2-((E)-styryl)-pyridin-4-yl]-1H-pyrrole-3-carboxylic acid amide,

5-[2-(1-Methyl-1H-indol-5-yl)-pyridin-4-yl]-2-piperazin-1-yl-1H-pyrrole-3-carbonitrile,

5-[2-(1-Methyl-1H-indol-5-yl)-pyridin-4-yl]-2-piperazin-1-yl-1H-pyrrole-3-carboxylic acid amide,

N-{4-[4-(4-Cyano-5-piperazin-1-yl-1H-pyrrol-2-yl)-pyridin-2-yl]-phenyl}-acetamide,

5-[2-(4-Acetylamino-phenyl)-pyridin-4-yl]-2-piperazin-1-yl-1H-pyrrole-3-carboxylic acid amide,

5-[2-(3-Dimethylamino-phenyl)-pyridin-4-yl]-2-piperazin-1-yl-1H-pyrrole-3-carbonitrile,

N-{3-[4-(4-Cyano-5-piperazin-1-yl-1H-pyrrol-2-yl)-pyridin-2-yl]-phenyl}-acetamide,

5-[2-(4-Dimethylamino-phenyl)-pyridin-4-yl]-2-piperazin-1-yl-1H-pyrrole-3-carbonitrile,

5-[2-(1H-Indol-6-yl)-pyridin-4-yl]-2-piperazin-1-yl-1H-pyrrole-3-carbonitrile,

5-[2-(1H-Indol-5-yl)-pyridin-4-yl]-2-piperazin-1-yl-1H-pyrrole-3-carbonitrile,

(E)-3-{4-[4-(4-Cyano-5-piperazin-1-yl-1H-pyrrol-2-yl)-pyridin-2-yl]-phenyl}-acrylic acid,

4-{(E)-2-[4-(4-Cyano-5-piperazin-1-yl-1H-pyrrol-2-yl)-pyridin-2-yl]-vinyl}-benzoic acid methyl ester,

5-{2-[1-(2-Morpholin-4-yl-ethyl)-1H-pyrazol-4-yl]-pyridin-4-yl}-2-piperazin-1-yl-1H-pyrrole-3-carbonitrile,

5-[5′-(2-Methoxy-ethoxy)-(2,3′]bipyridinyl-4-yl]-2-piperazin-1-yl-1H-pyrrole-3-carbonitrile,

5-{2-[3-(3-Hydroxy-propyl)-phenyl]pyridin-4-yl}-2-piperazin-1-yl-1H-pyrrole-3-carbonitrile,

{3-[4-(4-Cyano-5-piperazin-1-yl-1H-pyrrol-2-yl)-pyridin-2-yl]-5-fluoro-phenoxy}-acetic acid,

5-[2-(4-Methanesulfonyl-phenyl)-pyridin-4-yl]-2-piperazin-1-yl-1H-pyrrole-3-carbonitrile trifluoroacetate,

5-[2-(4-Methanesulfonyl-phenyl)-pyridin-4-yl]-2-piperazin-1-yl-1H-pyrrole-3-carboxylic acid amide,

5-[2-(3-Methanesulfonyl-phenyl)-pyridin-4-yl]-2-piperazin-1-yl-1H-pyrrole-3-carbonitrile trifluoroacetate,

5-[2-(3-Methanesulfonyl-phenyl)-pyridin-4-yl]-2-piperazin-1-yl-1H-pyrrole-3-carboxylic acid amide,

5-[2-(3-Acetyl-phenyl)-pyridin-4-yl]-2-piperazin-1-yl-1H-pyrrole-3-carbonitrile trifluoroacetate,

2-Piperazin-1-yl-5-[2-(1H-pyrazol-4-yl)-pyridin-4-yl]-1H-pyrrole-3-carbonitrile trifluoroacetate,

5-[2-(1-Benzyl-1H-pyrazol-4-yl)-pyridin-4-yl]-2-piperazin-1-yl-1H-pyrrole-3-carbonitrile trifluoroacetate,

5-[2-(1-Benzyl-1H-pyrazol-4-yl)-pyridin-4-yl]-2-piperazin-1-yl-1H-pyrrole-3-carboxylic acid amide,

2-Piperazin-1-yl-5-{2-[4-(pyrrolidine-1-carbonyl)-phenyl]-pyridin-4-yl}-1H-pyrrole-3-carbonitrile trifluoroacetate,

2-Piperazin-1-yl-5-{2-[4-(pyrrolidine-1-carbonyl)-phenyl]-pyridin-4-yl}-1H-pyrrole-3-carboxylic acid amide,

5-{2-[4-Chloro-3-(pyrrolidine-1-carbonyl)-phenyl]-pyridin-4-yl}-2-piperazin-1-yl-1H-pyrrole-3-carbonitrile trifluoroacetate,

5-{2-[4-Chloro-3-(pyrrolidine-1-carbonyl)-phenyl]-pyridin-4-yl}-2-piperazin-1-yl-1H-pyrrole-3-carboxylic acid amide,

2-Piperazin-1-yl-5-{2-[3-(pyrrolidine-1-carbonyl)-phenyl]-pyridin-4-yl}-1H-pyrrole-3-carbonitrile trifluoroacetate,

2-Piperazin-1-yl-5-{2-[3-(pyrrolidine-1-carbonyl)-phenyl]-pyridin-4-yl}-1H-pyrrole-3-carboxylic acid amide,

4-[4-(4-Cyano-5-piperazin-1-yl-1H-pyrrol-2-yl)-pyridin-2-yl]-benzoic acid ethyl ester trifluoroacetate,

5-{2-[3-Nitro-5-(pyrrolidine-1-carbonyl)-phenyl]-pyridin-4-yl}-2-piperazin-1-yl-1H-pyrrole-3-carbonitrile trifluoroacetate,

5-[3-(3-Cyclopentylcarbamoyl-phenyl)-pyridin-4-yl]-2-piperazin-1-yl-1H-pyrrole-3-carboxylic acid amide,

5-{2-[2-Fluoro-5-(pyrrolidine-1-carbonyl)-phenyl]-pyridin-4-yl}-2-piperazin-1-yl-1H-pyrrole-3-carboxylic acid amide,

N-(2-Cyano-ethyl)-3-[4-(4-cyano-5-piperazin-1-yl-1H-pyrrol-2-yl)-pyridin-2-yl]-benzamide trifluoroacetate,

4-[4-(4-Cyano-5-piperazin-1-yl-1H-pyrrol-2-yl)-pyridin-2-yl]-N-(2,2,2-trifluoro-ethyl)-benzamide trifluoroacetate,

5-{2-[4-(Morpholine-4-sulfonyl)-phenyl]-pyridin-4-yl}-2-piperazin-1-yl-1H-pyrrole-3-carbonitrile trifluoroacetate,

5-{2-[4-(Morpholine-4-sulfonyl)-phenyl]-pyridin-4-yl}-2-piperazin-1-yl-1H-pyrrole-3-carboxylic acid amide,

5-{2-[3-Nitro-5-(pyrrolidine-1-carbonyl)-phenyl]-pyridin-4-yl}-2-piperazin-1-yl-1H-pyrrole-3-carboxylic acid amide,

5-{2-[3-(5-Methyl-[1,3,4]oxadiazol-2-yl)-phenyl]-pyridin-4-yl}-2-piperazin-1-yl-1H-pyrrole-3-carbonitrile trifluoroacetate,

4-[4-(4-Cyano-5-piperazin-1-yl-1H-pyrrol-2-yl)-pyridin-2-yl]-N-cyclopentyl-benzamide trifluoroacetate,

5-[2-(4-Cyclopentylcarbamoyl-phenyl)-pyridin-4-yl]-2-piperazin-1-yl-1H-pyrrole-3-carboxylic acid amide,

5-{2-[4-(5-Methyl-[1,3,4]oxadiazol-2-yl)-phenyl]pyridin-4-yl}-2-piperazin-1-yl-1H-pyrrole-3-carbonitrile trifluoroacetate,

5-{2-[3-(5-Methyl-[1,3,4]oxadiazol-2-yl)-phenyl]pyridin-4-yl}-2-piperazin-1-yl-1H-pyrrole-3-carboxylic acid amide,

2-Piperazin-1-yl-5-{2-[4-(2,2,2-trifluoro-ethylcarbamoyl)-phenyl]-pyridin-4-yl}-1H-pyrrole-3-carboxylic acid amide,

Morpholine-4-carboxylic acid {4-[4-(4-carbamoyl-5-piperazin-1-yl-1H-pyrrol-2-yl)-pyridin-2-yl]-phenyl}-amide,

5-[2-(4-Methyl-3,4-dihydro-2H-benzo[1,4]oxazin-6-yl)-pyridin-4-yl]-2-piperazin-1-yl-1H-pyrrole-3-carbonitrile trifluoroacetate,

(S)-3-Amino-5′-{2-[(E)-2-(4-fluoro-phenyl)-vinyl]-pyridin-4-yl}-2,3,4,5-tetrahydro-1′H-[1,2′]bipyrrolyl-3-carbonitrile trifluoroacetate,

(S)-3-Amino-5′-{2-[(E)-2-(4-fluoro-phenyl)-vinyl]-pyridin-4-yl}-2,3,4,5-tetrahydro-1′H[1,2′]bipyrrolyl-3′-carboxylic acid amide,

(R)-3-Amino-5′-{2-[(E)-2-(4-fluoro-phenyl)-vinyl]-pyridin-4-yl}-2,3,4,5-tetrahydro-1′H-[1,2′]bipyrrolyl-3-carbonitrile trifluoroacetate,

(R)-3-Amino-5′-{2-[(E)-2-(4-fluoro-phenyl)-vinyl]-pyridin-4-yl}-2,3,4,5-tetrahydro-1′H[1,2′]bipyrrolyl-3′-carboxylic acid amide,

2-[1,4]Diazepan-1-yl-5-{2-[(E)-2-(4-fluoro-phenyl)-vinyl]-pyridin-4-yl}-1H-pyrrole-3-carbonitrile trifluoroacetate,

2-[1,4]Diazepan-1-yl-5-{2-[(E)-2-(4-fluoro-phenyl)-vinyl]-pyridin-4-yl}-1H-pyrrole-3-carboxylic acid amide,

2-(4-Amino-piperidin-1-yl)-5-{2-[(E)-2-(4-fluoro-phenyl)-vinyl]-pyridin-4-yl}-1H-pyrrole-3-carbonitrile trifluoroacetate,

2-(4-Amino-piperidin-1-yl)-5-{2-[(E)-2-(4-fluoro-phenyl)-vinyl]-pyridin-4-yl}-1H-pyrrole-3-carboxylic acid amide,

5-{2-[(E)-2-(4-Fluoro-phenyl)-vinyl]-pyridin-4-yl}-2-(4-hydroxy-piperidin-1-yl)-1H-pyrrole-3-carbonitrile,

5-{2-[(E)-2-(4-Fluoro-phenyl)-vinyl]-pyridin-4-yl}-2-(3-hydroxy-piperidin-1-yl)-1H-pyrrole-3-carbonitrile,

5-{2-[(E)-2-(4-Morpholin-4-ylmethyl-phenyl)-vinyl]-pyridin-4-yl}-2-piperazin-1-yl-1H-pyrrole-3-carbonitrile trifluoroacetate,

5-{2-[(E)-2-(4-Morpholin-4-ylmethyl-phenyl)-vinyl]-pyridin-4-yl}-2-piperazin-1-yl-1H-pyrrole-3-carboxylic acid amide hydrobromide,

4-{(E)-2-[4-(4-Cyano-5-piperazin-1-yl-1H-pyrrol-2-yl)-pyridin-2-yl]-vinyl}-N,N-diethyl-benzamide trifluoroacetate,

5-{2-[(E)-2-(4-Diethylcarbamoyl-phenyl)-vinyl]-pyridin-4-yl}-2-piperazin-1-yl-1H-pyrrole-3carboxylic acid amide,

5-(2-{(E)-2-[4-(Morpholine-4-carbonyl)-phenyl]-vinyl}-pyridin-4-yl)-2-piperazin-1-yl-1H-pyrrole-3-carbonitrile,

5-(2-{(E)-2-[4-(Morpholine-4-carbonyl)-phenyl]-vinyl}-pyridin-4-yl)-2-piperazin-1-yl-1H-pyrrole-3-carboxylic acid amide,

5-{2-[(E)-2-(4-(Methoxyphenyl)-vinyl]-pyridin-4-yl}-2-piperazin-1-yl-1H-pyrrole-3-carbonitrile,

2-Piperazin-1-yl-5-[2-((E)-2-pyridin-4-yl-vinyl)-pyridin-4-yl]-1H-pyrrole-3-carbonitrile,

2-Piperazin-1-yl-5-[2-((E)-2-pyridin-4-yl-vinyl)-pyridin-4-yl]-1H-pyrrole-3-carboxylic acid amide,

2-Piperazin-1-yl-5-[2-((E)-2-pyridin-3-yl-vinyl)-pyridin-4-yl]-1H-pyrrole-3-carbonitrile,

2-Piperazin-1-yl-5-[2-((E)-2-pyridin-3-yl-vinyl)-pyridin-4-yl]-1H-pyrrole-3-carboxylic acid amide,

2-Piperazin-1-yl-5-[2-((E)-2-pyridin-2-yl-vinyl)-pyridin-4-yl]-1H-pyrrole-3-carbonitrile,

2-Piperazin-1-yl-5-[2-((E)-2-pyridin-2-yl-vinyl)-pyridin-4-yl]-1H-pyrrole-3-carboxylic acid amide,

N-Hydroxy-2-piperazin-1-yl-5-[2-((E)-styryl)-pyridin-4-yl]-1H-pyrrole-3-carboxamidine

5-(2-Phenethyl-pyridin-4-yl)-2-piperazin-1-yl-1H-pyrrole-3-carboxamidine,

2-(4-Methyl-piperazin-1-yl)-5-[2-((E)-styryl)-pyridin-4-yl]-1H-pyrrole-3-carboxylic acid amide,

4-{3-Cyano-5-[2-((E)-styryl)-pyridin-4-yl]-1H-pyrrol-2-yl}-piperazine-1-carboxylic acid benzylamide,

2-Piperazin-1-yl-5-(2-quinolin-3-yl-pyridin-4-yl)-1H-pyrrole-3-carboxamidine,

2-(4-Formyl-piperazin-1-yl)-5-[2-(2-[1,4]oxazepan-4-yl-pyrimidin-5-yl)-pyridin-4-yl]-1H-pyrrole-3-carbonitrile,

5-[2-(4-Morpholin-4-yl-phenylamino)-pyridin-4-yl]-2-piperazin-1-yl-1H-pyrrole-3-carbonitrile hydrochloride,

5-{2-[4-(Morpholine-4-carbonyl)-phenylamino]-pyridin-4-yl}-2-piperazin-1-yl-1H-pyrrole-3-carbonitrile trifluoroacetate,

5-{2-[3-(Morpholine-4-sulfonyl)-phenylamino]-pyridin-4-yl}-2-piperazin-1-yl-1H-pyrrole-3-carbonitrile trifluoroacetate,

5-{2-[3-(Morpholine-4-sulfonyl)-phenylamino]-pyridin-4-yl}-2-piperazin-1-yl-1H-pyrrole-3-carboxylic acid amide,

5-{2-[4-(Morpholine-4-sulfonyl)-phenylamino]-pyridin-4-yl}-2-piperazin-1-yl-1H-pyrrole-3-carbonitrile trifluoroacetate,

5-{2-[4-(Morpholine-4-sulfonyl)-phenylamino]-pyridin-4-yl}-2-piperazin-1-yl-1H-pyrrole-3-carboxylic acid amide,

5-(2-Imidazol-1-yl-pyridin-4-yl)-2-piperazin-1-yl-1H-pyrrole-3-carbonitrile,

5-[2-(4-Phenyl-imidazol-1-yl)-pyridin-4-yl]-2-piperazin-1-yl-1H-pyrrole-3-carbonitrile,

5-{2-[(E)-2-(4-Fluoro-phenyl)-vinyl]-pyridin-4-yl}-1-methyl-2-piperazin-1-yl-1H-pyrrole-3-carbonitrile trifluoroacetate,

5-{2-[(E)-2-(4-Fluoro-phenyl)-vinyl]-pyridin-4-yl}-2-(4-methanesulfonyl-piperazin-1-yl)-1H-pyrrole-3-carbonitrile,

5-{2-[(E)-2-(4-Fluoro-phenyl)-vinyl]-pyridin-4-yl}-2-(4-methanesulfonyl-piperazin-1-yl)-1H-pyrrole-3-carboxylic acid amide,

4-(3-Carbamoyl-5-{2-[(E)-2-(4-morpholin-4-ylmethyl-phenyl)-vinyl]-pyridin-4-yl}-1H-pyrrol-2-yl)-piperazine-1-carboxylic acid benzyl ester,

2-Piperazin-1-yl-5-(6′-pyrrolidin-1-yl-[2,3′]bipyridinyl-4-yl)-1H-pyrrole-3-carbonitrile trifluoroacetate,

2-Piperazin-1-yl-5-(6′-pyrrolidin-1-yl-[2,3′]bipyridinyl-4-yl)-1H-pyrrole-3-carboxylic acid amide,

5-(2-{2-[(2-Methoxy-ethyl)-methyl-amino]-pyrimidin-5-yl}-pyridin-4-yl)-2-piperazin-1-yl-1H-pyrrole-3-carbonitrile trifluoroacetate,

5-[6′-(1-Methyl-piperidin-4-yloxy)-(2,3′]bipyridinyl-4-yl]-2-piperazin-1-yl-1H-pyrrole-3-carbonitrile trifluoroacetate,

5-(6′-Morpholin-4-yl-[2,3′]bipyridinyl-4-yl)-2-piperazin-1-yl-1H-pyrrole-3-carbonitrile trifluoroacetate,

5-[2-(2-Morpholin-4-yl-pyrimidin-5-yl)-pyridin-4-yl]-2-piperazin-1-yl-1H-pyrrole-3-carbonitrile trifluoroacetate,

5-(6′-Morpholin-4-yl-[2,3′]bipyridinyl-4-yl)-2-piperazin-1-yl-1H-pyrrole-3-carboxylic acid amide,

2-Piperazin-1-yl-5-(3,4,5,6-tetrahydro-2H-[1,2′;5′,2″]terpyridin-4″-yl)-1H-pyrrole-3-carboxylic acid amide,

5-[6′-(4-Methyl-piperazin-1-yl)-[2,3′]bipyridinyl-4-yl]-2-piperazin-1-yl-1H-pyrrole-3-carboxylic acid amide,

5-{2-[2-(4-Methyl-piperazin-1-yl)-pyrimidin-5-yl]-pyridin-4-yl}-2-piperazin-1-yl-1H-pyrrole-3-carbonitrile bis trifluoroacetate,

5-{2-[2-(4-Methyl-piperazin-1-yl)-pyrimidin-5-yl]-pyridin-4-yl}-2-piperazin-1-yl-1H-pyrrole-3-carboxylic acid amide,

5-[6′-(4-Cyclopentyl-piperazin-1-yl)-[2,3′]bipyridinyl-4-yl]-2-piperazin-1-yl-1H-pyrrole-3-carbonitrile trifluoroacetate,

5-[6′-(4-Methyl-[1,4]diazepan-1-yl)-[2,3′]bipyridinyl-4-yl]-2-piperazin-1-yl-1H-pyrrole-3-carbonitrile trifluoroacetate,

5-[6′-(4-Benzyl-piperazin-1-yl)-[2,3′]bipyridinyl-4-yl]-2-piperazin-1-yl-1H-pyrrole-3-carbonitrile hydrochloride,

5-[6′-(4-Benzyl-piperazin-1-yl)-[2,3′]bipyridinyl-4-yl]-2-piperazin-1-yl-1H-pyrrole-3-carboxylic acid amide,

5-[6′-(4-Cyclopentyl-piperazin-1-yl)-[2,3′]bipyridinyl-4-yl]-2-piperazin-1-yl-1H-pyrrole-3-carboxylic acid amide,

5-[2-(2-[1,4]Oxazepan-4-yl-pyrimidin-5-yl)-pyridin-4-yl]-2-piperazin-1-yl-1H-pyrrole-3-carbonitrile trifluoroacetate,

5-[2-(2-Azepan-1-yl-pyrimidin-5-yl)-pyridin-4-yl]-2-piperazin-1-yl-1H-pyrrole-3-carbonitrile trifluoroacetate,

5-{2-[2-(Isobutyl-methyl-amino)-pyrimidin-5-yl]-pyridin-4-yl}-2-piperazin-1-yl-1H-pyrrole-3-carbonitrile trifluoroacetate,

2-Piperazin-1-yl-5-[2-(2-pyrrolidin-1-yl-pyrimidin-5-yl)-pyridin-4-yl]-1H-pyrrole-3-carbonitrile trifluoroacetate,

2-Piperazin-1-yl-5-[2-(2-piperidin-1-yl-pyrimidin-5-yl)-pyridin-4-yl]-1H-pyrrole-3-carbonitrile trifluoroacetate,

5-[2-(2-Methylamino-pyrimidin-5-yl)-pyridin-4-yl]-2-piperazin-1-yl-1H-pyrrole-3-carbonitrile trifluoroacetate,

2-Piperazin-1-yl-5-[2-(2-pyrrolidin-1-yl-pyrimidin-5-yl)-pyridin-4-yl]-1H-pyrrole-3-carboxylic acid amide,

2-Piperazin-1-yl-5-[2-(2-piperidin-1-yl)-pyrimidin-5-yl-pyridin-4-yl]-1H-pyrrole-3-carboxylic acid amide

5-{2-[2-((2R,6S)-2,6-Dimethyl-morpholin-4-yl)-pyrimidin-5-yl]-pyridin-4-yl}-2-piperazin-1-yl-1H-pyrrole-3-carbonitrile trifluoroacetate,

2-Piperazin-1-yl-5-{2-[2-(3-trifluoromethyl-5,6-dihydro-8H-[1,2,4]triazolo[4,3-a]pyrazin-7-yl)-pyrimidin-5-yl]-pyridin-4-yl}-1H-pyrrole-3-carbonitrile trifluoroacetate,

5-{2-[(E)-2-(4-Fluoro-phenyl)-vinyl]-pyridin-4-yl}-2-methyl-1H-pyrrole-3-carbonitrile,

5-{2-[(E)-2-(4-Fluoro-phenyl)-vinyl]-pyridin-4-yl}-2-methyl-1H-pyrrole-3-carboxylic acid amide,

2-Methyl-5-[6′-(4-methyl-piperazin-1-yl)-[2,3′]bipyridinyl-4-yl]-1H-pyrrole-3-carbonitrile

2-Methyl-5-{2-[(E)-2-(4-morpholin-4-ylmethyl-phenyl)-vinyl]-pyridin-4-yl}-1H-pyrrole-3-carbonitrile,

5-[6′-(4-Benzyl-piperazin-1-yl)-[2,3′]bipyridinyl-4-yl]-2-methyl-1H-pyrrole-3-carbonitrile,

2-Methyl-5-(2-{(E)-2-[4-(morpholine-4-carbonyl)-phenyl]-vinyl}-pyridin-4-yl)-1H-pyrrole-3-carbonitrile,

2-Methyl-5-[6′-(1-methyl-piperidin-4-yloxy)-[2,3′]bipyridinyl-4-yl]-1H-pyrrole-3-carbonitrile,

5-(2-{2-[(2-Methoxy-ethyl)-methyl-amino]-pyrimidin-5-yl}-pyridin-4-yl)-2-methyl-1H-pyrrole-3-carbonitrile,

5-{2-[4-Methoxy-3-(3-methoxy-propoxy)-phenyl]-pyridin-4-yl}-2-methyl-1H-pyrrole-3-carbonitrile,

5-{2-[4-Methoxy-phenyl]-pyridin-4-yl}-2-methyl-1H-pyrrole-3-carbonitrile,

4-Methyl-5-[2-(2-[1,4]oxazepan-4-yl-pyrimidin-5-yl)-pyridin-4-yl]-2-piperazin-1-yl-1H-pyrrole-3-carboxylic acid amide,

5-[6′-(4-Cyclopentyl-piperazin-1-yl)-[2,3′]bipyridinyl-4-yl]-4-methyl-2-piperazin-1-yl-1H-pyrrole-3-carbonitrile,

4-Methyl-5-{2-[(E)-2-(4-morpholin-4-ylmethyl-phenyl)-vinyl]-pyridin-4-yl}-2-piperazin-1-yl-1H-pyrrole-3-carbonitrile,

2-Isopropyl-5-[2-((E)-styryl)-pyridin-4-yl]-1H-pyrrole-3-carboxylic acid ethyl ester,

2-Isopropyl-5-[2-((E)-styryl)-pyridin-4-yl]-1H-pyrrole-3-carboxylic acid amide,

2-Isopropyl-5-{2-[(E)-2-(4-morpholin-4-ylmethyl-phenyl)-vinyl]-pyridin-4-yl}-1H-pyrrole-3-carbonitrile,

2-Piperidin-4-yl-5-[2-((E)-styryl)-pyridin-4-yl]-1H-pyrrole-3-carbonitrile,

2-Piperidin-4-yl-5-[2-((E)-styryl)-pyridin-4-yl]-1H-pyrrole-3-carboxylic acid,

2-Piperidin-4-yl-5-[2-((E)-styryl)-pyridin-4-yl]-1H-pyrrole-3-carboxylic acid amide,

2-Piperidin-4-yl-5-[2-((E)-styryl)-pyridin-4-yl]-1H-pyrrole-3-carboxylic acid benzylamide,

a) 2-(2-Amino-ethyl)-5-[2-((E)-styryl)-pyridin-4-yl]-1H-pyrrole-3-carbonitrile,

5-[2-((E)-Styryl)-pyridin-4-yl]-2-(1,2,3,6-tetrahydro-pyridin-4-yl)-1H-pyrrole-3-carbonitrile,

5-[2-(2-Morpholin-4-yl-pyrimidin-5-yl)-pyridin-4-yl]-2-piperidin-4-yl-1H-pyrrole-3-carbonitrile,

2-(2-Amino-ethyl)-5-(2-quinolin-3-yl-pyridin-4-yl)-1H-pyrrole-3-carbonitrile,

2-Aminomethyl-5-[2-((E)-styryl)-pyridin-4-yl]-1H-pyrrole-3-carbonitrile,

2-Aminomethyl-5-(2-quinolin-3-yl-pyridin-4-yl)-1H-pyrrole-3-carbonitrile,

5-(2-Quinolin-3-yl-pyridin-4-yl)-2-(1,2,3,6-tetrahydro-pyridin-4-yl)-1H-pyrrole-3-carbonitrile,

2-(2-Amino-ethyl)-5-[2-((E)-styryl)-pyridin-4-yl]-1H-pyrrole-3-carboxylic acid,

2-(2-Amino-ethyl)-5-(2-quinolin-3-yl-pyridin-4-yl)-1H-pyrrole-3-carboxylic acid,

2-(2-Amino-ethyl)-5-(2-phenyl-pyridin-4-yl)-1H-pyrrole-3-carboxylic acid,

2-(2-Hydroxy-ethyl)-5-[2-((E)-styryl)-pyridin-4-yl]-1H-pyrrole-3-carboxylic acid,

2-Aminomethyl-5-[2-((E)-styryl)-pyridin-4-yl]-1H-pyrrole-3-carboxylic acid,

2-Aminomethyl-5-(2-quinolin-3-yl-pyridin-4-yl)-1H-pyrrole-3-carboxylic acid,

2-(2-Amino-ethyl)-5-[2-(2-[1,4]oxazepan-4-yl-pyrimidin-5-yl)-pyridin-4-yl]-1H-pyrrole-3-carboxylic acid,

2-(2-Amino-ethyl)-5-[2-(4-methoxy-phenyl)-pyridin-4-yl]-1H-pyrrole-3-carboxylic acid,

2-(2-Amino-ethyl)-5-(5′-methoxy-[2,3′]bipyridinyl-4-yl)-1H-pyrrole-3-carboxylic acid,

2-(2-Amino-ethyl)-5-[3-(3-methanesulfonyl-phenyl)-pyridin-4-yl]-1H-pyrrole-3-carboxylic acid,

2-(2-Amino-ethyl)-5-(6′-methoxy-[2,3′]bipyridinyl-4-yl)-1H-pyrrole-3-carboxylic acid,

2-(2-Amino-ethyl)-5-[2-(2,3-dihydro-benzo[1,4]dioxin-6-yl)-pyridin-4-yl]-1H-pyrrole-3-carboxylic acid,

2-(2-Amino-ethyl)-5-(2-quinolin-6-yl-pyridin-4-yl)-1H-pyrrole-3-carboxylic acid,

2-(2-Amino-ethyl)-5-{2-[(E)-2-(4-morpholin-4-ylmethyl-phenyl)-vinyl]-pyridin-4-yl}-1H-pyrrole-3-carboxylic acid,

2-(2-Amino-ethyl)-5-[2-((E)-styryl)-pyridin-4-yl]-1H-pyrrole-3-carboxylic acid amide,

5-[2-((E)-Styryl)-pyridin-4-yl]-2-(1,2,3,6-tetrahydro-pyridin-4-yl)-1H-pyrrole-3-carboxylic acid amide,

2-Aminomethyl-5-[2-((E)-styryl)-pyridin-4-yl]-1H-pyrrole-3-carboxylic acid amide,

2-(2-Dimethylamino-ethyl)-5-[2-((E)-styryl)-pyridin-4-yl]-1H-pyrrole-3-carbonitrile.

The invention in a third aspect provides a compound of formula (I) or (II) or a pharmaceutically-acceptable and -cleavable ester, or acid addition salt thereof for use as a pharmaceutical.

The invention in a fourth aspect provides the use of a compound of formula (I) or (II) or a pharmaceutically-acceptable and -cleavable ester, or acid addition salt thereof in the manufacture of a medicament for the treatment of an autoimmune disease or condition.

The invention in a fifth aspect provides the use of a compound of formula (I) or (II) or a pharmaceutically-acceptable and -cleavable ester, or acid addition salt thereof in the manufacture of a medicament for the treatment of proliferative disease.

The invention in a sixth aspect provides the use of a compound of formula (I) or (II) or a pharmaceutically-acceptable and -cleavable ester, or acid addition salt thereof in the manufacture of a medicament for the treatment of cancer.

The invention in a seventh aspect provides the use of a compound of formula (I) or (II) or a pharmaceutically-acceptable and -cleavable ester, or acid addition salt thereof for the treatment of cytokine mediated, e.g. TNF alpha mediated and/or MK2 related conditions.

The invention in a eighth aspect provides a method of treatment of cytokine mediated, e.g. TNF alpha mediated and/or MK2 related conditions comprising administering an effective amount of a compound of formula (I) or (II) or a pharmaceutically-acceptable and -cleavable ester, or acid addition salt thereof to a patient in need of such treatment.

The invention in a ninth aspect provides a pharmaceutical composition comprising a compound of formula (I) or (II) or a pharmaceutically-acceptable and -cleavable ester, or acid addition salt thereof in association with a pharmaceutically acceptable excipient, diluent or carrier.

In an tenth aspect the invention provides a process for preparing a compound of formula (I) or (II) in free or salt form, comprising the step of:

(a) reacting a compound of formula X with an appropriate boronic acid of formula XI or an ester thereof in the presence of a suitable catalyst:

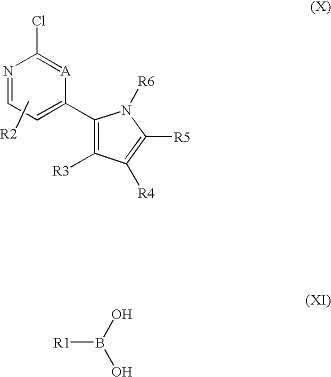

or

(b) for compounds of formula (I) wherein R4 is CONH₂, hydrolysis of a compound of formula XV:

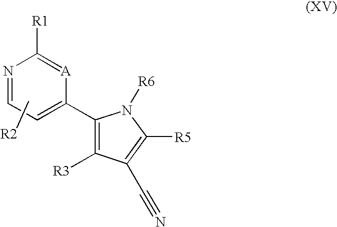

or

(c) for compounds of formula (I) wherein R4 is —C═NH—NHOH, reaction of a compound of formula XV as defined above with NH₂OH.

In step (a), a Pd catalyst may be used, for example PdCl₂(PPh₃)₂/Na₂CO₃ in a suitable solvent e.g. propanol, under reflux conditions.

In step (b), hydrolysis may be carried out using sulphuric acid.

In step (c), the reaction may be suitably carried out by heating the compound in alcoholic solution with an aqueous solution of NH₂OH.

Where appropriate, protecting groups may be used during the above transformations, the groups later being removed according to well-known chemical procedures.

The compounds of formula X and XV can themselves be prepared according to the following synthesis schemes:

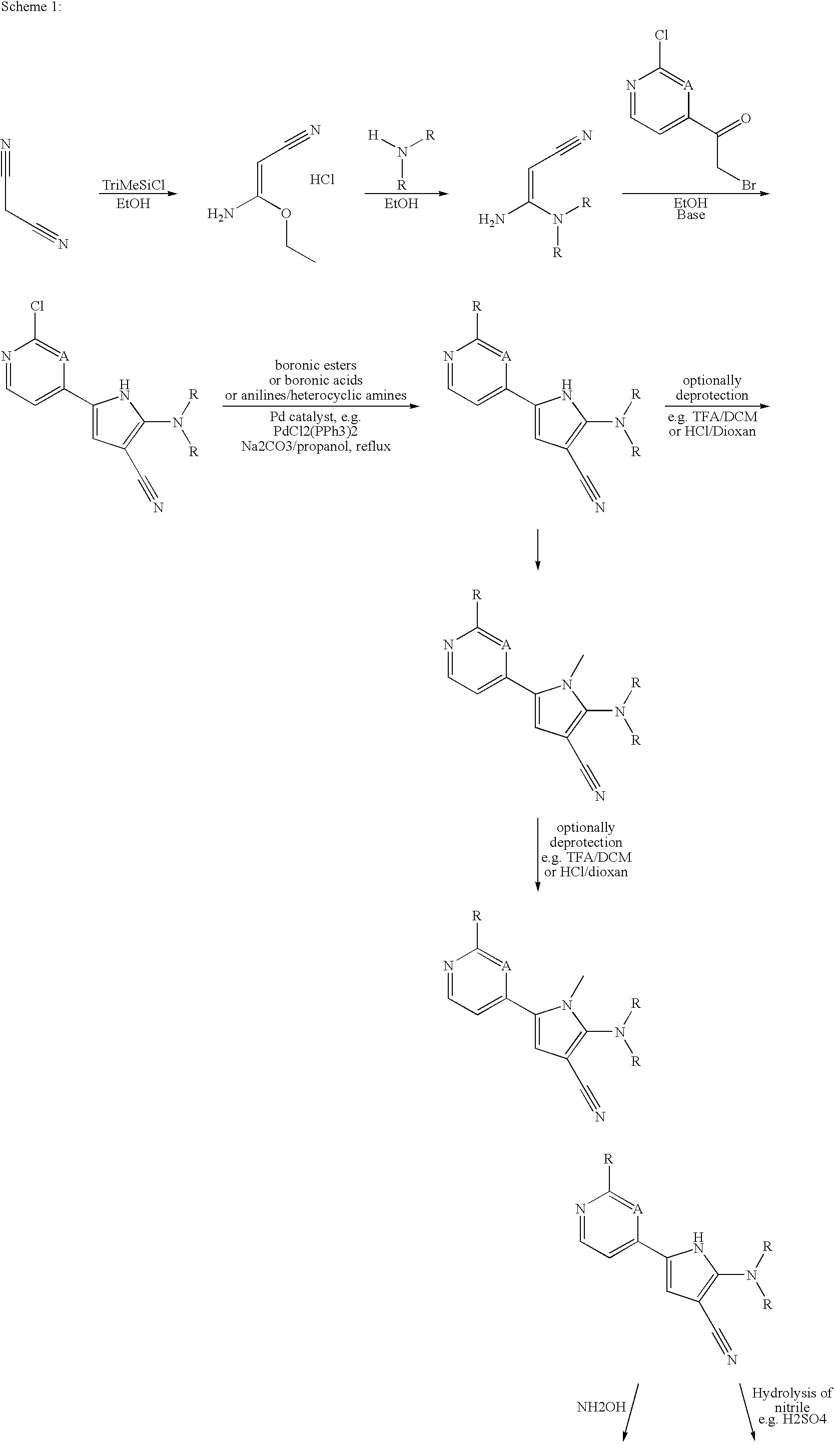

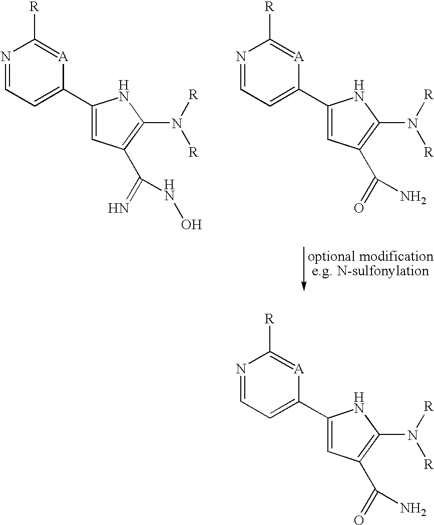

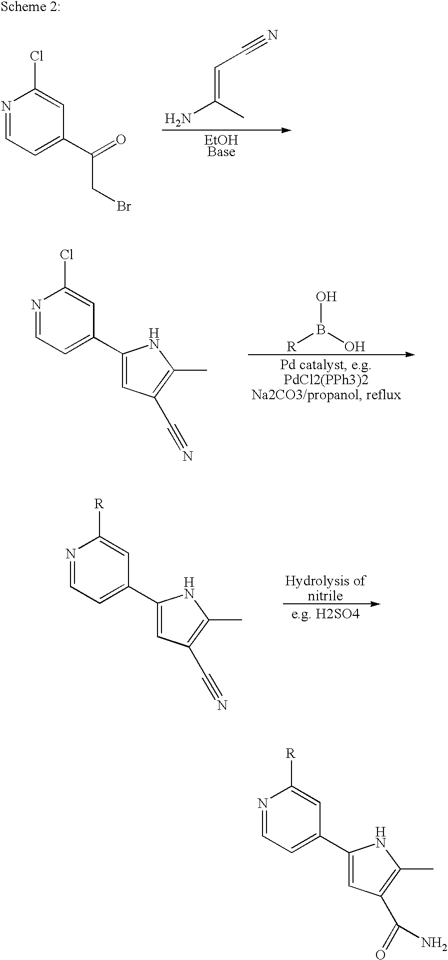

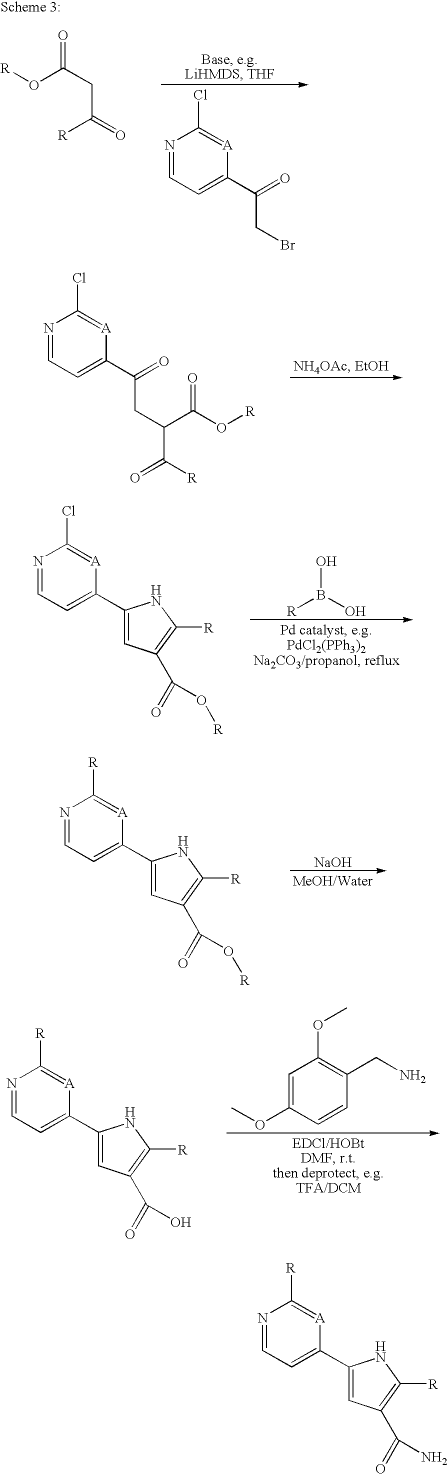

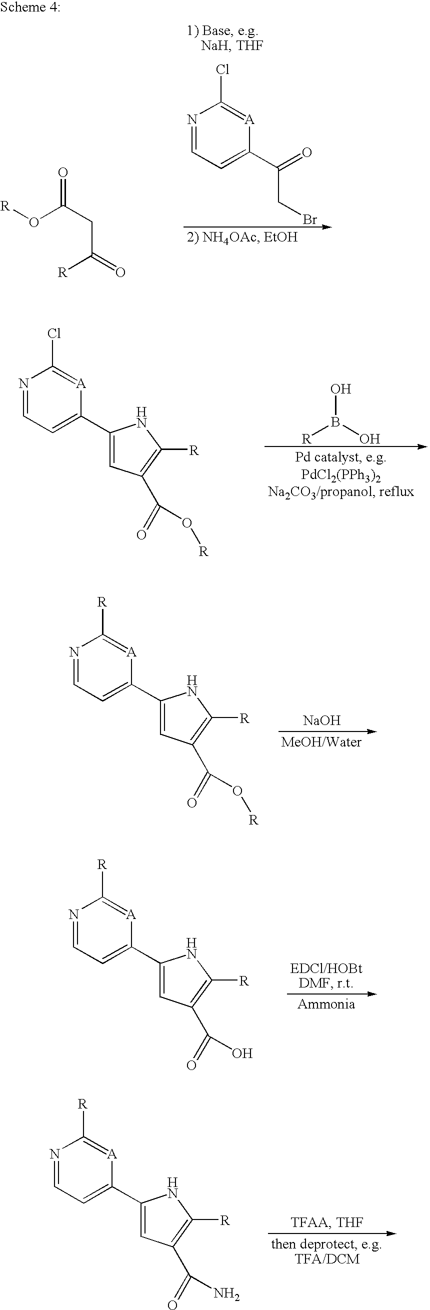

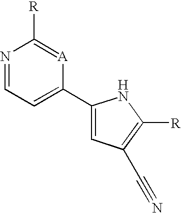

The compounds of formula (I) in free form may be converted into salt forms in conventional manner and vice-versa.

The compounds of the invention can be recovered from the reaction mixture and purified in conventional manner. Isomers, such as enantiomers, may be obtained in conventional manner, e.g. by fractional crystallization or asymmetric synthesis from corresponding asymmetrically substituted, e.g. optically active starting materials.

Agents of the invention may be prepared by processes described below, which are intended to be non-limiting examples:

### EXPERIMENTAL SECTION

### Abbreviations

Ac acetyl

AcOH acetic acid

Boc tert-butoxycarbonyl

brine sodium chloride solution in water saturated at room temperature

tBu tert-butyl

CH₂Cl₂ methylene chloride

conc. concentrated

DMAP 4-dimethylaminopyridine

DMF N,N-dimethylformamide

DMSO dimethyl sulfoxide

dppf (diphenylphosphino)ferrocene

EDCI N-(3-dimethylaminopropyl)-N′-ethyl-carbodiimide

Et ethyl

EtOAc ethyl acetate

EtOH ethanol

HCl_{conc} concentrated hydrochloric acid (37% in water)

Me methyl

MeOH methanol

Meldrum's acid 2,2-dimethyl-1,3-dioxane-4,6-dione

min, min. minute(s)

MS mass spectrometry

Na₂SO₄ sodium sulfate

NBS N-bromosuccinimide

NEt₃ triethylamine

NH₃ ammonia

NH_{3conc} concentrated ammonia (25% in water)

NMR Nuclear Magnetic Resonance

OAc acetate

PPh₃ triphenylphosphine

SiO₂ silica

TBME tert-butyl methyl ether

THF tetrahydrofuran

TFAA trifluoroacetic-acid anhydride

### Synthesis of Novel boronates

### a) Heteroaryl-bromides

### (5-Bromo-pyrimidin-2-yl)-(2-methoxy-ethyl)-methyl-amine

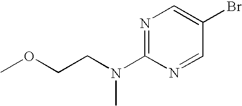

2 g of 5-bromo-2-chloropyrimidine, 1.1 g of N-(methoxyethyl)methylamine, 1.72 g of potassium carbonate are heated overnight in 30 ml acetonitrile. After evaporation of the solvent, the residue is extracted with ethyl acetate/water. The organic phase is dried over sodium sulfate and dried to yield a yellow solid.

¹H-NMR (400 MHZ, DMSO-d6): 3.08 (s, 3H), 3.22 (s, 3H), 3.48 (t, 2H), 3.71 (t, 2H), 8.38 (s, 2H).

MS (ESI⁺) m/z: 246, 248 [MH]⁺.

In analogy the following compounds are prepared:

### 1-(5-Bromo-pyridin-2-yl)-4-cyclopentyl-piperazine

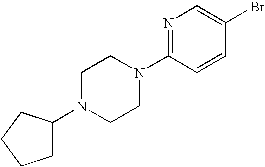

¹H-NMR (400 MHZ, DMSO-d6): 1.30-1.42 (m, 2H), 1.47-1.68 (m, 4H), 1.76-1.86 (m, 2H), 2.47 (t, 4H), 3.27-3.34 (m, 1H), 3.43 (t, 4H), 6.79 (d, 1H), 7.64 (dd, 1H), 8.13 (d, 1H).

MS (ESI⁺) m/z: 310, 312 [MH]⁺.

### 1-Benzyl-4-(5-bromo-pyridin-2-yl)-piperazine

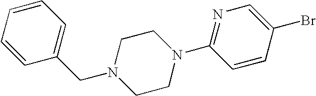

¹H-NMR (400 MHZ, DMSO-d6): 2.46 (t, 4H), 3.48 (t, 4H), 3.50 (s, 2H), 6.79 (d, 1H), 7.20-7.28 (m, 1H), 7.29-7.84 (m, 4H), 7.65 (dd, 1H), 8.12 (d, 1H).

MS (ESI⁺) m/z: 332, 334 [MH]⁺.

### 4-(5-Bromo-pyrimidin-2-yl)-[1,4]oxazepane

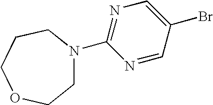

¹H-NMR (400 MHZ, DMSO-d6): 1.85 (p, 2H), 3.63 (t, 2H), 3.71 (t, 2H), 3.82 (m, 4H), 8.44 (s, 2H).

MS (ESI⁺) m/z: 258, 260 [MH]⁺.

### 1-(5-Bromo-pyrimidin-2-yl)-azepane

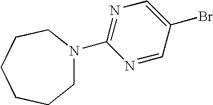

¹H-NMR (400 MHZ, DMSO-d6): 1.48 (m, 4H), 1.71 (m, 4H), 3.68 (t, 2H), 8.40 (s, 2H).

MS (ESI⁺) m/z: 256, 258 [MH]⁺.

### (5-Bromo-pyrimidin-2-yl)-isobutyl-methyl-amine

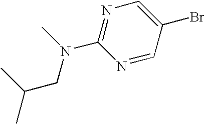

¹H-NMR (400 MHZ, DMSO-d6): 0.83 (d, 6H), 2.02 (h, 1H), 3.05 (s, 3H), 3.40 (d, 2H), 8.37 (s, 2H).

MS (ESI⁺) m/z: 244, 246 [MH]⁺.

### 4-(5-Bromo-pyrimidin-2-yl)-2,6-dimethyl-morpholine

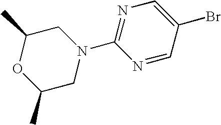

¹H-NMR (400 MHZ, DMSO-d6): 1.13 (d, 6H), 2.53 (m, 2H), 3.53 (m, 2H), 4.40 (m, 2H), 8.46 (s, 2H).

MS (ESI⁺) m/z: 272, 274 [MH]⁺.

### 7-(5-Bromo-pyrimidin-2-yl)-3-trifluoromethyl-5,6,7,8-tetrahydro-[1,2,4]triazolo[4,3-a]pyrazine

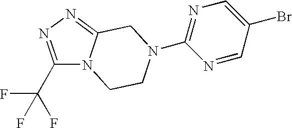

MS (ESI⁺) m/z: 349, 351 [MH]⁺.

### 1-(5-Bromo-pyridin-2-yl)-4-methyl-[1,4]diazepane

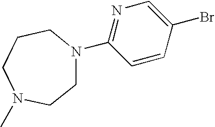

2 g of 2,5-dibromopyridine, 3.15 ml of 1-methylhomopiperazine are heated for 3 hours at 110° C. After addition of ethyl acetate and saturated sodium bicarbonate, the resulting mixture is extracted and the organic phase is dried over sodium sulfate. The residue is purified over silica gel (ethyl acetate/methanol/ammonium hydroxide [96:2:2]) and yields the title compound as a yellow oil.

¹H-NMR (400 MHZ, DMSO-d6): 1.85 (p, 2H), 2.42 (t, 2H), 2.54 (t, 2H), 3.53 (t, 2H), 3.66 (t, 2H), 6.57 (d, 1H), 7.57 (dd, 1H), 8.07 (d, 1H).

MS (ESI⁺) m/z: 272 [MI-1]⁺.

### 3-Bromo-5-(2-methoxy-ethoxy)-pyridine

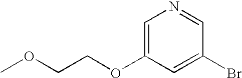

2-Methoxyethanol (2.7 ml; 33.8 mmol) is added dropwise to a suspension of NaH (55% suspension in oil; 1.62 g; 37.14 mmol) in DMF (60 ml). After stirring for 30 minutes 3,5-dibromopyridine (4.0 g; 16.88 mmol) is introduced and the mixture heated to 50° C. for 1 hour. The reaction mixture is poured on water and extracted with ethyl acetate. The organic phase is dried over Na₂SO₄ and evaporated to dryness. Chromatography (SiO₂; Hexanes/acetone 85:15) yields the title compound as yellow solid.

1H-NMR (400MHz; DMSO-d6): 8.31 (d, 1H); 8.28 (d, 1H); 7.73 (t, 1H); 4.23 (dd, 2H); 3.67 (dd, 2H); 3.32 (s, 3H).

MS (m/z) ES+: 232, 234 (MH+).

### b) Boronates

### (2-Methoxy-ethyl)-methyl-[5-(4,4,5,5-tetramethyl-[1,3,2]dioxaborolan-2-yl)-pyrimidin-2-yl]-amine

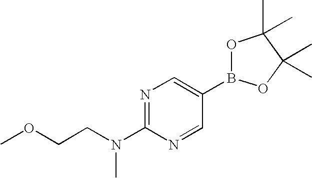

3 ml of 1.6 M n-butyllithium in THF is added at −78° C. to 1 g of (5-bromo-pyrimidin-2-yl)-(2-methoxy-ethyl)-methyl-amine dissolved in 10 ml dry THF. After one hour at −78° C., 1 ml of 2-isopropoxy-4,4,5,5-tetramethyl-[1,3,2]dioxaborolane is added drop wise. The reaction mixture is kept at low temperature for two more hours and overnight at room temperature. After addition of ammonium chloride the reaction mixture is extracted with ethyl acetate, the organic phase dried over sodium sulfate and evaporated to yield a solid.

¹H-NMR (400 MHZ, DMSO-d6): 1.29 (s, 12H), 3.16 (s, 3H), 3.25 (s, 3H), 3.52 (t, 2H), 3.80 (t, 2H), 8.46 (s, 2H).

MS (ESI⁺) m/z: 294 [MH]⁺.

Using the general procedure described above the following compounds are prepared:

### 2-Pyrrolidin-1-yl-5-(4,4,5,5-tetramethyl-[1,3,2]dioxaborolan-2-yl)-pyridine

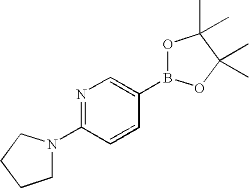

MS (ESI⁺) m/z: 233[MH]⁺

### 1-Cyclopentyl-4-[5-(4,4,5,5-tetramethyl-[1,3,2]dioxaborolan-2-yl)-pyridin-2-yl]-piperazine

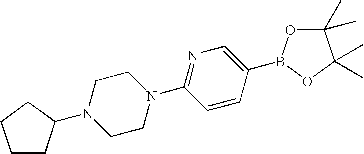

MS (ESI⁺) m/z: 358 [MH]⁺.

### 1-Methyl-4-[5-(4,4,5,5-tetramethyl-[1,3,2]dioxaborolan-2-yl)-pyridin-2-yl]-[1,4]diazepane

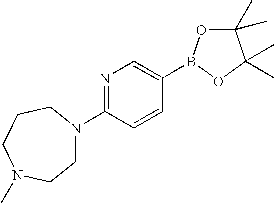

¹H-NMR (400 MHZ, DMSO-d6): 1.28 (s, 12H), 1.88 (m, 2H), 2.25 (s, 3H), 2.43 (m, 2H), 2.59 (m, 2H), 3.61 (m, 2H), 3.74 (m, 2H), 6.59 (d, 1H), 7.63 (dd, 1H), 8.28(d, 1H).

### 1-Benzyl-4-[5-(4,4,5,5-tetramethyl-[1,3,2]dioxaborolan-2-yl)-pyridin-2-yl]-piperazine

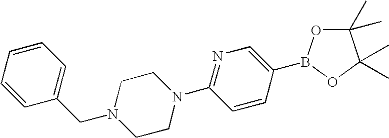

MS (ESI⁺) m/z: 380 [MH]⁺.

### 4-[5-(4,4,5,5-Tetramethyl-[1,3,2]dioxaborolan-2-yl)-pyrimidin-2-yl]-[1,4]oxazepane

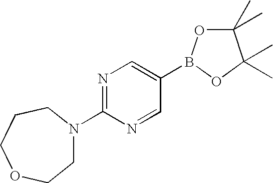

MS (ESI⁺) m/z: 306 [MH]⁺.

### 1-[5-(4,4,5,5-Tetramethyl-[1,3,2]dioxaborolan-2-yl)-pyrimidin-2-yl]-azepane

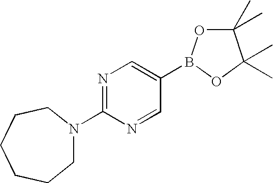

¹H-NMR (400 MHZ, DMSO-d6): 1.27 (s, 12H), 1.47 (m, 2H), 1.60 (m, 2H), 3.72 (t, 4H), 8.43 (s, 2H).

### Isobutyl-methyl-[5-(4,4,5,5-tetramethyl-[1,3,2]dioxaborolan-2-yl)-pyrimidin-2-yl]-amine

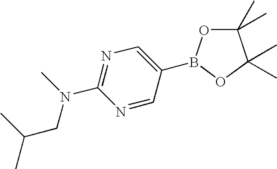

MS (ESI⁺) m/z: 292 [MH]⁺.

### 2-Pyrrolidin-1-yl-5-(4,4,5,5-tetramethyl-[1,3,2]dioxaborolan-2-yl)-pyrimidine

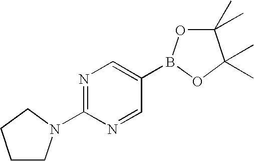

MS (ESI⁺) m/z: 275 [MH]⁺. 193 [MH]⁺:boronic acid

### 2-Piperidin-1-yl-5-(4,4,5,5-tetramethyl-[1,3,2]dioxaborolan-2-yl)-pyrimidine

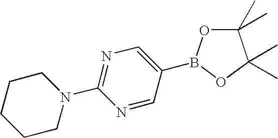

MS (ESI⁺) m/z: 289 [MH]⁺. 207 [MH]⁺:boronic acid

### 2,6-Dimethyl-4-[5-(4,4,5,5-tetramethyl-[1,3,2]dioxaborolan-2-yl)-pyrimidin-2-yl]-morpholine

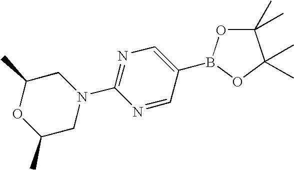

MS (ESI⁺) m/z: 320 [MH]⁺. 237 [MH]⁺:boronic acid

### 7-[5-(4,4,5,5-Tetramethyl-[1,3,2]dioxaborolan-2-yl)-pyrimidin-2-yl]-3-trifluoromethyl-5,6,7,8tetrahydro[1,2,4]triazolo[4,3-a]pyrazine

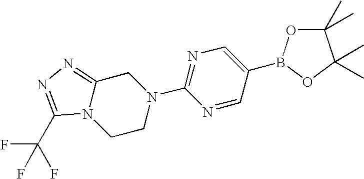

To 150 mg 7-(5-Bromo-pyrimidin-2-yl)-3-trifluoromethyl-5,6,7,8-tetrahydro-[1,2,4]triazolo[4,3-a]pyrazine dissolved in 4 ml dioxan are added 218 mg of bis(pinacolato)diboron, 35 mg of 1,1′-bis(diphenylphosphino)ferrocene-palladium(II) dichloride dichloromethane complex, and 227 mg of potassium acetate. The reaction mixture is degassed under nitrogen and heated at 120° C. overnight. The residual reaction mixture is dissolved in water/ethyl acetate and filtered over Hyflo. The organic phase is washed twice with water, brine and dried over sodium sulfate. The resulting solid is taken up in hexane, filtered and dried. The resulting grey solid is used as such for the Suzuki reaction.

MS (ESI⁺) m/z: 397 [MH]⁺. 315 [MH]⁺. boronic acid

### 3-(2-Methoxy-ethoxy)-5-(4,4,5,5-tetramethyl-[1,3,2]dioxaborolan-2-yl)-pyridine

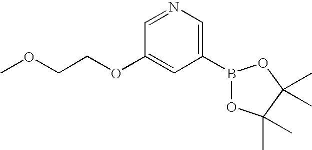

5-Bromo-3-(2-methoxy-ethoxy)-pyridine (6.7 g; 28.9 mmol), bis(pinacolato)diboron (8.8 g; 34.7 mmol), Pd(dppf)₂Cl (660mg; 0.81 mmol) and KOAc (8.5 g; 86.7 mmol) in DMF (240 ml) are heated to 160° C. for 20 minutes. The reaction mixture is evaporated, dissolved in TBME, filtered and evaporated again to deliver the target compound as a semi-crystalline red-brown solid, which is used in the next step without further purification.

MS (ESI⁺) m/z: 280 [MH]⁺.

### EXAMPLE 1

### 5′-{2-[(E)-2-(4-Fluoro-phenyl)-vinyl]-pyridin-4-yl}-2,3,4,5-tetrahydro-1′H-[1,2′]bipyrrolyl-3′carbonitrile

### a) 2-Cyanoacetimidic acid ethylester hydrochlorid

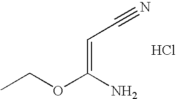

2-Cyanoacetimidic acid ethylester hydrochlorid is synthesized as outlined in Phys. Chem. News 9 (2003) 137-139.

### b) 3-Amino-3-pyrrolidin-1-yl-acrylonitrile

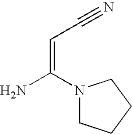

3-Amino-3-pyrrolidin-1-yl-acrylonitrile is prepared from 2-cyanoacetimidic acid ethylester hydrochloride and pyrrolidine in a similar way as described by Cocco, M. T.; Congiu, C.; Maccioni, A.; Plumitallo, A.; Schivo, M. L.; Palmieri, G. Synthesis and biological activity of some pyrrole derivatives. I. Farmaco, Edizione Scientifica (1988) 43(1), 103-12. The crude reaction mixture is dried and used as such for the next step.

### c) 1-(2-Chloro-pyridin-4-yl)-ethanone hydrobromide

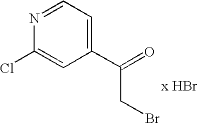

2-Bromo-1-(2-chloro-pyridin-4-yl)-ethanone hydrobromide is synthesized as outlined in WO 2004/058762. Crystallisation from ether gives the title product as off white solid.

¹H-NMR (400 MHZ, DMSO-d6): 5.02 (s, 2H), 7.84 (d, 1H), 7.98 (s, 1H), 8.66 (d, 1H).

MS (ESI⁺) m/z: 234 (80%), 236 (100%), 238 (25%) [MH]⁺.

### d) 5′-(2-Chloro-pyridin-4-yl)-2,3,4,5-tetrahydro-1′H-[1,2′]bipyrrolyl-3′-carbonitrile

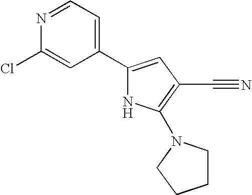

2-Bromo-1-(2-chloro-pyridin-4-yl)-ethanone hydrobromide (2.29 g) is added to a mixture of 716 mg of NaHCO₃ and 677 mg of 3-Amino-3-pyrrolidin-1-yl-acrylonitrile in 6 ml EtOH. The reaction mixture is refluxed for 5 mn and then stirred for 3 days. After filtration and washing with water a red solid (679 mg) is obtained. The filtrate is extracted with dichloromethane, washed with water/brine and dried over Na₂SO₄. The resulting red solid (130 mg) and 300 mg of the precipitate obtained by filtration are purified via HPLC (acetonitrile/H₂O, X-Terra RP-18) as a white solid.

¹H-NMR (400 MHZ, DMSO-d6): 1.98 (m, 4H), 3.52 (m, 4H), 7.13 (d, 1H), 7.54 (dd, 1H), 7.71 (s, 1H), 8.18 (d, 1H), 10.45 (s, 1H, NH).

MS (ESI⁺) m/z: 273 [MH]⁺.

### e) 5′-{2-[(E)-2-(4-Fluoro-phenyl)-vinyl]-pyridin-4-yl}-2,3,4,5-tetrahydro-1′H-[1,2′]bipyrrolyl-3′carbonitrile

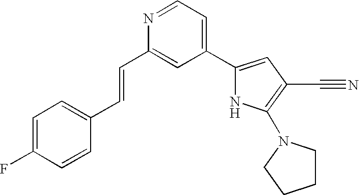

trans-2(-4-Fluoro-phenyl)-vinyl boronic acid (58 mg, 0.35 mmol) and (80 mg, 0.293 mmol) of 5′-(2-chloro-pyridin-4-yl)-2,3,4,5-tetrahydro-1′H-[1,2′]bipyrrolyl-3′-carbonitrile are dissolved in 2 ml n-propanol. The solution is degassed by introduction of a stream of argon, Pd(PPh₂)₂Cl₂ (10.5 mg, 0.014 mmol) and 77 μl of 2N Na₂CO₃ are added and the mixture is heated for 10 mn at 145° C. in a microwave oven. After filtration of the reaction mixture over celite and evaporation of the solvent, the residue is diluted with ethyl acetate, washed with saturated ammonium chloride, brine and dried over Na₂SO₄. The residual solid (237 mg) is purified by reverse phase HPLC (Gilson, X-Terra, acetonitrile/water) and yields a yellow solid.

¹H-NMR (400 MHZ, DMSO-d6): 1.98 (m, 4H), 3.53 (m, 4H), 6.98 (s, 1H), 7.15-7.26 (m, 3H), 7.42 (dd, 1H), 7.62-7.71 (m, 4H), 8.38 (d, 1H), 10.46 (s, 1H, NH).

MS (ESI⁺) m/z: 359 [MH]⁺.

### EXAMPLE 2

### 5′-{2-[(E)-2-(4-Fluoro-phenyl)-vinyl]-pyridin-4-yl}-2,3,4,5-tetrahydro-1′H-[1,2′]bipyrrolyl-3′carboxylic acid amide

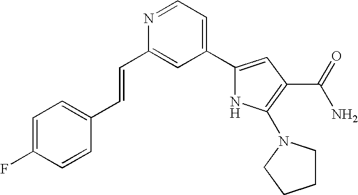

5′-{2-[(E)-2-(4-Fluoro-phenyl)-vinyl]-pyridin-4-yl}-2,3,4,5-tetrahydro-1′H-[1,4′]bipyrrolyl-3′carbonitrile (14.7 mg) is dissolved in 0.5 ml concentrated sulfuric acid and stirred at room temperature for 4 hours. The reaction mixture is poured on on icy solution of potassium carbonate and maintained at pH 9. After extraction with ethyl acetate, the organic layer is washed with brine, dried with sodium sulfate and evaporated to yield a white solid.

¹H-NMR (400 MHZ, DMSO-d6): 1.91 (m, 4H), 3.38 (m, 4H), 6.36-6.91 (NH₂, 2H), 7.07 (d, 1H), 7.06 (s, 1H), 7.15-7.24 (m, 3H), 7.37 (dd, 1H), 7.61-7.71 (m, 4H), 8.37 (d,1H), 10.32 (s, 1H, NH).

MS (ESI⁺) m/z: 377 [MH]⁺.

### EXAMPLE 3

### 5-{2-[(E)-2-(4-Fluoro-phenyl)-vinyl]-pyridin-4-yl}-2-morpholin-4-yl-1H-pyrrole-3-carbonitrile

### a) 5-(2-Chloro-pyridin-4-yl)-2-morpholin-4-yl-1H-pyrrole-3-carbonitrile

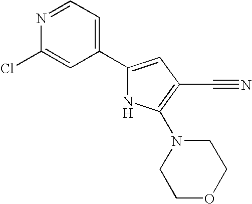

The substance is prepared in a similar fashion as example 1d .

¹H-NMR (400 MHZ, DMSO-d6): 3.42 (t, 4H), 3.75 (t, 4H), 7.18 (d, 1H), 7.59 (dd, 1H), 7.73 (s, 1H), 8.24 (d,1H), 11.05 (s, 1H, NH).

MS (ESI⁺) m/z: 289 [MH]⁺.

### b) 5-{2-[(E)-2-(4-Fluoro-phenyl)-vinyl]-pyridin-4-yl}-2-morpholin-4-yl-1H-pyrrole-3-carbonitrile

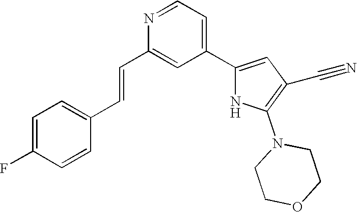

The substance is prepared in a similar fashion as example 1 starting from trans-2(-4-fluoro-phenyl)-vinyl boronic acid and 5-(2-Chloro-pyridin-4-yl)-2-morpholin-4-yl-1H-pyrrole-3-carbonitrile.

¹H-NMR (400 MHZ, DMSO-d6): 3.42 (t, 4H), 3.77 (t, 4H), 6.88 (bs, 1H), 7.04 (d, 1H), 7.14-7.24 (m, 3H), 7.45 (dd, 1H), 7.62-7.71 (m, 3H), 7.75 (s, 1H), 8.45 (d, 1H),11.1 (s, 1H).

MS (ESI⁺) m/z:375 [MH]⁺.

### EXAMPLE 4

### 5-{2-[(E)-2-(4-Fluoro-phenyl)-vinyl]-pyridin-4-yl}-2-morpholin-4-yl-1H-pyrrole-3-carboxylic acid amide

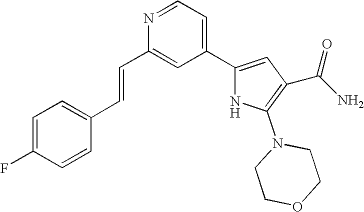

The substance is prepared in a similar fashion as example 2 starting from 5-{2-[(E)-2-(4-fluoro-phenyl)-vinyl]-pyridin-4-yl}-2-morpholin-4-yl-1H-pyrrole-3-carbonitrile.

¹H-NMR (400 MHZ, DMSO-d6): 3.14 (t, 4H), 3.75 (t, 4H), 6.88 (bs, 1H), 7.01 (d, 1H), 7.18-7.26 (m, 3H), 7.47 (dd, 1H), 7.53 (bs, 1H), 7.65-7.71 (m, 3H), 7.78 (s, 1H), 8.44 (d, 1H), 11.33 (s, 1H).

MS (ESI⁺) m/z: 393 [MH]⁺.

The following compounds are prepared in analogy to example 1:

### EXAMPLE 5

### 2-(2-Amino-ethylamino)-5-[2-((E)-styryl)-pyridin-4-yl]-1H-pyrrole-3-carbonitrile

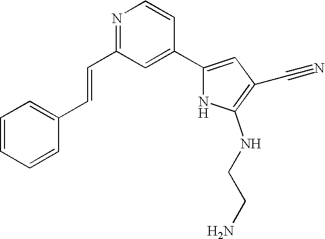

¹H-NMR (400 MHZ, DMSO-d6): 3.02-3.11 (m, 2H), 3.80-3.84 (m, 2H), 7.28 (d, 1H), 7.49 (dd, 1H), 7.52 (d, 2H), 7.68 (d, 2H), 7.70 (s, 1H), 7.92 (d, 1H), 8.06 (s, 3H, NH₃⁺), 8.30 (d, 1H), 8.42 (d, 1H), 8.79 (s, 1H), 11.84 (s, 1H, NH), 14.53 (s, 1H, NH).

MS (ESI⁺) m/z: 330 [MH]⁺.

### EXAMPLE 6

### 2-(3-Hydroxy-propylamino)-5-[2-((E)-styryl)-pyridin-4-yl]-1H-pyrrole-3-carbonitrile

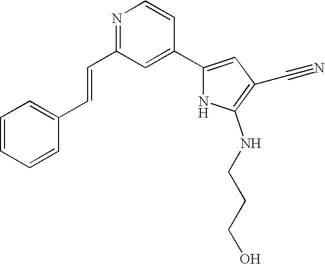

¹H-NMR (400 MHZ, DMSO-d6): 1.78 (t, 2H), 3.42-3.58 (m, 4H), 4.67 (s, 1H), 7.06 (s, 1H), 7.31 (d, 1H), 7.47 (dd, 1H), 7.51 (d, 2H), 7.61 (s, 1H), 7.68 (d, 2H), 7.74 (d, 1H), 8.01 (d, 1H), 8.30 (s, 1H), 8.41 (d, 1H), 11.35 (s, 1H, NH).

MS (ESI⁺) m/z: 345 [MH]⁺.

### EXAMPLE 7

### 2-(2-Hydroxy-ethylamino)-5-[2-((E)-styryl)-pyridin-4-yl]-1H-pyrrole-3-carbonitrile

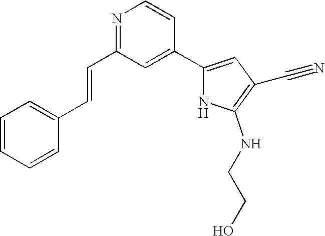

¹H-NMR (400 MHZ, DMSO-d6): 3.41 (q, 2H), 3.62 (q, 2H), 4.97 (t, (1H), 7.40 (t, 1H), 7.01 (s, 1H), 7.24 (d, 1H), 7.33-7.39 (m, 2H), 7.44 (dd, 2H), 7.65-7.68 (m, 3H), 7.71 (s, 1H), 8.44 (d, 1H), 10.89 (s, 1H, NH).

MS (ESI⁺) m/z: 331 [MH]⁺.

### EXAMPLE 8

### 5-{2-[(E)-2-(4-Fluoro-phenyl)-vinyl]-pyridin-4-yl)-2-piperazin-1-yl-1H-pyrrole-3-carbonitrile trifluoroacetate

### a) 4-1-Amino-2-cyano-vinyl)-piperazine-1-carboxylic acid tert-butyl ester

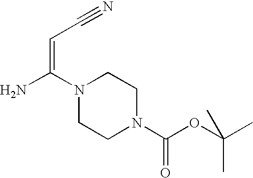

2-Cyanoacetimidic acid ethylester hydrochloride (0.8 g) is dissolved in anhydrous ethanol and after addition of 1.32 g of 1-BOC piperazine the mixture is stirred for one night at 0° C. The precipitate is filtered and washed with diethylether to give a white solid.

¹H-NMR (400 MHZ, DMSO-d6): 1.41 (s, 9H), 3.03 (t, 4H), 3.50 (t, 4H), 5.91 (bs, 1H), 9.12 (bs, 2H).

MS (ESI⁺) m/z: 253 [MH]⁺,197:MH⁺—C₄H₈.

### b) 4-[5-(2-Chloro-pyridin-4-yl)-3-cyano-1H-pyrrol-2-yl]-piperazine-1-carboxylic acid tert-butyl ester

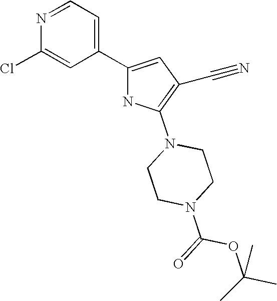

2-Bromo-1-(2-chloro-pyridin-4-yl)-ethanone hydrobromide (694 mg) is added to a mixture of 216 mg of NaHCO₃ and 500 mg of 4-(1-amino-2-cyano-vinyl)-piperazine-1-carboxylic acid tert-butyl ester hydrochloride in 6 ml EtOH. The reaction mixture is heated at reflux for 10 mn and then left at room temperature for three days. After removal of the solvent the resulting residue is dissolved in dichloromethane, washed with water/brine and dried over sodium sulfate. After removal of the solvent an orange solid is obtained. The purification by reverse phase HPLC (Gilson, X-Terra, acetonitrile/water) yields an orange solid.

¹H-NMR (400 MHZ, DMSO-d6): 1.43(s, 9H), 3.4 (m, 4H), 3.48 (m, 4H), 7.18 (s, 1H), 7.58 (d, 1H), 7.73(s, 1H), 8.24 (d, 1H).

MS (ESI⁺) m/z: 388 [MH]⁺.

### c) 4-(3-Cyano-5-{2-[(E)-2-(4-fluoro-phenyl)-vinyl]-pyridin-4-yl}-1H-pyrrol-2-yl)-piperazine-1-carboxylic acid tert-butyl ester

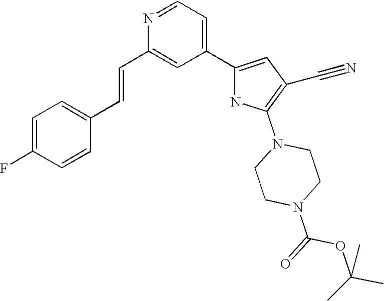

trans-2(-4-Fluoro-phenyl)-vinyl boronic acid (51 mg) and 60 mg of 4-[5-(2-chloro-pyridin-4-yl)-3-cyano-1H-pyrrol-2-yl]-piperazine-1-carboxylic acid tert-butyl ester are dissolved in 2 ml n-propanol. The solution is degassed by introduction of a stream of argon, Pd(PPh₂)₂Cl₂ (5.4 mg, 0.007 mmol) and 200 μl of 2N Na₂CO₃ are added and the mixture is heated for 10 min at 145° C. in a microwave oven. After filtration of the reaction mixture over celite and evaporation of the solvent the resulting solid is purified by reverse phase HPLC (Gilson, X-Terra, acetonitrile/water) and to yield a yellow solid.

¹H-NMR (400 MHZ, DMSO-d6): 1.43 (s, 9H), 3.38 (m, 4H), 3.49 (m, 4H), 7.04 (s, 1H), 7.17 (d, 1H), 7.22-7.27 (m, 2H), 7.45 (d, 1H), 7.63-7.74 (m, 4H), 8.45 (d, 1H), 11.2 (s, 1H).

MS (ESI⁺) m/z: 474 [MH]⁺.

### d) 5-{2-[(E)-2-(4-Fluoro-phenyl)-vinyl]-pyridin-4-yl}-2-piperazin-1-yl-1H-pyrrole-3-carbonitrile trifluoroacetate

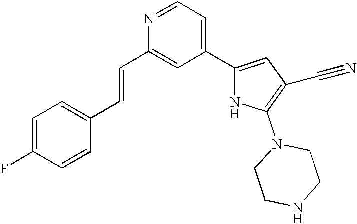

4-(3-Cyano-5-{2-[(E)-2-(4-fluoro-phenyl)-vinyl]-pyridin-4-yl}-1H-pyrrol-2-yl)-piperazine-1-carboxylic acid tert-butyl ester (45 mg) is dissolved in 0.5 ml CH₂Cl₂. 0.3 ml of trifluoroacetic acid is added. After stirring the reaction mixture overnight at room temperature, the solvents are removed under vacuum to yield an orange solid.

¹H-NMR (400 MHZ, DMSO-d6): 3.32 (m, 4H), 3.68 (bt, 4H), 7.20 (d, 1H), 7.30 (m, 2H), 7.40 (bm, 1H), 7.66-7.79 (m, 4H), 8.04 (bm, 1H), 8.04 (d, 1H), 8.83 (bs, 2H, NH₂⁺), 11.5 (s, 1H, NH).

MS (ESI⁺) m/z: 374 [MH]⁺.

### EXAMPLE 9

### 5-{2-[(E)-2-(4-Fluoro-phenyl)-vinyl]-pyridin-4-yl}-2-piperazin-1-yl-1H-pyrrole-3-carboxylic acid amide

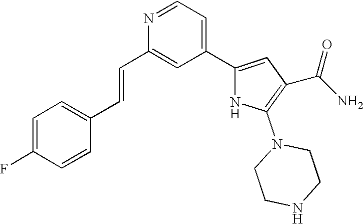

5-{2-[(E)-2-(4-Fluoro-phenyl)-vinyl]-pyridin-4-yl}-2-piperazin-1-yl-1H-pyrrole-3-carbonitrile trifluoroacetate (45 mg) is dissolved in 1.5 ml concentrated sulfuric acid and stirred for one night at room temperature. The reaction mixture is poured on on icy solution of potassium carbonate and maintained at pH 7. After extraction with ethyl acetate, the organic layer is washed with brine, dried with sodium sulfate and evaporated to yield a white solid.

¹H-NMR (400 MHZ, DMSO-d6): 2.86 (t, 4H), 3.06 (t, 4H), 6.89 (bs, 1H), 7.05 (d, 1H), 7.18-7.26 (m, 3H), 7.48 (dd, 1H), 7.62 (bs, 1H), 7.64-7.71 (m, 3H), 7.78 (s, 1H), 8.44 (d, 1H), 11.33 (s,1H).

MS (ESI⁺) m/z:) (71%) 372 [MH]⁺, (100%) 375 [MH]⁺—NH3, (28%) 414 [M+Na]⁺.

Using 4-[5-(2-chloro-pyridin-4-yl)-3-cyano-1H-pyrrol-2-yl]-piperazine-1-carboxylic acid tert-butyl ester and the precedures described above the following compounds are prepared:

### EXAMPLE 10

### 2-Piperazin-1-yl-5-(2-quinolin-3-yl-pyridin-4-yl)-1H-pyrrole-3-carbonitrile trifluoroacetate

### a) 4-(3-Cyano-5-{2-[(E)-2-(4-fluoro-phenyl)-vinyl]-pyridin-4-yl}-1H-pyrrol-2-yl)-piperazine-1-carboxylic acid tert-butyl ester

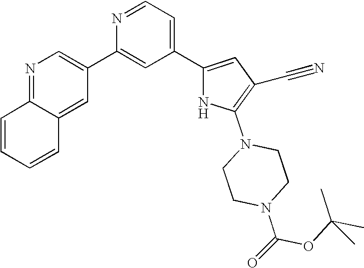

¹H-NMR (400 MHZ, DMSO-d6): 1.44 (s, 9H), 3.42 (m, 4H), 3.56 (m, 4H), 7.27 (s, 1H), 7.62-7.69 (m, 2H), 7.8 (m, 1H), 8.08 (m, 2H), 8.39 (s, 1H) 8.64 (d, 1H), 9.03 (m, 1H), 9.67 (d, 1H), 11.25 (s, 1H, NH).

MS (ESI⁺) m/z: 481[MH]⁺.

### b) 2-Piperazin-1-yl-5-(2-quinolin-3-yl-pyridin-4-yl)-1H-pyrrole-3-carbonitrile trifluoroacetate

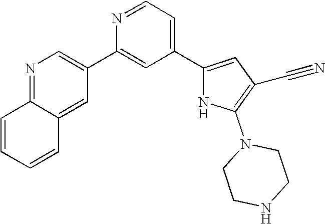

¹H-NMR (400 MHZ, DMSO-d6): 3.32 (m, 4H), 3.66 (m, 4H), 7.36 (d, 1H), 7.66-7.72 (m, 2H), 7.83 (m, 1H), 8.11 (m, 2H), 8.42 (s,1H) 8.68 (d, 1H), 8.82 (bs, 2H, NH₂⁺),9.06 (m, 1H), 9.65 (d, 1H), 11.46 (s, 1H, NH).

MS (ESI⁺) m/z: 381[MH]⁺.

### EXAMPLE 11

### 2-Piperazin-1-yl-5-(2-quinolin-3-yl-pyridin-4-yl)-1H-pyrrole-3-carboxylic acid amide

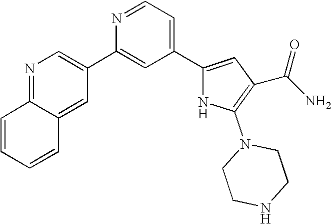

¹H-NMR (400 MHZ, DMSO-d6): 2.87 (m, 4H), 3.07 (m, 4H), 6.92 (bs, 1H, CONH₂), 7.26 (s, 1H), 7.66-7.83 (m, 4H), 7.66 (bs, CONH₂), 8.06-8.15 (m, 2H), 8.43-8.61 (m, 2H), 9.07 (s, 1H), 9.68 (s, 1H), 11.43 (s, 1H, NH).

MS (ESI⁺) m/z: 399 [MH]⁺.

MS (ESI⁻) m/z: 397 [M−H]⁻.

### EXAMPLE 12

### 5-(2-Benzofuran-2-yl-pyridin-4-yl)-2-piperazin-1-yl-1H-pyrrole-3-carbonitrile trifluoroacetate

### a) 4-[5-(2-Benzofuran-2-yl-pyridin-4-yl)-3-cyano-1H-pyrrol-2-yl]-piperazine-1-carboxylic acid tert-butyl ester

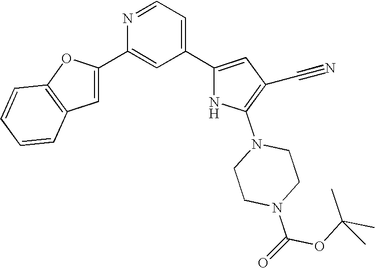

¹H-NMR (400 MHZ, DMSO-d6): 1.44 (s, 9H), 3.43 (m, 4H), 3.51 (m, 4H), 7.18 (s, 1H), 7.29 (t, 1H), 7.31 (t 1H), 7.55 (s, 1H), 7.57 (dd, 1H), 7.67 (d, 1H), 7.74 (d 1H), 8.18 (s 1H), 8.54 (d, 1H), 11.34 (s, 1H, NH).

MS (ESI⁺) m/z: 470 [MH]⁺.

MS (ESI⁻) m/z: 468 [M−H]⁻.

### b) 5-(2-Benzofuran-2-yl-pyridin-4-yl)-2-piperazin-1-yl-1H-pyrrole-3-carbonitrile trifluoroacetate

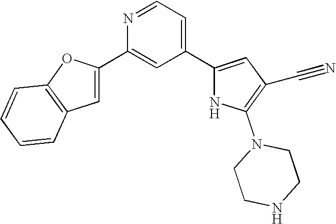

¹H-NMR (400 MHZ, DMSO-d6): 3.32 (m, 4H), 3.66 (m, 4H), 7.27 (d, 1H), 7.31(t, 1H), 7.40 (t, 1H), 7.61-7.64 (m, 2H), 7.67 (dd, 1H), 7.74 (d, 1H), 8.18 (s, 1H), 8.54 (d, 1H), 8.84 (s, 2H, NH₂ ⁺), 11.52 (s, 1H, NH).

MS (ESI⁺) m/z: 370 [MH]⁺.

MS (ESI⁻) m/z: 368 [M−H]⁻.

### EXAMPLE 13

### 2-Piperazin-1-yl-5-[2-((E}-styryl)-pyridin-4-yl]-1H-pyrrole-3-carbonitrile

### a) 4-{3-Cyano-5-[2-((E)-styryl)-pyridin-4-yl]-1H-pyrrol-2-yl}-piperazine-1-carboxylic acid tert-butyl ester

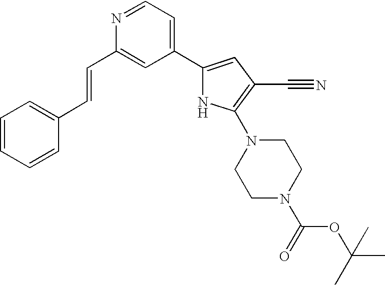

¹H-NMR (400 MHZ, DMSO-d6): 1.44 (s, 9H), 3.40-3.43 (m, 4H), 3.48-3.50 (m, 4H), 7.05 (s, 1H), 7.21 (d, 1H), 7.29-7.34 (m, 1H), 7.40 (t, 2H), 7.50 (dd, 1H), 7.64 (dd, 2H), 7.70 (d, 1H), 7.88 (s, 1H). 8.43 (d, 1H), 11.44 (s, 1H).

MS (ESI⁺) m/z: 456 [MH]⁺.

### b) 2-Piperazin-1-yl-5-[2-((E)-styryl)-pyridin-4-yl]-1H-pyrrole-3-carbonitrile

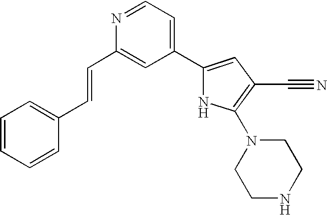

¹H-NMR (400 MHZ, DMSO-d6): 3.24 (m, 4H), 3.93 (m, 4H), 7.43 (s, 1H), 7.44 (d, 1H), 7.48 (d, 2H), 7.65 (d, 2H), 7.66 (s, 1H), 8.01 (d, 1H), 8.40 (d, 1H), 8.46 (d, 1H), 9.00 (s, 1H), 9.67 (s, 2H, NH₂⁺), 12.64 (s, 1H, NH).

MS (ESI⁺) m/z: 356 [MH]⁺.

### EXAMPLE 14

### 2-Piperazin-1-yl-5-[2-((E)-styryl)-pyridin-4-yl]-1H-pyrrole-3-carboxylic acid amide

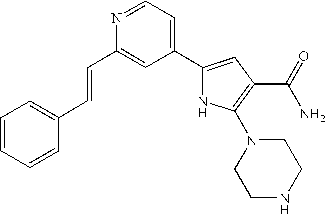

¹H-NMR (400 MHZ, DMSO-d6): 2.87 (t, 4H), 3.05 (t, 4H), 6.91 (bs, 1H), 7.08 (s, 1H), 7.26 (d, 2H), 7.33 (t, 1H), 7.43 (t, 2H), 7.51 (dd, 1H), 7.67 (d, 2H), 7.68 (d, 1H), 7.78 (s, 1H), 7.84 (s, 1H), 8.46 (d, 1H), 11.36 (s,1H).

MS (ESI⁺) m/z: 374 [MH]⁺.

### EXAMPLE 15

### 5-[2-(1-Methyl-1H-indol-5-yl)-pyridin-4-yl]-2-piperazin-1-yl-1H-pyrrole-3-carbonitrile

### a) 4-{3-Cyano-5-[2-(1-methyl-1H-indol-5-yl)-pyridin-4-yl]-1H-pyrrol-2-yl}-piperazine-1-carboxylic acid tert-butyl ester

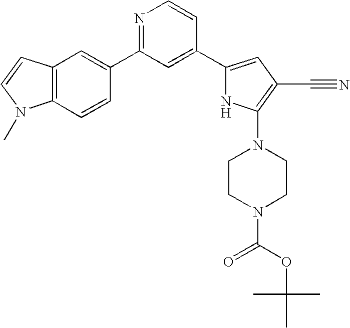

¹H-NMR (400 MHZ, DMSO-d6): 1.44 (s, 9H), 3.41 (m, 4H), 3.53 (m, 4H), 3.83 (s, 3H), 6.52 (d, 1H), 7.15 (s, 1H), 7.35 (d, 1H), 7.44 (dd, 1H), 7.52 (d, 1H), 8.00 (dd, 1H), 8.15 (s,1H), 8.43 (d, 1H), 8.49 (d, 1H), 11.28 (s, 1H, NH).

MS (ESI⁺) m/z: 483 [MH]⁺.

### b) 5-[2-(1-Methyl-1H-indol-5-yl)-pyridin-4-yl]-2-piperazin-1-yl-1H-pyrrole-3-carbonitrile

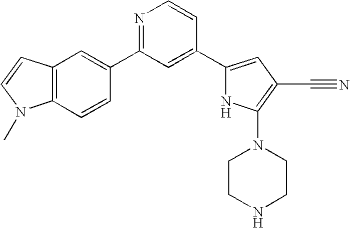

¹H-NMR (400 MHZ, DMSO-d6): 3.32 (m, 4H) 3.72 (m, 4H), 3.87 (s, 3H), 6.60 (s, 1H), 7.48 (d, 1H), 7.66 (s, 1H), 7.68 (s, 1H), 7.81 (d, 1H), 7.84 (d, 1H) 8.29 (s, 1H), 8.34 (s, 1H), 8.55 (d, 1H), 9.02 (s, 2H, NH₂⁺), 11.28 (s, 1H, NH).

MS (ESI⁺) m/z: 383 [MH]⁺.

### EXAMPLE 16

### 5-[2-(1-Methyl-1H-indol-5-yl)-pyridin-4-yl]-2-piperazin-1-yl-1H-pyrrole-3-carboxylic acid amide

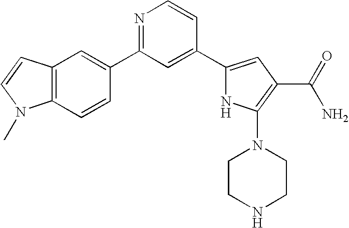

¹H-NMR (400 MHZ, DMSO-d6): 2.88 (t, 4H), 3.08 (t, 4H), 3.85 (s, 3H), 6.54 (d, 1H), 6.91 (sb, 1H), 7.14 (s, 1H), 7.36 (d, 1H), 7.47 (dd, 1H), 7.52 (d, 1H), 7.68 (sb, 1H), 8.01 (dd, 1H), 8.20 (s,1H), 8.36 (d, 1H), 8.48 (d, 1H), 11.41 (s, 1H, NH).

MS (ESI⁺) m/z: 401 [MH]⁺.

### EXAMPLE 17

### N-{4-[4-(4-Cyano-5-piperazin-1-yl-1H-pyrrol-2-yl)-pyridin-2-yl]-phenyl}-acetamide

### a) 4-{5-[2-(4-Acetylamino-phenyl)-pyridin-4-yl]-3-cyano-1H-pyrrol-2-yl}-piperazine-1-carboxylic acid tert-butyl ester

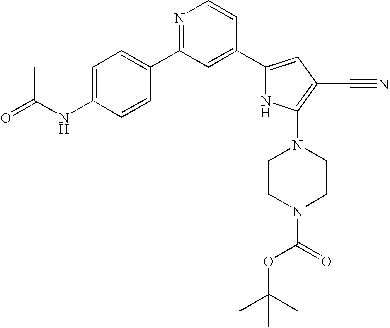

¹H-NMR (400 MHZ, DMSO-d6): 1.46 (s, 9H), 2.10 (s, 3H), 3.42 (m, 4H), 3.52 (m, 4H), 7.17 (s, 1H), 7.50 (d, 1H), 7.71 (d, 2H), 8.09 (d, 2H), 8.10 (s, 1H), 8.51 (d, 1H), 10.09 (s, 1H), 11.25 (s, 1H, NH).

MS (ESI⁺) m/z: 487 [MH]⁺.

### b) N-{4-[4-(4-Cyano-5-piperazin-1-yl-1H-pyrrol-2-yl)-pyridin-2-yl]-phenyl}-acetamide

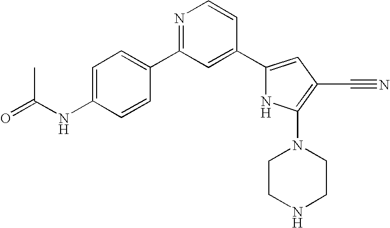

¹H-NMR (400 MHZ, DMSO-d6): 2.13 (s, 3H), 3.31 (m, 4H), 3.86 (m, 4H), 7.72 (s, 1H), 7.83 (d, 2H), 8.00 (m, 1H), 8.14 (d, 2H), 8.54 (d, 1H), 8.62 (s, 1H), 9.30 (s, 2H, NH₂⁺), 10.40 (s, 1H), 12.35 (s, 1H, NH).

MS (ESI⁺) m/z: 387 [MH]⁺.

### EXAMPLE 18

### 5-[2-(4-Acetylamino-phenyl)-pyridin-4-yl]-2-piperazin-1-yl-1H-pyrrole-3-carboxylic acid amide

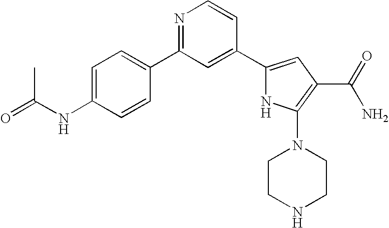

¹H-NMR (400 MHZ, DMSO-d6): 2.10 (s, 3H), 3.27 (m, 4H), 3.43 (m, 4H), 7.25 (s, 1H), 7.49 (s, 1H), 7.72 (s, 1H), 8.07 (d, 2H), 8.15 (m, 1H), 8.51 (d, 1H), 8.92 (s, 2H), 9.30 (s, 2H, NH₂⁺), 10.12 (s, 1H), 11.36 (s, 1H, NH).

MS (ESI⁺) m/z: 405 [MH]⁺.

### EXAMPLE 19

### 5-[2-(3-Dimethylamino-phenyl)-pyridin-4-yl]-2-piperazin-1-yl-1H-pyrrole-3-carbonitrile

### a) 4-{3-Cyano-5-[2-(3-dimethylamino-phenyl)-pyridin-4-yl]-1H-pyrrol-2-yl}-piperazine-1-carboxylic acid tert-butyl ester

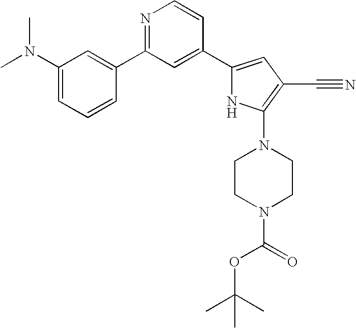

¹H-NMR (400 MHZ, DMSO-d6): 1.46 (s, 9H), 2.99 (s, 6H), 3.41 (m, 4H), 3.52 (m, 4H), 6.84 (d, 1H), 7.19 (s, 1H), 7.31 (t, 1H), 7.39 (d, 1H), 7.49 (s, 1H), 7.54 (d, 1H), 8.08 (s, 1H), 8.54 (d, 1H), 11.27 (s, 1H, NH).

MS (ESI⁺) m/z: 473 [MH]⁺.

### b) 5-[2-(3-Dimethylamino-phenyl)-pyridin-4-yl]-2-piperazin-1-yl-1H-pyrrole-3-carbonitrile

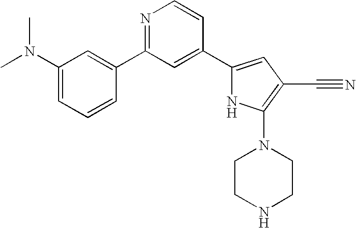

¹H-NMR (400 MHZ, DMSO-d6): 3.06 (s, 6H), 3.26 (m, 4H), 3.86 (m, 4H), 6.91 (d, 1H), 7.30-7.39 (m, 3H), 7.49 (s, 1H), 7.80 (s, 1H), 8.38 (s, 1H), 8.47 (d, 1H), 9.20-9.40 (sb, 2H, NH₂⁺), 12.00 (s, 1H, NH).

MS (ESI⁺) m/z: 373 [MH]⁺.

### EXAMPLE 20

### N-{3-[4-(4-Cyano-5-piperazin-1-yl-1H-pyrrol-2-yl)-pyridin-2-yl]-phenyl}-acetamide

### a) 4-{5-[3-(3-Acetylamino-phenyl)-pyridin-4-yl]-3-cyano-1H-pyrrol-2-yl}-piperazine-1-carboxylic acid tert-butyl ester

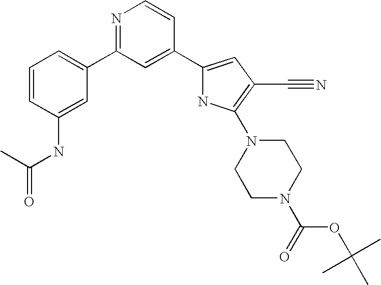

¹H-NMR (400 MHZ, DMSO-d6): 1.46 (s, 9H), 2.09 (s, 3H), 3.38-3.43 (m, 4H), 3.49-3.54 (m, 4H), 7.17 (s, 1H), 7.43 (t, 1H), 7.56 (d, 1H), 7.75 (t, 2H), 8.10 (s, 1H), 8.30 (s, 1H), 8.54 (d, 1H), 10.10 (s, 1H), 11.30 (s, 1H, NH).

MS (ESI⁺) m/z: 487 [MH]⁺.

### b) N-{3-[4-(4-Cyano-5-piperazin-1-yl-1H-pyrrol-2-yl)-pyridin-2-yl]-phenyl}-acetamide

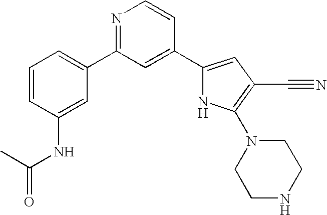

¹H-NMR (400 MHZ, DMSO-d6): 2.12 (s, 3H), 3.25-3.32 (m, 4H), 3.56-3.57 (m, 4H), 7.55 (s, 1H), 7.54-7.56 (m, 3H), 8.09 (s, 1H), 8.26 (s, 1H), 8.46 (s, 1H), 8.60 (d, 1H), 9.35-9.50 (m, 2H, NH₂⁺), 10.34 (s, 1H), 12.21 (s, 1H, NH).

MS (ESI⁺) m/z: 387 [MH]⁺.

### EXAMPLE 21

### 5-[2-(4-Dimethylamino-phenyl)-pyridin-4-yl]-2-piperazin-1-yl-1H-pyrrole-3-carbonitrile

### a) 4-{3-Cyano-5-[2-(4-dimethylamino-phenyl)-pyridin-4-yl]-1H-pyrrol-2-yl}-piperazine-1-carboxylic acid tert-butyl ester

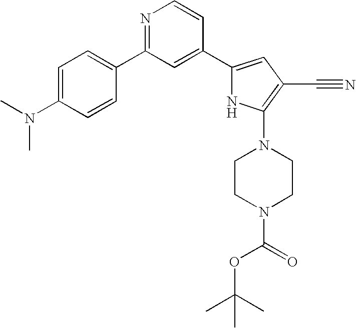

¹H-NMR (400 MHZ, DMSO-d6): 1.46 (s, 9H), 3.00 (s, 6H), 3.41 (m, 4H), 3.52 (m, 4H), 6.81 (d, 1H), 7.13 (s, 1H), 7.31 (t, 1H), 7.39 (d, 1H), 8.01 (d, 2H), 8.43 (d, 1H), 11.26 (s, 1H, NH).

MS (ESI⁺) m/z: 473 [MH]⁺.

### b) 5-[2-(4-Dimethylamino-phenyl)-pyridin-4-yl]-2-piperazin-1-yl-1H-pyrrole-3-carbonitrile

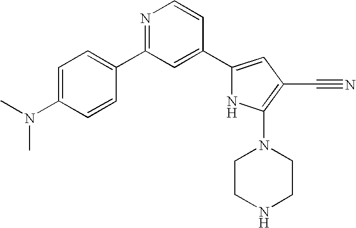

¹H-NMR (400 MHZ, DMSO-d6): 3.08 (s, 6H), 3.29 (m, 4H), 3.89 (m, 4H), 6.88 (d, 2H), 7.73 (s, 1H), 7.91 (d, 1H), 8.15 (d, 2H), 8.35 (d, 1H), 8.65 (s, 1H), 9.43 (s, 2H, NH₂⁺), 12.51 (s, 1H, NH).

MS (ESI⁺) m/z: 373 [MH]⁺.

### EXAMPLE 22

### 5-[2-(1H-Indol-6-yl)-pyridin-4-yl]-2-piperazin-1-yl-1H-pyrrole-3-carbonitrile

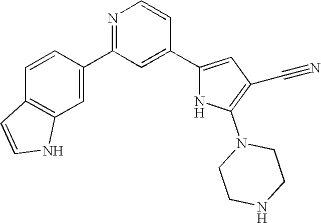

¹H-NMR (400 MHZ, DMSO-d6): 3.29 (m, 4H), 3.84 (s, 4H), 6.58 (s, 1H), 7.51-7.63 (m, 3H), 7.71-7.80 (m, 2H) 8.02 (s, 1H), 8.20 (s, 1H), 8.51 (d, 1H), 9.35 (s, 2H, NH₂⁺), 11.63 (s, 1H, NH), 12.22 (s, 1H, NH).

MS (ESI⁺) m/z: 369 [MH]⁺.

### EXAMPLE 23

### 5-[2-(1H-Indol-5-yl)-pyridin-4-yl]-2-piperazin-1-yl-1H-pyrrole-3-carbonitrile

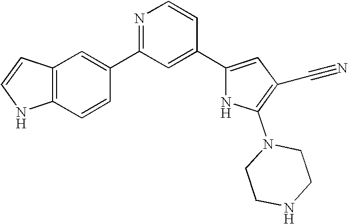

¹H-NMR (400 MHZ, DMSO-d6): 3.72 (m, 4H) 3.85 (m, 4H), 6.61 (s, 1H), 7.47 (s, 1H), 7.53 (s, 1H), 7.61 (s, 1H), 7.84 (d, 1H) 7.88 (d, 1H) 8.38 (s, 1H), 8.50 (s, 1H), 8.62 (d, 1H), 9.28 (s, 2H, NH₂⁺), 11.46 (s, 1H, NH), 12.48 (s, 1H, NH).

MS (ESI⁺) m/z: 369 [MH]⁺.

### EXAMPLE 24

### (E)-3-{4-[4-(4-Cyano-5-piperazin-1-yl-1H-pyrrol-2-yl)-pyridin-2-yl]-phenyl}-acrylic acid

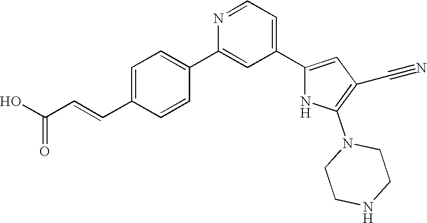

¹H-NMR (400 MHZ, DMSO-d6): 3.31 (m, 4H) 3.90 (m, 4H), 4.0 (s, br, 1H), 6.68 (d, 1H), 7.46 (s, 1H), 7.65 (d, 1H), 7.87 (d, 2H), 8.06 (d, 2H), 8.30 (s, 1H), 8.47 (d, 1H), 9.54 (2H, NH₂⁺), 12.73 (s, 1H, NH).

MS (ESI⁺) m/z: 400 [MH]⁺.

### EXAMPLE 25

### 4-{(E)-2-[4-(4-Cyano-5-piperazin-1-yl-1H-pyrrol-2-yl)-pyridin-2-yl]-vinyl}-benzoic acid methyl ester

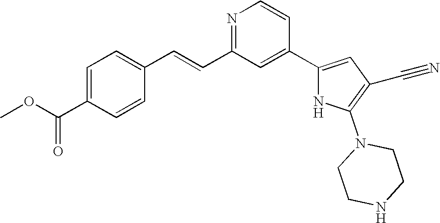

¹H-NMR (400 MHZ, DMSO-d6): 3.28 (m, 4H), 3.61 (m, 4H), 3.83 (s, 3H), 6.80 (d, 1H), 7.03 (d, 1H), 7.13 (s, 1H), 7.42 (d, 2H), 7.59 (s, 1H), 7.60 (d, 1H), 7.84 (d, 2H), 8.48 (d, 1H), 8.81 (s, 2H, NH₂⁺), 11.37 (s, 1H, NH).

MS (ESI⁺) m/z: 414 [MH]⁺.

### EXAMPLE 26

### 5-{2-[1-(2-Morpholin-4-yl-ethyl)-1H-pyrazol-4-yl]-pyridin-4-yl}-2-piperazin-1-yl-1H-pyrrole-3-carbonitrile

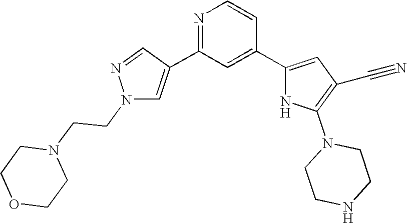

¹H-NMR (400 MHZ, DMSO-d6): 2.41 (t, 4H), 2.74 (t, 2H), 2.78-2.80 (m, 4H), 3.27 (m, 4H), 3.42 (s, 3H, NH⁺), 3.54 (t, 4H), 4.25 (t, 2H), 6.92 (s, 1H), 7.30 (s, 1H), 7.79 (s, 1H), 7.95 (s, 1H), 8.25 (s, 2H).

MS (ESI⁺) m/z: 433 [MH]⁺.

### EXAMPLE 27

### 5-[5′-(2-Methoxy-ethoxy)-[2,3′]bipyridinyl-4-yl]-2-piperazin-1-yl-1H-pyrrole-3-carbonitrile

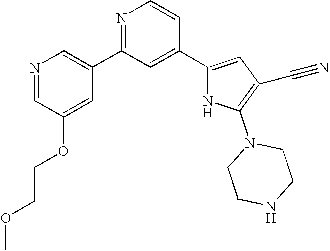

¹H-NMR (400 MHZ, DMSO-d6): 3.30 (m, 4H), 3.36 (s, 3H), 3.74 (m, 2H), 3.81 (m, 4H), 4.42 (m, 2H), 7.58 (s, 1H) 7.96 (s, 1H), 8.40 (s, 1H), 8.57 (s, 1H), 8.66 (d, 1H), 8.74 (s, 1H), 9.03 (s, 1H), 9.22 (s, 2H, NH₂⁺), 12.22 (s, 1H, NH).

MS (ESI⁺) m/z: 405 [MH]⁺.

### EXAMPLE 28

### 5-{2-[3-(3-Hydroxy-propyl)-phenyl]pyridin-4-yl}-2-piperazin-1-yl-1H-pyrrole-3-carbonitrile

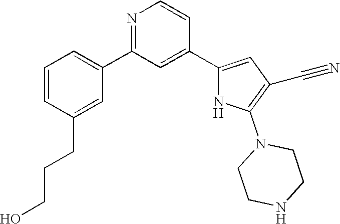

¹H-NMR (400 MHZ, DMSO-d6): 1.78-1.88 (m, 2H), 2.76 (t, 2H), 3.26-3.35 (m, 4H), 3.47 (t, 2H), 3.77 (s, br, 1H, OH), 3.80-3.87 (m, 4H), 7.46 (d, 1H) 7.55 (dd, 1H), 7.74 (s, 1H), 7.96 (d, 1H), 8.02 (s, 1H), 8.05 (s, 1H), 8.60 (s, 1H), 8.62 (s, 1H), 9.29 (s, 2H, NH₂⁺), 12.28 (s, 1H, NH).

MS (ESI⁺) m/z: 388 [MH]⁺.

### EXAMPLE 29

### {3-[4-(4-Cyano-5-piperazin-1-yl-1H-pyrrol-2-yl)-pyridin-2-yl]-5-fluoro-phenoxy}-acetic acid

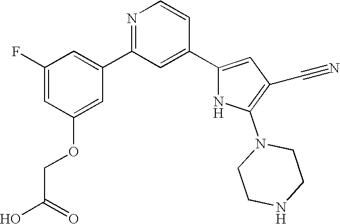

¹H-NMR (400 MHZ, DMSO-d6): 3.25-3.33 (m, 4H), 3.78-3.85 (m, 4H), 4.90 (s, 2H), 7.08 (d, 1H) 7.60 (s, 1H), 7.67 (s, 1H), 7.68 (d, 1H), 7.97 (s, 1H), 8.58 (s, 1H), 8.60 (d, 1H), 9.35 (s, 2H, NH₂⁺), 12.23 (s, 1H, NH), 13.12 (s, br, 1H, OH).

MS (ESI⁺) m/z: 422 [MH]⁺.

### EXAMPLE 30

### 5-[2-(4-Methanesulfonyl-phenyl)-pyridin-4-yl]-2-piperazin-1-yl-1H-pyrrole-3-carbonitrile trifluoroacetate

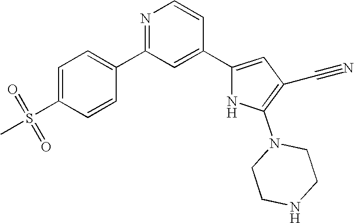

¹H-NMR (400 MHZ, DMSO-d6): 3.28 (s, 3H), 3.31 (m, 4H), 3.64 (m, 4H), 7.31 (d, 1H), 7.66 (dd, 1H), 8.07 (d, 2H), 8.27 (s, 1H), 8.36 (d, 2H), 8.63 (d, 1H), 8.83 (s, 2H, NH₂⁺), 11.46 (s, 1H, NH-pyrrole).

MS (ESI⁺) m/z: 408 [MH]⁺.

MS (ESI⁻) m/z: 406 [MH]⁻.

### EXAMPLE 31

### 5-[2-(4-Methanesulfonyl-phenyl)-pyridin-4-yl]-2-piperazin-1-yl-1H-pyrrole-3-carboxylic acid amide

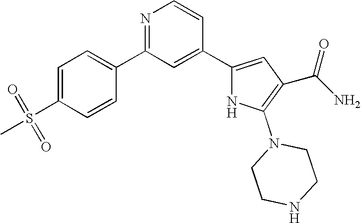

¹H-NMR (400 MHZ, DMSO-d6): 2.85 (m, 4H), 3.06 (m, 4H), 3.28 (s, 3H), 6.92 (bs,1H, NH-amide), 7.23 (d, 1H), 7.62 (bs, 1H, NH-amide), 7.67 (dd, 1H), 8.05 (d, 2H), 8.31 (s, 1H), 8.40 (d, 2H), 8.57 (d, 1H), 11.38 (s, 1H, NH).

MS (ESI⁺) m/z: 426 [MH]⁺.

MS (ESI⁻) m/z: 424 [MH]⁻.

### EXAMPLE 32

### 5-[2-(3-Methanesulfonyl-phenyl)-pyridin-4-yl]-2-piperazin-1-yl-1H-pyrrole-3-carbonitrile trifluoroacetate

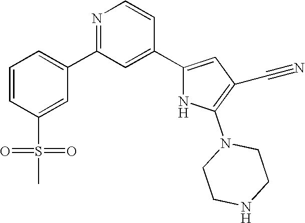

¹H-NMR (400 MHZ, DMSO-d6): 3.30 (s, 3H), 3.33 (m, 4H), 3.64 (m, 4H), 7.33 (m,1H), 7.65 (dd, 1H), 7.81 (t, 1H), 8.01 (d, 1H), 8.26 (s, 1H), 8.44 (d, 1H), 8.64 (m, 2H), 8.84 (s, 2H, NH₂⁺), 11.50 (s, 1H, NH).

MS (ESI⁺) m/z: 408 [MH]⁺.

MS (ESI⁻) m/z: 406 [MH]⁻.

### EXAMPLE 33

### 5-[2-(3-Methanesulfonyl-phenyl)-pyridin-4-yl]-2-piperazin-1-yl-1H-pyrrole-3-carboxylic acid amide

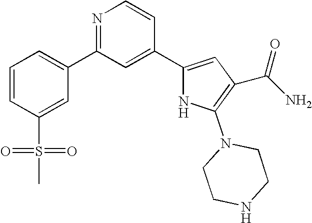

¹H-NMR (400 MHZ, DMSO-d6): 2.86 (m, 4H), 3.05 (m, 4H), 3.29 (s, 3H), 6.91 (bs, 1H NH-amide), 7.23 (s,1H), 7.64 (bs, 1H, NH-amide), 7.65 (dd, 1H), 7.79 (t, 1H), 7.98 (m, 1H), 8.29 (s, 1H), 8.48 (dd, 1H), 8.57 (d, 1H), 8.67 (m, 1H), 11.42 (s, 1H, NH pyrrole).

MS (ESI⁺) m/z: 426 [MH]⁺.

MS (ESI⁻) m/z: 425 [MH]⁻.

### EXAMPLE 34

### 5-[2-(3-Acetyl-phenyl)-pyridin-4-yl]-2-piperazin-1-yl-1H-pyrrole-3-carbonitrile trifluoroacetate

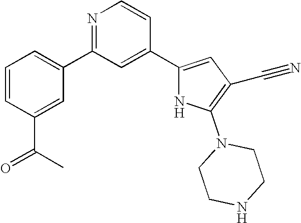

¹H-NMR (400 MHZ, DMSO-d6):2.64 (s, 3H), 3.32 (m, 4H), 3.67 (m 4H), 7.08 (s, 1H), 7.54 (m, 1H), 7.63 (t, 1H), 8.00 (d, 1H) 8.10 (s, 1H), 8.31 (d, 1H), 8.59-8.60 (m, 2H).

MS (ESI⁺) m/z: 372 [MH]⁺.

### EXAMPLE 35

### 2-Piperazin-1-yl-5-[2-(1H-pyrazol-4-yl)-pyridin-4-yl]-1H-pyrrole-3-carbonitrile trifluoroacetate

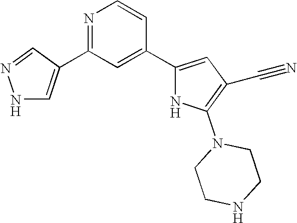

¹H-NMR (400 MHZ, DMSO-d6):3.31 (t, 4H), 3,67 (t, 4H), 7.04 (s, 1H), 7.36 (dd, 1H), 7.81 (s, 1H), 8.12 (s, 2H), 8.40 (d, 1H).

MS (ESI⁺) m/z: 320 [MH]⁺.

### EXAMPLE 36

### 5-[2-(1-Benzyl-1H-pyrazol-4-yl)-pyridin-4-yl]-2-piperazin-1-yl-1H-pyrrole-3-carbonitrile trifluoroacetate

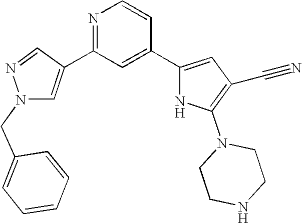

¹H-NMR (400 MHZ, DMSO-d6):3.30 (t, 4H), 3,66 (t, 4H), 5.37 (s, 1H), 7.00 (s, 1H), 7.28-7.34 (m, 6H), 7.77 (s, 1H), 8.02 (s, 1H), 8.23 (s, 1H), 8.40 (d, 1H).

MS (ESI⁺) m/z: 410 [MH]⁺.

### EXAMPLE 37

### 5-[2-(1-Benzyl-1H-pyrazol-4-yl)-pyridin-4-yl]-2-piperazin-1-yl-1H-pyrrole-3-carboxylic acid amide

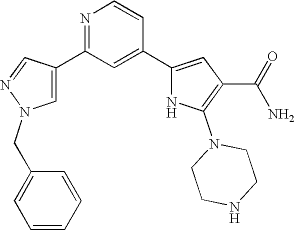

¹H-NMR (400 MHZ, DMSO-d6): 2.84 (m, 4H), 3.02 (m, 4H), 5.38 (s, 2H), 6.88 (bs, 1H), 7.11 (s, 1H), 7.29 (m, 5H), 7.41 (dd, 1H), 7.65 (bs, 1H), 7.89 (s, 1H), 8.07 (s, 1H), 8.36 (d, 1H), 8.38(s, 1H), 11.30 (s, 1H, NH).

MS (ESI⁺) m/z: 428 [MH]⁺.

### EXAMPLE 38

### 2-Piperazin-1-yl-5-{2-[4-(pyrrolidine-1-carbonyl)-phenyl]-pyridin-4-yl}-1H-pyrrole-3-carbonitrile trifluoroacetate

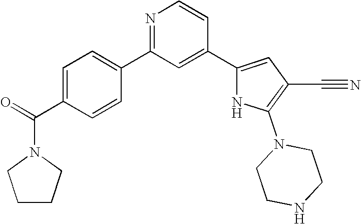

¹H-NMR (400 MHZ, DMSO-d6): 1.86 (m, 4H), 3.31 (m, 4H), 3.42 (t, 2H), 3.48 (t, 2H), 3.65 (m, 4H), 7.38 (s, 1H), 7.67 (m, 3H), 8.14 (d, 2H), 8.23 (s, 1H), 8.60 (d, 1H), 8.80 (bs, 2H, NH₂⁺), 11.45 (s, 1H, NH).

MS (ESI⁺) m/z: 427 [MH]⁺.

MS (ESI⁻) m/z: 425 [MH]⁻.

### EXAMPLE 39

### 2-Piperazin-1-yl-5-{2-[4-(pyrrolidine-1-carbonyl)-phenyl]-pyridin-4-yl}-1H-pyrrole-3-carboxylic acid amide

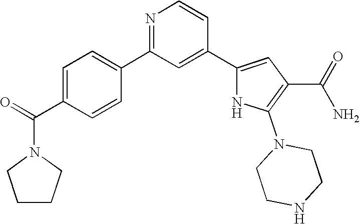

¹H-NMR (400 MHZ, DMSO-d6): 1.85 (m, 4H), 2.85 (m, 4H), 3.05 (m, 4H), 3.45 (m, 4H), 6.90 (bs, 1H), 7.17 (s, 1H), 7.54 (bs, 1H), 7.59 (d, 1H), 7.63 (d, 2H), 8.20 (d, 2H), 8.23 (s, 1H), 8.53 (d, 1H) 11.39 (s, 1H, NH).

MS (ESI⁺) m/z: 445 [MH]⁺.

### EXAMPLE 40

### 5-{2-[4-Chloro-3-(pyrrolidine-1-carbonyl)-phenyl]-pyridin-4-yl}-2-piperazin-1-yl-1H-pyrrole-3-carbonitrile trifluoroacetate

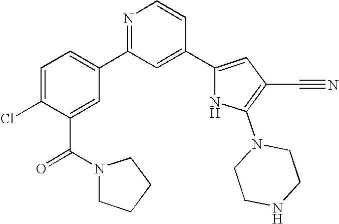

¹H-NMR (400 MHZ, DMSO-d6): 1.85-195 (m, 4H), 3.18 (t, 2H), 3.32 (m, 4H), 3.54 (t, 2H), 3.65 (m, 4H), 7.35 (s, 1H), 7.65 (dd, 1H), 7.70 d, 1H), 8.13 (d, 1H), 8.21 (dd, 1H), 8.24 (s, 1H), 8.83 (bs, 2H, NH₂⁺), 11.47 (s, 1H, NH).

MS (ESI⁺) m/z: 461 [MH]⁺.

### EXAMPLE 41

### 5-(2-[4-Chloro-3-(pyrrolidine-1-carbonyl)-phenyl]-pyridin-4-yl}-2-piperazin-1-yl-1H-pyrrole-3-carboxylic acid amide

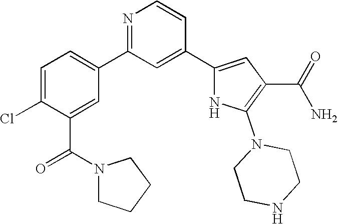

¹H-NMR (400 MHZ, DMSO-d6): 1.85 (m, 4H), 2.85 (m, 4H), 3.05 (m, 4H), 3.16 (t, 2H), 3.52 (t, 2H), 6.90 (bs, 1H, NH-amide), 7.22 (s, 1H), 7.63 (m, 2H), 8.15 (m,1H), 8.23 (m, 2H), 8.51 (m, 1H), 11.38 (s, 1H, NH-pyrrole).

MS (ESI⁺) m/z: 479 [MH]⁺.

MS (ESI⁻) m/z: 477 [MH]⁻.

### EXAMPLE 42

### 2-Piperazin-1-yl-5-{2-[3-(pyrrolidine-1-carbonyl)-phenyl]-pyridin-4-yl}-1H-pyrrole-3-carbonitrile trifluoroacetate

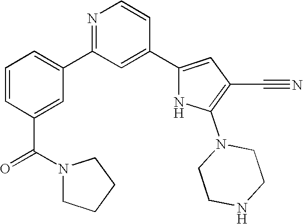

¹H-NMR (400 MHZ, DMSO-d6): 1.88 (m, 4H), 3.33 (m, 4H), 3.43 (t, 2H), 3.52 (t, 2H), 3.66 (m, 4H), 7.39 (s, 1H), 7.60-7.68 (m, 3H), 8.19-8.24 (m 3H), 8.60 (d, 1H), 8.82 (bs, 2H, NH₂⁺), 11.50 (s, 1H, NH).

MS (ESI⁺) m/z: 427 [MH]⁺.

MS (ESI⁻) m/z: 425 [MH]⁻.

### EXAMPLE 43

### 2-Piperazin-1-yl-5-{2-[3-(pyrrolidine-1-carbonyl)-phenyl]-pyridin-4-yl}-1H-pyrrole-3-carboxylic acid amide

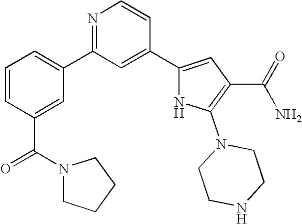

¹H-NMR (400 MHZ, DMSO-d6): 1.86 (m, 4H), 2.85 (m, 4H), 3.05 (m, 4H), 3.42 (t, 2H), 3.50 (t, 2H), 6.90 (bs, 1H), 7.18 (s, 1H), 7.57 (m, 3H), 7.64 (bs, 1H), 8.24 (m, 3H), 8.53 (d, 1H), 11.39 (s, 1H, NH).

MS (ESI⁺) m/z: 445 [MH]⁺.

### EXAMPLE 44

### 4-[4-(4-Cyano-5-piperazin-1-yl-1H-pyrrol-2-yl)-pyridin-2-yl]-benzoic acid ethyl ester trifluoroacetate

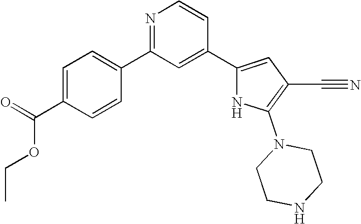

¹H-NMR (400 MHZ, DMSO-d6): 1.38 (t, 3H), 3.34 (m, 4H), 3.67 (m, 4H), 4.36 (q, 2H), 7.33 (d, 1H), 7.65 (dd, 1H), 8.09 (d, 2H), 8.27 (m, 3H), 8.63 (d, 1H), 8.83 (bs, 2H, NH₂⁺), 11.48 (s, 1H, NH-pyrrole).

MS (ESI⁺) m/z: 402 [MH]⁺.

MS (ESI⁺) m/z: 400 [MH]⁻.

### EXAMPLE 45

### 5-{2-[3-Nitro-5-(pyrrolidine-1-carbonyl)-phenyl]-pyridin-4-yl}-2-piperazin-1-yl-1H-pyrrole-3-carbonitrile trifluoroacetate

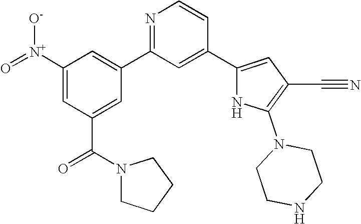

¹H-NMR (400 MHZ, DMSO-d6): 1.82 (m, 4H), 3.31 (m, 4H), 3.44 (t, 2H), 3.54 (t, 2H), 3.63 (m, 4H), 7.37 (d, 1H), 7.67 (dd, 1H), 8.35 (m, 2H), 8.66 (m, 2H), 8.80 (bs, 2H, NH₂⁺), 9.02 (m, 1H), 11.48 (s, 1H, NH).

MS (ESI⁺) m/z: 472 [MH]⁺.

### EXAMPLE 46

### 5-[2-(3-Cyclopentylcarbamoyl-phenyl)-pyridin-4-yl]-2-piperazin-1-yl-1H-pyrrole-3-carboxylic acid amide

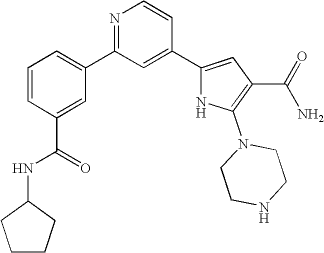

¹H-NMR (400 MHZ, DMSO-d6): 1.56 (m, 4H), 1.72 (m, 2H), 1.92 (m, 2H), 3.25 (m, 4H), 3.37 (m, 4H), 4.25 (m, 1H), 6.84 (bs, 1H, NH-amide), 7.25 (d, 1H), 7.30 (bs 1H, NH-amide), 7.57 (m, 2H), 7.87 (d, 1H), 7.16 (s, 1H), 8.21 (dd, 1H), 8.41 (dd, 1H), 8.52 (m, 1H), 8.56 (d, 1H), 11.33 (s, 1H, NH).

MS (ESI⁺) m/z: 459 [MH]⁺.

MS (ESI⁻) m/z: 457 [MH]⁻.

### EXAMPLE 47

### 5-{2-[2-Fluoro-5-(pyrrolidine-1-carbonyl)-phenyl]pyridin-4-yl}-2-piperazin-1-yl-1H-pyrrole-3-carboxylic acid amide

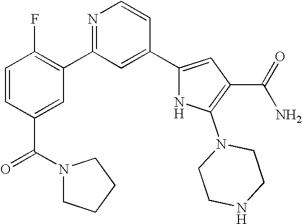

¹H-NMR (400 MHZ, DMSO-d6): 1.85 (m, 4H), 2.83 (m, 4H), 3.03 (m, 4H), 3.43 (m, 4H), 6.89 (bs, 1H, NH-amide), 7.08 (s, 1H), 7.40 (m, 1H), 7.63 (m, 2H), 7.63 (bs 1H, NH-amide), 7.96 (d, 1H), 8.00 (s, 1H), 8.56 (d,1H), 11.38 (s, 1H, NH-pyrrole).

MS (ESI⁺) m/z: 463 [MH]⁺.

MS (ESI⁻) m/z: 461 [MH]⁻.

### EXAMPLE 48

### N-(2-Cyano-ethyl)-3-[4-(4-cyano-5-piperazin-1-yl-1H-pyrrol-2-yl)-pyridin-2-yl]-benzamide trifluoroacetate

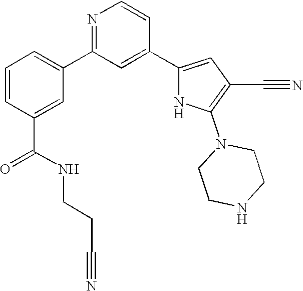

¹H-NMR (400 MHZ, DMSO-d6): 2.82 (t, 2H), 3.31 (m, 4H), 3.35-3.64 (m, 6H), 7.29 (d, 1H), 7.62 (m, 2H), 7.92 (d, 1H), 8.20 (s, 1H), 8.25 (d, 1H), 8.60 (m, 2H), 8.81 (bs, 2H, NH₂⁺), 8.96 (t, 1H, NH-amide), 11.49 (s, 1H, NH-pyrrole).

MS (ESI⁺) m/z: 426 [MH]⁺.

MS (ESI⁻) m/z: 424 [MH]⁻.

### EXAMPLE 49

### 4-[4-(4-Cyano-5-piperazin-1-yl-1H-pyrrol-2-yl)-pyridin-2-yl]-N-(2,2,2-trifluoro-ethyl)-benzamide trifluoroacetate

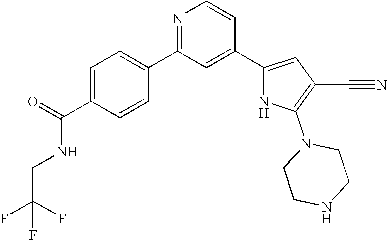

¹H-NMR (400 MHZ, DMSO-d6): 3.34 (m, 4H, hidden by H₂O), 3.63 (m, 4H) 4.12 (m, 2H), 7.26 d, 1H), 7.61 (dd, 1H), 8.08 (d, 2H), 8.24 (m, 3H), 8.60 (d, 1H), 8.78 (bs, 2H, NH₂⁺), 9.17 (t, 1H, NH-amide), 11.42 (s, 1H, NH-pyrrole).

MS (ESI⁺) m/z: 455 [MH]⁺.

MS (ESI⁻) m/z: 453 [MH]⁻.

### EXAMPLE 50

### 5-{2-[4-(Morpholine-4-sulfonyl)-phenyl]pyridin-4-yl}-2-piperazin-1-yl-1H-pyrrole-3-carbonitrile trifluoroacetate

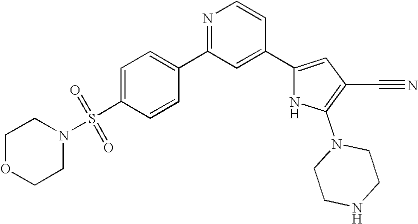

¹H-NMR (400 MHZ, DMSO-d6): 2.92 (m, 4H), 3.32 (m, 4H), 3.64 (m, 8H), 7.29 (d, 1H), 7.66 (dd, 1H), 7.88 (d, 2H), 8.26 (s, 1H), 8.36 (d, 2H), 8.63 (d, 1H), 8.78 (bs, 2H, NH₂⁺), 11.43 (s, 1H, NH-pyrrole).

MS (ESI⁺) m/z: 479 [MH]⁺.

MS (ESI⁻) m/z: 477 [M−H]⁻.

### EXAMPLE 51

### 5-{2-[4-(Morpholine-4-sulfonyl)-phenyl]-pyridin-4-yl}-2-piperazin-1-yl-1H-pyrrole-3-carboxylic acid amide

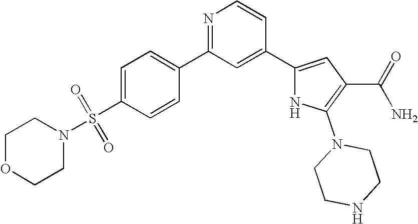

¹H-NMR (400 MHZ, DMSO-d6): 2.87 (m, 4H), 2.92 (m, 4H), 3.09 (m, 4H), 3.65 (m, 4H), 6.91 (bs, 1H, NH-amide), 7.22 (d, 1H), 7.62 (bs, 1H, NH-amide), 7.65 (dd, 1H), 7.86 (d, 2H), 8.31(s, 1H), 8.40 (d, 2H), 8.58 (d, 1H),11.39 (s, 1H, NH-pyrrole).

MS (ESI⁺) m/z: 497 [MH]⁺.

MS (ESI⁻) m/z: 495 [M−H]⁻.

### EXAMPLE 52

### 5-{2-[3-Nitro-5-(pyrrolidine-1-carbonyl)-phenyl]-pyridin-4-yl}-2-piperazin-1-yl-1H-pyrrole-3-carboxylic acid amide

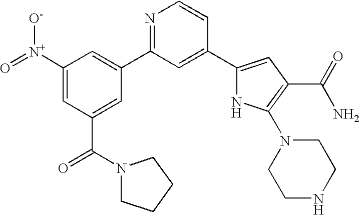

¹H-NMR (400 MHZ, DMSO-d6): 1.88 (m, 4H), 2.86 (m, 4H), 3.05 (m, 4H), 3.45 (t, 2H), 3.54 (t, 2H), 6.91 (bs, 1H, NH-amide), 7.29 (s, 1H), 7.63 (bs, 1H, NH-amide), 7.68 (d, 1H), 8.32 (m, 1H), 8.39 (s, 1H), 8.59 (d, 1H), 8.70 (t, 1H), 9.04 (t, 1H), 11.41 (s, 1H, NH-pyrrole).

MS (ESI⁺) m/z: 490 [MH]⁺.

MS (ESI⁻) m/z: 488 [M−H]⁻.

### EXAMPLE 53

### 5-{2-[3-(5-Methyl-[1,3,4]oxadiazol-2-yl)-phenyl]pyridin-4-yl}-2-piperazin-1-yl-1H-pyrrole-3-carbonitrile trifluoroacetate

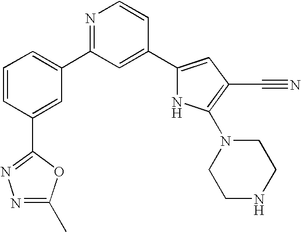

¹H-NMR (400 MHZ, DMSO-d6): 2.65 (s, 3H), 3.33 (m, 4H), 3.67 (m, 4H), 7.37 (s, 1H), 7.68 (dd, 1H), 7.11 (t, 1H), 8.07 (d, 1H), 8.29 (s, 1H), 8.35 (d, 1H), 8.64 (d, 1H), 8.75 (m, 1H), (8.83 (bs, 2H, NH₂⁺), 11.51 (s, 1H, NH-pyrrole).

MS (ESI⁺) m/z: 412 [MH]⁺.

MS (ESI⁻) m/z: 410 [M−H]⁻.

### EXAMPLE 54

### 4-[4-(4-Cyano-5-piperazin-1-yl-1H-pyrrol-2-yl)-pyridin-2-yl]-N-cyclopentyl-benzamide trifluoroacetate

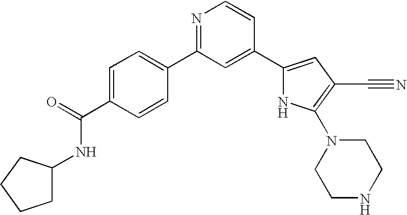

¹H-NMR (400 MHZ, DMSO-d6): 1.59 (m, 4H), 1.75 (m, 2H), 1.93 (m, 2H), 3.33 (m, 4H), 3.67 (m, 4H), 4.28 (m, 1H), 7.37 (s, 1H), 7.62 (m, 1H), 8.01 (d, 2H), 8.19 (d, 2H), 8.25 (m, 1H), 8.38 (d, 1H), 8.61 (d, 1H), 8.84 (bs, 2H, NH₂⁺), 11.49 (s, 1H, NH-pyrrole).

MS (ESI⁺) m/z: 441 [MH]⁺.

MS (ESI⁻) m/z: 439 [M−H]⁻.

### EXAMPLE 55

### 5-[2-(4-Cyclopentylcarbamoyl-phenyl)-pyridin-4-yl]-2-piperazin-1-yl-1H-pyrrole-3-carboxylic acid amide

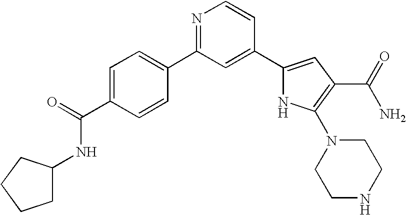

¹H-NMR (400 MHZ, DMSO-d6): 1.56 (m, 4H), 1.72 (m, 2H), 1.91 (m, 2H), 2.86 (m, 4H), 3.06 (m, 4H), 4.24 (m, 1H), 6.91 (bs, 1H, NH-amide), 7.19 (s, 1H), 7.61 (dd, 1H), 7.64 (bs, 1H, NH-amide), 7.97 (d, 2H), 8.21 (d, 2H), 8.22 (m, 1H), 8.34 (d, 1H), 8.54 (d, 1H), 11.38 (s, 1H, NH-pyrrole).

MS (ESI⁺) m/z: 459 [MH]⁺.

MS (ESI⁻) m/z: 459 [M−H]⁻.

### EXAMPLE 56

### 5-{2-[4-(5-Methyl-[1,3,4]oxadiazol-2-yl)-phenyl]-pyridin-4-yl}-2-piperazin-1-yl-1H-pyrrole-3-carbonitrile trifluoroacetate

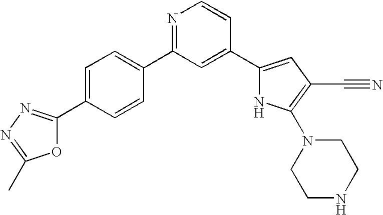

¹H-NMR (400 MHZ, DMSO-d6): 2.64 (s, 3H), 3.33 (m, 4H), 3.70 (m, 4H), 7.34 (s, 1H), 7.67 (dd, 1H), 8.14 (d, 2H), 8.28 (s, 1H), 8.35 (d, 1H), 8.63 (d, 1H), 8.79 (bs, 2H, NH₂⁺), 11.46 (s, 1H, NH-pyrrole).

MS (ESI⁺) m/z: 412 [MH]⁺.

MS (ESI⁻) m/z: 410 [M−H]⁻.

### EXAMPLE 57

### 5-{2-[3-(5-Methyl-[1,3,4]oxadiazol-2-yl)-phenyl]pyridin-4-yl}-2-piperazin-1-yl-1H-pyrrole-3-carboxylic acid amide

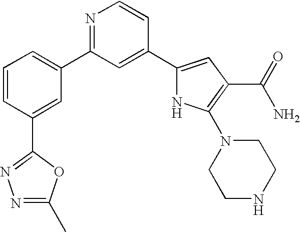

¹H-NMR (400 MHZ, DMSO-d6): 2.63 (s, 3H), 2.86 (m, 4H), 3.06 (m, 4H), 6.91 (bs, 1H, NH-amide), 7.21 (s, 1H), 7.63 (dd, 1H), 7.63 (bs, 1H, NH-amide), 7.73 (t, 1H), 8.03 (d, 1H), 8.29 (m, 1H), 8.37 (d, 1H), 8.56 (d, 1H), 8.76 (m, 1H), 11.42 (s, 1H, NH-pyrrole).

MS (ESI⁺) m/z: 430 [MH]⁺.

MS (ESI⁻) m/z: 428 [M−H]⁻.

### EXAMPLE 58

### 2-Piperazin-1-yl-5-{2-[4-(2,2,2-trifluoro-ethylcarbamoyl)-phenyl]-pyridin-4-yl}-1H-pyrrole-3-carboxylic acid amide

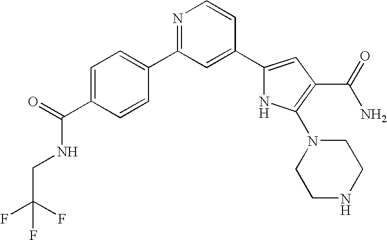

¹H-NMR (400 MHZ, DMSO-d6): 2.86 (m, 4H), 3.06 (m, 4H), 4.12 (m, 1H), 6.91 (bs, 1H, NH-amide), 7.20 (s, 1H), 7.63 (bs, 1H, NH-amide), 7.63 (dd, 1H), 8.03 (d, 2H), 8.27 (d, 2H), 8.27 (m, 1H), 8.55 (d, 1H), 9.17(t, 1H, NH-amide), 11.38 (s, 1H, NH-pyrrole).

MS (ESI⁺) m/z: 361 [MH]⁺.

MS (ESI⁻) m/z:359 [M−H]⁻.

### EXAMPLE 59

### Morpholine-4-carboxylic acid {4-[4-(4-carbamoyl-5-piperazin-1-yl-1H-pyrrol-2-yl)-pyridin-2-yl]-phenyl}-amide

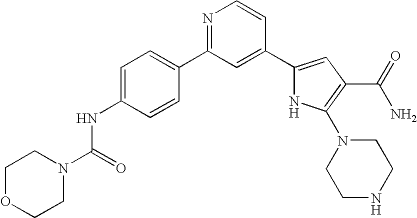

¹H-NMR (400 MHZ, DMSO-d6): 2.86 (m, 4H), 3.05 (m, 4H), 3.46 (t, 4H), 3.62 (t, 4H) 6.91 (bs, 1H, NH-amide) 7.14 (d, 1H), 7.59 (dd, 1H), 7.63 (d, 2H), 7.67 (bs, 1H, NH-amide), 8.05 (d, 2H), 8.14 (s, 1H), 8.47 (d, 1H), 8.71(s, 1H) 11.37 (s, 1H, NH-pyrrole).

MS (ESI⁺) m/z: 476 [MH]⁺.

### EXAMPLE 60

### 5-[2-(4-Methyl-3,4-dihydro-2H-benzo[1,4]oxazin-6-yl)-pyridin-4-yl]-2-piperazin-1-yl-1H-pyrrole-3-carbonitrile trifluoroacetate

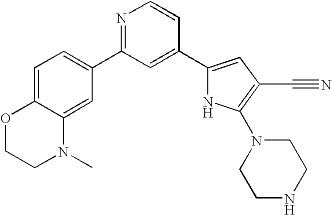

¹H-NMR (400 MHZ, DMSO-d6): 3.00 (s, 3H), 3, 31-3.43 (m, 6H+H₂O), 6.88 (m, 1H), 7.49 (m, 1H), 7.59 (dd, 1H), 7.67 (s, 1H), 7.73 (d, 1H), 8.20 (s, 1H), 8.46 (d, 1H), 8.95 (bs, 2H, NH₂⁺), 11.58 (s, 1H, NH-pyrrole).

MS (ESI⁺) m/z: 401 [MH]⁺.

MS (ESI⁻) m/z: 399 [M−H]⁻.

### EXAMPLE 61

### (S)-3-Amino-5′-{2-[(E)-2-(4-fluoro-phenyl)-vinyl]-pyridin-4-yl}-2,3,4,5-tetrahydro-1′H-[1,2′]bipyrrolyl-3-carbonitrile trifluoroacetate

### a) [(S)-5′-(2-Chloro-pyridin-4-yl)-3′-cyano-2,3,4,5-tetrahydro-1′H-[1,2′]bipyrrolyl-3-yl]-carbamic acid tert-butyl ester

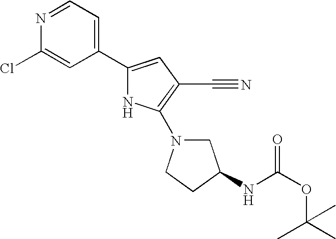

2-Bromo-1-(2-chloro-pyridin-4-yl)-ethanone hydrobromide (1.300 g) is added in 6 ml ethanol to a mixture of 406 mg of NaHCO₃ and 838 mg of [(S)-1-((Z)-1-amino-2-cyano-vinyl)-pyrrolidin-3-yl]-carbamic acid tert-butyl ester (prepared in a similar fashion as example 1b starting from (S)-pyrrolidin-3-yl-carbamic acid tert-butyl ester). The reaction mixture is refluxed for 5 minutes and then stirred for 3 days. After removal of the solvent the resulting residue is dissolved in ethyl acetate, washed with water/brine and dried over sodium sulfate. After removal of the solvent 250 mg of the resulting red solid (1.436 g) are purified via HPLC (acetonitrile/H₂O, x-Terra RP-18) to yield a yellow solid.

¹H-NMR (400 MHZ, DMSO-d6): 1.41 (s, 9H), 1.91 (m, 1H), 2.14 (m, 1H), 3.38 (dd, 1H), 3.56 (m, 1H), 3.69 (m, 2H), 4.13 (m, 1H), 7.14 (d, 1H), 7.28 (d, 1H, NH), 7.51 (dd, 1H), 7.71 (1H), 8.18 (d, 1H), 10.47 (s, 1H).

MS (ESI⁺) m/z: 388 [MH]⁺.

### b) (S)-3′-Cyano-5′-{2-[(E)-2-(4-fluoro-phenyl)-vinyl]-pyridin-4-yl}-2,3,4,5-tetrahydro-1′H-[1,2′]bipyrrolyl-3-yl)-carbamic acid tert-butyl ester

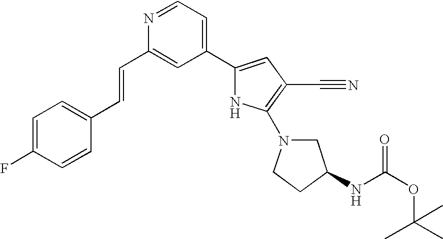

Prepared as described in example 8 from trans-2(-4-fluoro-phenyl)-vinyl boronic acid and [(S)-5′-(2-Chloro-pyridin-4-yl)-3′-cyano-2,3,4,5-tetrahydro-1′H-[1,2′]bipyrrolyl-3-yl]-carbamic acid tert-butyl ester.

¹H-NMR (400 MHZ, DMSO-d6): 1.41 (s, 9H), 1.94 (m, 1H), 2.16 (m, 1H),3.38 (dd, 1H), 3.56 (m, 1H), 3.72 (m, 2H), 6.99 (s, 1H), 7.15 (d, 1H), 7.21-7.28 (m, 3H), 7.43 (dd, 1H), 7.62-7.71 (m, 4H), 8.39 (d, 1H), 10.48 (s, 1H).

MS (ESI⁺) m/z: 474 [MH]⁺.

### c) (S)-3-Amino-5′-{2-[(E)-2-(4-fluoro-phenyl)-vinyl]-pyridin-4-yl}-2,3,4,5-tetrahydro-1′H-[1,2′]bipyrrolyl-3-carbonitrile trifluoroacetate

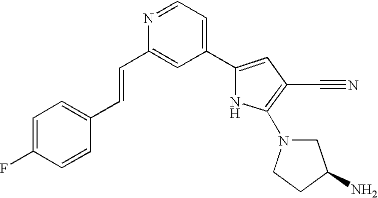

Prepared in analogy to example 8 from (S)-3′-cyano-5′-{2-[(E)-2-(4-fluoro-phenyl)-vinyl]-pyridin-4-yl}-2,3,4,5-tetrahydro-1′H-[1,2′]bipyrrolyl-3-yl)-carbamic acid tert-butyl ester

¹H-NMR (400 MHZ, DMSO-d6): 2.12 (m, 1H), 2.36 (m, 1H), 3.70 (m, 2H), 3.80 (m, 1H), 3.89 (m, 1H), 4.02 (m, 1H), 7.18 (d, 1H), 7.33 (m, 2H), 7.49 (1H), 7.69-7.78 (m, 4H), 8.08 (4H,1H+NH₃⁺), 8.48 (d, 1H), 10.62 (s, 1H).

MS (ESI⁺) m/z: 374 [MH]⁺.

### EXAMPLE 62

### (S)-3-Amino-5′-{2-[(E)-2-(4-fluoro-phenyl)-vinyl]-pyridin-4-yl}-2,3,4,5-tetrahydro-1′H[1,2′]bipyrrolyl-3′-carboxylic acid amide

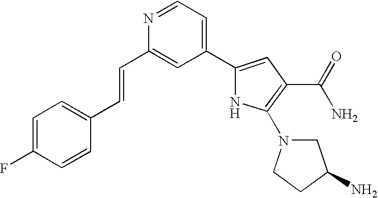

Prepared according to example 9 from (S)-3-amino-5′-{2-[(E)-2-(4-fluoro-phenyl)-vinyl]-pyridin-4-yl)-2,3,4,5-tetrahydro-1′H-[1,2′]bipyrrolyl-3-carbonitrile trifluoroacetate

¹H-NMR (400 MHZ, DMSO-d6): 1.65 (m, 1H), 2.03 (m, 1H), 3.01 (q, 1H), 3.31-3.59 (m, 4H), 7.06 (s, 1H), 7.14-7.24 (m, 3H), 7.35 (dd, 1H), 7.61-7.71 (m, 6H), 8.36 (d, 1H), NH pyrrol not visible.

MS (ESI⁺) m/z: 392 [MH]⁺.

### EXAMPLE 63

### (R)-3-Amino-5′-{2-[(E)-2-(4-fluoro-phenyl)-vinyl]-pyridin-4-yl)-2,3,4,5-tetrahydro-1′H-[1,2′]bipyrrolyl-3-carbonitrile trifluoroacetate

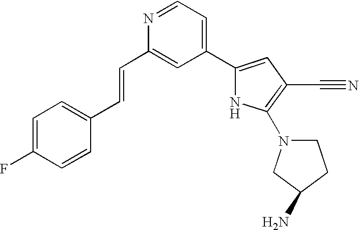

Prepared as described in example 61, starting from (R)-pyrrolidin-3-yl-carbamic acid tert-butyl ester.

¹H-NMR (400 MHZ, DMSO-d6): 2.12 (m, 1H), 2.36 (m, 1H), 3.70 (m, 2H), 3.80 (m, 1H), 3.89 (m, 1H), 4.02 (m, 1H), 7.18 (d, 1H), 7.33 (m, 2H), 7.49 (1H), 7.69-7.78 (m, 4H), 8.08 (4H, 1H+NH₃⁺), 8.48 (d, 1H), 10.62 (s, 1H).

MS (ESI⁺) m/z: 374 [MH]⁺.

### EXAMPLE 64

### (R)-3-Amino-5′-{2-[(E)-2-(4-fluoro-phenyl)-vinyl]-pyridin-4-yl}-2,3,4,5-tetrahydro-1′H[1,2′]bipyrrolyl-3′-carboxylic acid amide

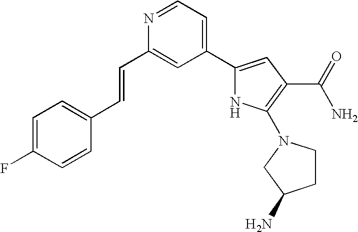

Prepared as described in example 62 starting from (R)-3-amino-5′-{2-[(E)-2-(4-fluoro-phenyl)-vinyl]-pyridin-4-yl)-2,3,4,5-tetrahydro-1′H-[1,2′]bipyrrolyl-3-carbonitrile trifluoroacetate

H-NMR (400 MHZ, DMSO-d6): 1.65 (m, 1H), 2.03 (m, 1H), 3.01 (q, 1H), 3.31-3.59 (m, 4H), 7.06 (s, 1H), 7.14-7.24 (m, 3H), 7.35 (dd, 1H), 7.61-7.71 (m, 6H), 8.36 (d, 1H), NH pyrrol not visible.

MS (ESI⁺) m/z: 392 [MH]⁺.

### EXAMPLE 65

### 2-[1,4]Diazepan-1-yl-5-{2-[(E)-2-(4-fluoro-phenyl)-vinyl]-pyridin-4-yl}-1H-pyrrole-3-carbonitrile trifluoroacetate

### a) 4-(3-Cyano-5-{2-[(E)-2-(4-fluoro-phenyl)-vinyl]-pyridin-4-yl}-1H-pyrrol-2-yl)-[1,4]diazepane-1-carboxylic acid tert-butyl ester

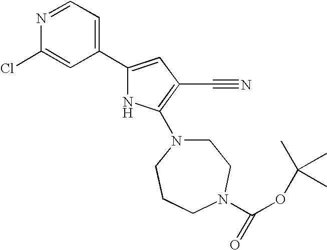

Prepared as outlined in example 1d.

MS (ESI⁺) m/z: 402 [MH]⁺.

MS (ESI⁻) m/z: 400 [M−H]⁻.

### b) 4-(3-Cyano-5-{2-[(E)-2-(4-fluoro-phenyl)-vinyl]-pyridin-4-yl}-1H-pyrrol-2-yl)-[1,4]diazepane-1-carboxylic acid tert-butyl ester

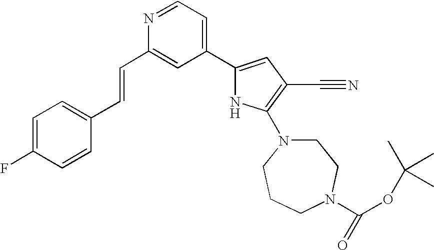

Prepared as described in example 1 starting from trans-2(-4-fluoro-phenyl)-vinyl boronic acid and 4-(3-Cyano-5-{2-[(E)-2-(4-fluoro-phenyl)-vinyl]-pyridin-4-yl}-1H-pyrrol-2-yl)-[1,4]diazepane-1-carboxylic acid tert-butyl ester.

¹H-NMR (400 MHZ, DMSO-d6): 1.26, 1.33 (s, 9H, rotamers), 1.32, 1,86 (m, 2H, rotamers), 3.35-3.76 (m, 8H), 6.68 (m, 1H), 7.15-7.25 (m, 3H), 7.42 (dd, 1H), 7.62-7.71 (m, 4H), 8.42 (d, 1H), 10.40 (s, 1H).

MS (ESI⁺) m/z: 488 [MH]⁺.

### c) 2-[1,4]Diazepan-1-yl-5-{2-[(E)-2-(4-fluoro-phenyl)-vinyl]-pyridin-4-yl}-1H-pyrrole-3-carbonitrile trifluoroacetate

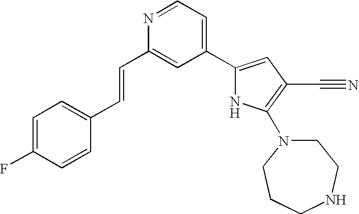

Prepared in a similar fashion as example 8 starting from 4-(3-cyano-5-{2-[(E)-2-(4-fluoro-phenyl)-vinyl]-pyridin-4-yl}-1H-pyrrol-2-yl)-[1,4]diazepane-1-carboxylic acid tert-butyl ester

¹H-NMR (400 MHZ, DMSO-d6): 2.13 (m, 2H), 3.28 (m, 2H), 3.38 (m, 1H), 3.72 (t, 2H), 3.88 (t, 2H), 7.18 (d, 1H), 7.3 (m, 2H), 7.48 (bs, 1H), 7.68-7.78 (m, 3H), 8.05 (bs, 1H), 8.48 (d, 1H), 8.73 (bs, 2H, NH₂⁺), 10.89 (s, 1H, NH).

MS (ESI⁺) m/z: 388 [MH]⁺.

### EXAMPLE 66

### 2-[1,4]Diazepan-1-yl-5-{2-[(E)-2-(4-fluoro-phenyl)-vinyl]-pyridin-4-yl)-1H-pyrrole-3-carboxylic acid amide

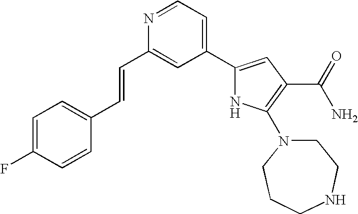

Prepared in a similar fashion as example 9 starting from 2-[1,4]diazepan-1-yl-5-{2-[(E)-2-(4-fluoro-phenyl)-vinyl]pyridin-4-yl}-1H-pyrrole-3-carbonitrile trifluoroacetate.

‘H-NMR (400 MHZ, DMSO-d6): 1.71, (m, 2H), 2.83-3.35 (m, 8H), 6.80 (bs, 1H), 7.02 (s, 1H), 7.16-7.26 (m, 3H), 7.43 (m, 1H), 7.64-7.77 (m, 5H), 8.40 (m 1H), 11.50 (1H).

MS (ESI⁺) m/z: 406 [MH]⁺.

### EXAMPLE 67

### 2-(4-Amino-piperidin-1-yl)-5-{2-[(E)-2-(4-fluoro-phenyl)-vinyl]-pyridin-4-yl}-1H-pyrrole-3-carbonitrile trifluoroacetate

### a) {1-[5-(2-Chloro-pyridin-4-yl)-3-cyano-1H-pyrrol-2-yl]-piperidin-4-yl}-carbamic acid tert-butyl ester

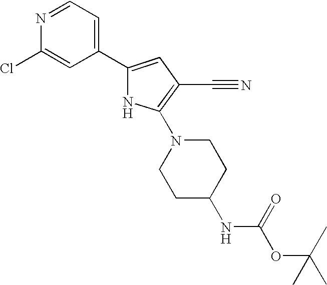

Prepared in analogy to example 1d.

¹H-NMR (400 MHZ, DMSO-d6): 1.40 (s, 9H), 1.51 (m, 2H), 1.84 (m, 2H), 3.09 (m, 2H), 3.46. (m, 1H), 3.83 (m, 2H), 6.91 (d, 1H, NH,carbamate), 7.14 (d, 1H), 7.55 (dd, 1H), 7.71 (s, 1H), 8.22 (d, 1H), 10.93 (s, 1H, NH).

MS (ESI⁺) m/z: 402 [MH]⁺.

MS (ESI⁻) m/z: 400 [M−H]⁻.

### b) [1-(3-Cyano-5-{2-[(E)-2-(4-fluoro-phenyl)-vinyl]-pyridin-4-yl}-1H-pyrrol-2-yl)-piperidin-4-yl]-carbamic acid tert-butyl ester

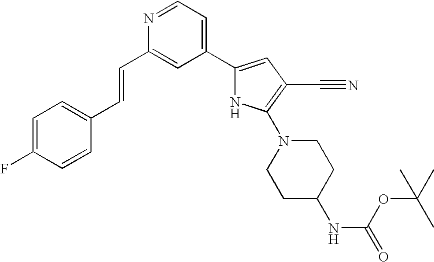

Prepared in a similar fashion as example 1 starting from trans-2(-4-fluoro-phenyl)-vinyl boronic acid and {1-[5-(2-Chloro-pyridin-4-yl)-3-cyano-1H-pyrrol-2-yl]-piperidin-4-yl}-carbamic acid tert-butyl ester.

¹H-NMR (400 MHZ, DMSO-d6): 1.40 (s, 9H) 1.51 (m, 2H), 1.84 (m, 2H), 3.09 (m, 2H), 3.46. (m, 1H), 3.83 (m, 2H), 6.91 (d, 1H, NH), 7.01 (d, 1H), 7.14-7.26 (m, 3H), 7.44 (dd, 1H), 7.63-7.73 (m, 4H) 8.43 (d, 1H), 10.93 (s, 1H, NH).

MS (ESI⁺) m/z: 488 [MH]⁺.

MS (ESI⁻) m/z: 486 [M−H]⁻.

### c) 2-(4-Amino-piperidin-1-yl)-5-{2-[(E)-2-(4-fluoro-phenyl)-vinyl]-pyridin-4-yl}-1H-pyrrole-3-carbonitrile trifluoroacetate

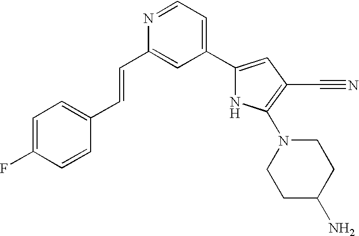

Prepared in a similar fashion as example 8 starting from [1-(3-cyano-5-{2-[(E)-2-(4-fluoro-phenyl)-vinyl]pyridin-4-yl}-1H-pyrrol-2-yl)-piperidin-4-yl]-carbamic acid tert-butyl ester.

¹H-NMR (400 MHZ, DMSO-d6): 1.55 (m, 2H), 2.01 (m, 2H), 3.18 (m, 2H), 3.31 (m, 1H), 4.01 (m, 2H), 7.19 (d, 1H), 7.31 (m, 2H), 7.46 (1H), 7.71-7.81 (m, 3H), 9.93 (3H), 8.10 (bs, 1H), 8.51 (d, 1H), 11.31 (s, 1H, NH).

MS (ESI⁺) m/z: 388 [MH]⁺.

### EXAMPLE 68

### 2-(4-Amino-piperidin-1-yl)-5-{2-[(E)-2-(4-fluoro-phenyl)-vinyl]-pyridin-4-yl}-1H-pyrrole-3-carboxylic acid amide

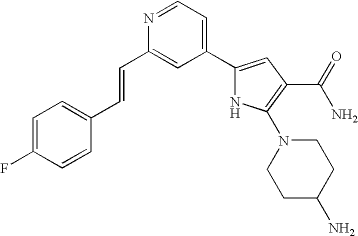

Prepared as shown in example 9 from 2-(4-amino-piperidin-1-yl)-5-{2-[(E)-2-(4-fluoro-phenyl)-vinyl]-pyridin-4-yl}-1H-pyrrole-3-carbonitrile trifluoroacetate.

¹H-NMR (400 MHZ, DMSO-d6): 1.41 (m, 2H), 1.85 (m, 2H), 2.75 (m, 1H), 3.0 (m, 2H), 3.18 (d, 2H), 7.19 (d, 1H), 6.91 (bs, 1H), 7.05 (s, 1H), 7.18-7.26 (m, 3H), 7.46 (dd, 1H), 7.65-7.77 (m, 4H), 8.44 (d, 1H), 11.34 (s, 1H, NH).

MS (ESI⁺) m/z: 406 [MH]⁺.

MS (ESI⁻) m/z: 404 [M−H]⁻.

### EXAMPLE 69

### 5-{2-[(E)-2-(4-Fluoro-phenyl)-vinyl]-pyridin-4-yl}-2-(4-hydroxy-piperidin-1-yl)-1H-pyrrole-3-carbonitrile

### a) 5-(2-Chloro-pyridin-4-yl)-2-(4-hydroxy-piperidin-1-yl)-1H-pyrrole-3-carbonitrile

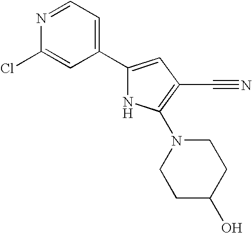

Prepared in a similar fashion as example 1d.

¹H-NMR (400 MHZ, DMSO-d6): 1.48 (m, 2H), 1.83 (m, 2H), 3.19 (m, 2H), 3.70 (m, 3H), 4.77 (d, 1H, OH), 7.16 (d, 1H), 7.57 (dd, 1H), 7.72 (s, 1H), 8.22 (dd, 1H), 10.95 (s, 1H, NH).

MS (ESI⁺) m/z: 303[MH]⁺.

MS (ESI⁻) m/z: 301[M−H]⁻.

### b) 5-{2-[(E)-2-(4-Fluoro-phenyl)-vinyl]-pyridin-4-yl}-2-(4-hydroxy-piperidin-1-yl)-1H-pyrrole-3-carbonitrile

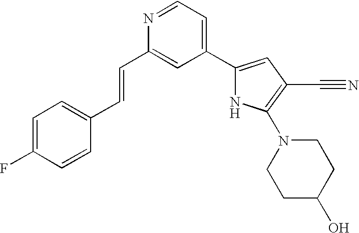

Prepared in a similar fashion as example 1 from trans-2(-4-fluoro-phenyl)-vinyl boronic acid and 5-(2-chloro-pyridin-4-yl)-2-(4-hydroxy-piperidin-1-yl)-1H-pyrrole-3-carbonitrile.

¹H-NMR (400 MHZ, DMSO-d6): 1.52 (m, 2H), 1.86 (m, 2H), 3.19 (m, 2H), 3.71 (m, 3H), 4.76 (d, 1H, OH), 7.01 (d, 1H), 7.14-7.26 (m, 3H), 7.44 (dd, 1H), 7.62-7.74 (m, 4H), 8.43 (d, 1H), 10.98 (s, 1H, NH)

MS (ESI⁺) m/z: 389 [MH]⁺.

MS (ESI⁻) m/z: 387 [M−H]⁻.

### EXAMPLE 70

### 5-{2-[(E)-2-(4-Fluoro-phenyl)-vinyl]-pyridin-4-yl}-2-(3-hydroxy-piperidin-1-yl)-1H-pyrrole-3-carbonitrile

### a) 5-(2-Chloro-pyridin-4-yl)-2-(3-hydroxy-piperidin-1-yl)-1H-pyrrole-3-carbonitrile

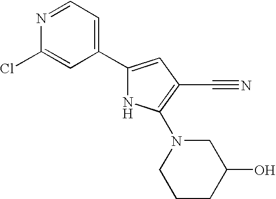

The title compound is prepared as outlined in example 1d.

‘H-NMR (400 MHZ, DMSO-d6): 1.36 (m, 1H), 1.57 (m, 1H), 1.85 (m, 2H), 2.81 (m, 1H), 3.06 (m, 1H), 3.63 (m, 2H), 3.79 (dd, 1H), 4.96 (s, 1H, OH), 7.14 (s, 1H), 7.57 (dd, 1H), 7.73 (d, 1H), 8.22. (d, 1H), 10.42 (s, 1H, NH)

MS (ESI⁺) m/z: 303 [MH]⁺.

MS (ESI⁻) m/z: 301 [M−H]⁻.

### b) 5-{2-[(E)-2-(4-Fluoro-phenyl)-vinyl]-pyridin-4-yl}-2-(3-hydroxy-piperidin-1-yl)-1H-pyrrole-3-carbonitrile

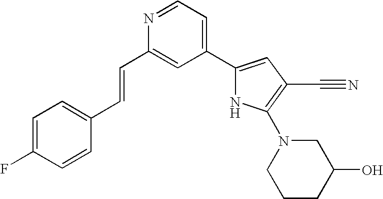

Prepared in a similar fashion as example 1 starting from trans-2(-4-fluoro-phenyl)-vinyl boronic acid and 5-(2-chloro-pyridin-4-yl)-2-(3-hydroxy-piperidin-1-yl)-1H-pyrrole-3-carbonitrile.

¹H-NMR (400 MHZ, DMSO-d6): 1.36 (m, 1H), 1.57 (m, 1H), 1.87 (m, 2H), 2.88 (m, 1H), 3.04 (m, 1H), 3.63 (m, 2H), 3.79 (dd, 1H), 4.96 (d, 1H, OH), 7.01 (d, 1H), 7.14-7.26 (m, 3H), 7.44 (dd, 1H), 7.62-7.75 (m, 4H), 8.43 (d, 1H), 10.98 (s, 1H, NH).

MS (ESI⁺) m/z: 389 [MH]⁺.

MS (ESI⁻) m/z: 387 [M−H]⁻.

### EXAMPLE 71

### 5-{2-[(E)-2-(4-Morpholin-4-ylmethyl-phenyl)-vinyl]-pyridin-4-yl)-2-piperazin-1-yl-1H-pyrrole-3-carbonitrile trifluoroacetate

### a) 4-(4-Ethynyl-benzyl)-morpholine

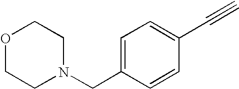

A mixture of 4-ethinyl benzylalcohol (2.0 g), morpholine (1.85 g), cyanomethyl-trimethyl-phosphonium iodide (5.5 g) and ethyl diisoproylamine (3.9 ml) in 30 ml propionitrile is refluxed for 16 hours. Aqueous NaHCO₃ solution is added and the mixture extracted with ethyl acetate. After removal of the solvent a brownish oil is obtained which crystallizes on standing.

¹H-NMR (400 MHZ, DMSO-d6): 2.33 (br s, 4H), 3.45 (s, 2H), 3.55 (br t, 4H), 4.12 (s, 1H), 7.29 (d, 2H), 7.40 (d, 2H).

MS (ESI⁺) m/z: 202 [MH]⁺.

### b) 4-{4-[(E)-2-(4,4,5,5-Tetramethyl-[1,3,2]dioxaborolan-2-yl)-vinyl]-benzyl}-morpholine

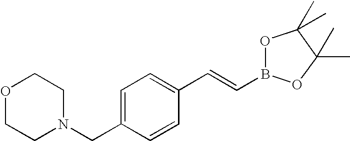

4,4,5,5-Tetramethyl-11,3,2-dioxaborolane (1.4 g) is added drop wise to a mixture of 4-(4-ethynyl-benzyl)-morpholine (1.5 g) and Rh(PPh₃)₂(CO)Cl (51 mg) in 50 ml dichloromethane. After 16 hours of stirring at room temperature another portion of catalyst (51 mg) is added and stirring is continued for another 20 hours. Aqueous NH₄Cl solution is added and the product is extracted into ethyl acetate. Chromatography on silica (ethyl acetate/hexanes, 7:3) yields a pale yellow oil which crystallizes on standing at room temperature.

¹H-NMR (400 MHZ, CDCl₃): 1.26 (s, 12H), 2.40-2.48 (m, 4H), 3.49 (s, 2H), 3.72 (br t, 4H), 6.15 (d, 1H), 7.30 (d, 2H), 7.38 (d, 1H), 7.45 (d, 2H).

MS (ESI⁺) m/z: 330 [MH]⁺.

### c) 4-(3-Cyano-5-{2-[(E)-2-(4-morpholin-4-ylmethyl-phenyl)-vinyl]-pyridin-4-yl}-1H-pyrrol-2-yl)-piperazine-1-carboxylic acid tert-butyl ester

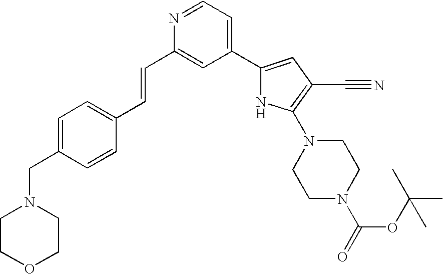

4-{4-[(E)-2-(4,4,5,5-Tetramethyl-[1,3,2]dioxaborolan-2-yl)-vinyl]-benzyl}-morpholine (185 mg) and (108) mg of 4-[5-(2-chloro-pyridin-4-yl)-3-cyano-1H-pyrrol-2-yl]-piperazine-1-carboxylic acid tert-butyl ester are dissolved in 3 ml n-propanol. The solution is degassed by introduction of a stream of argon. Pd(PPh₂)₂Cl₂ (9.8.mg) and 350 μl of 2N Na₂CO₃ are added and the mixture is heated for 15 mn at 145° C. in a microwave oven. After filtration of the reaction mixture over celite and evaporation of the solvent the resulting oil (350 mg) is purified by reverse phase HPLC (Gilson, X-Terra, acetonitrile/water) and yields a yellow solid.

¹H-NMR (400 MHZ, DMSO-d6): 1.44 (s, 9H), 2.38 (m, 4H), 3.34-3.62 (m, 12H), 3.48 (s, 2H), 7.05 (s, 1H), 7.18 (d, 1H), 7.34 (d, 2H), 7.44 (dd, 1H), 7.59 (dd, 2H), 7.64 (d, 1H), 7.76 (s, 1H), 8.45 (d, 1H), 11.20 (s, 1H, NH).

MS (ESI⁺) m/z: 554 [MH]⁺.

MS (ESI⁻) m/z: 553 [M−H]⁻.

### d) 5-{2-[(E)-2-(4-Morpholin-4-ylmethyl-phenyl)-vinyl]-pyridin-4-yl}-2-piperazin-1-yl-1H-pyrrole-3-carbonitrile trifluoroacetate

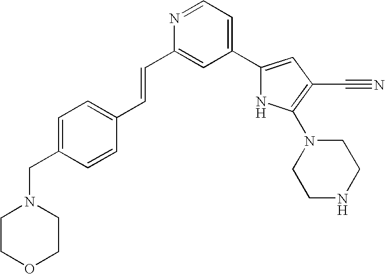

52.9 mg of 4-(3-cyano-5-{2-[(E)-2-(4-morpholin-4-ylmethyl-phenyl)-vinyl]-pyridin-4-yl}-1H-pyrrol-2-yl)-piperazine-1-carboxylic acid tert-butyl ester are stirred overnight with trifluoroacetic acid (290 μl) in 2 ml dichloromethane. The residue obtained after evaporation of the solvent is redissolved and redried three times in ethanol and then lyophilised with tert-butylalcohol to yield an orange solid.

¹H-NMR (400 MHZ, DMSO-d6, 120° C.): 2.85 (m, 4H), 3.1-3.4 (H₂0, m, 4H, hidden), 3.67 (m, 4H), 3.73 (m, 4H), 3.98 (bs, 2H), 7.00 (s, 1H), 7.24 (d, 1H), 7.44 (m, 3H), 7.62 (m, 2H), 7.64 (d, 1H), 7.75 (s, 1H), 8.46 (d, 1H).

MS (ESI⁺) m/z: 455 [MH]⁺.

MS (ESI⁻) m/z: 453 [M−H]⁺.

### EXAMPLE 72

### 5-{2-[(E)-2-(4-Morpholin-4-ylmethyl-phenyl)-vinyl]-pyridin-4-yl}-2-piperazin-1-yl-1H-pyrrole-3-carboxylic acid amide hydrobromide

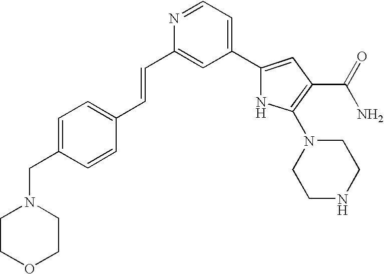

27.9 mg of 4-(3-carbamoyl-5-{2-[(E)-2-(4-morpholin-4-ylmethyl-phenyl)-vinyl]-pyridin-4-yl}-1H-pyrrol-2-yl)-piperazine-1-carboxylic acid benzyl ester are dissolved in 0.5 ml dichloromethane. After addition of 0.5 ml of hydrobromic acid (33% in acetic acid), the mixture is stirred overnight at room temperature.

The solid that is formed is filtered, redissolved in tert.butylalcohol and lyophilised.

¹H-NMR (400 MHZ, DMSO-d6,): 3.14 (m, 2H), 3.30 (m, 4H), 3.50-3.70 (m, 8H, part. hidden by water), 3.98 (m, 2H), 7.01 (bs, 1H), 7.00 (s, 1H), 7.32 (d, 1H), 7.61 (d, 2H), 7.78 (d, 2H), 8.01 (d, 1H), 8.32 (bs, 1H), 8.52 (d, 1H), 8.78 (bs, 2H), 9.96 (bs, 1H).

MS (ESI⁺) m/z: 473 [MH]⁺

### EXAMPLE 73

### 4-{(E)-2-[4-(4-Cyano-5-piperazin-1-yl-1H-pyrrol-2-yl)-pyridin-2-yl]-vinyl)-N,N-diethyl-benzamide trifluoroacetate

### a) N,N-Diethyl-4-ethynyl-benzamide

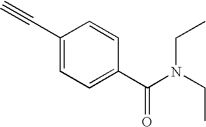

4-Ethynylbenzoic-acid sodium salt (1.0 g, 5.77 mmol), HOBT (1.0 g, 6.51 mmol) and diethylamine (1.2 ml, 11 mmol) are suspended in 50 ml CH₂Cl₂/THF (1:1), then EDC hydrochloride (1.3 g, 6.78 mmol) is added at room temperature. The resulting clear reaction mixture is stirred over night, quenched with saturated aqueous NaHCO₃ solution and extracted with ethyl acetate. The organic layer is washed with water and brine, dried over Na₂SO₄ and concentrated in vacuo. Silica gel purification (hexanes/ethyl acetate) affords the product as a colorless solid.

¹H-NMR (400 MHZ, CDCl₃): 1.00-1.10 (m, 3H), 1.15-1.25 (m, 3H), 3.11 (s, 1H), 3.15-3.25 (m, 2H), 3.47-3.57 (m, 2H), 7.31 (d, 2H), 7.49 (d, 2H).

MS (ESI⁺) m/z: 202 [MH]⁺.

### b) N,N-Diethyl-4-[(E)-2-(4,4,5,5-tetramethyl-[1,3,2]dioxaborolan-2-yl)-vinyl]-benzamide

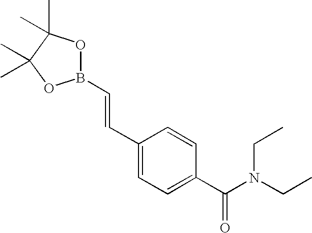

Under Ar N,N-diethyl-4-ethynyl-benzamide (900 mg, 4.34 mmol) and Wilkinson's catalyst (RhCl(PPh₃)₃) (85 mg, 0.08 mmol) are dissolved in CH₂Cl₂. A solution of pinacolborane (1.2 g, 9.2 mmol) in 3 ml CH₂Cl₂ is slowly added and the resulting dark red reaction mixture is allowed to stir at room temperature for 24 h. The reaction is quenched with ice-water and extracted with ethyl acetate. The organic layer is washed with water and brine, dried over Na₂SO₄ and concentrated in vacuo. After filtration over silica gel (hexanes/ethyl acetate) the product is used in the next step without further purification.

MS (ESI⁺) m/z: 330 [MH]⁺.

### c) 4-(3-Cyano-5-{2-[(E)-2-(4-diethylcarbamoyl-phenyl)-vinyl]-pyridin-4-yl}-1H-pyrrol-2-yl)piperazine-1-carboxylic acid tert-butyl ester

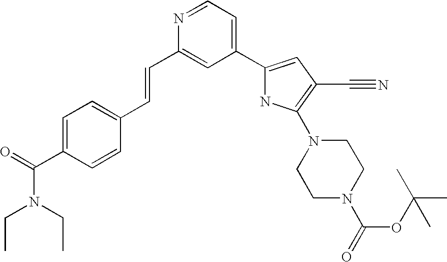

N,N-Diethyl-4-[(E)-2-(4,4,5,5-tetramethyl-[1,3,2]dioxaborolan-2-yl)-vinyl]-benzamide (245 mg) and (144) mg of 4-[5-(2-chloro-pyridin-4-yl)-3-cyano-1H-pyrrol-2-yl]-piperazine-1-carboxylic acid tert-butyl ester are dissolved in 3 ml n-propanol. The solution is degassed by introduction of a stream of argon. Pd(PPh₂)₂Cl₂ (13.mg) and 470 μl of 2N Na₂CO₃ are added and the mixture is heated for 15 mn at 145° C. in a microwave oven. After filtration of the reaction mixture over celite and evaporation of the solvent the resulting oil (300 mg) is purified by reverse phase HPLC (Gilson, X-Terra, acetonitrile/water) and yields a yellow solid.

¹H-NMR (400 MHZ, DMSO-d6): 1.10 (6H), 1.43 (s, 9H), 3.34-3.62 (m, 12H), 7.05 (s, 1H), 7.22-7.35 (m, 3H), 7.46 (d, 1H), 7.64-7.70 (m, 3H), 7.77 (s, 1H), 8.45 (d, 1H), 11.21 (s, 1H, NH).

MS (ESI⁺) m/z: 555 [MH]⁺.

MS (ESI⁻) m/z: 553 [M−H]⁻.

### d) 4-{(E)-2-[4-(4-Cyano-5-piperazin-1-yl-1H-pyrrol-2-yl)-pyridin-2-yl]-vinyl)-N,N-diethyl-benzamide trifluororacetate

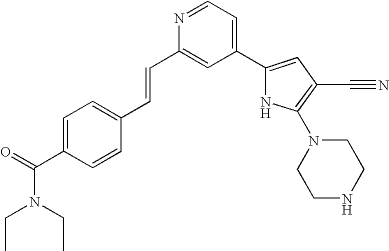

52,9 mg of 4-(3-cyano-5-{2-[(E)-2-(4-diethylcarbamoyl-phenyl)-vinyl]-pyridin-4-yl}-1H-pyrrol-2-yl) piperazine-1-carboxylic acid tert-butyl ester are stirred overnight with trifluoroacetic acid (300 μl) in 2 ml dichloromethane. The residue obtained after evaporation of the solvent yields the title compound as red solid.

¹H-NMR (400 MHZ, DMSO-d6): 1.10 (6H), 3.34-3.65 (m, 12H), 7.26-7.42 (m 4H), 7.58 (1H), 7.64-7.74 (m, 3H), 7.77 (1H), 8.52 (d, 1H), 8.80 (bs, 2H, NH₂⁺), 11.41 (s, 1H, NH).

MS (ESI⁺) m/z: 455 [MH]⁺.

MS (ESI⁻) m/z: 453 [M−H]⁻

### EXAMPLE 74

### 5-{2-[(E)-2-(4-Diethylcarbamoyl-phenyl)-vinyl]-pyridin-4-yl}-2-piperazin-1-yl-1H-pyrrole-3carboxylic acid amide

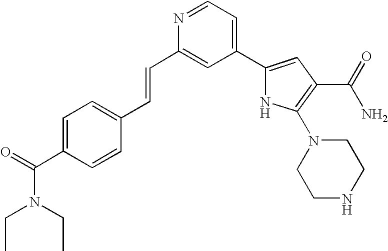

Prepared in a similar fashion as example 9 from 4-{(E)-2-[4-(4-cyano-5-piperazin-1-yl-1H-pyrrol-2-yl)-pyridin-2-yl]-vinyl}-N,N-diethyl-benzamide trifluororacetate.

¹H-NMR (400 MHZ, DMSO-d6): 1.10 (6H), 2.75 (m, 4H), 3.02 (m, 4H) 3.22-3.42 (m, 4H), 6.90 (bs, 1H, CONH₂), 7.08 (1H), 7.23 (d, 1H), 7.38 (d, 2H), 7.50 (dd, 1H), 7.61 (bs, 1H, CONH₂), 7.67-7.72 (m, 3H), 7.82 (1H), 8.46 (d, 1H), 11.3 (s, NH).

MS (ESI⁺) m/z: 473 [MH]⁺

The following compounds are prepared as shown in example 73/74:

### EXAMPLE 75

### 5-(2-{(E)-2-[4-(Morpholine-4-carbonyl)-phenyl]-vinyl}-pyridin-4-yl)-2-piperazin-1-yl-1H-pyrrole-3-carbonitrile

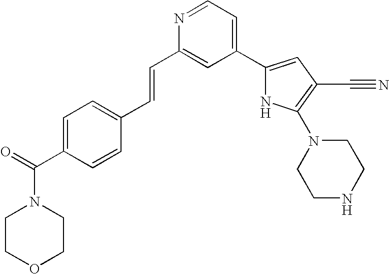

¹H-NMR (400 MHZ, DMSO-d6): 2.86 (m, 4H), 3.37 (m, 4H), 3.50-3.62 (bm, 8H), 7.05 (s, 1H), 7.28 (dd, 1H), 7.38-7.50 (m, 3H), 7.65-7.75 (m, 3H), 7.80 (s, 1H), 8.46 (d, 1H), 11.1 (bs, 1H).

MS (ES): 469 [MH]⁺.

### EXAMPLE 76

### 5-(2-{(E)-2-[4-(Morpholine-4-carbonyl)-phenyl]-vinyl}-pyridin-4-yl)-2-piperazin-1-yl-1H-pyrrole-3-carboxylic acid amide

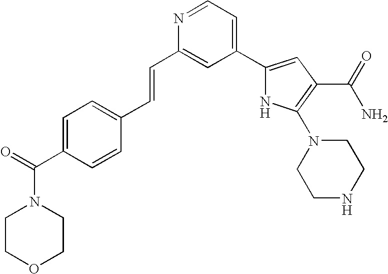

¹H-NMR (400 MHZ, DMSO-d6): 2.87 (m, 4H), 3.05 (m, 4H), 3.50-3.62 (bm, 8H), 6.92 (bs, 1H); 7.09 (s, 1H), 7.34 (d, 1H), 7.47 (m, 2H), 7.65 (bs, 1H), 7.74 (m, 2H), 7.85 (s, 1H), 8.47 (m, 1H), 11.39 (s, 1H).

MS (ES): 487 [MH]⁺.

### EXAMPLE 77

### 5-{2-[(E)-2-(4-(Methoxyphenyl)-vinyl]-pyridin-4-yl}-2-piperazin-1-yl-1H-pyrrole-3-carbonitrile

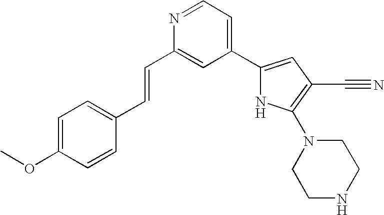

¹H-NMR (400 MHZ, DMSO-d6): 2.84 (m, 4H), 3.34 (m, 4H), 3.79 (s, 3H), 6.97 (m, 2H), 7.01 (s, 1H), 7.06 (d, 1H), 7.41 (m, 1H), 7.56-7.63 (m, 3H), 7.71 (d, 1H), 8.40 (d, 1H), 11.0 (bs, 1H).

MS (ES): 386 [MH]⁺.

### EXAMPLE 78

### 2-Piperazin-1-yl-5-[2-((E)-2-pyridin-4-yl)-vinyl)-pyridin-4-yl]-1H-pyrrole-3-carbonitrile

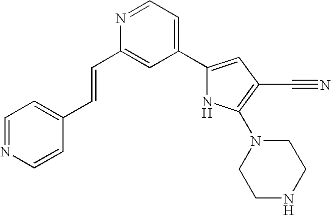

¹H-NMR (400 MHZ, DMSO-d6): 2.86 (m, 4H), 3.36 (m, 4H), 7.05 (s, 1H), 7.44-7.52 (m, 3H), 7.58-7.66 (m, 3H), 7.82 (d, 1H), 8.47 (d, 1H), 8.56 (d, 2H), 11.03 (bs, 1H).

MS (ES): 357 [MH]⁺.

### EXAMPLE 79

### 2-Piperazin-1-yl-5-[2-((E)-2-pyridin-4-yl-vinyl)-pyridin-4-yl]-1H-pyrrole-3-carboxylic acid amide

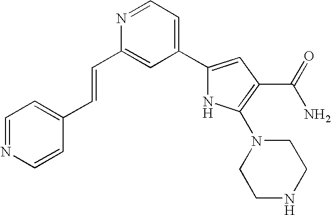

¹H-NMR (400 MHZ, DMSO-d6): 2.84 (m, 4H), 2.95-3.19 (m, 4H), 6.88 (bs, 2H), 7.08 (s, 1H), 11.37 (s, 1H)7.52 (m, 1H), 7.55 (m, 1H), 7.59-7.67 (m, 4H), 7.87 (s, 1H), 8.47 (d, 1H), 8.57 (m, 2H).

MS (ES): 375 [MH]⁺.

### EXAMPLE 80

### 2-Piperazin-1-yl-5-[2-((E)-2-pyridin-3-yl-vinyl)-pyridin-4-yl]-1H-pyrrole-3-carbonitrile

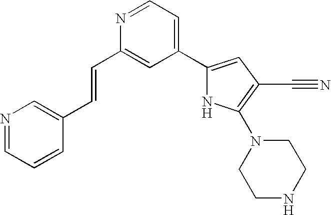

¹H-NMR (400 MHZ, DMSO-d6): 2.84 (m, 4H), 2.95-3.19 (m, 4H), 6.88 (bs, 2H), 7.08 (s, 1H), 11.37 (s, 1H), 7.52 (m, 1H), 7.55 (m, 1H), 7.59-7.67 (m, 4H), 7.87 (s, 1H), 8.47 (d, 1H), 8.57 (m, 2H).

MS (ES): 357 [MH]⁺.

### EXAMPLE 81

### 2-Piperazin-1-yl-5-[2-((E)-2-pyridin-3-yl-vinyl)-pyridin-4-yl]-1H-pyrrole-3-carboxylic acid amide

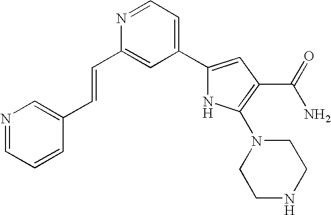

¹H-NMR (400 MHZ, DMSO-d6): 2.97 (m, 4H), 3.05-3.19 (m, 4H), 6.85 (bs, 2H), 7.10 (s, 1H), 7.37 (d, 1H). 7.40-7.50 (m, 2H), 7.70 (d, 1H), 7.84 (m, 1H), 8.10 (m, 1H), 8.46-8.51 (m, 2H), 8.82 (m, 1H), 11.34 (s, 1H).

MS (ES): 375 [MH]⁺.

### EXAMPLE 82

### 2-Piperazin-1-yl-5-[2-((E)-2-pyridin-2-yl-vinyl)-pyridin-4-yl]-1H-pyrrole-3-carbonitrile

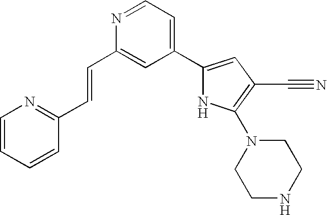

¹H-NMR (400 MHZ, DMSO-d6): 2.87 (m, 4H), 3.36 (m, 4H), 7.07 (s, 1H), 7.29 (m, 1H), 7.48 (m, 1H), 7.62 (m, 1H), 7.65-7.67 (m, 2H), 7.80 (dd, 1H), 7.87 (m, 1H), 8.46 (m, 1H), 8.60 (m, 1H), 10.97 (bs, 1H).

MS (ES): 357 [MH]⁺.

### EXAMPLE 83

### 2-Piperazin-1-yl-5-[2-((E)-2-pyridin-2-yl-vinyl)-pyridin-4-yl]-1H-pyrrole-3-carboxylic acid amide

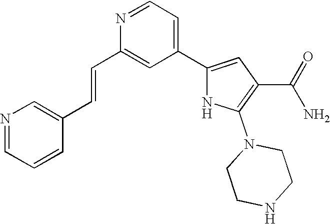

¹H-NMR (400 MHZ, DMSO-d6): 2.88 (m, 4H), 3.07 (m, 4H), 6.91 (bs, 2H), 7.12 (s, 1H), 7.31 (m, 1H), 7.53 (m, 1H), 7.63 (m, 1H), 7.68-7.71 (m, 2H), 7.82 (m, 1H), 7.93 (s, 1H), 8.61 (d, 1H), 11.35 (d, 1H).

MS (ES): 375 [MH]⁺.

### EXAMPLE 84

### N-Hydroxy-2-piperazin-1-yl-5-[2-((E)-styryl)-pyridin-4-yl]-1H-pyrrole-3-carboxamidine

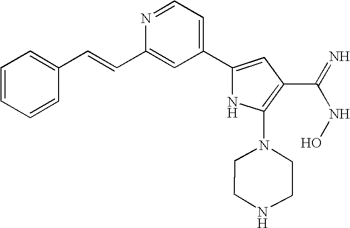

4-{3-Cyano-5-[2-((E)-styryl)-pyridin-4-yl]-1H-pyrrol-2-yl}-piperazine-1-carboxylic acid tert-butyl ester (50 mg; 0.11 mmol) is dissolved in 1 ml EtOH and 542 μl (8.2 mmol) NH₂OH solution (50% in H₂O) is added. The mixture the mixture is heated for 15 min at 140° C. in a microwave oven. Additional 200 μl NH₂OH solution is added and heating is repeated for 5 min at 140° C. to complete conversion. The reaction mixture is concentrated in vacuo. The residue is dissolved in 1 ml of 4 N HCl in doxane at rt and stirred for 1 h. The suspension is filtered to give red crystals.

¹H-NMR (400 MHZ, DMSO-d6): 3.27 (m, 4H), 3.56 (m, 4H), 7.27 (s, 1H), 7.40 (d, 1H), 7.45 (d, 2H), 7.49 (t, 2H), 7.49 (s, 1H), 7.57 (s, 1H), 7.66 (s, 1H), 7.92 (d, 1H), 8.31 (d, 1H), 8.49 (d, 1H), 8.85 (bs, 1H), 9.52 (s, 1H), 11.10 (s, 1H), 12.62 (s, 1H).

MS (ESI⁺) m/z: 389 [MH]⁺.

### EXAMPLE 85

### 5-(2-Phenethyl-pyridin-4-yl)-2-piperazin-1-yl-1H-pyrrole-3-carboxamidine

### a) 4-[3-Carbamimidoyl-5-(2-phenethyl-pyridin-4-yl)-1H-pyrrol-2-yl]-piperazine-1-carboxylic acid tert-butyl ester

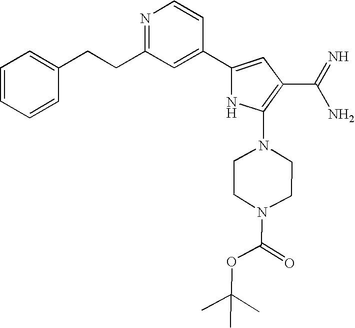

To a solution of N-hydroxy-2-piperazin-1-yl-5-[2-((E)-styryl)-pyridin-4-yl]-1H-pyrrole-3-carboxamidine (80 mg, 0.16 mmol) in 5 ml of AcOH is added zinc dust (214 mg, 3.28 mmol). The reaction mixture is heated to 70° C. for 15 h. After filtration and evaporation the product is purified by silica gel chromatography (ethyl acetate, MeOH gradient).

¹H-NMR (400 MHZ, DMSO-d6): 1.49 (s, 9H), 3.00 (m, 4H), 3.02 (m, 2H), 3.05 (m, 2H), 3.25-3.75 (m, 4H, NH), 3.54 (m, 4H), 7.00 (s, 1H), 7.14-7.19 (m, 1H), 7.22-7.28 (m, 4H), 7.34 (d, 1H), 7.41 (s, 1H), 8.24 (d, 1H).

MS (ESI⁺) m/z: 475 [MH]⁺.

### b) 5-(2-Phenethyl-pyridin-4-yl)-2-piperazin-1-yl-1H-pyrrole-3-carboxamidine

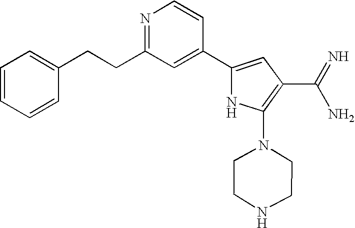

Deprotection of 4-[3-carbamimidoyl-5-(2-phenethyl-pyridin-4-yl)-1H-pyrrol-2-yl]-piperazine-1-carboxylic acid tert-butyl ester as described in example 8 yields the title compound.

¹H-NMR (400 MHZ, DMSO-d6): 3.15 (m, 2H), 3.26 (m, 2H), 3.35 (m, 10H, NH₂⁺), 7.21 (t, 1H), 7.28-7.31 (m, 4H), 7.77 (s, 1H), 8.01 (d, 1H), 8.28 (s, 1H), 8.61 (d, 1H), 8.72 (s, 1H, NH), 8.83 (s, 1H, NH), 9.47 (s, 1H, NH), 12.75 (s, 1H, NH).

MS (ESI⁻) m/z: 373 [M−H]⁻.

### EXAMPLE 86

### 2-(4-Methyl-piperazin-1-yl)-5-[2-((E)-styryl)-pyridin-4-yl]-1H-pyrrole-3-carboxylic acid amide

### a) 2-(4-Methyl-piperazin-1-yl)-5-[2-((E)-styryl)-pyridin-4-yl]-1H-pyrrole-3-carbonitrile

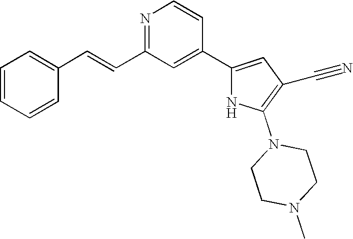

2-Piperazin-1-yl-5-[2-((E)-styryl)-pyridin-4-yl]-1H-pyrrole-3-carbonitrile hydrochloride (example 13, 100 mg, 0.26 mmol) is suspended in 5 ml of methanol and treated with 72 μl (1.28 mmol) AcOH, 58 μl (0.78 mmol) aqueous formaldehyde solution (37%) and NaBH₃CN (64 mg, 1.02 mmol). The reaction mixture is stirred for 17 h at room temperature, diluted with ethyl acetate and washed with saturated aqueous NaHCO₃ and brine. The organic layer is dried over Na₂SO₄ and concentrated. The residue is redisolved in ethyl acetate and treated with 1 ml of HCl in dioxane (4 M). The hydrochloride salt is filtered, washed with dioxane and dried under reduced pressure.

¹H-NMR (400 MHZ, DMSO-d6): 2.86 (s, 3H), 3.20-3.30 (m, 4H), 3.50-3.60 (m, 2H), 4.25-4.35 (m, 2H), 7.34 (d, 1H), 7.40-7.55 (m, 2H), 7.68 (d, 2H), 7.99 (d, 1H), 8.26 (d, 1H), 8.54 (d, 1H), 8.81 (s, 1H), 10.84 (s, 1H, NH⁺), 12.38 (s, 1H, NH).

MS (ESI⁺) m/z: 370 [MH]⁺.

### b) 2-(4-Methyl-piperazin-1-yl)-5-[2-((E)-styryl)-pyridin-4-yl]-1H-pyrrole-3-carboxylic acid amide

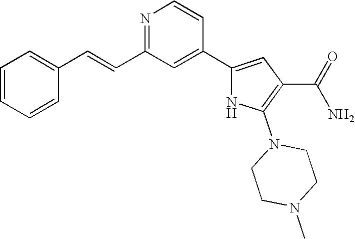

Prepared in a similar fashion as example 9 starting from 43 mg of 2-(4-methyl-piperazin-1-yl)-5-[2-((E)-styryl)-pyridin-4-yl]-1H-pyrrole-3-carbonitrile hydrochloride. Purification by reverse phase HPLC (Waters X-Terra, acetonitrile/water) yields the title compound.

¹H-NMR (400 MHZ, DMSO-d6): 2.89 (s, 3H), 3.15-3.75 (m, 8H), 6.92 (s, 2H), 7.25 (s, 1H), 7.29 (d, 1H), 7.36 (t, 1H), 7.45 (t, 2H), 7.51 (d, 1H), 7.69 (d, 2H), 7.73 (d, 1H), 7.84 (s, 1H), 8.51 (d, 1H), 11.32 (s, 1H, NH).

MS (ESI⁺) m/z: 388 [MH]⁺.

### EXAMPLE 87

### 4-{3-Cyano-5-[2-((E)-styryl)-pyridin-4-yl]-1H-pyrrol-2-yl}piperazine-1-carboxylic acid benzylamide

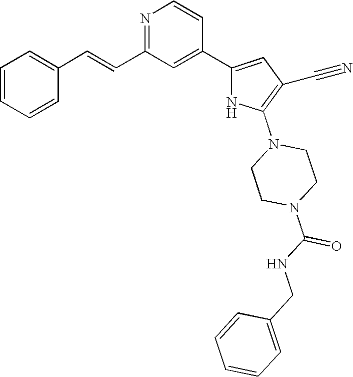

2-Piperazin-1-yl-5-[2-((E)-styryl)-pyridin-4-yl]-1H-pyrrole-3-carbonitrile hydrochloride (Example 13) (100 mg, 0.26 mmol) is dissolved in 3 ml of THF and treated with 130 μl (0.8 mmol) diisopropylethyl amine and 45 mg (0.34 mmol) benzylisocyanate. The reaction mixture is stirred for 17 h at room temperature, diluted with ethyl acetate and washed with water and brine. The organic layer is dried over Na₂SO₄ and concentrated. The residue is purified by silica gel chromatography (hexanes/ethyl acetate).

¹H-NMR (400 MHZ, DMSO-d6): 3.28-3.32 (m, 2H), 3.39-3.42 (m, 2H), 3.513.55 (m, 2H), 4.25-4.35 (m, 2H), 7.05 (s, 1H), 7.18-7.47 (m, 14H), 7.65 (s, 1H), 7.66 (d, 1H), 7.80 (s, 1H), 8.45 (d, 1H), 11.21 (s, 1H, NH).

MS (ESI⁺) m/z: 489 [MH]⁺.

### EXAMPLE 88

### 2-Piperazin-1-yl-5-(2-quinolin-3-yl-pyridin-4-yl)-1H-pyrrole-3-carboxamidine

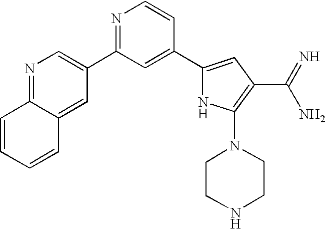

Prepared as described in example 85.

¹H-NMR (400 MHZ, DMSO-d6): 3.25-3.75 (m, 10H), 7.50 (s, 1H), 7.65 (d, 1H), 7.69 (t, 1H), 7.83 (t, 1H), 8.10 (d, 1H), 8.13 (d, 1H), 8.46 (s, 1H), 8.54 (s, 2H, NH), 8.75 (d, 1H), 9.07 (s, 1H), 9.66 (s, 1H), 12.27 (s, 1H, NH).

MS (ESI⁺) m/z: 398 [MH]⁺.

### EXAMPLE 89

### 2-(4-Formyl-piperazin-1-yl)-5-[2-(2-[1,4]oxazepan-4-yl-pyrimidin-5-yl)-pyridin-4-yl]-1H-pyrrole-3-carbonitrile

### a) 5-(2-Chloro-pyridin-4-yl)-2-piperazin-1-yl-1H-pyrrole-3-carbonitrile

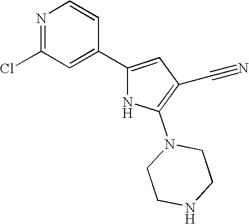

To 5 g of 4-[5-(2-chloro-pyridin-4-yl)-3-cyano-1H-pyrrol-2-yl]-piperazine-1-carboxylic acid tert-butyl ester dissolved in 20 ml dichloromethane are added 10 ml of trifluoroacetic acid. The mixture is stirred at room temperature overnight. After evaporation of the solvent and extraction with ethyl acetate/sat. sodium carbonate, the organic phase is dried to yield 7.24 g of an orange solid.

¹H-NMR (400 MHZ, DMSO-d6): 3.27 (m, 4H), 3.65 (m, 4H), 7.21 (m, 1H), 7.63 (dd, 1H), 7.77 (s, 1H), 8.27 (dd, 1H), 9.11 (bs, 2H, NH₂⁺), 11.48 (s, 1H, NH-pyrrole).

MS (ESI⁺) m/z: 288 [MH]⁺.

MS (ESI⁻) m/z: 286 [MH]⁻.

### b) 5-(2-Chloro-pyridin-4-yl)-2-(4-formyl-piperazin-1-yl)-1H-pyrrole-3-carbonitrile

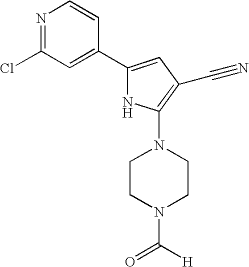

5-(2-Chloro-pyridin-4-yl-2-piperazin-1-yl-1H-pyrrole-3-carbonitrile hydrochloride (97 mg, 0.30 mmol) is dissolved in 1.5 ml of CH₂Cl₂ and after addition of 0.17 ml (1.50 mmol) N-methylmorpholine and 23 μl (0.60 mmol) formic acid, 2-chloro-4,6-dimethoxytriazine (105 mg, 0.60 mmol) and DMAP (3.7 mg, 0.03 mmol) the mixture is stirred for 15 min at 80° C. under microwave conditions. Then the mixture is diluted with ethyl acetate, washed with saturated NaHCO₃- and NaCl-solution, dried over Na₂SO₄ and evaporated. The crude product is purified by recrystallization from ethyl acetate.

¹H-NMR (400 MHZ, DMSO-d6): 3.45-3.55 (m, 8H), 6.95 (s, 1H), 7.42 (d, 1H), 7.50 (s, 1H), 8.02 (d, 1H), 8.04 (s, 1H), 11.14 (s, 1H, NH).

MS (ESI⁺) m/z: 316 [MH]⁺.

### c) 2-(4-Formyl-piperazin-1-yl)-5-[2-(2-[1,4]oxazepan-4-yl-pyrimidin-5-yl)-pyridin-4-yl]-1H-pyrrole-3-carbonitrile

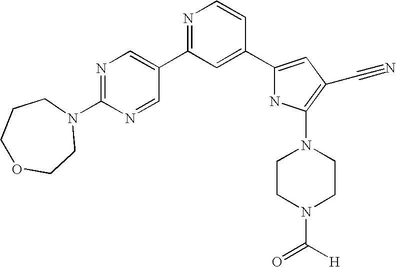

Prepared in a similar fashion as example 1e starting from 4-[5-(4,4,5,5-tetramethyl-[1,3,2]dioxaborolan-2-yl)-pyrimidin-2-yl]-[1,4]oxazepane and 5-(2-chloro-pyridin-4-yl-2-(4-formyl-piperazin-1-yl)-1H-pyrrole-3-carbonitrile.

¹H-NMR (400 MHZ, DMSO-d6): 1.86-1.91 (m, 2H), 3.45-3.96 (m, 16H), 7.48 (s, 1H), 7.69 (d, 1H), 8.09 (s, 1H), 8.16 (s, 1H), 8.54 (d, 1H), 8.99 (s, 2H), 11.25 (s, 1H, NH).

MS (ESI⁺) m/z: 459 [MH]⁺.

### EXAMPLE 90

### 5-[2-(4-Morpholin-4-yl-phenylamino)-pyridin-4-yl]-2-piperazin-1-yl-1H-pyrrole-3-carbonitrile hydrochloride

### a) 4-{3-Cyano-5-[2-(4-morpholin-4-yl-phenylamino)-pyridin-4-yl]-1H-pyrrol-2-yl}-piperazine-1-carboxylic acid tert-butyl ester

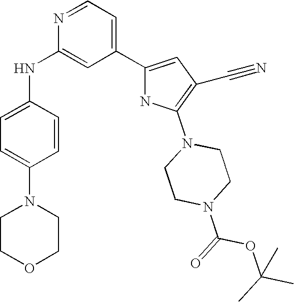

138 mg of 4-morpholinoaniline, 150 mg of 4-[5-(2-chloro-pyridin-4-yl)-3-cyano-1H-pyrrol-2-yl]-piperazine-1-carboxylic acid tert-butyl ester are dissolved in 6 ml DMF. The solution is degassed by introduction of a stream of argon. Pd₂(dba)₃ (7 mg), 7 mg of 2-dicyclohexylphosphino-2′,4′,6′-methoxybiphenyl and 314 mg of Cs₂CO₃ are added and the mixture is heated for 10 mn at 160° C. in a microwave oven. The entire reaction is duplicated 3 times, the combined reaction mixtures are extracted with ethyl acetate/water and dried over sodium sulfate. The resulting crude solid (713 mg) is purified by chromatography (acetonitrile/water).

¹H-NMR (400 MHZ, DMSO-d6): 1.43 (s, 9H), 3.01 (t, 4H), 3.35 (m, 4H), 3.47 (m, 4H), 3.73 (t, 4H), 6.73 (d, 1H), 6.83-6.88 (m, 6H), 7.45 (d, 2H), 8 (d, 1H), 8.64 (s, 1H), 11.21 (s, 1H).

MS (ESI⁺) m/z: 530[MH]⁺, MS (ESI⁻) m/z: 528[M−H]⁻.

### b) 5-[2-(4-Morpholin-4-yl-phenylamino)-pyridin-4-yl]-2-piperazin-1-yl-1H-pyrrole-3-carbonitrile hydrochloride

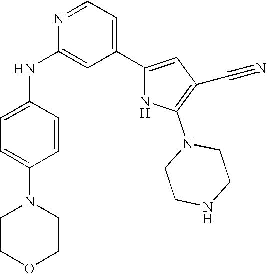

19 mg of 4-{3-cyano-5-[2-(4-morpholin-4-yl-phenylamino)-pyridin-4-yl]-1H-pyrrol-2-yl}-piperazine-1-carboxylic acid tert-butyl ester dissolved in 2 ml 4 M HCl in dioxane are stirred at room temperature overnight. The reaction mixture is dried in vacuo which yields the product as HCl salt.

¹H-NMR (400 MHZ, DMSO-d6): 3.2 (m, 4H), 3.3 (m, 4H), 3.7 (m, 4H), 3.8 (m, 4H), 7.1 (d, 2H), 7.20-7.25 (m, 4H), 7.3 (s, 1H), 7.8 (d, 1H), 9.3 (bs, 2H, NH₂⁺), 10.2 (bs, 1H, NH pyrrole), 11.2 (s, 1H, NH).

MS (ESI⁺) m/z: 4308 MH]⁺.

MS (ESI⁻) m/z: 428[M−H]⁻.

Following the procedure of example 90 the following compounds are synthesized:

### EXAMPLE 91

### 5-{2-[4-(Morpholine-4-carbonyl)-phenylamino]-pyridin-4-yl}-2-piperazin-1-yl-1H-pyrrole-3-carbonitrile trifluoroacetate

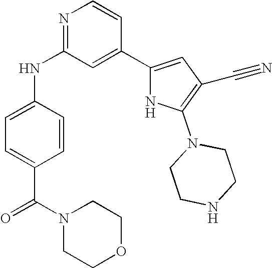

¹H-NMR (400 MHZ, DMSO-d6): 3.38 (m, 4H), 3.48 (m, 4H), 3.55 (m, 8H), 6.95 (s, 1H), 7.08 (m, 2H), 7.35 (d, 2H), 7.66 (d, 2H), 8.11 (d, 1H), 8.78 (bs, 2H, NH₂⁺), 9.48 (s, 1H, NH).

MS (ESI⁺) m/z: 458 [MH]⁺.

MS (ESI⁻) m/z: 456 [M−H]⁻.

### EXAMPLE 92

### 5-{2-[3-(Morpholine-4-sulfonyl)-phenylamino]-pyridin-4-yl}-2-piperazin-1-yl-1H-pyrrole-3-carbonitrile trifluoroacetate

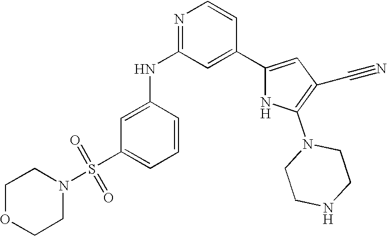

¹H-NMR (400 MHZ, DMSO-d6): 2.92 (m, 4H), 3.30 (m, 4H), 3.61 (m, 4H), 3.66 (m, 4H), 6.94 (s, 1H), 7.03 (s, 1H), 7.12 (dd, 1H), 7.25 (d, 1H), 7.55 (t,1H), 9.93 (dd, 1H), 8.11 (s, 1H), 8.14 (d, 1H), 8.83 (bs, 2H, NH₂⁺), 9.58 (s, 1H, NH), 11.43 (s, 1H, NH-pyrrole).

MS (ESI⁺) m/z: 494 [MH]⁺.

MS (ESI⁻) m/z: 492 [M−H]⁻.

### EXAMPLE 93

### 5-{2-[3-(Morpholine-4-sulfonyl)-phenylamino]-pyridin-4-yl}-2-piperazin-1-yl-1H-pyrrole-3-carboxylic acid amide

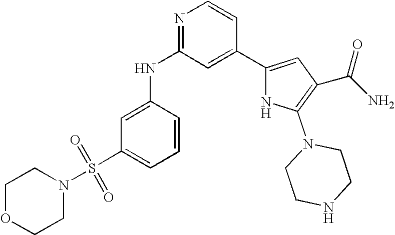

¹H-NMR (400 MHZ, DMSO-d6): 2.83 (m, 4H), 2.91 (m, 4H), 3.01 (m, 4H), 3.64 (m, 4H), 6.83 (d, 1H), 6.88 (bs, 1H, NH-amide), 7.00 (s, 1H), 7.12 (dd, 1H), 7.18 (d, 1H), 7.51 (t, 1H), 7.65 (bs 1H, NH-amide), 7.95 (d, 1H), 8.11 (d, 1H), 8.16 (m, 1H), 9.39 (s, 1H, NH), 11.36 (s, 1H, NH-pyrrole).

MS (ESI⁺) m/z: 512 [MH]⁺.

MS (ESI⁻) m/z: 510 [MH]⁻.

### EXAMPLE 94

### 5-{2-[4-(Morpholine-4-sulfonyl)-phenylamino]-pyridin-4-yl}-2-piperazin-1-yl-1H-pyrrole-3-carbonitrile trifluoroacetate

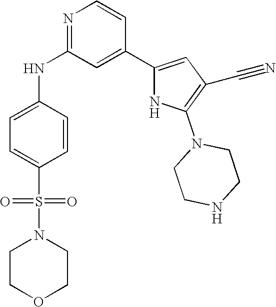

¹H-NMR (400 MHZ, DMSO-d6): 2.85 (m, 4H), 3.30 (m, 4H), 3.62 (m, 8H), 6.92 (d, 1H), 7.08 (s, 1H), 7.15 (dd, 1H), 7.62 (d, 2H), 7.88 (d, 2H), 8.19 (dd, 1H), 8.81 (bs, 2H, NH₂⁺), 9.68 (s, 1H, NH), 11.43 (s, 1H, NH-pyrrole).

MS (ESI⁺) m/z: 494 [MH]⁺.

MS (ESI⁻) m/z: 492 [M−H⁻.

### EXAMPLE 95

### 5-{2-[4-(Morpholine-4-sulfonyl)-phenylamino]-pyridin-4-yl}-2-piperazin-1-yl-1H-pyrrole-3-carboxylic acid amide

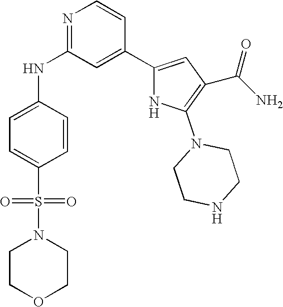

¹H-NMR (400 MHZ, DMSO-d6): 2.83 (m, 8H), 3.02 (m, 4H), 3.63 (m, 4H), 6.85 (s, 1H), 6.89 (bs, 1H, NH-amide), 7.08 (s, 1H), 7.15 (dd, 1H), 7.60 (d, 2H), 7.64 (bs, 1H, NH-amide) 7.90 (d, 2H), 8.15 (d, 1H), 9.55 (s, 1H—NH), 11.37 (s, 1H, NH-pyrrole).

MS (ESI⁺) m/z: 512 [MH]⁺.

MS (ESI⁻) m/z: 510 [MH]⁻.

### EXAMPLE 96

### 5-(2-Imidazol-1-yl-pyridin-4-yl)-2-piperazin-1-yl-1H-pyrrole-3-carbonitrile

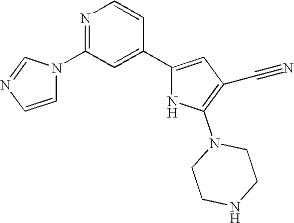

A solution of 110 mg (0.38 mmol) 5-(2-chloro-pyridin-4-yl)-2-piperazin-1-yl-1H-pyrrole-3-carbonitrile hydrochloride (example 89) and 189 mg (2.78 mmol) imidazole in 0.7 ml of NMP is heated to 240° C. for 45 min. under microwave conditions. The crude product is purified by reverse phase HPLC (Gilson, X-Terra, acetonitrile/water).

¹H-NMR (400 MHZ, DMSO-d6): 3.33 (m, 4H), 3.67 (m, 4H), 7.34 (d, 1H), 7.66 (s, 1H), 7.75 (d, 1H), 8.13 (s, 1H), 8.27 (s, 1H), 8.50 (d, 1H), 9.02 (s, 2H, NH₂⁺), 9.38 (s, 1H), 11.61 (s, 1H, NH).

MS (ESI⁺) m/z: 320 [MH]⁺.

### EXAMPLE 97

### 5-[2-(4-Phenyl-imidazol-1-yl)-pyridin-4-yl]-2-piperazin-1-yl-1H-pyrrole-3-carbonitrile

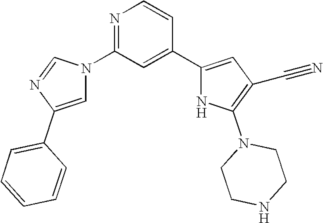

Prepared as described in example 96 starting from 5-(2-chloro-pyridin-4-yl)-2-piperazin-1-yl-1H-pyrrole-3-carbonitrile hydrochloride.

¹H-NMR (400 MHZ, DMSO-d6): 3.43 (m, 4H), 3.67 (m, 4H), 7.32 (t, 1H), 7.35 (s, 1H), 7.46 (d, 2H), 7.65 (d, 1H), 7.91 (d, 2H), 8.05 (s, 1H), 8.48 (d, 1H), 8.51 (s, 1H), 8.74 (s, 1H), 8.88 (s, 2H, NH₂⁺), 11.48 (s, 1H, NH).

MS (ESI⁺) m/z: 396 [MH]⁺.

### EXAMPLE 98

### 5-{2-[(E)-2-(4-Fluoro-phenyl)-vinyl]-pyridin-4-yl}-1-methyl-2-piperazin-1-yl-1H-pyrrole-3-carbonitrile trifluoroacetate

### a) 4-[5-(2-Chloro-pyridin-4-yl)-3-cyano-1-methyl-1H-pyrrol-2-yl]-piperazine-1-carboxylic acid tert-butyl ester

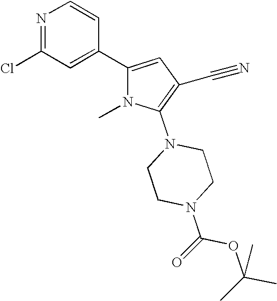

To 400 mg of 4-[5-(2-chloro-pyridin-4-yl)-3-cyano-1H-pyrrol-2-yl]-piperazine-1-carboxylic acid tert-butyl ester in 6 ml THF are added 60 mg of Sodium hydride and 0.077 ml of methyl iodide at 0° C. After 3 days at room temperature, the reaction is quenched with saturated sodium carbonate and extracted with dichloromethane. The resulting yellow solid is purified by flash chromatography (silica, ethyl acetate/cyclohehane 1/9).

¹H-NMR (400 MHZ, DMSO-d6): 1.44 (s, 9H), 3.23 (m, 4H), 3.51 (m, 4H), 3.57 (s, 3H), 6.90 (s, 1H), 7.50 (dd, 1H), 7.58 (bs, 1H), 8.39 (d, 1H).

MS (ESI⁺) m/z: 402 [MH]⁺, 424 [M+Na]⁺, 346 [M−C4 H8]⁺, 302 [M−Boc]⁺.

### b) 3-Cyano-5-{2-[(E)-2-(4-fluoro-phenyl)-vinyl]-pyridin-4-yl}-1-methyl-1H-pyrrol-2-yl)-piperazine-1-carboxylic acid tert-butyl ester

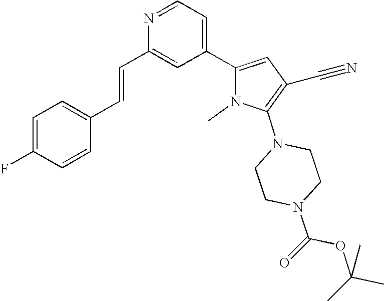

Prepared as shown in example 8 starting from trans-2(-4-fluoro-phenyl)-vinyl boronic acid (51 mg) and 4-[5-(2-chloro-pyridin-4-yl)-3-cyano-1-methyl-1H-pyrrol-2-yl]-piperazine-1-carboxylic acid tert-butyl ester to yield a yellow solid.

MS (ESI⁺) m/z: 488 [MM⁺.

MS (ESI⁻) m/z: 532 [M+HCOO]⁻.

### c) 5-{2-[(E)-2-(4-Fluoro-phenyl)-vinyl]-pyridin-4-yl}-1-methyl-2-piperazin-1-yl-1H-pyrrole-3-carbonitrile trifluoroacetate

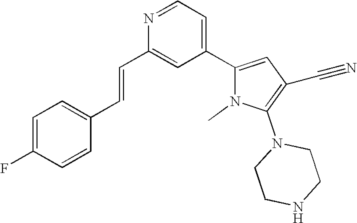

Prepared in a similar fashion as example 8 starting from 3-cyano-5-{2-[(E)-2-(4-fluoro-phenyl)-vinyl]-pyridin-4-yl}-1-methyl-1H-pyrrol-2-yl)-piperazine-1-carboxylic acid tert-butyl ester.

¹H-NMR (400 MHZ, DMSO-d6): 3.3 (m, 4H), 3.4 (m, 4H), 3.6 (s, 3H), 6.8 (s, 1H), 7.2-7.3 (m, 3H), 7.3 (dd, 1H), 7.6 (bs, 1H), 7.7 (m, 3H), 8.6 (d, 1H), 8.3-8.7 (bs, 2H, NH₂⁺).

MS (ESI⁺) m/z: 388 [MH]⁺.

### EXAMPLE 99

### 5-{2-[(E)-2-(4-Fluoro-phenyl)-vinyl]-pyridin-4-yl}-2-(4-methanesulfonyl-piperazin-1-yl)-1H-pyrrole-3-carbonitrile

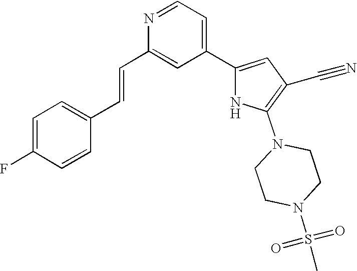

To 38 mg of 5-{2-[(E)-2-(4-fluoro-phenyl)-vinyl]-pyridin-4-yl}-2-piperazin-1-yl-1H-pyrrole-3-carboxylic acid amide in pyridine are added 0.008 ml of methanesulfonyl chloride and 0.050 ml of N-ethyldiisopropylamine. After one week the reactants are added again twice. The solvent is evaporated, the residue extracted with ethyl acetate/water and dried over sodium sulfate. The resulting residue is purified by HPLC (acetonitril/water) to yield a yellow solid.

¹H-NMR (400 MHZ, DMSO-d6): 2.96 (s, 3H), 6.92 (bs, 1H, CONH₂), 7.10 (s, 1H) 7.22-7.25 (m, 3H), 7.45 (bs, 1H), 7.46 (dd, 1H), 7.68-7.78 (m 3H), 8.45 (d, 1H), 11.37 (bs, 1H, NH).

MS (ESI⁺) m/z: 470 [MH]⁺.

MS (ESI⁻) m/z: 468 [M−H]⁻

### EXAMPLE 100

### 5-{2-[(E)-2-(4-Fluoro-phenyl)-vinyl]-pyridin-4-yl}-2-(4-methanesulfonyl-piperazin-1-yl)-1H-pyrrole-3-carboxylic acid amide

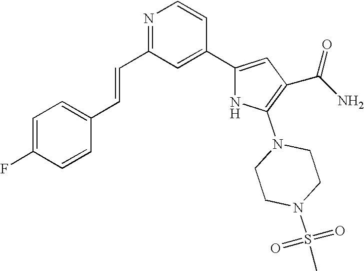

To 38 mg of 5-{2-[(E)-2-(4-fluoro-phenyl)-vinyl]-pyridin-4-yl}-2-piperazin-1-yl-1H-pyrrole-3-carboxylic acid amide (example 9) dissolved in pyridine are added 8.3 μl of methane sulfonyl chloride and 50 μl of N-ethyldisopropylamine. The reagents are added again twice with a trace of DMAP and left at room temperature. The reaction mixture is evaporated. The residue is dissolved in ethyl acetate, extracted with water, dried over sodium sulfate and purified by HPLC (Gilson) to yield the title compound as a yellow solid.

¹H-NMR (400 MHZ, DMSO-d6): 2.96 (s, 3H), 3.22 (m, 4H), 3.29 (m, 4H), 6.92 (s, 1H, NH-amide), 7.10 (s, 1H, NH-amide), 7.22 (m, 3H), 7.45 (s, 1H, NH-amide) 7.46 (dd, 1H), 7.70 (m, 3H), 8.45 (d, 1H), 11.37 (s, 1H-pyrrole).

MS (ESI⁺) m/z: 470 [MH]⁺.

MS (ESI⁻) m/z: 468 [MH]⁻.

### EXAMPLE 101

### 4-(3-Carbamoyl-5-{2-[(E)-2-(4-morpholin-4-ylmethyl-phenyl)-vinyl]-pyridin-4-yl}-1H-pyrrol-2-yl)-piperazine-1-carboxylic acid benzyl ester

### a) 5-(2-Chloro-pyridin-4-yl)-2-piperazin-1-yl-1H-pyrrole-3-carboxylic acid amide

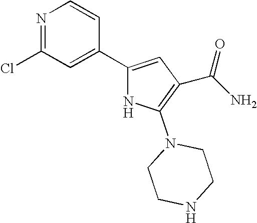

To 1.0 g of 5-(2-chloro-pyridin-4-yl)-2-piperazin-1-yl-1H-pyrrole-3-carbonitrile (example 88) are added 4 ml of conc. sulfuric acid. The mixture is stirred at room temperature for several days. The reaction mixture is poured on ice, basified to pH 9 and the water evaporated. The product is used as such for the next step.

### b) 4-[3-Carbamoyl-5-(2-chloro-pyridin-4-yl)-1H-pyrrol-2-yl]-piperazine-1-carboxylic acid benzyl ester

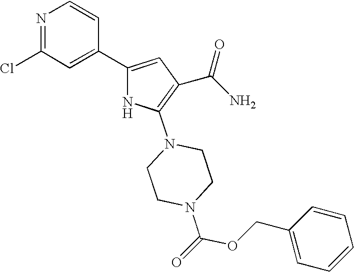

To 1.0 g of 5-(2-chloro-pyridin-4-yl)-2-piperazin-1-yl-1H-pyrrole-3-carboxylic acid amide in 1 ml water are added 1.31 ml of a 50% solution of benzyl chioroformate in toluene and potassium carbonate. After 2 days of stirring the reaction mixture is diluted with dichloromethane and extracted with water. The organic fraction is evaporated, dried and the residue purified by HPLC (Gilson-RP-18, acetonitrile/water).

¹H-NMR (400 MHZ, DMSO-d6): 3.10 (m, 4H), 3.58 (m, 4H), 5.12 (s, 2H), 6.89 (bs, 1H, NH-amide), 7.18 (s, 1H), 7.30-7.37 (m, 5H), 7.43 (s, 1H, NH-amide), 7.57 (dd, 2H), 7.73 (s, 1H), 8.24 (m, 1H), 11.30 (s, 1H, NH pyrrole).

MS (ESI⁺) m/z: 440 [MH]⁺.

### d) 4-(3-Carbamoyl-5-{2-[(E)-2-(4-morpholin-4-ylmethyl-phenyl)-vinyl]-pyridin-4-yl}-1H-pyrrol-2-yl)-piperazine-1-carboxylic acid benzyl ester

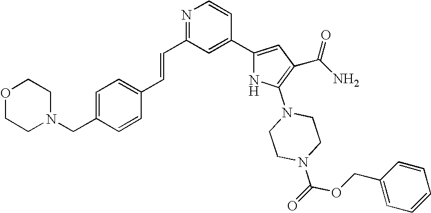

200 mg of 4-[3-carbamoyl-5-(2-chloro-pyridin-4-yl)-1H-pyrrol-2-yl]piperazine-1-carboxylic acid benzyl ester are dissolved in 3 ml n-propanol and degassed .After addition of 4-{4-[(E)-2-(4,4,5,5-tetramethyl-[1,3,2]dioxaborolan-2-yl)-vinyl]-benzyl}-morpholine (225 mg), bis(triphenylphosphine)palladium(II)chloride (15 mg), 0.5 ml of a 2 N solution of sodium carbonate the mixture is heated at 145° C. during 15 min. After filtration over celite, dilution with dichloromethane, water extraction, dessication over sodium sulfate, the 192 mg residue is purified by flash chromatoghaphy (silica gel: ethylacetate/hexane 1:9) followed by a HPLC (Gilson RP-18 acetonitrile/water) as final purification.

¹H-NMR (400 MHZ, DMSO-d6): 2.36 (m, 4H), 3.11 (m, 4H), 3.48 (s, 2H), 3.58 (m, 8H), 5.13 (s, 2H), 6.89 (bs, 1H, NH-amide), 7.09 (s, 1H), 7.21 (d, 1H), 7.33-7.37 (m, 7H), 7.45 (dd, 1H), 7.50 (s, 1H, NH-amide), 7.59 (d, 2H), 7.68 (d, 1H), 7.78 (s, 1H), 8.43 (d, 1H).

MS (ESI⁺) m/z: 607 [MH]⁺.

MS (ESI⁻) m/z: 605 [MH]⁻.

### EXAMPLE 102

### 2-Piperazin-1-yl-5-(6′-pyrrolidin-1-yl-[2,3′bipyridinyl-4-yl)-1H-pyrrole-3-carbonitrile trifluoroacetate

### a) 4-[3-Cyano-5-(6′-pyrrolidin-1-yl-[2,3′bipyridinyl-4-yl)-1H-pyrrol-2-yl]-piperazine-1-carboxylic acid tert-butyl ester

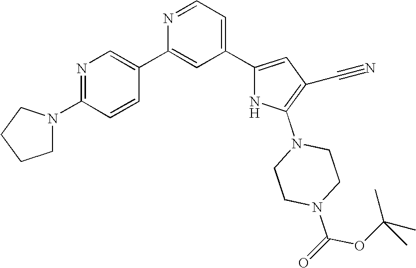

2-Pyrrolidin-1-yl-5-(4,4,5,5-tetramethyl-[1,3,2]dioxaborolan-2-yl)-pyridine (425 mg) and 300 mg of 4-[5-(2-chloro-pyridin-4-yl)-3-cyano-1H-pyrrol-2-yl]-piperazine-1-carboxylic acid tert-butyl ester are dissolved in 8 ml n-propanol. The solution is degassed by introduction of a stream of argon. Pd(PPh₂)₂Cl₂ (55 mg) and 1 ml of 2 N Na₂CO₃ are added and the mixture is heated for 15 min at 145° C. in a microwave oven. The reaction mixture is extracted with ethyl acetate/water/brine and died over sodium sulfate. After evaporation of the solvent the residue is purified by reverse phase HPLC (acetonitrile/water) and yields a yellow solid.

¹H-NMR (400 MHZ, DMSO-d6): 1.43 (s, 9H), 1.97 (m, 4H),3.38-3.45 (m, 12H), 6.54 (d, 1H), 7.13 (d, 1H), 7.39 (dd, 1H), 8.02 (s, 1H), 8.18 (dd, 1H), 8.43 (d, 1H), 8.87 (d, 1H), 11.16 (s, 1H).

MS (ESI⁺) m/z: 500 [MH]⁺.

### b) 2-Piperazin-1-yl-5-(6′-pyrrolidin-1-yl-[2,3′]bipyridinyl-4-yl)-1H-pyrrole-3-carbonitrile trifluoroacetate

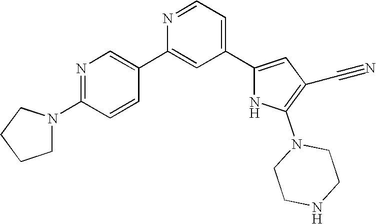

Prepared as described in example 8 starting from 212 mg of 4-[3-cyano-5-(6′-pyrrolidin-1-yl-[2,3′]bipyridinyl-4-yl)-1H-pyrrol-2-yl]-piperazine-1-carboxylic acid tert-butyl ester.

¹H-NMR (400 MHZ, DMSO-d6): 2.03 (m, 4H), 3.32 (m, 4H), 3.56 (m, 4H), 3.69 (m, 4H), 6.94 (d, 1H), 7.53 (s, 1H), 7.71 (d, 1H), 8.21 (s, 1H), 8.34 (d, 1H), 8.58 (d, 1H), 8.69 (d, 1H), 9.00 (bs, 2H), 11.16 (s, 1H).

MS (ESI⁺) m/z: 400 [MH]⁺.

MS (ESI⁻) m/z: 398 [M−H]⁻.

### EXAMPLE 103

### 2-Piperazin-1-yl-5-(6′-pyrrolidin-1-yl-[2,3′]bipyridinyl-4-yl)-1H-pyrrole-3-carboxylic acid amide

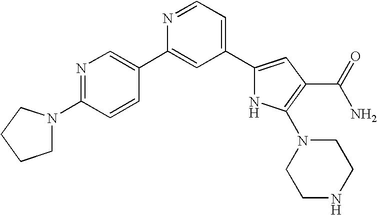

Prepared in analogy to example 9 starting from 50 mg of 2-piperazin-1-yl-5-(6′-pyrrolidin-1-yl-[2,3′]bipyridinyl-4-yl)-1H-pyrrole-3-carbonitrile trifluoroacetate. The crude product is purified by HPLC (reverse phase, acetonitrile/water).

¹H-NMR (400 MHZ, DMSO-d6): 1.97 (m, 4H), 2.85 (m, 4H),3.03 (m, 4H), 3.46 (m, 4H), 6.55 (d, 1H), 6.88 (bs, 1H, CONH₂), 7.11 (s, 1H), 7.43 (dd, 1H), 7.64 (bs, 1H), 8.17 (d, 1H), 8.21 (dd, 1H), 8.42 (d, 1H), 8.87 (d, 1H), 11.30 (s, 1H).

MS (ESI⁺) m/z: 418 [MH]⁺.

MS (ESI⁻) m/z: 416 [M−H]

### EXAMPLE 104

### 5-(2-{2-[(2-Methoxy-ethyl)-methyl-amino]-pyrimidin-5-yl}-pyridin-4-yl)-2-piperazin-1-yl-1H-pyrrole-3-carbonitrile trifluoroacetate

### a) 4-[3-Cyano-5-(2-{2-[(2-methoxy-ethyl)-methyl-amino]-pyrimidin-5-yl}-pyridin-4-yl)-1H-pyrrol-2-yl]-piperazine-1-carboxylic acid tert-butyl ester

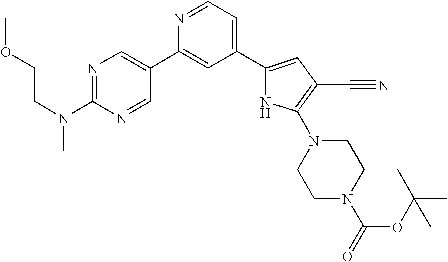

(2-Methoxy-ethyl)-methyl-[5-(4,4,5,5-tetramethyl-[1,3,2]dioxaborolan-2-yl)-pyrimidin-2-yl]-amine (604 mg) and 400 mg of 4-[5-(2-chloro-pyridin-4-yl)-3-cyano-1H-pyrrol-2-yl]-piperazine-1-carboxylic acid tert-butyl ester are dissolved in 3 ml n-propanol. The solution is degassed by introduction of a stream of argon. Pd(PPh₂)₂Cl₂ (72 mg) and 1.29 ml of 2 N Na₂CO₃ are added and the mixture is heated for 15 mn at 145° C. in a microwave oven. The reaction mixture is extracted with ethylacetate/water/brine and died over sodium sulfate.

After evaporation of the solvent the residue is purified by normal phase HPLC (ethyl acetate/cyclohexane).

¹H-NMR (400 MHZ, DMSO-d6): 1.44 (s, 9H), 3.21 (s, 3H), 3.27 (s, 3H), 3.40 (m, 4H), 3.50 (m, 4H), 3.56 (t, 2H), 3.84 (t, 2H), 7.16 (s, 1H), 7.46 (dd, 1H), 8.05 (s, 1H), 7.47 (d, 1H), 9.03 (s, 2H), 11.13 (s, 1H, NH).

MS (ESI⁺) m/z: 519 8 MH]⁺.

MS (ESI⁻) m/z: 517 [M−H]⁻

### b) 5-(2-{2-[(2-Methoxy-ethyl)-methyl-amino]-pyrimidin-5-yl}-pyridin-4-yl)-2-piperazin-1-yl-1H-pyrrole-3-carbonitrile trifluoroacetate

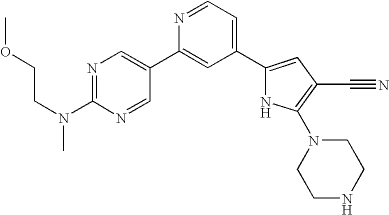

201 mg of 4-[3-cyano-5-(2-{2-[(2-methoxy-ethyl)-methyl-amino]-pyrimidin-5-yl}-pyridin-4-yl)-1H-pyrrol-2-yl]-piperazine-1-carboxylic acid tert-butyl ester are stirred overnight with 2 ml trifluoroacetic acid in 2 ml dichloromethane. Evaporation of the solvent yields the title compound.

¹H-NMR (400 MHZ, DMSO-d6): 3.22 (s, 3H), 3.26 (s, 3H), 3.32 (m, 4H), 3.57 (t, 2H), 3.66 (m, 4H), 3.86 (t, 2H), 7.39 (s, 1H), 7.62 (dd, 1H), 8.13 (s, 1H), 8.53 (d, 1H), 8.86 (bs, 2H, NH₂⁺), 9.00 (s, 2H), 11.40 (s, 1 H, NH).

MS (ESI⁺) m/z: 419 [MH]⁺.

MS (ESI⁻) m/z: 417 [M−H]⁻

### EXAMPLE 105

### 5-[6′-(1-Methyl-piperidin-4-yloxy)-[2,3′]bipyridinyl-4-yl]-2-piperazin-1-yl-1H-pyrrole-3-carbonitrile trifluoroacetate

### a) 5-Bromo-2-(1-methyl-piperidin-4-yloxy)-pyridine . . .

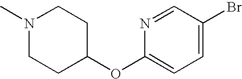

To a solution of 5-bromo-2-pyridone (2.0 g, 11.5 mmol), 4-hydroxy-methylpiperidine (1.3 g, 11.5 mmol) and triphenylphosphine (3.6 g, 13.8 mmol) in 50 ml THF is added dropwise DEAD (2.2 ml, 13.8 mmol) at room temperature. The solution is stirred at room temperature for 16 hours, diluted with ethyl acetate (300 ml) and extracted with 1 N HCl. The combined aquous phase is brought to pH 9 using Na₂CO₃ and then extracted with ethyl acetate. The crude product is purified by silica gel chromatography (ethyl acetate/methanol) to yield the title compound.

¹H-NMR (400 MHZ, DMSO-d6): 1.60-1.72 (m, 2H), 1.90-2.00 (m, 2H), 2.08-2.19 (m, 2H), 2.19 (s, 3H), 2.60-2.68 (m, 2H), 4.92 (quint., 1H), 6.78 (d, 1H), 7.87 (dd, 1H), 8.25 (d, 1H).

MS (ESI⁺) m/z: 271/273 [MH]⁺.

### b) 2-(1-Methyl-piperidin-4-yloxy)-5-(4,4,5,5-tetramethyl-[1,3,2]dioxaborolan-2-yl)-pyridine

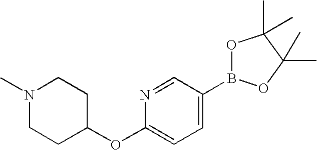

To a solution of 5-bromo-2-(1-methyl-piperidin-4-yloxy)-pyridine (3.1 g, 11.4 mmol) in THF (100 ml) at −78° C. is added dropwise n-BuLi (8.6 ml, 1.6M solution in hexanes, 13.7 mmol). The reaction mixture is stirred at −78° C. for 30 minutes, then 2-isopropoxy-4,4,5,5-tetramethyl[1,3,2]dioxaborolane (2.6 g, 13.7 mmol) is added. The reaction mixture is kept at −78° C. for 2 hours then warmed gradually to room temperature. Saturated NH₄Cl solution is added and the mixture is extracted with ethyl acetate. After removal of the solvent 2.8 g of the title compound is obtained, which is used in the following reaction steps without further purification.

MS (ESI⁺) m/z: 319 [MH]⁺.

### c) 5-[6′-(1-Methyl-piperidin-4-yloxy)-[2,3′]bipyridinyl-4-yl]-2-piperazin-1-yl-1H-pyrrole-3-carbonitrile-trifluoroacetate

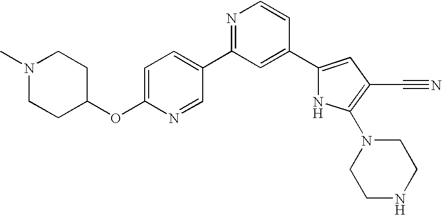

The title compound is obtained via Suzuki coupling and BOC-deprotection as described previously (example 8).

¹H-NMR (400 MHZ, DMSO-d6): 1.75-1.90 (m, 1H), 2.00-2.22 (m, 2H), 2.30-2.40 (m, 1H), 2.85 (dd, 3H), 3.15-3.60 (m, 12H), 5.38 (bs, 1H), 6.98 (t, 1H), 7.30 (s, 1H), 7.59 (d, 1H), 8.15 (d, 1H), 8.39 (ddd, 1H), 8.56 (d, 1H), 8.83 (bs, 1H), 8.90 (d, 1H), 11.42 (s, 1H).

MS (ESI⁺) m/z: 444 [MH]⁺.

Similarly the following compounds are prepared:

### EXAMPLE 106

### 5-(6′-Morpholin-4-yl-[2,3′]bipyridinyl-4-yl)-2-piperazin-1-yl-1H-pyrrole-3-carbonitrile trifluoroacetate

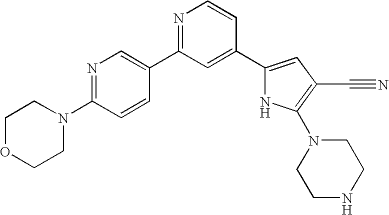

¹H-NMR (400 MHZ, DMSO-d6): 3.30 (m, 4H), 3.62 (m, 4H), 3.71 (m, 8H), 7.05 (d, 1H), 7.59 (s, 1H), 7.71 (d, 1H), 8.29 (m, 2H), 8.52 (d, 1H), 8.83 (d, 1H), 8.92 (s, 2H, NH₂⁺), 11.05 (s, 1H, NH).

MS (ESI⁺) m/z: 416 [MH]⁺.

MS (ESI⁻) m/z: 414 [MH]⁻.

### EXAMPLE 107

### 5-[2-(2-Morpholin-4-yl-pyrimidin-5-yl)-pyridin-4-yl]-2-piperazin-1-yl-1H-pyrrole-3-carbonitrile trifluoroacetate

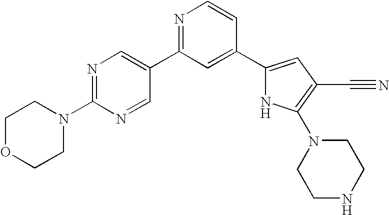

¹H-NMR (400 MHZ, DMSO-d6): 3.31 (m, 4H), 3.65 (m, 4H), 3.72 (m, 4H), 3.81 (m, 4H), 7.35 (s, 1H), 7.58 (d, 1H), 8.12 (s, 1H), 8.53 (d, 1H), 8.72 (s, 2H, NH₂⁺), 9.06 (s, 2H).

MS (ESI⁺) m/z: 417 [MH]⁺.

MS (ESI⁻) m/z: 415 [MH]⁻.

### EXAMPLE 108

### 5-(6′-Morpholin-4-yl-[2,3′]bipyridinyl-4-yl)-2-piperazin-1-yl-1H-pyrrole-3-carboxylic acid amide

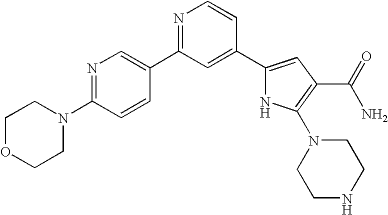

¹H-NMR (400 MHZ, DMSO-d6): 2.85 (m, 4H), 3.04 (m, 4H), 3.55 (m, 4H), 3.72 (m, 4H), 6.89 (s, 1H, NH-amide), 6.93 (d, 1H), 7.14 (s, 1H), 7.48 (dd, 1H), 7.63 (s, 1H, NH-amide), 8.11 (s, 1H), 8.30 (dd, 1H), 8.44 (d, 1H), 8.92 (d, 1H), 11.30 (s, 1H, NH).

MS (ESI⁺) m/z: 434 [MH]⁺.

MS (ESI⁻) m/z: 432 [MH]⁻.

### EXAMPLE 109

### 2-Piperazin-1-yl-5-(3,4,5,6-tetrahydro-2H-[1,2′;5′,2″]terpyridin-4″-yl)-1H-pyrrole-3-carboxylic acid amide

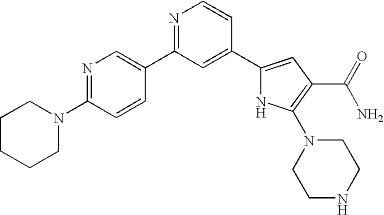

¹H-NMR (400 MHZ, DMSO-d6): 1.54 (m, 4H), 1.63 (m, 2H), 2.85 (m, 4H), 3.05 (m, 4H), 3.61 (m, 4H), 6.89 (s, 1H, NH-amide), 6.89 (d, 1H), 7.12 (s, 1H), 7.45 (d, 1H), 7.63 (s, 1H, NH-amide), 8.08 (s, 1H), 8.21 (dd, 1H), 8.43 (d, 1H), 8.89 (s, 1H).

MS (ESI⁺) m/z: 432 [MH]⁺.

MS (ESI⁻) m/z: 430 [MH]⁻.

### EXAMPLE 110

### 5-[6′-(4-Methyl-piperazin-1-yl)-[2,3′]bipyridinyl-4-yl]-2-piperazin-1-yl-1H-pyrrole-3-carboxylic acid amide

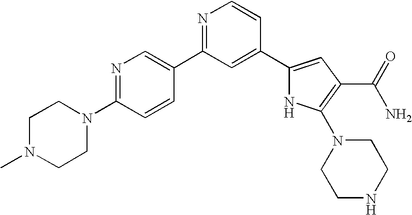

¹H-NMR (400 MHZ, DMSO-d6): 2.23 (s, 3H), 2.42 (m, 4H), 2.84 (m, 4H), 3.05 (m, 4H), 3.58 (m, 4H), 6.88 (s, 1H, NH-amide), 6.94 (d, 1H), 7.13 (s, 1H), 7.47 (d, 1H), 7.63 (s, 1H, NH-amide), 8.10 (s, 1H), 8.26 (dd, 1H), 8.44 (d, 1H), 8.90 (s, 1H).

MS (ESI⁺) m/z: 447 [MH]⁺.

MS (ESI⁻) m/z: 445 [MH]⁻.

### EXAMPLE 111

### 5-{2-[2-(4-Methyl-piperazin-1-yl)-pyrimidin-5-yl]-pyridin-4-yl}-2-piperazin-1-yl-1H-pyrrole-3-carbonitrile bis trifluoroacetate

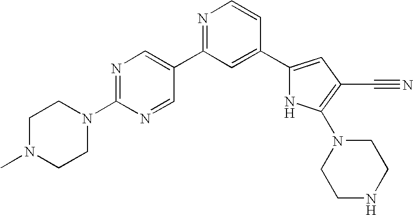

¹H-NMR (400 MHZ, DMSO-d6): 2.86 (s, 3H), 3.08 (m, 2H), 3.31 (m, 6H), 3.55 (m, 2H), 3.64 (m, 4H), 4.70 (m, 2H), 7.30 (d, 1H), 7.59 (dd, 1H), 8.13 (s, 1H), 8.56(d, 1H), 8.86 (bs, 2H), 9.13 (s, 2H), 9.90 (bs, 1H), 11.41 (s, 1H).

MS (ESI⁺) m/z: 430 [MH]⁺.

MS (ESI⁻) m/z: 428 [MH]⁻.

### EXAMPLE 112

### 5-{2-[2-(4-Methyl-piperazin-1-yl)-pyrimidin-5-yl]-pyridin-4-yl}-2-piperazin-1-yl-1H-pyrrole-3-carboxylic acid amide

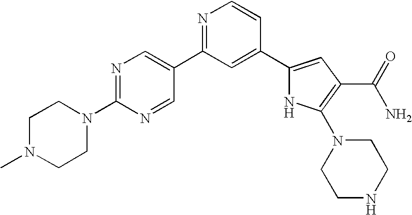

¹H-NMR (400 MHZ, DMSO-d6): 2.23 (s, 3H), 3.38 (m, 4H), 2.94 (m, 4H), 3.12 (m, 4H) 3.84 (m, 4H), 6.33 (bs, 1H, NH-amide), 7.19 (d, 1H), 7.50 (d, 1H), 7.53 (bs, 1H, NH-amide), 8.10 (s, 1H), 8.47 (dd, 1H), 9.07 (s, 2H), 11.23 (s, 1H).

MS (ESI⁺) m/z: 446 [MH]⁺.

MS (ESI⁻) m/z: 448 [MH]⁻.

### EXAMPLE 113

### 5-[6′-(4-Cyclopentyl-piperazin-1-yl)-[2,3′]bipyridinyl-4-yl]-2-piperazin-1-yl-1H-pyrrole-3-carbonitrile trifluoroacetate

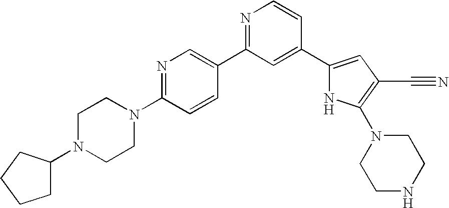

¹H-NMR (400 MHZ, DMSO-d6): 1.50 (m, 2H), 1.74 (m, 4H), 2.05 (m, 2H), 3.12 (m, 2H), 3.20-3.30 (m, 5H), 3.71 (m, 8H), 4.58 (d, 2H), 7.14 (d, 1H), 7.48 (s, 1H), 7.68 (d, 1H), 8.19 (s, 1H), 8.27 (dd, 1H), 8.54 (d, 1H), 8.89 (d, 1H), 8.98 (bs, 2H, NH₂⁺), 9.55 (bs, 1H, NH⁺), 11.56 (s, 1H, NH).

MS (ESI⁺) m/z: 483 [MH]⁺.

MS (ESI⁻) m/z: 481 [MH]⁻.

### EXAMPLE 114

### 5-[6′-(4-Methyl-[1,4]diazepan-1-yl)-[2,3′]bipyridinyl-4-yl]-2-piperazin-1-yl-1H-pyrrole-3-carbonitrile trifluoroacetate

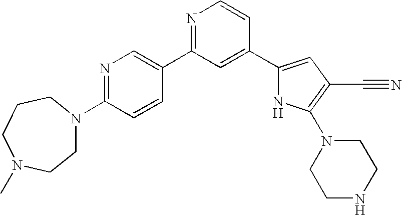

¹H-NMR (400 MHZ, DMSO-d6): 2.20 (m, 2H), 2.85 (s, 3H), 3.21 (m, 2H) 3.31 (m, 4H), 3.21-3.71 (m, 11H), 6.94 (d, 1H), 7.6 (s, 1H), 7.71 (d, 1H), 8.21 (s, 1H), 8.24 (d, 1H), 8.57 (d, 1H), 8.88 (d, 1H), 8.99 (bs, 2H, NH₂⁺), 9.84 (bs, 1H), 11.63 (s, 1H, NH).

MS (ESI⁺) m/z: 443 [MH]⁺.

MS (ESI⁻) m/z: 441 [MH]⁻.

### EXAMPLE 115

### 5-[6′-(4-Benzyl-piperazin-1-yl)-[2,3′]bipyridinyl-4-yl]-2-piperazin-1-yl-1H-pyrrole-3-carbonitrile hydrochloride

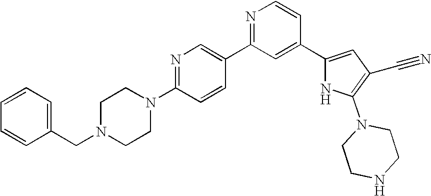

¹H-NMR (400 MHZ, DMSO-d6): 3.23 (m, 4H), 3.30 (t, 4H), 3.46-3.55 (m, 4H), 3.83 (t, 4H), 4.31 (s, 2H), 7.02 (d, 1H), 7.23 (s, 1H), 7.45 (m, 3H), 7.63-7.67 (m, 3H), 8.37 (s, 1H), 8.40 (dd, 1H), 8.44 (d, 1H), 8.98 (d, 1H), 11.87 (s, 1H, NH).

MS (ESI⁺) m/z: 505 [MH]⁺.

### EXAMPLE 116

### 5-[6′-(4-Benzyl-piperazin-1-yl)-[2,3′]bipyridinyl-4-yl]-2-piperazin-1-yl-1H-pyrrole-3-carboxylic acid amide

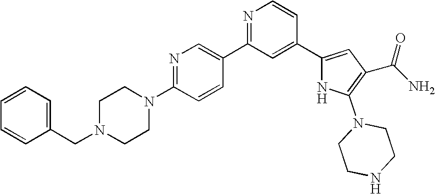

¹H-NMR (400 MHZ, DMSO-d6): 2.86 (m, 4H), 3.05 (m, 4H), 3.30 (m, 4H), 3.53 (s, 2H), 3.58 (m, 4H), 6.87 (bs, 1H, NH-amide), 7.91 (d, 1H), 7.12 (s, 1E), 7.35 (m, 7H), 7.46 (dd, 1H), 7.64 (bs, NH-amide), 8.09 (s, 1H), 8.24 (dd, 1H), 8.43 (m, 1H), 8.89 (s, 1H), 11.31 (s, 1H, NH).

MS (ESI⁺) m/z: 523 [MH]⁺.

### EXAMPLE 117

### 5-[6′-(4-Cyclopentyl-piperazin-1-yl)-[2,3′]bipyridinyl-4-yl]-2-piperazin-1-yl-1H-pyrrole-3-carboxylic acid amide

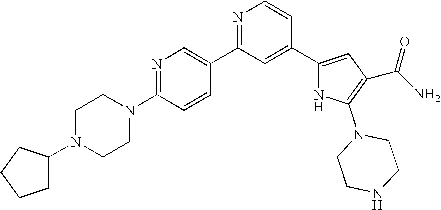

¹H-NMR (400 MHZ, DMSO-d6): 1.58 (m, 2H), 1,74 (m, 4H), 2.10 (m, 2H), 3.12 (m, 2H), 3.25-3.65 (m, 13H), 4.58 (d, 2H), 6.85 (bs, 1H, NH-amide). 7.07 (d, 1H), 7.22 (bs, 1H, NH-amide), 7.47 (m, 1H), 8.07 (s, 1H), 8.32 (dd, 1H), 8.48 (d, 1H), 8.74 (bs, 2H, NH₂⁺), 8.84 (s, 1H), 9.65 (bs, 1H), 11.24 (b, 1H, NH-pyrole).

MS (ESI⁺) m/z: 500 [MH]⁺.

### EXAMPLE 118

### 5-[2-(2-[1,4]Oxazepan-4-yl-pyrimidin-5-yl)-pyridin-4-yl]-2-piperazin-1-yl-1H-pyrrole-3-carbonitrile trifluoroacetate

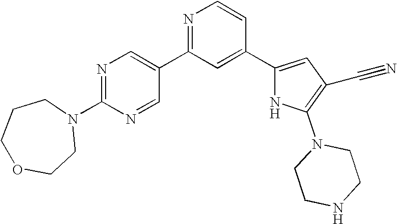

¹H-NMR (400 MHZ, DMSO-d6): 1.88 (m, 2H), 3.31-3-91 (m, 16H), 7.33 (s, 1H), 7.08 (dd, 1H), 8.11 (s, 1H), 8.53 (d, 1H), 8.87 (bs, 2H, NH₂⁺), 9.02 (s, 2H), 11.40 (s, 1H, NH-pyrrole).

MS (ESI⁺) m/z: 431 [MH]⁺.

MS (ESI⁻) m/z: 429 [MH]⁻.

### EXAMPLE 119

### 5-[2-(2-Azepan-1-yl-pyrimidin-5-yl)-pyridin-4-yl]-2-piperazin-1-yl-1H-pyrrole-3-carbonitrile trifluoroacetate

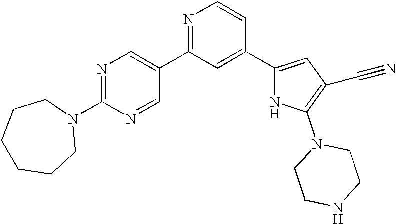

¹H-NMR (400 MHZ, DMSO-d6): 1.60 -1.87 (m, 8H), 3.31 (m, 6H), 3.68 (m, 6H), 7.38 (s, 1H), 7.67 (d, 1H), 8.13 (s, 1H), 8.52 (d, 1H), 8.93,(bs, 2H, NH₂⁺), 9.00 (s, 2H), 11.45 (s, 1H, NH-pyrrole).

MS (ESI⁺) m/z: 429 [MH]⁺.

MS (ESI⁻) m/z: 427 [MH]⁻.

### EXAMPLE 120

### 5-{2-[2-(Isobutyl-methyl-amino)-pyrimidin-5-yl]-pyridin-4-yl}-2-piperazin-1-yl-1H-pyrrole-3-carbonitrile trifluoroacetate

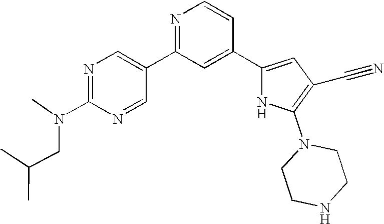

¹H-NMR (400 MHZ, DMSO-d6): 2.14 (m, 1H), 3.34 (m, 4H), 3.57 (d, 2H), 3.76 (m, 4H), 7.71 (s, 1H), 7.88 (d, 1H), 8.28(s, 1H), 8.59 (d, 1H), 8.95 (s, 2H), 9.10 (bs, 2H, NH₂⁺), 11.66 (s, 1H, NH-pyrrole).

MS (ESI⁺) m/z: 417 [MH]⁺.

MS (ESI⁺) m/z: 415 [MH]⁻.

### EXAMPLE 121

### 2-Piperazin-1-yl-5-[2-(2-pyrrolidin-1-yl-pyrimidin-5-yl)-pyridin-4-yl]-1H-pyrrole-3-carbonitrile trifluoroacetate

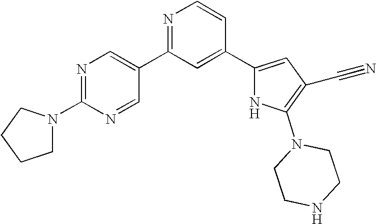

¹H-NMR (400 MHZ, DMSO-d6): 1.99 (m, 1H), 3.34 (m, 4H), 3.58 (m, 4H), 3.69 (m, 4H), 7.47 (s, 1H), 7.67 (d, 1H), 8.16 (s, 1H), 8.55 (d, 1H), 8.99 (bs, 2H, NH₂⁺), 9.01 (s, 2H), 11.47 (s, 1H, NH-pyrrole).

MS (ESI⁺) m/z: 401 [MH]⁺.

MS (ESI⁻) m/z: 399 [MH]⁻.

### EXAMPLE 122

### 2-Piperazin-1-yl-5-[2-(2-piperidin-1-yl-pyrimidin-5-yl)-pyridin-4-yl]-1H-pyrrole-3-carbonitrile trifluoroacetate

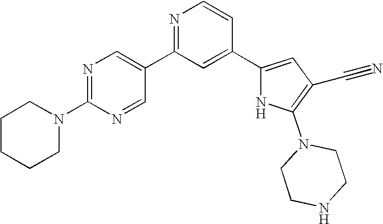

¹H-NMR (400 MHZ, DMSO-d6): 1.59 (m, 4H), 1.70 (s, 2H), 3.33 (m, 4H), 3.69 (m, 4H), 3.87 (m, 4H), 7.45 (s, 1H), 7.65 (d, 1H), 8.16 (s, 1H), 8.55 (d, 1H), 8.95 (bs, 2H, NH₂⁺), 9.00 (s, 2H), 11.48 (s, 1H, NH-pyrrole).

MS (ESI⁺) m/z: 415 [MH]⁺.

MS (ESI⁻) m/z: 413 [MH]⁻.

### EXAMPLE 123

### 5-[2-(2-Methylamino-pyrimidin-5-yl)-pyridin-4-yl]-2-piperazin-1-yl-1H-pyrrole-3-carbonitrile trifluoroacetate

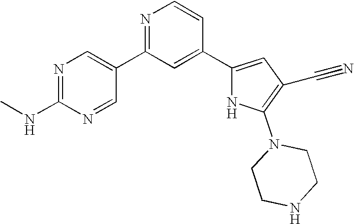

¹H-NMR (400 MHZ, DMSO-d6): 2.90 (s, 3H), 3.33 (m, 4H), 3.67 (m, 4H), 7.38 (s, 1H), 7.60 (m, 1H), 7.64 (s, 1H), 8.11 (s, 1H), 8.53 (d, 1H), 8.83 (bs, 2H, NH₂⁴), 8.98 (s, 2H), 11.38 (s, 1H, NH-pyrrole).

MS (ESI⁺) m/z: 361 [MH]⁺.

MS (ESI⁻) m/z: 359 [MH]⁻.

### EXAMPLE 124

### 2-Piperazin-1-yl-5-[2-(2-pyrrolidin-1-yl-pyrimidin-5-yl)-pyridin-4-yl]-1-pyrrole-3-carboxylic acid amide

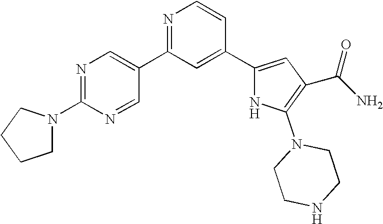

¹H-NMR (400 MHZ, DMSO-d6): 1.98 (m, 4H), 2.88 (m, 4H), 3.06 (m, 4H), 3.58 (m, 4H), 6.93 (bs, 1H, NH-amide), 7.19 (s, 1H), 7.51 (dd, 1H), 7.65 (bs, 1H, NH-amide), 8.12 (s, 1H), 8.48 (m, 1H), 9.09 (s, 2H), 11.38 (s, 1H, NH-pyrrole).

MS (ESI⁺) m/z: 419 [MH]⁺.

MS (ESI⁻) m/z: 417 [MH]⁻.

### EXAMPLE 125

### 2-Piperazin-1-yl-5-[2-(2-piperidin-1-yl-pyrimidin-5-yl)-pyridin-4-yl]-1H-pyrrole-3-carboxylic acid amide

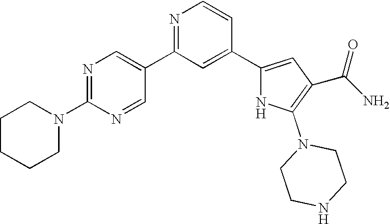

¹H-NMR (400 MHZ, DMSO-d6): 1.57 (m, 4H), 1.68 (m, 2H), 2.87 (m, 4H), 3.05 (m, 4H), 3.85 (m, 4H), 6.94 (bs, 1H, NH-amide), 7.12 (s, 1H), 7.52 (dd, 1H), 7.66 (bs, 1H, NH-amide) 8.13 (s, 1H), 8.48 (m, 1H), 9.08 (s, 2H), 11.33 (s, 1H, NH-pyrrole).

MS (ESI⁺) m/z: 433 [MH]⁺.

MS (ESI⁻) m/z: 431 [MH]⁻.

### EXAMPLE 126

### 5-{2-[2-((2R,6S)-2,6-Dimethyl-morpholin-4-yl)-pyrimidin-5-yl]-pyridin-4-yl}-2-piperazin-1-yl-1H-pyrrole-3-carbonitrile trifluoroacetate

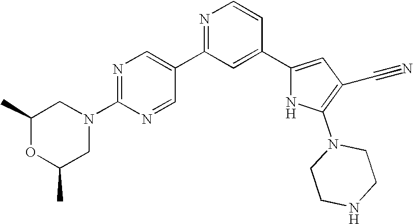

¹H-NMR (400 MHZ, DMSO-d6): 1.21 (d, 6H), 2.67 (m, 2H), 3.34 (m, 4H), 3.61 (m, 2H), 3.70 (m, 4H), 4.62 (d, 2H), 7.52 (d, 1H), 7.73 (d, 1H), 8.21 (s, 1H), 8.60 (d, 1H), 9.01 (bs, 2H, NH₂⁺), 9.05 (s, 2H), 11.56 (s, 1H, NH-pyrrole).

MS (ESI⁺) m/z: 445 [MH]⁺.

MS (ESI⁻) m/z: 443 [MH]⁻.

### EXAMPLE 127

### 2-Piperazin-1-yl-5-{2-[2-(3-trifluoromethyl-5,6-dihydro-8H-[1,2,4]triazolo[4,3-a]pyrazin-7-yl)-pyrimidin-5-yl]-pyridin-4-yl}-1H-pyrrole-3-carbonitrile trifluoroacetate

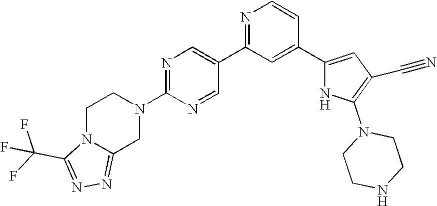

¹H-NMR (400 MHZ, DMSO-d6): 3.32 (m, 4H), 3.36 (m, 4H), 6.88 (m, 1H), 4.35 (m, 2H), 4.40 (m, 2H), 5.28 (s, 2H), 7.37 (s, 1H), 7.64 (dd, 1H), 8.19 (s, 1H), 8.59 (d, 1H), 8.82 (bs, 2H, NH₂⁺), 9.19 (s, 2H), 11.40 (s, 1H, NH-pyrrole).

MS (ESI⁺) m/z: 522 [MH]⁺.

MS (ESI⁻) m/z: 520 [MH]⁻.

### EXAMPLE 128

### 5-{2-[(E)-2-(4-Fluoro-phenyl)-vinyl]-pyridin-4-yl}-2-methyl-1H-pyrrole-3-carbonitrile

### a) 5-(2-Chloro-pyridin-4-yl)-2-methyl-1H-pyrrole-3-carbonitrile

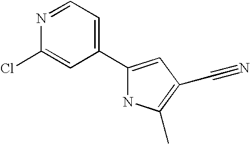

2-Bromo-1-(2-chloro-pyridin-4-yl)-ethanone hydrobromide (500 mg) is added to a mixture of 173 mg of NaHCO₃ and 677 mg of 3-aminocrotononitrile in 8 ml EtOH. The reaction mixture is stirred at room temperature for one and half hour and then heated at reflux overnight. After removal of the solvent the resulting residue is dissolved in dichloromethane, washed with water/brine. After removal of the solvent, an orange oil is obtained and purified via HPLC (acetonitrile/H₂O, X-Terra RP-18) to give a white solid.

¹H-NMR (400 MHZ, DMSO-d6): 2.40 (s, 3H), 7.28 (s, 1H), 7.12 (d, 1H), 7.76 (s, 8.33 (d, 1H), 12.28 (s, 1H, NH).

MS (ESI⁻) m/z: 216 [M−H]⁻

### b) 5-{2-[(E)-2-(4-Fluoro-phenyl)-vinyl]-pyridin-4-yl}-2-methyl-1H-pyrrole-3-carbonitrile

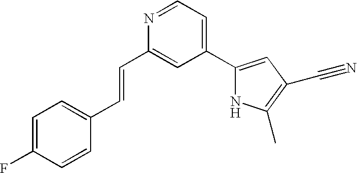

Trans-2(-4-fluoro-phenyl)-vinyl boronic acid (190.6 mg, 1.15 mmol) and 125 mg, 0.57 mmol) of 5-(2-chloro-pyridin-4-yl)-2-methyl-1H-pyrrole-3-carbonitrile are dissolved in 2 ml n-propanol. The solution is degassed by introduction of a stream of argon, Pd(PPh₂)₂Cl₂ (20 mg, 0.028 mmol) and 0.7 ml of 2 N Na₂CO₃ are added and the mixture is heated for 10 min at 145° C. in a microwave oven. After filtration of the reaction mixture over celite and evaporation of the solvent the resulting solid is purified by reverse phase HPLC (Gilson, X-Terra, acetonitrile/water) and yields a white solid.

¹H-NMR (400 MHZ, DMSO-d6): 2.42 (s, 3H), 7.15-7.26 (m, 4H), 7.46 (d, 1H), 7.64-7.72 (m, 3H), 7.78 (d, 1H), 8.49 (d, 1H), 12.21 (s, 1H, NH).

MS (ESI⁺) m/z: 304 [MH]⁺.

MS (ESI⁻) m/z: 302 [M−H]⁻

### EXAMPLE 129

### 5-{2-[(E)-2-(4-Fluoro-phenyl)-vinyl]-pyridin-4-yl}-2-methyl-1H-pyrrole-3-carboxylic acid amide

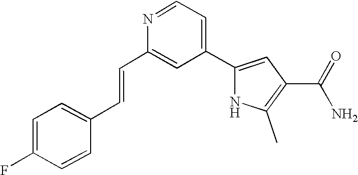

5-{2-[(E)-2-(4-Fluoro-phenyl)-vinyl]-pyridin-4-yl}-2-methyl-1H-pyrrole-3-carbonitrile (20 mg) is dissolved in 1.5 ml concentrated sulfuric acid and stirred at room temperature. The reaction mixture is poured on an icy solution of potassium carbonate and maintained at pH 7-8. After extraction with ethyl acetate, the organic layer is washed with brine, dried with sodium sulfate and evaporated to yield a white solid.

¹H-NMR (400 MHZ, DMSO-d6): 2.50 (s, 3H), 6.71 (bs, 2H, NH₂), 7.17-7.26 (m, 4H), 7.36 (dd, 1H), 7.62-7.72 (m, 4H), 8.45 (d, 1H), 11.64 (s, 1H, NH).

MS (ESI⁺) m/z: 322 [MH]⁺

### EXAMPLE 130

### 2-Methyl-5-[6′-(4-methyl-piperazin-1-yl)-[2,3′]bipyridinyl-4-yl]-1H-pyrrole-3-carbonitrile

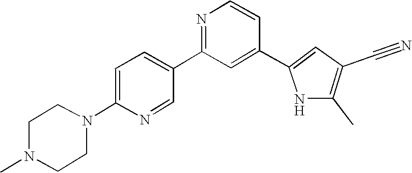

Suzuki coupling of 5-(2-chloro-pyridin-4-yl)-2-methyl-1H-pyrrole-3-carbonitrile (example 128) and 1-methyl-4-[5-(4,4,5,5-tetramethyl-[1,3,2]dioxaborolan-2-yl)-pyridin-2-yl]-piperazine (WO 2007/039285) yields the title compound.

¹H-NMR (400 MHZ, DMSO-d6): 2.25 (s, 3H), 2.44 (t, 4H), 2.50 (s, 3H), 3.61 (t, 4H), 6.96 (d, 1H), 7.28 (d, 1H), 7.48 (d, 1H), 8.11 (s, 1H), 8.26 (dd, 1H), 8.53 (d, 1H), 8.90 (s, 1H), 12.24 (s, 1H).

MS (ESI⁺) m/z: 359 [MH]⁺.

### EXAMPLE 131

### 2-Methyl-5-{2-[(E)-2-(4-morpholin-4-ylmethyl-phenyl)-vinyl]-pyridin-4-yl}-1H-pyrrole-3-carbonitrile

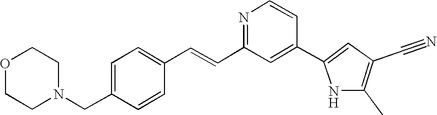

The title compound is synthesized via Suzuki coupling of 5-(2-chloro-pyridin-4-yl)-2-methyl-1H-pyrrole-3-carbonitrile (example 128) and 4-{4-[(E)-2-(4,4,5,5-tetramethyl-[1,3,2]dioxaborolan-2-yl)-vinyl]-benzyl}-morpholine.

¹H-NMR (400 MHZ, DMSO-d6): 22.35-2.43 (m, 4H), 2.44 (s, 3H), 3.50 (s, 2H), 3.60 (t, 4H), 7.18 (s, 1H), 7.23 (d, 1H), 7.37 (d, 2H), 7.48 (d, 1H), 7.61 (d, 2H), 7.68 (d, 1H), 7.82 (s, 1H), 8.52 (d, 1H), 12.26 (bs, 1H).

MS (ESI⁺) m/z: 385 [MH]⁺.

### EXAMPLE 132

### 5-[6′-(4-Benzyl-piperazin-1-yl)-[2,3′]bipyridinyl-4-yl]-2-methyl-1H-pyrrole-3-carbonitrile

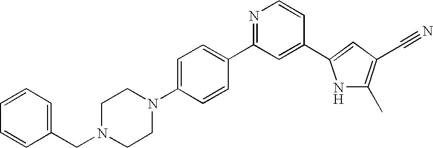

¹H-NMR (400 MHZ, DMSO-d6): 2.43 (s, 3H), 2.43-2.48 (m, 4H), 3.56-3.63 (m, 4H), 6.93 (d, 1H), 7.25 (s, 1H), 7.27 (t, 1H), 7.30-7.36 (m, 4H), 7.45 (d, 1H), 8.09 (s, 1H), 8.23 (dd, 1H), 8.52 (d, 1H), 8.88 (d, 1H), 12.22 (s, 1H).

MS (ESI⁺) m/z: 435 [MH]⁺.

### EXAMPLE 133

### 2-Methyl-5-(2-{(E)-2-[4-(morpholine-4-carbonyl)-phenyl]-vinyl}-pyridin-4-yl)-1H-pyrrole-3-carbonitrile

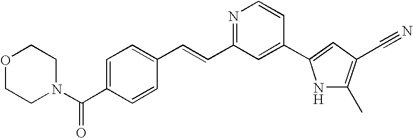

MS (ESI⁺) m/z: 399 [MH]⁺.

### EXAMPLE 134

### 2-Methyl-5-[6′-(1-methyl-piperidin-4-yloxy)-[2,3′bipyridinyl-4-yl]-1H-pyrrole-3-carbonitrile

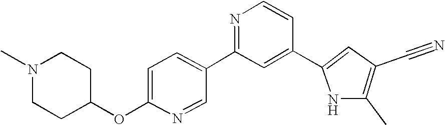

¹H-NMR (400 MHZ, DMSO-d6): 1.65-1.75 (m, 2H), 1.90-2.05 (m, 2H), 2.15-2.22 (m, 2H), 2.19 (s, 3H), 2.44 (s, 3H), 2.60-2.70 (m, 2H), 5.00-5.09 (m, 1H), 6.92 (d, 1H), 7.28 (s, 1H), 7.54 (d, 1H), 8.17 (s, 1H), 8.36 (dd, 1H), 8.56 (d, 1H), 8.89 (d, 1H), 12.24 (s, 1H).

MS (ESI⁺) m/z: 374 [MH]⁺.

### EXAMPLE 135

### 5-(2-{2-[(2-Methoxy-ethyl)-methyl-amino]-pyrimidin-5-yl}-pyridin-4-yl)-2-methyl-1H-pyrrole-3-carbonitrile

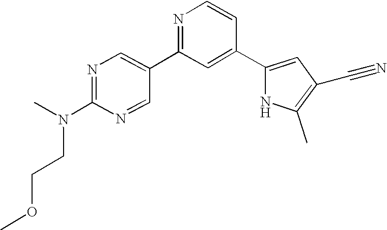

¹H-NMR (400 MHZ, DMSO-d6): 2.45 (s, 3H), 3.23 (s, 3H), 3.29 (s, 3H), 3.58 (t, 2H), 3.87 (t, 2H), 7.28 (s, 1H), 7.51 (dd, 1H), 8.12 (s, 1H), 8.54 (d, 1H), 9.05 (s, 2H), 11.22 (s, 1H, NH-pyrrole).

MS (ESI⁺) m/z: 349 [MH]⁺.

MS (ESI⁻) m/z: 347 [MH]⁻.

### EXAMPLE 136

### 5-{2-[4-Methoxy-3-(3-methoxy-propoxy)-phenyl]-pyridin-4-yl}-2-methyl-1H-pyrrole-3-carbonitrile

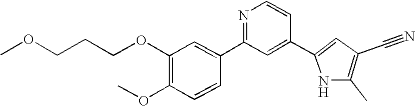

¹H-NMR (400 MHZ, DMSO-d6): 2.02 (quint., 2H), 2.46 (s, 3H), 3.33 (s, 3H), 3.53 (t, 2H), 3.86 (s, 3H), 4.13 (t, 2H), 7.11 (d, 1H), 7.31 (s, 1H), 7.51 (d, 1H), 7.74 (dd, 1H), 7.77 (d, 1H), 8.14 (s, 1H), 8.58 (d, 1H), 12.31 (s, 1H).

MS (ESI⁺) m/z: 378 [MH]⁺.

### EXAMPLE 137

### 5-{2-[4-Methoxy-phenyl]-pyridin-4-yl}-2-methyl-1H-pyrrole-3-carbonitrile

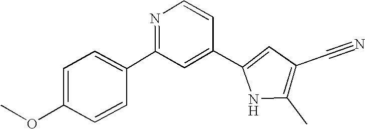

¹H-NMR (400 MHZ, DMSO-d6): 2.44 (s, 3H), 3.83 (s, 3H), 7.07 (d, 2H), 7.27 (s, 1H), 7.50 (d, 1H), 8.11 (d, 2H), 8.14 (s, 1H), 8.56 (d, 1H), 12.30 (s, 1H).

MS (ESI⁺) m/z: 290 [MH]⁺.

### EXAMPLE 138

### 4-Methyl-5-[2-(2-[1,4]oxazepan-4-yl-pyrimidin-5-yl)-pyridin-4-yl]-2-piperazin-1-yl-1H-pyrrole-3-carboxylic acid amide

### a) 2-Bromo-1-(2-chloro-pyridin-4-yl)-propan-1-one

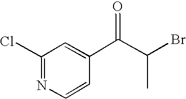

Prepared in analogy to Example 1c.

MS (ESI⁺) m/z: 248 [MH]⁺.

### b) 4-[5-(2-Chloro-pyridin-4-yl)-3-cyano-4-methyl-1H-pyrrol-2-yl]-piperazine-1-carboxylic acid tert-butyl ester

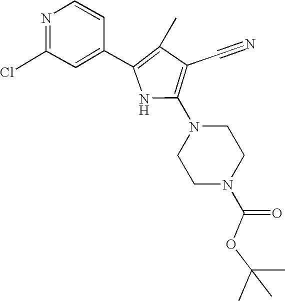

Prepared as described in example 8 starting from 2-bromo-1-(2-chloro-pyridin-4-yl)-propan-1-one hydrobromide and 4-(1-amino-2-cyano-vinyl)-piperazine-1-carboxylic acid tert-butyl ester hydrochloride.

MS (ESI⁺) m/z: 402 [MH]⁺.

### c) 4-{3-Cyano-4-methyl-5-[2-(2-[1,4]oxazepan-4-yl-pyrimidin-5-yl)-pyridin-4-yl]-1H-pyrrol-2-yl}-piperazine-1-carboxylic acid tert-butyl ester

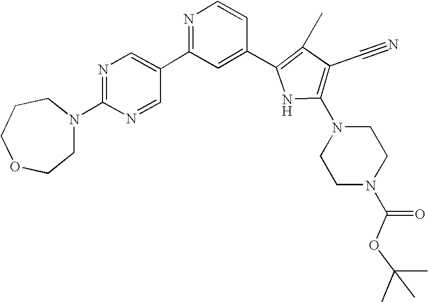

Prepared as described in example 8 starting from 4-[5-(4,4,5,5-tetramethyl-[1,3,2]dioxaborolan-2-yl)-pyrimidin-2-yl]-[1,4]oxazepane and 4-[5-(2-chloro-pyridin-4-yl)-3-cyano-4-methyl-1H-pyrrol-2-yl]-piperazine-1-carboxylic acid tert-butyl ester.

¹H-NMR (400 MHZ, CDCl₃): 1.48 (s, 9H), 1.77 (s, 2H), 1.99-2.05 (m, 4H), 2.33 (s, 3H), 3.38 (s, 2H), 3.55-3.58 (m, 2H), 3.72-3.77 (m, 2H), 3.81-3.86 (m, 2H), 3.93-3.99 (m, 4H), 7.10 (d, 1H), 7.45 (s, 1H), 8.43 (s, 1H), 8.55 (d, 1H), 8.86 (s, 2H).

MS (ESI⁺) m/z: 545 [MH]⁺.

### d) 4-Methyl-5-[2-(2-[1,4]oxazepan-4-yl-pyrimidin-5-yl)-pyridin-4-yl]-2-piperazin-1-yl-1H-pyrrole-3-carbonitrile

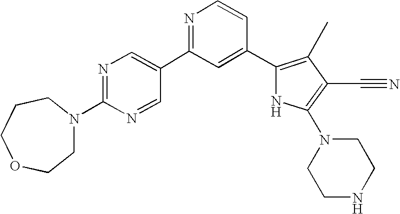

Prepared in a similar fashion as example 8d starting from 4-{3-cyano-4-methyl-5-[2-(2-[1,4]oxazepan-4-yl-pyrimidin-5-yl)-pyridin-4-yl]-1H-pyrrol-2-yl}-piperazine-1-carboxylic acid tert-butyl ester.

¹H-NMR (400 MHZ, DMSO-d6): 1.86-1.91 (m, 2H), 2.43 (s, 3H), 3.22-3.29 (m, 4H), 3.61-3.97 (m, 12H), 7.77 (d, 1H), 8.35 (s, 1H), 8.48 (d, 1H), 8.64 (s, 1H), 9.19 (s, 2H), 9.38 (s, br, 2H, NH₂⁺).

MS (ESI⁻) m/z: 445 [MH]⁺.

### e) 4-Methyl-5-[2-(2-[1,4]oxazepan-4-yl-pyrimidin-5-yl)-pyridin-4-yl]-2-piperazin-1-yl-1H-pyrrole-3-carboxylic acid amide

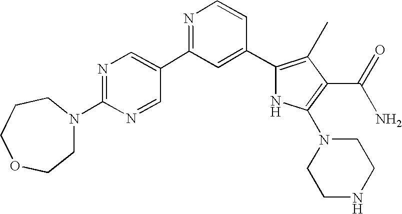

¹H-NMR (400 MHZ, DMSO-d6): 1.89 (t, 2H), 2.42 (s, 3H), 2.96 (m, 4H), 3.07 (m, 4H), 3.64 (t, 2H), 3.75 (t, 2H), 3.91-3.94 (m, 4H), 6.54-7.00 (s, br, 2H, NH₂⁺), 6.86 (s, 1H), 7.32 (d, 1H), 7.78 (s, 1H), 7.83 (s, 1H), 8.55 (d, 1H), 9.05 (s, 2H), 11.13 (s, 1H, NH).

MS (ESI⁺) m/z: 463 [MH]⁺.

The following compounds are prepared as described in example 138:

### EXAMPLE 139

### 5-[6′-(4-Cyclopentyl-piperazin-1-yl)-[2,3′]bipyridinyl-4-yl]-4-methyl-2-piperazin-1-yl-1H-pyrrole-3-carbonitrile

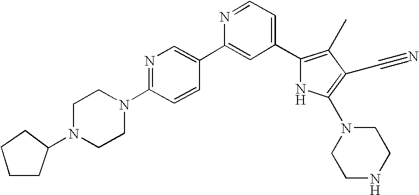

¹H-NMR (400 MHZ, DMSO-d6): 1.59 (m, 2H), 1.75 (m, 4H), 2.00-2.01 (m, 2H), 2.35 (s, 3H), 3.05-3.35 (m, 8H), 3.55-3.70 (m, 6H), 4.57 (d, 2H), 7.14 (d, 1H), 7.48 (d, 1H), 7.95 (s, 1H), 8.31 (d, 1H), 8.59 (d, 1H), 8.91 (d, 1H), 9.00 (s, 2H, NH₂⁺), 10.04 (s, 1H, NH⁺), 11.34 (s, 1H, NH).

MS (ESI⁺) m/z: 497 [MH]⁺.

### EXAMPLE 140

### 4-Methyl-5-{2-[(E)-2-(4-morpholin-4-ylmethyl-phenyl)-vinyl]-pyridin-4-yl}-2-piperazin-1-yl-1H-pyrrole-3-carbonitrile

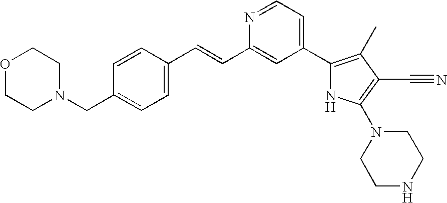

¹H-NMR (400 MHZ, DMSO-d6, 120° C.): 2.45 (s, 1H), 3.03-3.33 (m, 8H), 3.77-4.01 (m, 8H), 4.38 (s, 2H), 7.53 (d, 1H), 7.57 (d, 1H), 7.92 (s, 1H), 7.69-7.83 (m, 6H), 8.54 (d, 1H), 9.43 (s, 2H, NH₂⁺), 11.51 (s, 1H, NH⁺), 12.01 (s, 1H).

MS (ESI⁺) m/z: 469 [MH]⁺.

### EXAMPLE 141

### 2-Isopropyl-5-[2-((E)-styryl)-pyridin-4-yl]-1H-pyrrole-3-carboxylic acid ethyl ester

### a) 2-[2-(2-Chloro-pyridin-4-yl)-2-oxo-ethyl]-4-methyl-3-oxo-pentanoic acid ethyl ester

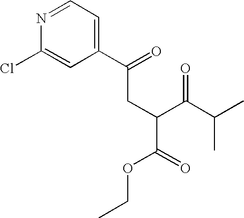

2-Bromo-1-(2-chloro-pyridin-4-yl)-ethanone hydrobromide (500 mg) is stirred in a mixture of 20 ml aqueous NaHCO₃ solution and ether to liberate the free base. The aqueous phase is extracted two more times with ether, the ether phase dried and concentrated.

Ethyl isobutyrate (337 mg) in 5 ml THF is added drop wise to a solution of LiHMDS (2.1 ml 1.0 M solution) in 5 ml THF at −78° C. After stirring for 2 hours at −78° C. 2-bromo-1-(2-chloro-pyridin-4-yl)-ethanone (see above) dissolved in 3 ml THF is added drop wise. The reaction mixture is allowed to warm to room temperature overnight. Aqueous NH₄Cl solution is added and the mixture is extracted with ethyl acetate. After removal of the solvent the title compound is obtained as a pale yellow oil.

¹H-NMR (400 MHZ, DMSO-d6): 1.07 (d, 3H), 1.10 (d, 3H), 1.19 (t, 3H), 2.98 (sept, 1H), 3.50-3.68 (m, 2H), 4.13 (q, 2H), 4.33 (t, 1H), 7.84 (d, 1H), 7.96 (s, 1H), 8.63 (d, 1H).

MS (ESI⁺) m/z: 312 [MH]⁺.

### b) 2-Isopropyl-5-(2-chloro-pyridin-4-yl)-1H-pyrrole-3-carboxylic acid ethyl ester

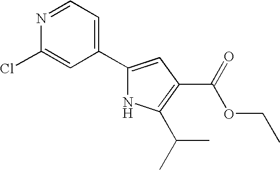

A solution of 2-[2-(2-chloro-pyridin-4-yl)-2-oxo-ethyl]-4-methyl-3-oxo-pentanoic acid ethyl ester (580 mg) and ammonium acetate (0.72 g) in 10 ml ethanol is refluxed for 2 hours. Aqueous NaHCO₃ solution is added and the mixture is extracted with ethyl acetate. Removal of the solvent provided essentially pure 2-isopropyl-5-(2-chloro-pyridin-4-yl)-1H-pyrrole-3-carboxylic acid ethyl ester as white solid.

¹H-NMR (400 MHZ, DMSO-d6): 1.26 (t, 3H), 1.28 (d, 6H), 3.82 (sept, 1H), 4.19 (q, 2H), 7.19 (d, 1H), 7.71 (dd, 1H), 7.88 (s 1H), 8.27 (d, 1H), 11.5 (s, 1H).

MS (ESI⁺) m/z: 293 [MH]⁺.

### c) 2-Isopropyl-5-[2-((E)-styryl)-pyridin-4-yl]-1H-pyrrole-3-carboxylic acid ethyl ester

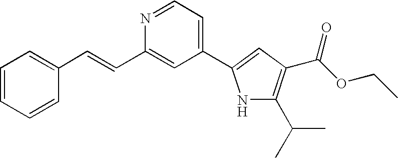

2-Isopropyl-5-(2-chloro-pyridin-4-yl)-1H-pyrrole-3-carboxylic acid ethyl ester (310 mg) and E-phenyl-vinyl boronic acid (156 mg) are dissolved in 6 ml n-propanol. The solution is degassed by introduction of a stream of argon, Pd(PPh₂)₂Cl₂ (37 mg) is added and the mixture is heated under reflux overnight. The reaction is quenched with saturated NaHCO₃ solution, extracted into ethyl acetate, the organic phase is dried over Na₂SO₄ and the solvent evaporated. Purification by reverse phase HPLC (Waters X-Terra, acetonitrile/water) yields the title compound.

¹H-NMR (400 MHZ, DMSO-d6): 1.29 (t, 3H), 1.32 (d, 6H), 3.85 (sept, 1H), 4.20 (q, 2H), 7.09 (d, 1H), 7.29 (d, 1H), 7.32 (t, 1H), 7.41 (t, 2H), 7.57 (dd, 1H), 7.66 (d, 2H), 7.70 (d, 1H), 7.88 (s, 1H), 8.49 (d, 1H), 11.45 (s, 1H).

MS (ESI⁺) m/z: 361 [MH]⁺.

### EXAMPLE 142

### 2-Isopropyl-5-[2-((E)-styryl)-pyridin-4-yl]-1H-pyrrole-3-carboxylic acid amide

### a) 2-Isopropyl-5-[2-((E)-styryl)-pyridin-4-yl]-1H-pyrrole-3-carboxylic acid

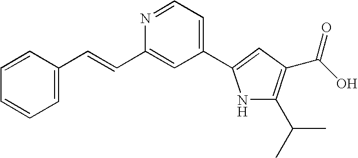

2-Isopropyl-5-[2-((E)-styryl)-pyridin-4-yl]-1H-pyrrole-3-carboxylic acid ethyl ester (example 141) (123 mg), dissolved in 2 ml ethanol is treated with 10 M NaOH (0.5 ml) at 40° C. for 24 hours. The reaction mixture is brought to pH 2 using 6 N HCl followed by evaporation. The crude material obtained is used for the next reaction step without further purification.

MS (ESI⁺) m/z: 331 [M−H]⁻.

### b) 2-Isopropyl-5-[2-((E)-styryl)-pyridin-4-yl]-1H-pyrrole-3-carboxylic acid 2,4-dimethoxy-benzylamide

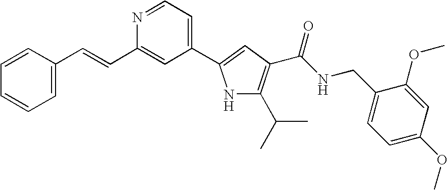

A solution of 2-isopropyl-5-[2-((E)-styryl)-pyridin-4-yl]-1H-pyrrole-3-carboxylic acid (110 mg) in DMF is treated with 2,4-dimethoxybenzylamine (55 mg), EDCl (76 mg), HOBt (54 mg) and triethylamine (0.15 ml). The mixture is stirred at room temperature for 16 hours, before being quenched by the addition of sat. NaHCO₃ solution. Extraction with ethyl acetate gives a crude product which is further purified by HPLC (Waters X-Terra, acetonitrile/water gradient). The title compound is obtained as white solid.

¹H-NMR (400 MHZ, DMSO-d6): 1.28 (d, 6H), 3.73 (s, 3H), 3.83 (s, 3H), 3.96 (sept, 1H), 4.29 (d, 2H), 6.46 (d, 1H), 6.53 (s, 1H), 7.08 (d, 1H), 7.22 (d, 1H), 7.24 (d, 1H), 7.30 (t, 1H), 7.39 (t, 2H), 7.43 (d, 1H), 7.63 (d, 1H), 7.65 (d, 2H), 7.75 (s, 1H), 7.95 (t, 1H), 8.47 (d, 1H), 11.2 (s, 1H).

MS (ESI⁺) m/z: 482 [MH]⁺.

### c) 2-Isopropyl-5-[2-((E)-styryl)-pyridin-4-yl]-1H-pyrrole-3-carboxylic acid amide

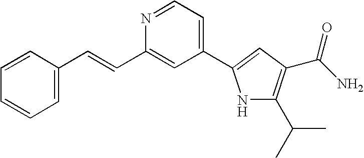

2-Isopropyl-5-[2-((E)-styryl)-pyridin-4-yl]-1H-pyrrole-3-carboxylic acid 2,4-dimethoxy-benzylamide (50 mg) is dissolved in dichloromethane (2 ml). Trifluoroacetic acid (0.5 ml) is added and the mixture is stirred at room temperature for 16 hours. After addition of aqueous NaHCO₃ solution (5 ml), the product is extracted into ethyl acetate. Removal of the solvent yields the title compound as white solid.

¹H-NMR (400 MHZ, DMSO-d6): 1.28 (d, 6H), 4.00 (sept, 1H), 7.17 (d, 1H), 7.26 (d, 1H), 7.32 (t, 1H), 7.42 (t, 2H), 7.44 (dd, 1H), 7.65 (t, 2H), 7.67 (d, 1H), 7.78 (s, 1H), 8.48 (d, 1H), 11.25 (s, 1 H), (2 protons (NH₂) obscured).

MS (ESI⁺) m/z: 332 [MH]⁺.

### EXAMPLE 143

### 2-Isopropyl-5-{2-[(E)-2-(4-morpholin-4-ylmethyl-phenyl)-vinyl]-pyridin-4-yl}-1H-pyrrole-3-carbonitrile

### a) 5-(2-Chloro-pyridin-4-yl)-2-isopropyl-1H-pyrrole-3-carbonitrile

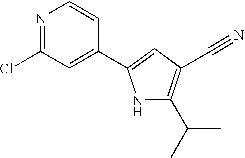

A mixture of 4-methyl-3-oxo-pentanenitrile (7.6 g, 68 mmol), tetraethoxysilane (31.6 ml, 136 mmol) and ammonium acetate (23.7 g, 307 mmol) in 300 ml ethanol is stirred under reflux for 5 hours. 1-(2-Chloro-pyridin-4-yl)-ethanone hydrobromide (example 1) (19.3 g, 61 mmol) and NaHCO₃ (17.2 g, 204 mmol) is added and reflux is continued for 16 hours. After cooling to room temperature the mixture is filtrated, the filtrate is evaporated and the crude product is purified by column chromatography (silica gel, ethyl acetate/hexanes).

¹H-NMR (400 MHZ, DMSO-d6): 1.36 (d, 6H), 3.17 (sept., 1H), 7.30 (s, 1H), 7.69 (d, 1H), 7.85 (s, 1H), 8.37 (d, 1H).

MS (ESI⁺) m/z: 246 [MH]⁺.

### b) 2-Isopropyl-5-{2-[(E)-2-(4-morpholin-4-ylmethyl-phenyl)-vinyl]-pyridin-4-yl}-1H-pyrrole-3-carbonitrile

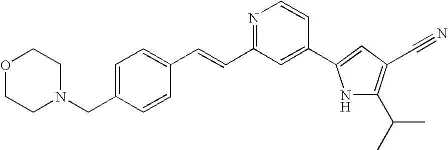

Suzuki coupling as described earlier yields the title compound.

¹H-NMR (400 MHZ, DMSO-d6): 1.38 (d, 6H), 2.36-2.40 (m, 4H), 3.20 (sept., 1H), 3.50 (s, 2H), 3.60 (t, 4H), 7.19 (s, 1H), 7.26 (d, 1H), 7.37 (d, 2H), 7.54 (d, 1H), 7.64 (d, 2H), 7.71 (d, 1H), 7.86 (s, 1H), 8.55 (d, 1H), 12.03 (bs, 1H).

MS (ESI⁺) m/z: 413 [MH]⁺.

### EXAMPLE 144

### 2-Piperidin-4-yl-5-[2-((E)-styryl)-pyridin-4-yl]-1H-pyrrole-3-carbonitrile

### a) 4-(2-Ethoxycarbonyl-acetyl)-piperidine-1-carboxylic acid tert-butyl ester

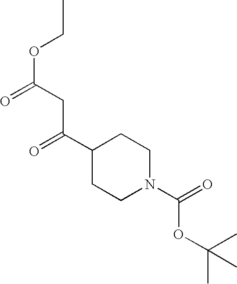

1-tert.Butyloxycarbonylpiperidine-4-carboxylic acid (1.20 g, 5.13 mmol) is dissolved in 25 ml of THF and cooled to 0° C. After the addition of 0.90 g (5.55 mmol) CDI the reaction mixture is stirred for 2 h at room temperature and allowed to stand over night. Ethylhydrogen malonate (1.0 g, 7.27 mmol) is dissolved in 10 ml of THF and treated with 5.7 ml of a 2 M solution of i-PrMgCl at 0° C. over a period of 30 min under Ar. The resulting solution is stirred for additional 20 min at 0° C., for 45 min at room temperature and for 45 min at 50° C. and then cooled to 0° C. again. The CDI activated carboxylic acid solution is slowly added over 30 min. The mixture is allowed to react for additional 3 h at room temperature. Ice is added and the reaction mixture is neutralized with HCl (1 N) and extracted with ethyl acetate. The organic layers are washed with sat. NaHCO₃ and brine and dried over Na₂SO₄. The crude product is purified by silica gel chromatography (hexanes-ethyl acetate gradient).

¹H-NMR (400 MHZ, DMSO-d6): 1.18 (t, 3H), 1.39 (s, 9H), 1.80 (d, 4H), 2.60-2.70 (m, 1H), 3.67 (s, 2H), 3.91 (d, 4H), 7.09 (q, 2H).

MS (ESI⁺) m/z: 322 [M+Na]⁺.

### b) 4-[5-(2-Chloro-pyridin-4-yl)-3-ethoxycarbonyl-1H-pyrrol-2-yl]-piperidine-1-carboxylic acid tert-butyl ester

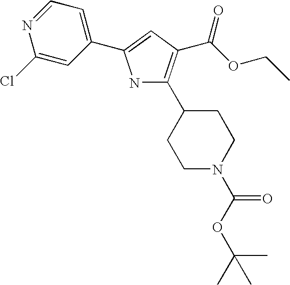

NaH (660 mg, 14.5 mmol) is added to a solution of 4-(2-ethoxycarbonyl-acetyl)-piperidine-1-carboxylic acid tert-butyl ester in 150 ml of THF at 0° C. the mixture is allowed to react for 15 min at room temperature to give a clear solution. A solution of 3.5 g (14.9 mmol) 1-(2-chloro-pyridin-4-yl)-ethanone (free base, example 1c) in THF is added. The reaction is stirred for 1 h at 0° C. and at room temperature over night. Ice is added, followed by extraction with ethyl acetate. The combined organic layers are washed with sat. NaHCO₃ and brine, dried over Na₂SO₄ and concentrated. The residue is dissolved in 50 ml of ethanol. After adding 4.20 g (54.5 mmol) NH₄OAc the reaction is stirred at room temperature over night. Ice is added, followed by extraction with ethyl acetate. The combined organic layers are washed with sat. NaHCO₃ and brine, dried over Na₂SO₄ and concentrated. The crude product is purified by silica gel chromatography (hexanes-ethyl acetate gradient).

¹H-NMR (400 MHZ, DMSO-d6): 1.27 (t, 3H), 1.47 (s, 9H), 1.75-1.81 (m, 2H), 1.90-1.97 (m, 2H), 2.85-2.95 (m, 2H), 3.86 (t, 1H), 4.09-4.14 (m, 2H), 4.31 (q, 2H), 7.09 (s, 1H), 7.39 (d, 1H), 7.52 (s, 1H), 8.28 (d, 1H), 9.91 (s, 1H, NH).

MS (ESI⁺) m/z: 434 [MH]⁺.

### c) 4-{3-Ethoxycarbonyl-5-[2-((E)-styryl)-pyridin-4-yl]-1H-pyrrol-2-y}-piperidine-1-carboxylic acid tert-butyl ester

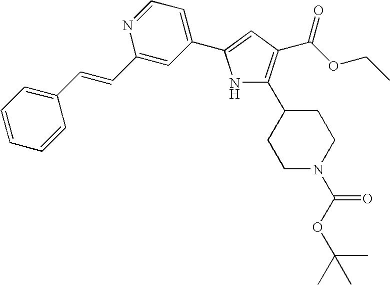

Prepared as described in example 8 starting from trans-phenylvinylboronic acid and 4-[5-(2-chloro-pyridin-4-yl)-3-ethoxycarbonyl-1H-pyrrol-2-yl]-piperidine-1-carboxylic acid tert-butyl ester.

¹H-NMR (400 MHZ, DMSO-d6): 1.31 (t, 3H), 1.46 (s, 9H), 1.69-1.77 (m, 2H), 1.81-1.95 (m, 2H), 2.70-2.91 (m, 2H), 3.66-3.76 (m, 1H), 4.17 (d, 2H), 4.23 (q, 2H), 7.15 (d, 1H), 7.31 (d, 1H), 7.36 (d, 1H), 7.44 (dd, 2H), 7.61 (d, 1H), 7.69 (d, 2H), 7.76 (d, 1H), 7.92 (s, 1H), 8.52 (d, 1H), 11.49 (s, 1H, NH).

MS (ESI⁺) m/z: 502 [MH]⁺.

### d) 4-{3-Carboxy-5-[2-((E)-styryl)-pyridin-4-yl]-1H-pyrrol-2-yl}-piperidine-1-carboxylic acid tert-butyl ester

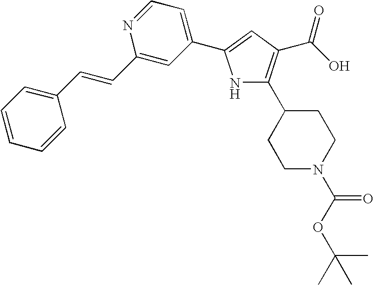

4-{3-Ethoxycarbonyl-5-[2-((E)-styryl)-pyridin-4-yl]-1H-pyrrol-2-yl}-piperidine-1-carboxylic acid tert-butyl ester (65 mg, 0.13 mmol) is dissolved in 4.0 ml of methanol and after addition of 3.4 ml of 1 N NaOH the mixture is stirred for 4 h at reflux. Then the organic solvents are removed under reduced pressure. The residue is diluted with water and washed with ethyl acetate. The aqueous layer is acidified (pH 4-5) with 2 N HCl and extracted with ethyl acetate, washed with brine and dried over Na₂SO₄.

¹H-NMR (400 MHZ, DMSO-d6): 1.45 (s, 9H), 1.69-1.76 (m, 2H), 1.79-1.94 (m, 2H), 2.71-2.88 (m, 2H), 3.68-3.79 (m, 1H), 4.10-4.20 (m, 2H), 7.13 (d, 1H), 7.29 (d, 1H), 7.35 (d, 1H), 7.43 (dd, 2H), 7.58 (d, 1H), 7.69 (s, 2H), 7.70 (d, 1H), 7.90 (s, 1H), 8.49 (d, 1H), 11.40 (s, 1H, NH), 11.98 (s, 1H, OH).

MS (ESI⁺) m/z: 474 [MH]⁺.

### e) 4-{3-Carbamoyl-5-[2-((E)-styryl)-pyridin-4-yl]-1H-pyrrol-2-yl}-piperidine-1-carboxylic acid tert-butyl ester

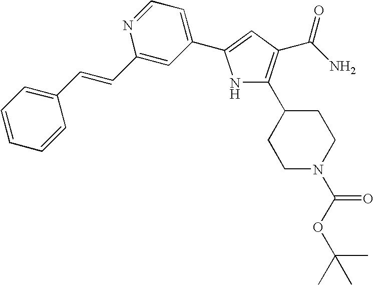

To a solution of 100 mg (0.211 mmol) 4-{3-carboxy-5-[2((E)-styryl)-pyridin-4-yl]-1H-pyrrol-2-yl}-piperidine-1-carboxylic acid tert-butyl ester in 10 ml of CH₂Cl₂ and 3 ml DMF is added HOBt (32 mg, 0.059 mmol) and EDC (45 mg, 0.117 mmol). The reaction mixture is stirred for 0.5 h at room temperature and treated with 0.05 ml (0.51 mmol) conc. NH₃. After vigorous stirring over night the reaction is diluted with ethyl acetate and washed with sat. NaHCO₃ and brine. The organic layer is dried over Na₂SO₄ and concentrated. The crude product is purified by silica gel chromatography (hexanes/ethyl acetate gradient).

¹H-NMR (400 MHZ, DMSO-d6): 1.43 (s, 9H), 1.65-1.72 (m, 2H), 1.77-1.88 (m, 2H), 2.66-2.82 (m, 2H), 3.81-3.90 (m, 1H), 4.08-4.16 (m, 2H), 6.79 (s, 1H, NH), 7.20 (d, 1H), 7.26 (d, 1H), 7.34 (s, br, 1H, NH), 7.42 (d, 1H), 7.44 (dd, 2H), 7.67 (d, 1H), 7.68 (d, 2H), 7.77 (s, 1H), 7.95 (s, 1H), 8.49 (d, 1H), 11.24 (s, 1H, NH).

MS (ESI⁺) m/z: 473 [MH]⁺.

### f) 4-{3-Cyano-5-[2-((E)-styryl)-pyridin-4-yl]-1H-pyrrol-2-yl}-piperidine-1-carboxylic acid tert-butyl ester

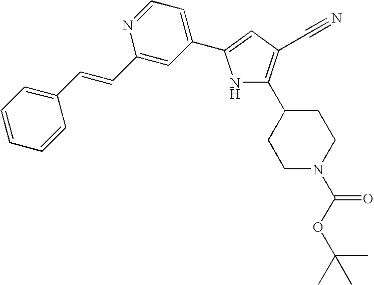

4-{3-Carbamoyl-5-[2-((E)-styryl)-pyridin-4-yl]-1H-pyrrol-2-yl}-piperidine-1-carboxylic acid tert-butyl ester (66 mg, 0.14 mmol) is dissolved in 3.0 ml of THF and after the addition of 0.11 ml (1.40 mmol) of pyridine cooled to 0° C. Then 49 μl (0.35 mmol) of trifluoroacetic acid anhydride is added and the mixture is stirred for 0.5 h at room temperature. Then the reaction is quenched with water and extracted with ethyl acetate (3×), washed with sat. NaHCO₃ and NaCl-solution, dried over Na₂SO₄ and evaporated. The crude product is purified by silica gel chromatography (hexanes-ethyl acetate gradient).

¹H-NMR (400 MHZ, DMSO-d6): 1.45 (s, 9H), 1.79-1.86 (m, 4H), 2.79-2.91 (m, 2H), 2.97-3.07 (m, 1H), 4.08-4.19 (m, 2H), 6.20 (s, 1H), 7.28 (d, 1H), 7.36 (d, 1H), 7.44 (dd, 2H), 7.55 (d, 1H), 7.69 (s, 2H), 7.71 (d, 1H), 7.87 (s, 1H), 8.55 (d, 1H), 12.02 (s, 1H, NH).

MS (ESI⁺) m/z: 455 [MH]⁺.

### g) 2-Piperidin-4-yl-5-[2-((E)-styryl)-pyridin-4-yl]-1H-pyrrole-3-carbonitrile

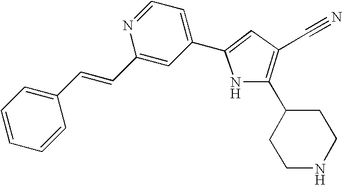

Prepared in a similar fashion as example 8 starting from 4-{3-cyano-5-[2-((E)-styryl)-pyridin-4-yl]-1H-pyrrol-2-yl}-piperidine-1-carboxylic acid tert-butyl ester.

¹H-NMR (400 MHZ, DMSO-d6): 2.09-2.18 (m, 4H), 2.99-3.13 (m, 2H), 3.25-3.34 (m, 1H), 3.39-3.49 (m, 2H), 7.38 (d, 1H), 7.47 (d, 1H), 7.52 (dd, 2H), 7.65 (s, 1H), 7.70 (d, 2H), 8.02 (s, 1H), 8.14 (d, 1H), 8.58 (s, 1H, NH₂⁺), 8.68 (s, 1H), 8.70 (s, 1H), 8.97 (s, 1H, NH₂⁺), 13.15 (s, 1H, NH).

MS (ESI⁺) m/z: 355 [MH]⁺.

### EXAMPLE 145

### 2-Piperidin-4-yl-5-[2-((E)-styryl)-pyridin-4-yl]-1H-pyrrole-3-carboxylic acid

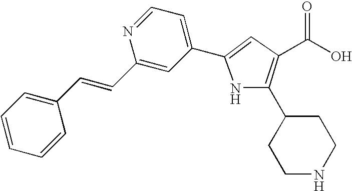

Deprotection of 4-{3-carboxy-5-[2-((E)-styryl)-pyridin-4-yl]-1H-pyrrol-2-yl}piperidine-1-carboxylic acid tert-butyl ester (example 144) as described in example 8 yields the title compound.

¹H-NMR (400 MHZ, DMSO-d6): 1.94-2.02 (m, 2H), 2.24-2.36 (m, 2H), 2.95-3.07 (m, 2H), 3.39-3.46 (m, 2H), 3.73-3.82 (m, 1H), 7.44 (d, 1H), 7.50 (d, 1H), 7.54 (dd, 2H), 7.70 (s, 1H), 7.71 (d, 2H), 8.21 (d, 1H), 8.26 (d, 1H), 8.71 (s, 2H, NH₂⁺), 8.80 (s, 1H), 9.07 (d, 1H), 12.37 (s, 1H, NH).

MS (ESI⁺) m/z: 374 [MH]⁺.

### EXAMPLE 146

### 2-Piperidin-4-yl-5-[2-((E)-styryl)-pyridin-4-yl]-1H-pyrrole-3-carboxylic acid amide

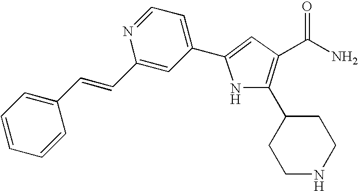

Prepared via deprotection of 4-{3-carbamoyl-5-[2-((E)-styryl)-pyridin-4-yl]-1H-pyrrol-2-yl}-piperidine-1-carboxylic acid tert-butyl ester (example 144) as described in example 8).

¹H-NMR (400 MHZ, DMSO-d6): 1.39-2.05 (m, 2H), 2.17-2.35 (m, 2H), 2.91-3.80 (m, 2H), 3.34-3.45 (m, 2H), 3.67-3.88 (m, 1H), 7.04 (s, 1H), 7.40-7.53 (m, 5H), 7.72 (d, 2H), 7.93 (d, 1H), 8.20 (d, 1H), 8.63 (d, 2H), 8.71 (s, 1H), 8.95 (d, 1H), 11.24 (s, 1H, NH).

MS (ESI⁺) m/z: 373 [MH]⁺.

### EXAMPLE 147

### 2-Piperidin-4-yl-5-[2((E)-styryl)-pyridin-4-yl]-1H-pyrrole-3-carboxylic acid benzylamide

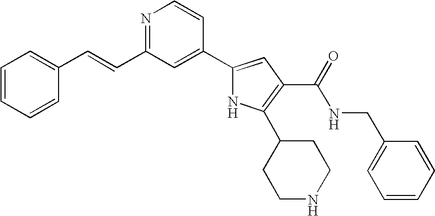

Prepared in a similar fashion as example 144 starting from 4-{3-carboxy-5-[2-((E)-styryl)-pyridin-4-yl]-1H-pyrrol-2-yl}-piperidine-1-carboxylic acid tert-butyl ester (example 144) and benzylamine, followed by BOC-deprotection.

¹H-NMR (400 MHZ, DMSO-d6): 1.94-2.02 (m, 2H), 2.21-2.37 (m, 2H), 2.94-3.05 (m, 2H), 3.37-3.44 (m, 2H), 3.75-3.85 (m, 1H), 4.46 (d, 2H), 7.23-7.30 (m, 1H), 7.33-7.38 (m, 4H), 7.41 (d, 1H), 7.48-7.55 (m, 3H), 7.71 (d, 2H), 7.77 (s, 1H), 7.93 (s, 1H, NH), 8.15 (d, 1H), 8.60 (s, 2H, NH₂⁺), 8.63 (d, 1H), 8.64 (s, 1H), 8.91 (d, 1H), 12.23 (s, 1H, NH).

MS (ESI⁺) m/z: 463 [MH]⁺.

The following compounds are prepared as outlined in example 144:

### EXAMPLE 148

### a) 2-(2-Amino-ethyl)-5-[2-((E)-styryl)-pyridin-4-yl]-1H-pyrrole-3-carbonitrile

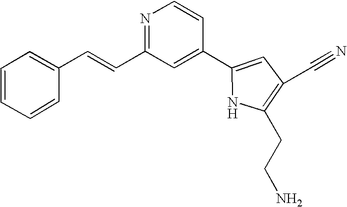

¹H-NMR (400 MHZ, DMSO-d6): 3.20 (t, 2H), 3.29-3.40 (m, 2H), 7.40 (d, 1H), 7.48 (d, 1H), 7.53 (dd, 2H), 7.67 (s, 1H), 7.71 (d, 2H), 8.05 (d, 1H), 8.15 (s, 3H, NH₃⁺), 8.21 (d, 1H), 8.70 (d, 1H), 8.81 (s, 1H), 13.62 (s, 1H, NH).

MS (ESI⁺) m/z: 315 [MH]⁺.

### EXAMPLE 149

### 5-[2-((E)-Styryl)-pyridin-4-yl]-2-(1,2,3,6-tetrahydro-pyridin-4-yl)-1H-pyrrole-3-carbonitrile

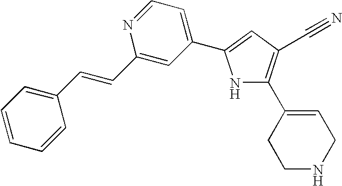

¹H-NMR (400 MHZ, DMSO-d6): 2.94-3.03 (m, 2H), 3.31-3.42 (m, 2H), 3.88 (s, 2H), 6.71 (s, 1H), 7.42 (d, 1H), 7.48 (d, 1H), 7.53 (dd, 2H), 7.71 (d, 2H), 7.79 (s, 1H), 8.15 (s, 1H), 8.24 (d, 1H), 8.72 (d, 1H), 8.92 (s, 1H), 9.28 (s, 2H, NH₂⁺), 12.99 (s, 1H, NH).

MS (ESI⁺) m/z: 353 [MH]⁺.

### EXAMPLE 150

### 5-[2-(2-Morpholin-4-yl-pyrimidin-5-yl)-pyridin-4-yl]-2-piperidin-4-yl-1H-pyrrole-3-carbonitrile

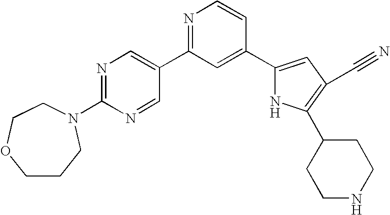

¹H-NMR (400 MHZ, DMSO-d6): 1.89-1.94 (m, 2H), 2.14-2.21 (m, 4H), 3.01-3.08 (m, 2H), 3.31-3.37 (m, 1H), 3.39-3.44 (m, 2H), 3.68 (t, 2H), 3.79 (t, 2H), 3.95-4.00 (m, 4H), 7.74 (s, 1H), 8.10 (d, 1H), 8.69 (d, 1H), 8.84 (s, 1H), 9.11-9.20 (m, 2H, NH₂⁺), 9.25 (s, 2H), 13.55 (s, 1H, NH).

MS (ESI⁺) m/z: 430 [MH]⁺.

### EXAMPLE 151

### 2-(2-Amino-ethyl)-5-(2-quinolin-3-yl-pyridin-4-yl)-1H-pyrrole-3-carbonitrile

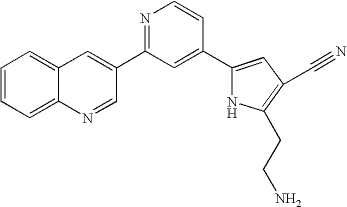

¹H-NMR (400 MHZ, DMSO-d6): 3.20 (t, 2H), 3.30-3.39 (m, 2H), 7.56 (s, 1H), 7.88 (dd, 1H), 8.00 (d, 1H), 8.04 (dd, 1H), 8.20 (s, 3H, NH₃⁺), 8.28 (d, 1H), 8.30 (d, 1H), 8.81 (d, 1H), 9.00 (s, 1H), 9.63 (s, 1H), 9.93 (s, 1H), 13.48 (s, 1H, NH).

MS (ESI⁺) m/z: 440 [MH]⁺.

### EXAMPLE 152

### 2-Aminomethyl-5-[2-((E)-styryl)-pyridin-4-yl]-1H-pyrrole-3-carbonitrile

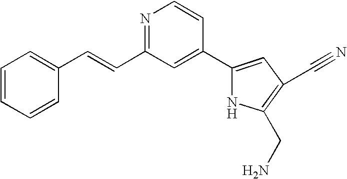

¹H-NMR (400 MHZ, DMSO-d6): 4.26-4.32 (m, 2H), 7.41 (d, 1H), 7.48 (d, 1H), 7.53 (dd, 2H), 7.69 (s, 1H), 7.70 (d, 2H), 8.00 (d, 1H), 8.12 (d, 1H), 8.64-8.73 (m, 4H), 8.75 (d, 1H), 14.00 (s, 1H, NH).

MS (ESI⁺) m/z: 301 [MH]⁺.

### EXAMPLE 153

### 2-Aminomethyl-5-(2-quinolin-3-yl-pyridin-4-yl)-1H-pyrrole-3-carbonitrile

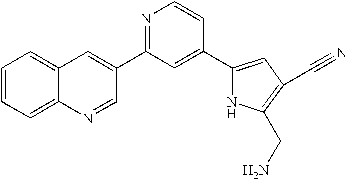

¹H-NMR (400 MHZ, DMSO-d6): 4.26-4.33 (m, 2H), 7.61 (s, 1H), 7.89 (dd, 1H), 7.95 (d, 1H), 8.04 (dd, 1H), 8.28-8.31 (m, 2H), 8.74 (s, 3H, NH₃⁺), 8.85 (d, 1H), 8.86 (s, 1H), 9.53 (s, 1H), 9.90 (s, 1H), 13.84 (s, 1H, NH).

MS (ESI⁺) m/z: 326 [MH]⁺.

### EXAMPLE 154

### 5-(2-Quinolin-3-yl-pyridin-4-yl)-2-(1,2,3,6-tetrahydro-pyridin-4-yl)-1H-pyrrole-3-carbonitrile

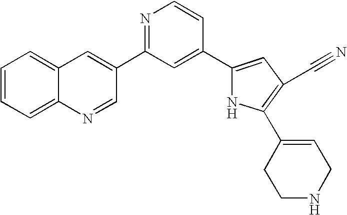

¹H-NMR (400 MHZ, DMSO-d6): 2.92-2.99 (m, 2H), 3.30-3.39 (m, 2H), 3.81-3.88 (m, 2H), 6.63 (s, 1H), 7.61 (s, 1H), 7.81 (dd, 1H), 7.95-8.00 (m, 2H), 8.21 (d, 1H), 8.22 (dd, 1H), 8.77 (d, 1H), 8.93 (s, 1H), 9.25 (s, 2H, NH₂⁺), 9.51 (s, 1H), 9.85 (s, 1H), 12.67 (s, 1H, NH).

MS (ESI⁺) m/z: 378 [MH]⁺.

### EXAMPLE 155

### 2-(2-Amino-ethyl)-5-[2-((E)-styryl)-pyridin-4-yl]-1H-pyrrole-3-carboxylic acid

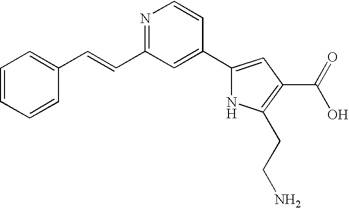

¹H-NMR (400 MHZ, DMSO-d6): 3.19-3.29 (m, 2H), 3.40 (t, 2H), 7.45 (d, 1H), 7.49 (d, 1H), 7.54 (dd, 2H), 7.69 (s, 1H), 7.70 (d, 2H), 8.15 (d, 1H), 8.16 (s, 3H, NH₃⁺), 8.30 (d, 1H), 8.64 (d, 1H), 8.89 (s, 1H), 12.43 (s, br, 1H, OH), 13.32 (s, 1H, NH).

MS (ESI⁺) m/z: 334 [MH]⁺.

### EXAMPLE 156

### 2-(2-Amino-ethyl)-5-(2-quinolin-3-yl-pyridin-4-yl)-1H-pyrrole-3-carboxylic acid

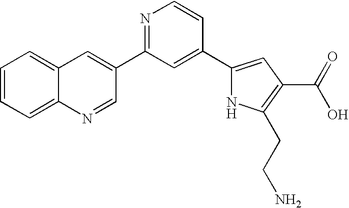

¹H-NMR (400 MHZ, DMSO-d6): 3.16-3.26 (m, 2H), 3.36 (t, 2H), 7.57 (s, 1H), 7.84 (dd, 1H), 7.99 (dd, 1H), 8.00 (d, 1H), 8.09 (s, 3H, NH₃⁺), 8.23 (d, 1H), 8.25 (d, 1H), 8.77 (d, 1H), 8.84 (s, 1H), 9.45 (s, 1H), 9.82 (s, 1H), 12.83 (s, 1H, NH), (OH hidden).

MS (ESI⁺) m/z: 359 [MH]⁺.

### EXAMPLE 157

### 2-(2-Amino-ethyl)-5-(2-phenyl-pyridin-4-yl)-1H-pyrrole-3-carboxylic acid

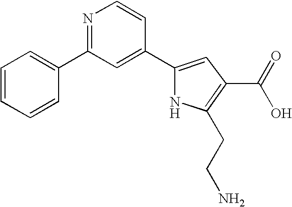

¹H-NMR (400 MHZ, DMSO-d6): 3.17-3.25 (m, 2H), 3.36 (t, 2H), 7.60-7.68 (m, 4H), 8.00-8.14 (m, 4H), 8.16-8.22 (m, 2H), 8.60 (s, 1H), 8.69 (d, 1H), 12.27 (s, br, 1H, OH), 12.91 (s, 1H, NH).

MS (ESI⁺) m/z: 308 [MH]⁺.

### EXAMPLE 158

### 2-(2-Hydroxy-ethyl)-5-[2-((E)-styryl)-pyridin-4-yl]-1H-pyrrole-3-carboxylic acid

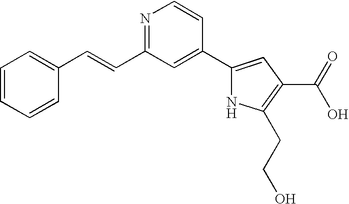

¹H-NMR (400 MHZ, DMSO-d6): 3.17 (t, 2H), 3.42 (s, br, 1H, OH), 3.71 (t, 2H), 7.40 (d, 1H), 7.49 (d, 1H), 7.54 (dd, 2H), 7.69 (s, 1H), 7.70 (d, 2H), 8.04 (d, 1H), 8.14 (d, 1H), 8.62 (d, 1H), 8.63 (s, 1H), 12.26 (s, br, 1H, OH), 13.60 (s, 1H, NH).

MS (ESI⁺) m/z: 335 [MH]⁺.

### EXAMPLE 159

### 2-Aminomethyl-5-[2-((E)-styryl)-pyridin-4-yl]-1H-pyrrole-3-carboxylic acid

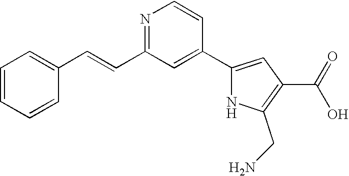

¹H-NMR (400 MHZ, DMSO-d6): 4.42-4.50 (m, 2H), 7.43 (d, 1H), 7.47 (d, 1H), 7.53 (dd, 2H), 7.69 (s, 1H), 7.70 (d, 2H), 8.12 (d, 1H), 8.22 (d, 1H), 8.51 (s, 3H, NH₃⁺), 8.71 (d, 1H), 8.80 (s, 1H), 12.90 (s, br, 1H, OH), 13.71 (s, 1H, NH).

MS (ESI⁺) m/z: 320 [MH]⁺.

### EXAMPLE 160

### 2-Aminomethyl-5-(2-quinolin-3-yl-pyridin-4-yl)-1H-pyrrole-3-carboxylic acid

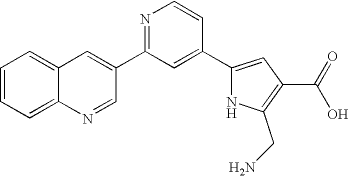

¹H-NMR (400 MHZ, DMSO-d6): 4.44-4.50 (m, 2H), 7.58 (s, 1H), 7.82-7.88 (m, 1H), 7.99 (dd, 1H), 8.04 (d, 1H), 8.28 (d, 1H), 8.30 (d, 1H), 8.65 (s, 3H, NH₃⁺), 8.79 (d, 1H), 8.94 (s, 1H), 9.32 (s, 1H), 9.88 (s, 1H), 13.62 (s, 1H, NH), (OH hidden).

MS (ESI⁺) m/z: 345 [MH]⁺.

### EXAMPLE 161

### 2-(2-Amino-ethyl)-5-[2-(2-[1,4]oxazepan-4-yl-pyrimidin-5-yl)-pyridin-4-yl]-1H-pyrrole-3-carboxylic acid

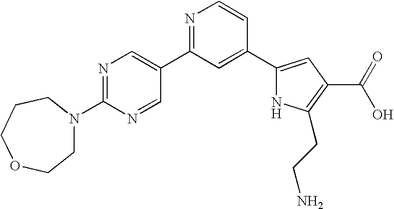

¹H-NMR (400 MHZ, DMSO-d6): 1.87-1.96 (m, 2H), 3.19-3.27 (m, 2H), 3.38 (t, 2H), 3.69 (t, 2H), 3.80 (t, 2H), 3.95-4.01 (m, 4H), 7.70 (s, 1H), 8.07 (s, 1H), 8.04-8.16 (m, 4H, OH, NH₃⁺), 8.63 (d, 1H), 8.75 (s, 1H), 9.22 (s, 2H), 13.24 (s, 1H, NH).

MS (ESI⁺) m/z: 409 [MH]⁺.

### EXAMPLE 162

### 2-(2-Amino-ethyl)-5-[2-(4-methoxy-phenyl)-pyridin-4-yl]-1H-pyrrole-3-carboxylic acid

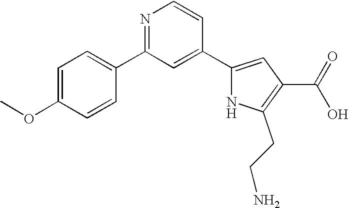

¹H-NMR (400 MHZ, DMSO-d6): 3.16-3.26 (m, 2H), 3.31-3.44 (m, 2H), 3.90 (s, 3H), 7.20 (d, 2H), 7.67 (s, 1H), 8.00-8.13 (m, 5H), 8.19 (d, 2H), 8.62 (d, 1H), 12.38 (s, br, 1H, OH), 12.99 (s, 1H, NH).

MS (ESI⁺) m/z: 338 [MH]⁺.

### EXAMPLE 163

### 2-(2-Amino-ethyl)-5-(5′-methoxy-[2,3′]bipyridinyl-4-yl)-1H-pyrrole-3-carboxylic acid

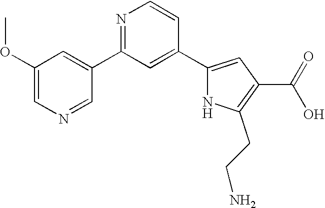

¹H-NMR (400 MHZ, DMSO-d6): 3.19-3.27 (m, 2H), 3.38 (t, 2H), 4.07 (s, 3H), 7.57 (s, 1H), 8.05 (d, 1H), 8.09-8.16 (m, 4H), 8.54 (s, 1H), 8.63 (s, 1H), 8.73 (d, 1H), 8.79 (s, 1H), 9.12 (s, 1H), 13.07 (s, 1H, NH).

MS (ESI⁺) m/z: 339 [MH]⁺.

### EXAMPLE 164

### 2-(2-Amino-ethyl)-5-[2-(3-methanesulfonyl-phenyl)-pyridin-4-yl]-1H-pyrrole-3-carboxylic acid

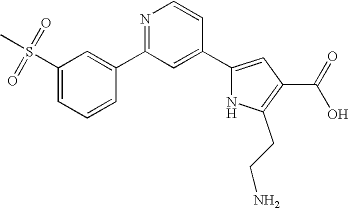

¹H-NMR (400 MHZ, DMSO-d6): 3.15-3.25 (m, 2H), 3.35 (t, 2H), 3.36 (s, 3H), 7.49 (s, 1H), 7.87 (dd, 1H), 7.91 (d, 1H), 8.01-8.11 (m, 5H), 8.09 (s, 1H), 8.57 (d, 1H), 8.71 (d, 1H), 8.73 (s, 1H), 12.69 (s, 1H, NH).

MS (ESI⁻) m/z: 384 [M]⁻.

### EXAMPLE 165

### 2-(2-Amino-ethyl)-5-(6′-methoxy-[2,3′]bipyridinyl-4-yl)-1H-pyrrole-3-carboxylic acid

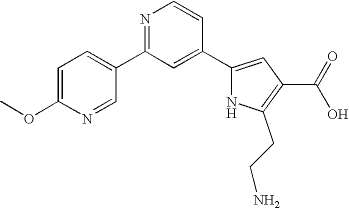

¹H-NMR (400 MHZ, DMSO-d6): 3.16-3.25 (m, 2H), 3.36 (t, 2H), 3.97 (s, 3H), 7.08 (d, 1H), 7.65 (s, 1H), 8.03-8.16 (m, 3H, NH₃⁺), 8.50 (s, 1H), 8.52 (s, 1H), 8.66 (d, 1H), 8.69 (s, 1H), 9.03 (s, 1H), 12.44 (s, br, 1H, OH), 13.09 (s, 1H, NH).

MS (ESI⁺) m/z: 339 [M]⁺.

### EXAMPLE 166

### 2-(2-Amino-ethyl)-5-[2-(2,3-dihydro-benzo[1,4]dioxin-6-yl)-pyridin-4-yl]-1H-pyrrole-3-carboxylic acid

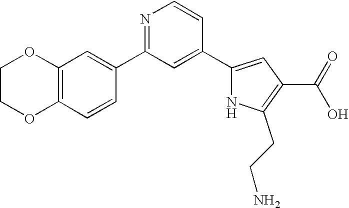

¹H-NMR (400 MHZ, DMSO-d6): 3.14-3.25 (m, 2H), 3.35 (t, 2H), 4.31-4.38 (m, 4H), 7.10 (d, 1H), 7.66 (s, 1H), 7.72 (d, 1H), 7.78 (s, 1H), 8.03 (s, 1H), 8.09 (m, 3H, NH₃⁺), 8.56 (s, 1H), 8.59 (d, 1H), 12.35 (s, br, 1H, OH), 12.97 (s, 1H, NH).

MS (ESI⁺) m/z: 366 [M]⁺.

### EXAMPLE 167

### 2-(2-Amino-ethyl)-5-(2-quinolin-6-yl-pyridin-4-yl)-1H-pyrrole-3-carboxylic acid

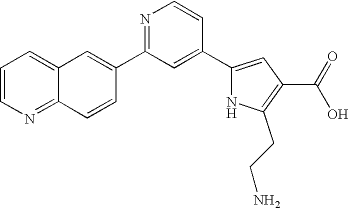

¹H-NMR (400 MHZ, DMSO-d6): 3.16-3.25 (m, 2H), 3.37 (t, 2H), 7.60 (s, 1H), 7.85-7.94 (m, 2H), 8.06 (s, 1H), 8.12 (s, 3H, NH₃⁺), 8.84 (d, 1H), 8.70-8.76 (m, 2H), 8.82 (s, 1H), 9.06 (s, 1H), 9.17 (d, 1H), 9.82 (s, 1H), 12.98 (s, 1H, NH), (OH hidden).

MS (ESI⁺) m/z: 359 [MH]⁺.

### EXAMPLE 168

### 2-(2-Amino-ethyl)-5-{2-[(E)-2-(4-morpholin-4-ylmethyl-phenyl)-vinyl]-pyridin-4-yl}-1H-pyrrole-3-carboxylic acid

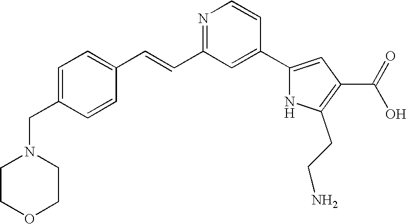

¹H-NMR (400 MHZ, DMSO-d6, 120° C.): 3.03-3.15 (m, 4H), 3.17-3.28 (m, 4H), 3.74-3.85 (m, 4H), 3.86-3.97 (m, 4H), 4.37 (s, 2H), 7.47 (d, 1H), 7.60 (d, 1H), 7.62 (s, 1H), 8.04 (s, 1H), 8.04-8.16 (m, 4H, NH⁺), 8.63 (d, 1H), 8.77 (s, 1H), 11.35 (s, 1H), 12.45 (s, 1H).

MS (ESI⁺) m/z: 433 [MH]⁺.

### EXAMPLE 169

### 2-(2-Amino-ethyl)-5-[2-((E)-styryl)-pyridin-4-yl]-1H-pyrrole-3-carboxylic acid amide

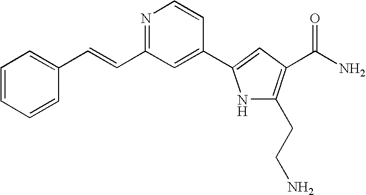

¹H-NMR (400 MHZ, DMSO-d6): 3.09-3.50 (m, 4H), 6.91 (s, br, 2H, NH₂), 7.25 (s, 1H), 7.34 (s, 1H), 7.37 (d, 1H), 7.74 (dd, 2H), 7.57 (d, 1H), 7.64 (d, 2H), 7.84 (d, 1H), 7.96 (s, br, 3H, NH₃⁺), 8.07 (s, 1H), 8.50 (d, 1H), 12.18 (s, 1H, NH).

MS (ESI⁺) m/z: 333 [MH]⁺.

### EXAMPLE 170

### 5-[2-((E)-Styryl)-pyridin-4-yl]-2-(1,2,3,6-tetrahydro-pyridin-4-yl)-1H-pyrrole-3-carboxylic acid amide

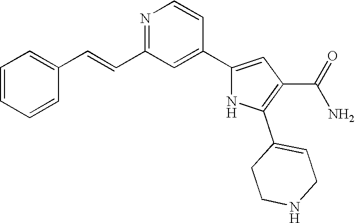

¹H-NMR (400 MHZ, DMSO-d6): 2.83.2.91 (m, 2H), 3.25-3.33 (m, 2H), 3.74-3.84 (m, 2H), 6.29 (s, 1H), 7.19 (s, 1H, NH), 7.43 (d, 1H), 7.50 (d, 1H), 7.52 (dd, 2H), 7.54 (s, 1H), 7.70 (s, 1H), 7.71 (d, 2H), 8.05 (d, 1H), 8.24 (d, 1H), 8.65 (d, 1H), 8.81 (s, 1H, NH), 9.25 (s, 2H, NH₂⁺), 12.47 (s, 1H, NH).

MS (ESI⁺) m/z: 371 [MH]⁺.

### EXAMPLE 171

### 2-Aminomethyl-5-[2-((E)-styryl)-pyridin-4-yl]-1 H-pyrrole-3-carboxylic acid amide

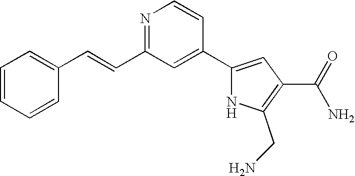

¹H-NMR (400 MHZ, DMSO-d6): 4.32-4.41 (m, 2H), 7.43 (d, 1H), 7.47 (d, 1H), 7.52 (dd, 2H), 7.70 (d, 2H), 7.78 (s, 1H), 7.84 (d, 1H), 7.95 (s, br, 3H, NH₃⁺), 8.21 (d, 1H), 8.54 (s, 2H), 8.68 (d, 1H), 8.74 (s, 1H), 13.70 (s, 1H, NH).

MS (ESI⁺) m/z: 319 [MH]⁺.

### EXAMPLE 172

### 2-(2-Dimethylamino-ethyl)-5-[2-((E)-styryl)-pyridin-4-yl]-1H-pyrrole-3-carbonitrile

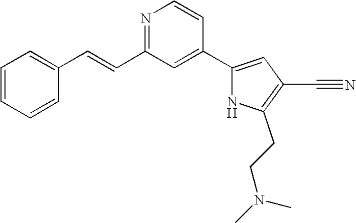

2-(2-Amino-ethyl)-5-[2((E)-styryl)-pyridin-4-yl]-1H-pyrrole-3-carbonitrile hydrochloride (example 147, 20 mg, 0.06 mmol) is dissolved in 1.0 ml MeOH and after addition of 18 μl (318 mmol) acetic acid and 12 μl (191 mmol) formaldehyde solution (36.5% in water) the mixture is stirred 10 min at room temperature before adding 16 mg (255 mmol) NaCNBH₃. The mixture is stirred overnight at room temperature. Then the reaction is diluted with ethyl acetate, washed with sat. NaHCO₃ and NaCl-solution, dried over Na₂SO₄ and evaporated. The crude product is purified by silica gel chromatography (ethyl acetate-methanol gradient).

¹H-NMR (400 MHZ, DMSO-d6): 2.89 (s, 6H), 3.31 (t, 2H), 3.44-3.66 (m, 2H), 7.36 (d, 1H), 7.47 (d, 1H), 7.52 (dd, 2H), 7.62 (s, 1H), 7.69 (s, 1H), 7.70 (d, 2H), 7.98 (s, 1H), 8.16 (d, 1H), 8.69 (d, 1H), 13.66 (s, 1H, NH).

MS (ESI⁺) m/z: 343 [MH]⁺.

Agents of the Invention possess MAPKAPK2 (MAP Kinase Activated Protein Kinase) inhibiting activity. (MAPKAPK2 and MK2 are used as synonyms) Thus the Agents of the Invention act to inhibit production of inflammatory cytokines, such as TNF-α, and also to potentially block the effects of these cytokines on their target cells. These and other pharmacological activities of the Agents of the Invention as may be demonstrated in standard test methods for example as described below:

MAPKAPK2 (or MK2) Kinase Assay

MAPAPK2 is pre-activated in kinase buffer (25 mM TRIS-HCL, pH 7.5, 25 mM beta-glycerophosphate, 0.1 mM sodium orthovanadate, 25 mM MgCl₂, 20 μM DTT) containing 5 μM ATP, 150 μg/ml human MK2 (HPLC purified in house), 30 μg/ml active human p38α (HPLC purified in house) for 30 min at 22° C. For the measurement of compound inhibition on activated MAPAPK2, each reaction contained test compound (10 μl; 0.5% DMSO final) or vehicle control, 250 nM Hsp27 peptide biotinyl-AYSRALSRQLSSGVSEIR—COOH as substrate (10 μl) and pre-activated MAPKAP2 kinase mix (10 μl) containing ATP (5 μM final). To define non-specific, reactions are performed in the absence of substrate. Following incubation at 22° C. for 45 min, kinase reactions are terminated with 125 μM EDTA (10 μl). Samples (10 μl) are transferred to black low volume 384-well plates (Greiner) prior to the detection of phosphorylated substrate by time-resolved fluorescence resonance energy transfer (TR-FRET). Phosphorylated Hsp27 is measured using an antibody mix (10 μl) containing a rabbit anti-phospho-Hsp27 (Ser⁸²) antibody (2.5 nM, Upstate) in conjunction with an anti-rabbit europium-labeled secondary antibody LANCE Eu-W1024 (2.5 nM; Perkin Elmer) as fluorescence donor along with streptavidin SureLight-APC (6.25 nM; Perkin Elmer) as a fluorescence acceptor. Following incubation at 22° C. for 90 min, plates are measured at 615 and 665 nm using a PHERAstar (BMG Labtech). The 615/665 nm ratio is determined following subtraction of background. Values are expressed as % inhibition using control values. Individual IC₅₀ values of compounds are determined by nonlinear regression after fitting of curves to the experimental data using Excel XL fit 4.0 (Microsoft).

For a number of compounds being identified by their example No. the efficacy is disclosed as being assessable by the above described MK2 kinase assay:

| Example No. | IC50 [μM] (micromolar) |
|---|---|
| 9 | 0.13 |
| 14 | 0.12 |
| 51 | 0.05 |
| 55 | 0.26 |
| 72 | 0.10 |
| 75 | 0.15 |
| 81 | 0.14 |
| 93 | 0.09 |
| 105 | 0.07 |
| 109 | 0.16 |
| 110 | 0.15 |
| 116 | 0.20 |
| 118 | 0.07 |
| 131 | 0.29 |
| 144 | 0.21 |
| 148 | 0.15 |
| 155 | 0.08 |
| 170 | 0.27 |

PDK-1 Kinase Assay

Enzyme activities: Enzyme activities are measured by mixing 10 μL of a 3-fold concentrated compound solution with 10 μL of the corresponding substrate mixture (peptidic substrate, ATP and [γ33P]ATP) and the reactions are initiated by the addition of 10 μL of a 3-fold concentrated solution of GST-PDK1 in assay buffer. The enzymatic reactions are stopped by the addition of 20 μL of 125 mM EDTA. The incorporation of 33P into the peptidic substrates are quantified by two different methods, filter binding (FB) and flash-plate (FP) method:

Filter binding assays: The assays are carried out in 96-well plates at room temperature for 10 min (FB) in a final volume of 30 μL including the following components: (a) 50 ng GST-PDK1, 20 mM Tris-HCl, pH 7.5, 10 mM MgCl₂, 1 mM DTT, 10 μM Na₃VO₄, 0.1 mM EGTA, 3 μg/mL of a non-biotinylated peptide, 1% DMSO and 10 μM ATP (γ-[³³P]-ATP 0.1 μCi); (b) 100 ng GST-PDK1, 20 mM Tris-HCl, pH 7.5, 10 mM MgCl₂, 1 mM DTT, 10 μM Na₃VO₄, 0.1 mM EGTA, 100 μg/mL of CASEIN, 1% DMSO and 10 μM ATP (γ[³³P]-ATP 0.1 μCi);

The capturing of the phosphorylated peptides by the FB method is performed as following: 40 μL of the stopped reaction mixture are transferred onto Immobilon-PVDF membranes previously soaked for 5 min with methanol, rinsed with water, soaked for 5 min with 0.5% H₃PO₄ and mounted on vacuum manifold with disconnected vacuum source. After spotting, vacuum is connected and each well rinsed with 200 μL 0.5% H₃PO₄. Free membranes are removed and washed 4 times on a shaker with 1% H₃PO₄ and once with ethanol. Membranes are counted after drying, mounting in Packard TopCount 96-well frame, and addition of 10 μL/well of Microscint™. The plates are eventually sealed and counted in a microplate scintillation counter (TopCount NXT, TopCount NXT HTS).

Flash plate method: Assays by the FP method are first carried out in a total volume of 30 μL at RT in conventional 96-well polystyrene-based plastic plates. The assays included 300 ng GST-PDK1, 20 mM Tris-HCl, pH 7.5, 10 mM MgCl₂, 1 mM DTT, 10 μM Na₃VO₄, 0.1 mM EGTA, 10 μg/mL of a biotinylated peptide, 1% DMSO and 10 μM ATP (γ-[³³P]-ATP 0.1 μCi). The reactions are stopped after 30 min by the addition of 20 μL of 125 mM EDTA. For the capturing of the phosphorylated substrates (60 min, RT), transfer steps to streptavidin-coated FPs are necessary. All assay plates are then washed three times with PBS and dried at room temperature. The plates are sealed and counted in a microplate scintillation counter (TopCount NXT, TopCount NXT HTS).

Calculation of IC₅₀'s: IC₅₀ values are calculated by linear regression analysis of the percentage inhibition of the compound either in duplicate, at four concentrations (usually 0.01, 0.1, 1 and 10 μM) or as 8 single point IC₅₀ starting at 10 μM following by 1:3 dilutions

Assay for Inhibition of TNF-α Release from hPBMCs

Human peripheral blood mononuclear cells (hPBMCs) are prepared from the peripheral blood of healthy volunteers using Ficoll-Plaque Plus (Amersham) density separation according to the method described within. Cells are seeded at a 1×10⁵ cells/well in 96-well plates in RPMI 1640 medium (Invitrogen) containing 10% (v/v) fetal calf serum (FCS). Cells are pre-incubated with serial dilutions of test compound (0.25% v/v DMSO final) for 30 min at 37° C. Cells are stimulated with the addition of IFNγ (10 ng/ml) and lipopolysaccharide (LPS) (5 μg/ml) per well and incubated for 3 h at 37° C. Following a brief centrifugation (250×g for 2 min), supernatant (10 μl) samples are taken from each well and measured against a TNFα calibration curve using a HTRF TNFα kit (CisBio) as described within. Individual IC₅₀ values of compounds are determined by nonlinear regression after fitting of curves to the experimental data using Excel XL fit 4.0 (Microsoft).

Assay for Inhibition of TNF-α Production in LPS Stimulated Mice

Injection of lipopolysaccharide (LPS) induces a rapid release of soluble tumour necrosis factor (TNF-α) into the periphery. This model is be used to analyse prospective blockers of TNF release in vivo.

LPS (20 mg/kg) is injected i.v. into OF1 mice (female, 8 week old). One (1) hour later blood is withdrawn from the animals and TNF levels are analysed in the plasma by an ELISA method using an antibody to TNF-α. Using 20 mg/kg of LPS levels of up to 15 ng of TNF-α/ml plasma are usually induced. Compounds to be evaluated are given either orally or s.c. 1 to 4 hours prior to the LPS injection. Inhibition of LPS-induced TNF-release is taken as the readout.

Agents of the Invention typically inhibit TNF production to the extent of up to about 50% or more in the above assay when administered at 30 mg/kg.

Agents of the invention are useful for the prevention and/or treatment of diseases, conditions and disorders that are mediated by TNF alpha and/or by MK2, including autoimmune diseases, inflammation and arthritis. The agents of the invention may also be used for example for the treatment of pain, headaches, or as an antipyretic for the treatment of fever.

In preferred uses, the agents of the invention may be used for the treatment of any of one or more of the following disorders: connective tissue and joint disorders, neoplasia disorders, cardiovascular disorders, ophthalmic disorders, respiratory disorders, gastrointestinal disorders, angiogenesis-related disorders, autoimmune and immunological disorders, allergic disorders, infectious diseases and disorders, endocrine disorders, metabolic disorders, neurological and neurodegenerative disorders, pain, hepatic and biliary disorders, musculoskeletal disorders, genitourinary disorders, gynaecological and obstetric disorders, injury and trauma disorders, muscle disorders, surgical disorders, dental and oral disorders, sexual dysfunction orders, dermatological disorders, hematological disorders, and poisoning disorders.

In other preferred embodiments, agents of the invention may be used for the prevention and treatment of autoimmune and inflammatory disorders such as arthritis (e.g. rheumatoid arthritis, psoriatic arthritis, juvenile chronic arthritis, reactive arthritis, arthritis deformans, gouty arthritis, osteoarthritis, lyme disease, autoimmune haematological disorders (e.g. hemolytic anaemia, aplastic anaemia, pure red cell anaemia and idiopathic thrombocytopenia), enterogenic spondyloarthropathies, ankylosing spondylitis, inflammatory bowel disease, ulcerative colitis, Crohn's disease, multiple sclerosis, lumbar spondylarthrosis, carpal tunnel syndrome, canine hip dysplasia, systemic lupus erythematosus, lupus nephritis, polychondritis, scleroderma, Wegener granulamatosis, Steven-Johnson syndrome, dermatomyositis, polymyositis, gout, tendonitis and bursitis, organ or transplant rejection (e.g for the treatment of recipients of heart, lung, combined heart lung, liver, kidney, pancreatic, skin or corneal transplants), graft-versus-host disease, sepsis, septic shock, Behcet's disease, uveitis (anterior and posterior), Muckle-Wells syndrome, psoriasis, cutaneous lupus erythematosus, dermatitis, atopic dermatitis, acne vulgaris, eczema, xerosis, type I diabetes, Graves disease, Sjogrens syndrome, blistering disorders (e.g. pemphigus vulgaris).

In other preferred embodiments be agents of the invention may be used for the prevention and treatment of neoplasia disorders such as acral lentiginous melanoma, actinic keratoses, adenocarcinoma, adenomas, familial adenomatous polyposis, familial polyps, colon polyps, polyps, adenosarcoma, adenosquamous carcinoma, adrenocortical carcinoma, AIDS-related lymphoma, anal cancer, astrocytic tumours, batholin gland carcinoma, basal cell carcinoma, bile duct cancer, bladder cancer, brainstem glioma, brain tumours, breast cancer, bronchial gland carcinomas, capillary carcinoma, carcinoids, carcinoma, carcinomasarcoma, cavernous, central nervous system lymphoma, cerebral astrocytoma, cholangiocarcinoma, chondrosarcoma, choroid plexus papilloma/carcinoma, clear cell carcinoma, skin cancer, brain cancer, colon cancer, colorectal cancer, cutaneous T-cell lymphoma, cystadenoma, endodermal sinus tumour, endometrial hyperplasia, endometrial stromal sarcoma, endometrioid adenocarcinoma, ependymal, epitheloid, esophagal cancer, Ewing's sarcoma, extragonal germ cell tumour, fibrolamellar, focal nodular hyperplasia, gallbladder cancer, gastrinoma, germ cell tumours, gestation trophoblastic tumour, glioblastoma, hemangioblastomas, hemangiomas, hepatic adenomas, hepatic adenomatosis, hepatocellular carcinoma, Hodgkin's lymphoma, hypopharyngeal cancer, hypothalamic and visual pathway glioma, insulinoma, intraepithelial neoplasia, interepithelial cell carcinoma, Kaposi's sarcoma, kidney cancer, laryngeal cancer, leiomyosarcoma, lentigo maligna melanomas, leukaemia-related disorders, lip and oral cavity cancer liver cancer, lung cancer, lymphoma, malignant mesothelial tumours, malignant thymoma, medulloblastoma, medulloepithelioma, melanoma, meningeal, merkel cell carcinoma, mesothelial, metastatic carcinoma, mucoepidermoid carcinoma, multiple myelomatplasma cell neoplasm, mycosis fungoides, myelodysplastic syndrome, myeloproliferative disorders, nasal cavity and paranasal sinus cancer, nasopharyngeal cancer, neuroblastoma, and neuroepithelial adenocarcinoma nodular melanoma, non-Hodgkin's lymphoma, oat cell carcinoma, oligodendroglial, oral cancer, oropharyngeal cancer, osteosarcoma, pancreatic polypeptide, ovarian cancer, ovarian germ cell cancer, pancreatic cancer, papillary serous adenocarcinoma, pineal cell, pituitary tumours, plasmacytoma, pseudosarcoma, pulmonary blastoma, parathyroid cancer, penile cancer, pheochromcytoma, dermal tumours, pituitary tumour, plasma cell neoplasm, pleuropulmonay blastoma, prostate cancer, rectal cancer, renal cell carcinoma, retinoblastoma, rhabdomysarcoma, sarcoma, serous carcinoma, small cell carcinoma, small intestine cancer, soft tissue carcinomas, somatostatin secreting tumour, squamous carcinoma, squamous cell carcinoma, submesothelial, superficial spreading carcinoma, supratentorial primitive neurectodermal tumours, thyroid cancer, undifferentiated carcinoma, urethral cancer, uterine sarcoma, uveal melanoma, verrucous carcinoma, vaginal cancer, vipoma, vulvar cancer, Waldonstrom's macroglobulinemia, well differentiated carcinoma, and Wilm's tumour.

Agents of the invention may further be used to treat or prevent cardiovascular disorders, for example myocardial ischaemia, hypertension, hypotension, heart arrhythmias, pulmonary hypertension, hypokalaemia, cardiac ischaemia, myocardial infarction, cardiac remodelling, cardiac fibrosis, myocardial necrosis, aneurysm, arterial fibrosis, embolism, vascular plaque inflammation, vascular plaque rupture, bacterial induced inflammation and viral induced inflammation, oedema, swelling, fluid accumulation, cirrhosis of the liver, Bartter's syndrome, myocarditis, arteriosclerosis, at atherosclerosis, calcification (such as vascular calcification and valvar calcification), coronary artery disease, acute coronary syndrome, heart failure, congestive heart failure, shock, arrhythmia, left ventricular hypertrophy, angina, diabetic nephropathy, kidney failure, eye damage, vascular diseases, migraine headaches, aplastic anaemia, cardiac damage, diabetic cardiac myopathy, renal insufficiency, renal injury, renal arteriography, peripheral vascular disease, left ventricular hypertrophy, cognitive dysfunction, stroke and headache.

In other preferred embodiments, agents of the invention may be used for the prevention and treatment of bone and muscle disorders such as sarcopenia, muscular dystrophy, cachexia or wasting syndrome associated with morbid TNF release (e.g. consequent to infection, cancer or organ dysfunction, especially AIDS-related cachexia), and osteoporosis.

In further preferred embodiments agents of the invention may be used for the prevention and treatment of respiratory disorders such as asthma and bronchitis, chronic obstructive pulmonary disease (COPD), cystic fibrosis, pulmonary oedema, pulmonary embolism, pneumonia, pulmonary sarcoisis, silicosis, pulmonary fibrosis, respiratory failure, acute respiratory distress syndrome, primary pulmonary hypertension and emphysema.

In further preferred embodiments, agents of the invention may be used for the prevention and treatment of the angiogenesis-related disorders selected from: angiofibroma, neovascular glaucoma, arteriovenous malformations, arthritis, Osler-Weber syndrome, atherosclerotic plaques, psoriasis, corneal graft neovascularisation, pyogenic granuloma, delayed wound healing, retrolental fibroplasias, diabetic retinopathy, stroke, cancer, AIDS complications, ulcers and infertility.

In further preferred embodiments, agents of the invention may be used for the prevention or treatment of infectious diseases and disorders such as viral infections, bacterial infections, prion infections, spiroketes infections, mycobacterial infections, rickettsial infections, chlamydial infections, parasitic infections and fungal infections.

In yet other preferred embodiments, agents of the invention may be used to the prevention and treatment of neurological and neurodegenerative disorders such as headaches, migraine, pain, dental pain, neuropathic and inflammatory pain, Alzheimer's disease, Parkinson's disease, dementia, memory loss, senility, amyotrophy, ALS, amnesia, seizures, multiple sclerosis, muscular dystrophy use, epilepsy, schizophrenia, depression, anxiety, attention deficit disorder, hyperactivity, spongiform encephalopathy, Creutzfeld-Jacob disease, Huntington's Chorea, ischaemia.

In yet other preferred embodiments, agents of the invention may be used for the prevention and treatment of autoimmune and inflammatory disorders such as arthritis (e.g. rheumatoid arthritis, psoriatic arthritis, juvenile chronic arthritis, reactive arthritis, arthritis deformans, gouty arthritis, osteoarthritis, lyme disease, enterogenic spondyloarthropathies, ankylosing spondylitis, inflammatory bowel disease, ulcerative colitis, Crohn's disease, multiple sclerosis, lumbar spondylarthrosis, carpal tunnel syndrome, systemic lupus erythematosus, lupus nephritis, polychondritis, scleroderma, Wegener granulamatosis, Steven-Johnson syndrome, dermatomyositis, polymyositis, gout, tendonitis and bursitis, organ or transplant rejection (e.g for the treatment of recipients of heart, lung, combined heart lung, liver, kidney, pancreatic, skin or corneal transplants), graft-versus-host disease, sepsis, septic shock, Behcet's disease, uveitis (anterior and posterior), Muckle-Wells syndrome, psoriasis, cutaneous lupus erythematosus, dermatitis, atopic dermatitis, acne vulgaris, eczema, xerosis, type I diabetes, Graves disease, Sjogrens syndrome, blistering disorders (e.g. pemphigus vulgaris), neoplasia disorders such as adenocarcinoma, adenosarcoma, basal cell carcinoma, bile duct cancer, bladder cancer, brain tumours, breast cancer, bronchial gland carcinomas, capillary carcinoma, skin cancer, brain cancer, colon cancer, colorectal cancer, cutaneous T-cell lymphoma, gallbladder cancer, glioblastoma, Hodgkin's lymphoma, interepithelial cell carcinoma, Kaposi's sarcoma, kidney cancer, lentigo maligna melanomas, leukaemia-related disorders, liver cancer, lung cancer, lymphoma, malignant mesothelial tumours, metastatic carcinoma, multiple myeloma/plasma cell neoplasm, myeloproliferative disorders, non-Hodgkin's lymphoma, ovarian cancer, pancreatic cancer, prostate cancer, cardiovascular disorders, for example myocardial ischaemia, cardiac ischaemia, myocardial infarction, cardiac fibrosis, vascular plaque inflammation, vascular plaque rupture, bacterial induced inflammation and viral induced inflammation, oedema, swelling, fluid accumulation, myocarditis, arteriosclerosis, atherosclerosis, coronary artery disease, acute coronary syndrome, heart failure, congestive heart failure, ventricular hypertrophy, bone and muscle disorders such as sarcopenia, muscular dystrophy, cachexia or wasting syndrome associated with morbid TNF release (e.g. consequent to infection, cancer or organ dysfunction, especially AIDS-related cachexia). respiratory disorders such as asthma and bronchitis, chronic obstructive pulmonary disease (COPD), cystic fibrosis, pulmonary oedema, pulmonary fibrosis, respiratory failure, acute respiratory distress syndrome, neurological and neurodegenerative disorders such as headaches, migraine, pain, dental pain, neuropathic and inflammatory pain, multiple sclerosis.

In yet other preferred embodiments, agents of the invention may be used for the prevention and treatment of arthritis (e.g. rheumatoid arthritis, psoriatic arthritis, juvenile chronic arthritis, reactive arthritis, arthritis deformans, gouty arthritis, osteoarthritis), enterogenic spondyloarthropathies, ankylosing spondylitis, inflammatory bowel disease, ulcerative colitis, Crohn's disease, multiple sclerosis, lumbar spondylarthrosis, systemic lupus erythematosus, lupus nephritis, scleroderma, gout, tendonitis and bursitis, organ or transplant rejection (e.g for the treatment of recipients of heart, lung, combined heart lung, liver, kidney, pancreatic, skin or corneal transplants), graft-versus-host disease, sepsis, septic shock, Behcet's disease, uveitis (anterior and posterior), Muckle-Wells syndrome, psoriasis, cutaneous lupus erythematosus, dermatitis, atopic dermatitis, eczema, blistering disorders (e.g. pemphigus vulgaris), myocardial ischaemia, vascular plaque inflammation, vascular plaque rupture, bacterial induced inflammation, atherosclerosis, coronary artery disease, acute coronary syndrome, congestive heart failure, sarcopenia, muscular dystrophy, cachexia or wasting syndrome associated with morbid TNF release (e.g. consequent to infection, cancer or organ dysfunction, especially AIDS-related cachexia), asthma and bronchitis, chronic obstructive pulmonary disease (COPD), cystic fibrosis, pulmonary oedema, pulmonary fibrosis, respiratory failure, acute respiratory distress syndrome, headaches, migraine, pain, dental pain, neuropathic and inflammatory pain.

In a more preferred embodiment, agents of the invention may be used for the prevention and treatment of arthritis (e.g. rheumatoid arthritis, psoriatic arthritis, juvenile chronic arthritis, reactive arthritis, arthritis deformans, gouty arthritis, osteoarthritis), ankylosing spondylitis, inflammatory bowel disease, ulcerative colitis, Crohn's disease, multiple sclerosis, systemic lupus erythematosus, lupus nephritis, scleroderma, gout, sepsis, septic shock, Behcet's disease, Muckle-Wells syndrome, psoriasis, cutaneous lupus erythematosus, dermatitis, atopic dermatitis, eczema, blistering disorders (e.g. pemphigus vulgaris), vascular plaque inflammation, bacterial induced inflammation, atherosclerosis, sarcopenia, cachexia or wasting syndrome associated with morbid TNF release (e.g. consequent to infection, cancer or organ dysfunction, especially AIDS-related cachexia), asthma and bronchitis, chronic obstructive pulmonary disease (COPD), headaches, migraine, neuropathic and inflammatory pain.

For all the above uses, an indicated daily dosage is in the range from about 0.03 to about 300 mg preferably 0.03 to 30, more preferably 0.1 to 10 mg of a compound of the invention. Agents of the Invention may be administered twice a day or up to twice a week.

The Agents of the Invention may be administered in free form or in pharmaceutically acceptable salt form. Such salts may be prepared in conventional manner and exhibit the same order of activity as the free compounds. The present invention also provides a pharmaceutical composition comprising an Agent of the Invention in free base form or in pharmaceutically acceptable salt form in association with a pharmaceutically acceptable diluent or carrier. Such compositions may be formulated in conventional manner. The Agents of the Invention may be administered by any conventional route, for example parenterally e.g. in form of injectable solutions, microemulsions or suspensions, enterally, e.g. orally, for example in the form of tablets, capsules or drinking solutions; sub-lingual, topically or transdermally, e.g. in form of a dermal cream or gel or for the purpose of administration to the eye in the form of an ocular cream, gel or eye-drop preparation, or it may be administered by inhalation.

The compounds of the invention may also be administered simultaneously, separately or sequentially in combination with one or more other suitable active agents selected from the following classes of agents: Anti IL-1 agents, e.g: Anakinra; anti cytokine and anti-cytokine receptor agents, e.g. anti IL-6 R Ab, anti IL-15 Ab, anti IL-17 Ab, anti IL-12 Ab; B-cell and T-cell modulating drugs, e.g. anti CD20 Ab; CTL4-Ig, disease-modifying anti-rheumatic agents (DMARDs), e.g. methotrexate, leflunamide, sulfasalazine; gold salts, penicillamine, hydroxychloroquine and chloroquine, azathioprine, glucocorticoids and non-steroidal anti-inflammatories (NSAIDs), e.g. cyclooxygenase inhibitors, selective COX-2 inhibitors, agents which modulate migration of immune cells, e.g. chemokine receptor antagonists, modulators of adhesion molecules, e.g. inhibitors of LFA-1, VLA-4.

1. A compound of formula (I) or a pharmaceutically acceptable salt thereof: 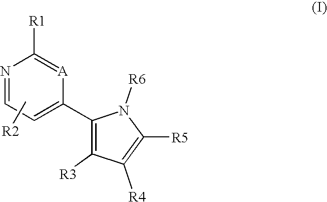
wherein A is CH or N;
R1 is aryl, heteroaryl, aryl-C₂-C₆ alkenyl, heteroaryl-C₂-C₆ alkenyl, C₃-C₇ cycloalkyl, C₃-C₇ cycloalkyl-C₂-C₆ alkenyl, aryl-C₂-C₆ alkynyl, heteroaryl-C₂-C₆ alkynyl, C₃-C₇ cycloalkyl-C₂-C₆ alkynyl, heterocycloalkyl, heterocycloalkyl C₂-C₈ alkenyl, heterocycloalkyl C₂-C₆ alkynyl, amino, C₁-C₆ alkylamino, arylamino, heteroarylamino, aryloxy, or heteroaryloxy, and
R1 is optionally substituted with halo, C₁-C₆ alkyl, cyano, heterocycloalkyl, heterocycloalkyl-C₁-C₆ alkyl, carbamoyl, carbonyl, carboxyl, carbonylamino, heterocycloalkylcarbonyl, amino, C₁-C₆ alkylamino, sulfonyl, C₁-C₆ alkylcarbonylamino, hydroxy, C₁-C₆ alkoxy, C₁-C₆ cycloalkyloxy, aryloxy or heteroaryloxy, each of which may be optionally substituted with C₁-C₆ alkyl, cycloalkyl, heterocycloalkyl, C₁-C₆ alkoxy, amino, cyano, halo, carboxyl, carboxyalkyl, carbamoyl, or hydroxyl;
R2 is H, aryl, heteroaryl, or aryl-C₂-C₆ alkenyl, and R2 is optionally substituted with halo, C₁-C₆ alkyl, cyano, heterocycloalkyl, heterocycloalkyl-C₁-C₆ alkyl, carbamoyl, carbonyl, carbonylamino, heterocycloalkylcarbonyl, amino, C₁-C₆ alkylamino, sulfonyl, C₁-C₆ alkylcarbonylamino, hydroxy, C₁-C₆ alkoxy, C₁-C₆ cycloalkyloxy, aryloxy, heteroaryloxy, each of which may be optionally substituted with C₁-C₆ alkyl, cycloalkyl, heterocycloalkyl, C₁-C₆ alkoxy, amino, cyano, halo, carboxyl, carboxyalkyl, carbamoyl, hydroxyl;
R3 is H, C₁-C₆ alkyl, cyano, amino, halo, CF₃ or CHF₂;
R4 is cyano, carbamoyl, sulfamoyl, amidino, N-hydroxy amidino, carboxyl, or carboxylic ester;
R5 is heterocycloalkyl or C₁-C₆ alkyl which is optionally substituted with C₁-C₆ alkyl, halo, cyano, hydroxyl, amino, sulfonyl, aryl, carbonyl, C₁-C₆ alkylcarbonyl, C₁-C₆ alkyloxycarbonyl, or C₁-C₆ alkyloxy,
each of which may be optionally substituted with C₁-C₆ alkyl, C₁-C₆ alkyloxy, hydroxyl, sulfonyl, aryl, halo, cyano, amino, alkylamino, or dialkylamino;
R6 is H or C₁-C₆ alkyl or sulfonyl, and
R6 is optionally substituted with C₁-C₆ alkyl, hydroxy, halo, cyano, amino, alkylamino, or dialkylamino.

2. The compound of claim 1, wherein R1 is aryl, heteroaryl, aryl-C₂-C₆ alkenyl, amino or arylamino, and
R1 is optionally substituted with halo, C₁-C₆ alkyl, heterocycloalkyl, heterocycloalkyl-C₁-C₆ alkyl, carbamoyl, carbonyl, carboxyl, C₁-C₆ alkoxy, heterocycloalkylcarbonyl, amino, C₁-C₆ alkylamino, sulfonyl, or Cₗ-C₆ alkylcarbonylamino, each of which may be optionally substituted with C₁-C₆ alkyl, C₁-C₆ alkoxy, or amino; and
R2 is H;

3. The compound of claim 2, wherein R1 is optionally substituted aryl-C₂-C₆ alkenyl.

4. The compound in accordance to claim 1, wherein R4 is selected from the group consisting of cyano, carbamoyl, amidino, N-hydroxy amidino, carboxyl, and carboxylic ester.

5. The compound according to claim 1 wherein said compound is:
5′-{2-[(E)-2-(4-Fluoro-phenyl)-vinyl]-pyridin-4-yl}-2,3,4,5-tetrahydro-1′H-[1,2′]bipyrrolyl-3′carbonitrile,
5′-{2-[(E)-2-(4-Fluoro-phenyl)-vinyl]-pyridin-4-yl}-2,3,4,5-tetrahydro-1′H-[1,2′]bipyrrolyl-3′carboxylic acid amide,
5-{2-[(E)-2-(4-Fluoro-phenyl)-vinyl]-pyridin-4-yl}-2-morpholin-4-yl-1H-pyrrole-3-carbonitrile,
5-{2-[(E)-2-(4-Fluoro-phenyl)-vinyl]-pyridin-4-yl}-2-morpholin-4-yl-1H-pyrrole-3-carboxylic acid amide,
2-(2-Amino-ethylamino)-5-[2-((E)-styryl)-pyridin-4-yl]-1H-pyrrole-3-carbonitrile,
2-(3-Hydroxy-propylamino)-5-[2-((E)-styryl)-pyridin-4-yl]-1H-pyrrole-3-carbonitrile,
2-(2-Hydroxy-ethylamino)-5-[2((E)-styryl)-pyridin-4-yl]-1H-pyrrole-3-carbonitrile,
5-{2-[(E)-2-(4-Fluoro-phenyl)-vinyl]-pyridin-4-yl}-2-piperazin-1-yl-1H-pyrrole-3-carbonitrile trifluoroacetate,
5-{2-[(E)-2-(4-Fluoro-phenyl)-vinyl]-pyridin-4-yl}-2-piperazin-1-yl-1H-pyrrole-3-carboxylic acid amide,
2-Piperazin-1-yl-5-(2-quinolin-3-yl-pyridin-4-yl)-1H-pyrrole-3-carbonitrile trifluoroacetate,
2-Piperazin-1-yl-5-(2-quinolin-3-yl-pyridin-4-yl)-1H-pyrrole-3-carboxylic acid amide,
5-(2-Benzofuran-2-yl-pyridin-4-yl)-2-piperazin-1-yl-1H-pyrrole-3-carbonitrile trifluoroacetate,
2-Piperazin-1-yl-5-[2-((E)-styryl)-pyridin-4-yl]-1H-pyrrole-3-carbonitrile,
2-Piperazin-1-yl-5-[2-((E)-styryl)-pyridin-4-yl]-1H-pyrrole-3-carboxylic acid amide,
5-[2-(1-Methyl-1H-indol-5-yl)-pyridin-4-yl]-2-piperazin-1-yl-1H-pyrrole-3-carbonitrile,
5-[2-(1-Methyl-1H-indol-5-yl)-pyridin-4-yl]-2-piperazin-1-yl-1H-pyrrole-3-carboxylic acid amide,
N-{4-[4-(4-Cyano-5-piperazin-1-yl-1H-pyrrol-2-yl)-pyridin-2-yl]-phenyl}-acetamide,
5-[2-(4-Acetylamino-phenyl)-pyridin-4-yl]-2-piperazin-1-yl-1H-pyrrole-3-carboxylic acid amide,
5-[2-(3-Dimethylamino-phenyl)-pyridin-4-yl]-2-piperazin-1-yl-1H-pyrrole-3-carbonitrile,
N-{3-[4-(4-Cyano-5-piperazin-1-yl-1H-pyrrol-2-yl)-pyridin-2-yl]-phenyl}-acetamide,
5-[2-(4-Dimethylamino-phenyl)-pyridin-4-yl]-2-piperazin-1-yl-1H-pyrrole-3-carbonitrile,
5-[2-(1H-Indol-6-yl)-pyridin-4-yl]-2-piperazin-1-yl-1H-pyrrole-3-carbonitrile,
5-[2-(1H-Indol-5-yl)-pyridin-4-yl]-2-piperazin-1-yl-1H-pyrrole-3-carbonitrile,
(E)-3-{4-[4-(4-Cyano-5-piperazin-1-yl-1H-pyrrol-2-yl)-pyridin-2-yl]-phenyl}-acrylic acid,
4-{(E)-2-[4-(4-Cyano-5-piperazin-1-yl-1H-pyrrol-2-yl)-pyridin-2-yl]-vinyl}benzoic acid methyl ester,
5-{2-[1-(2-Morpholin-4-yl-ethyl)-1H-pyrazol-4-yl]-pyridin-4-yl}-2-piperazin-1-yl-1H-pyrrole-3-carbonitrile,
5-[5′-(2-Methoxy-ethoxy)-[2,3′]bipyridinyl-4-yl]-2-piperazin-1-yl-1H-pyrrole-3-carbonitrile,
5-{2-[3-(3-Hydroxy-propyl)-phenyl]-pyridin-4-yl}-2-piperazin-1-yl-1H-pyrrole-3-carbonitrile,
{3-[4-(4-Cyano-5-piperazin-1-yl-1H-pyrrol-2-yl)-pyridin-2-yl]-5-fluoro-phenoxy}-acetic acid,
5-[2-(4-Methanesulfonyl-phenyl)-pyridin-4-yl]-2-piperazin-1-yl-1H-pyrrole-3-carbonitrile trifluoroacetate,
5-[2-(4-Methanesulfonyl-phenyl)-pyridin-4-yl]-2-piperazin-1-yl-1H-pyrrole-3-carboxylic acid amide,
5-[2-(3-Methanesulfonyl-phenyl)-pyridin-4-yl]-2-piperazin-1-yl-1H-pyrrole-3-carbonitrile trifluoroacetate,
5-[2-(3-Methanesulfonyl-phenyl)-pyridin-4-yl]-2-piperazin-1-yl-1H-pyrrole-3-carboxylic acid amide,
5-[2-(3-Acetyl-phenyl)-pyridin-4-yl]-2-piperazin-1-yl-1H-pyrrole-3-carbonitrile trifluoroacetate,
2-Piperazin-1-yl-5-[2-(1H-pyrazol-4-yl)-pyridin-4-yl]-1H-pyrrole-3-carbonitrile trifluoroacetate,
5-[2-(1-Benzyl-1H-pyrazol-4-yl)-pyridin-4-yl]-2-piperazin-1-yl-1H-pyrrole-3-carbonitrile trifluoroacetate,
5-[2-(1-Benzyl-1H-pyrazol-4-yl)-pyridin-4-yl]-2-piperazin-1-yl-1H-pyrrole-3-carboxylic acid amide,
2-Piperazin-1-yl-5-{2-[4-(pyrrolidine-1-carbonyl)-phenyl]-pyridin-4-yl}-1H-pyrrole-3-carbonitrile trifluoroacetate,
2-Piperazin-1-yl-5-{2-[4-(pyrrolidine-1-carbonyl)-phenyl]-pyridin-4-yl}-1H-pyrrole-3-carboxylic acid amide,
5-{2-[4-Chloro-3-(pyrrolidine-1-carbonyl)-phenyl]-pyridin-4-yl}-2-piperazin-1-yl-1H-pyrrole-3-carbonitrile trifluoroacetate,
5-{2-[4-Chloro-3-(pyrrolidine-1-carbonyl)-phenyl]-pyridin-4-yl}-2-piperazin-1-yl-1H-pyrrole-3-carboxylic acid amide,
2-Piperazin-1-yl-5-{2-[3-(pyrrolidine-1-carbonyl)-phenyl]-pyridin-4-yl}-1-pyrrole-3-carbonitrile trifluoroacetate,
2-Piperazin-1-yl-5-{2-[3-(pyrrolidine-1-carbonyl)-phenyl]-pyridin-4-yl}-1H-pyrrole-3-carboxylic acid amide,
4-[4-(4-Cyano-5-piperazin-1-yl-1H-pyrrol-2-yl)-pyridin-2-yl]-benzoic acid ethyl ester trifluoroacetate,
5-{2-[3-Nitro-5-(pyrrolidine-1-carbonyl)-phenyl]-pyridin-4-yl}-2-piperazin-1-yl-1H-pyrrole-3-carbonitrile trifluoroacetate,
5-[2-(3-Cyclopentylcarbamoyl-phenyl)-pyridin-4-yl]-2-piperazin-1-yl-1H-pyrrole-3-carboxylic acid amide,
5-{2-[2-Fluoro-5-(pyrrolidine-1-carbonyl)-phenyl]-pyridin-4-yl)-2-piperazin-1-yl-1H-pyrrole-3-carboxylic acid amide,
N-(2-Cyano-ethyl)-3-[4-(4-cyano-5-piperazin-1-yl-1H-pyrrol-2-yl)-pyridin-2-yl]-benzamide trifluoroacetate,
4-[4-(4-Cyano-5-piperazin-1-yl-1H-pyrrol-2-yl)-pyridin-2-yl]-N-(2,2,2-trifluoro-ethyl)-benzamide trifluoroacetate,
5-{2-[4-(Morpholine-4-sulfonyl)-phenyl]-pyridin-4-yl}-2-piperazin-1-yl-1H-pyrrole-3-carbonitrile trifluoroacetate,
5-{2-[4-(Morpholine-4-sulfonyl)-phenyl]-pyridin-4-yl}-2-piperazin-1-yl-1H-pyrrole-3-carboxylic acid amide,
5-{2-[3-Nitro-5-(pyrrolidine-1-carbonyl)-phenyl]-pyridin-4-yl}-2-piperazin-1-yl-1H-pyrrole-3-carboxylic acid amide,
5-{2-[3-(5-Methyl-[1,3,4]oxadiazol-2-yl)-phenyl]-pyridin-4-yl}-2-piperazin-1-yl-1H-pyrrole-3-carbonitrile trifluoroacetate,
4-[4-(4-Cyano-5-piperazin-1-yl-1H-pyrrol-2-yl)-pyridin-2-yl]-N-cyclopentyl-benzamide trifluoroacetate,
5-[2-(4-Cyclopentylcarbamoyl-phenyl)-pyridin-4-yl]-2-piperazin-1-yl-1H-pyrrole-3-carboxylic acid amide,
5-{2-[4-(5-Methyl-[1,3,4]oxadiazol-2-yl)-phenyl)-pyridin-4-yl}-2-piperazin-1-yl-1H-pyrrole-3-carbonitrile trifluoroacetate,
5-{2-[3-(5-Methyl-[1,3,4]oxadiazol-2-yl)-phenyl]-pyridin-4-yl}-2-piperazin-1-yl-1H-pyrrole-3-carboxylic acid amide,
2-Piperazin-1-yl-5-{2-[4-(2,2,2-trifluoro-ethylcarbamoyl)-phenyl]-pyridin-4-yl)-1H-pyrrole-3-carboxylic acid amide,
Morpholine-4-carboxylic acid {4-[4-(4-carbamoyl-5-piperazin-1-yl-1H-pyrrol-2-yl)-pyridin-2-yl]-phenyl}-amide,
5-[2-(4-Methyl-3,4-dihydro-2H-benzo[1,4]oxazin-6-yl)-pyridin-4-yl]-2-piperazin-1-yl-1H-pyrrole-3-carbonitrile trifluoroacetate,
(S)-3-Amino-5′-{2-[(E)-2-(4-fluoro-phenyl)-vinyl]-pyridin-4-yl}-2,3,4,5-tetrahydro-1′H-[1,2′]bipyrrolyl-3-carbonitrile trifluoroacetate,
(S)-3-Amino-5′-{2-[(E)-2-(4-fluoro-phenyl)-vinyl]-pyridin-4-yl}-2,3,4,5-tetrahydro-1′H[1,2′]bipyrrolyl-3′-carboxylic acid amide,
(R)-3-Amino-5′-{2-[(E)-2-(4-fluoro-phenyl)-vinyl]-pyridin-4-yl}-2,3,4,5-tetrahydro-1′H-[1,2′[bipyrrolyl-3-carbonitrile trifluoroacetate,
(R)-3-Amino-5′-{2-[(E)-2-(4-fluoro-phenyl)-vinyl]-pyridin-4-yl}-2,3,4,5-tetrahydro-1′H-[1,2′[bipyrrolyl-3′-carboxylic acid amide,
2-[1,4]Diazepan-1-yl-5-{2-[(E)-2-(4-fluoro-phenyl)-vinyl]-pyridin-4-yl}-1H-pyrrole-3-carbonitrile trifluoroacetate,
2-[1,4]Diazepan-1-yl-5-{2-[(E)-2-(4-fluoro-phenyl)-vinyl]-pyridin-4-yl}-1H-pyrrole-3-carboxylic acid amide,
2-(4-Amino-piperidin-1-yl)-5-{2-[(E)-2-(4-fluoro-phenyl)-vinyl]-pyridin-4-yl}-1H-pyrrole-3-carbonitrile trifluoroacetate,
2-(4-Amino-piperidin-1-yl)-5-{2-[(E)-2-(4-fluoro-phenyl)-vinyl]-pyridin-4-yl}-1H-pyrrole-3-carboxylic acid amide,
5-{2-[(E)-2-(4-Fluoro-phenyl)-vinyl]-pyridin-4-yl)-2-(4-hydroxy-piperidin-1-yl)-1H-pyrrole-3-carbonitrile,
5-{2-[(E)-2-(4-Fluoro-phenyl)-vinyl]-pyridin-4-yl}-2-(3-hydroxy-piperidin-1-yl)-1H-pyrrole-3-carbonitrile,
5-{2-[(E)-2-(4-Morpholin-4-ylmethyl-phenyl)-vinyl]-pyridin-4-yl}-2-piperazin-1-yl-1H-pyrrole-3-carbonitrile trifluoroacetate,
5-{2-[(E)-2-(4-Morpholin-4-ylmethyl-phenyl)-vinyl]-pyridin-4-yl}-2-piperazin-1-yl-1H-pyrrole-3-carboxylic acid amide hydrobromide,
4-{(E)-2-[4-(4-Cyano-5-piperazin-1-yl-1H-pyrrol-2-yl)-pyridin-2-yl]-vinyl}-N,N-diethyl-benzamide trifluoroacetate,
5-{2-[(E)-2-(4-Diethylcarbamoyl-phenyl)-vinyl]-pyridin-4-yl}-2-piperazin-1-yl-1H-pyrrole-3carboxylic acid amide,
5-(2-{(E)-2-[4-(Morpholine-4-carbonyl)-phenyl]-vinyl)-pyridin-4-yl)-2-piperazine-1-yl-1H-pyrrole-3-carbonitrile,
5-(2-{(E)-2-[4-(Morpholine-4-carbonyl)-phenyl]-vinyl}-pyridin-4-yl)-2-piperazin-1-yl-1H-pyrrole-3-carboxylic acid amide,
5-{2-[(E)-2-(4-(Methoxyphenyl)-vinyl]-pyridin-4-yl}-2-piperazin-1-yl-1H-pyrrole-3-carbonitrile,
2-Piperazin-1-yl-5-[2-((E)-2-pyridin-4-yl-vinyl)-pyridin-4-yl]-1H-pyrrole-3-carbonitrile,
2-Piperazin-1-yl-5-[2-((E)-2-pyridin-4-yl-vinyl)-pyridin-4-yl]-1H-pyrrole-3-carboxylic acid amide,
2-Piperazin-1-yl-5-[2-((E)-2-pyridin-3-yl-vinyl)-pyridin-4-yl]-1H-pyrrole-3-carbonitrile,
2-Piperazin-1-yl-5-[2-((E)-2-pyridin-3-yl-vinyl)-pyridin-4-yl]-1H-pyrrole-3-carboxylic acid amide,
2-Piperazin-1-yl-5-[4-((E)-2-pyridin-2-yl-vinyl)-pyridin-4-yl]-1H-pyrrole-3-carbonitrile,
2-Piperazin-1-yl-5-[2-((E)-2-pyridin-2-yl-vinyl)-pyridin-4-yl]-1H-pyrrole-3-carboxylic acid amide,
N-Hydroxy-2-piperazin-1-yl-5-[2-((E)-styryl)-pyridin-4-yl]-1H-pyrrole-3-carboxamidine,
5(2-Phenethyl-pyridin-4-yl)-2-piperazin-1-yl-1H-pyrrole-3-carboxamidine,
2-(4-Methyl-piperazin-1-yl)-5-[2-((E)-styryl)-pyridin-4-yl]-1H-pyrrole-3-carboxylic acid amine,
4-{3-Cyano-5-[2-((E)-styryl)-pyridin-4-yl]-1H-pyrrol-2-yl}-piperazine-1-carboxylic acid benzylamide,
2-Piperazin-1-yl-5-(2-quinolin-3-yl-pyridin-4-yl)-1H-pyrrole-3-carboxamidine,
2-(4-Formyl-piperazin-1-yl)-5-[2-(2-[1,4]oxazepan-4-yl-pyrimidin-5-yl)-pyridin-4-yl]-1H-pyrrole-3-carbonitrile,
5-[2-(4-Morpholin-4-yl-phenylamino)-pyridin-4-yl]-2-piperazin-1-yl-1H-pyrrole-3-carbonitrile hydrochloride,
5-{2-[4-(Morpholine-4-carbonyl)-phenylamino]-pyridin-4-yl}-2-piperazin-1-yl-1H-pyrrole-3-carbonitrile trifluoroacetate,
5-{2-[3-(Morpholine-4-sulfonyl)-phenylamino]-pyridin-4-yl}-2-piperazin-1-yl-1H-pyrrole-3-carbonitrile trifluoroacetate,
5-{2-[3-(Morpholine-4-sulfonyl)-phenylamino]-pyridin-4-yl}-2-piperazin-1-yl-1H-pyrrole-3-carboxylic acid amide,
5-{2-[4-(Morpholine-4-sulfonyl)-phenylamino]-pyridin-4-yl)-2-piperazin-1-yl-1H-pyrrole-3-carbonitrile trifluoroacetate,
5-{2-[4-(Morpholine-4-sulfonyl)-phenylamino]-pyridin-4-yl}-2-piperazin-1-yl-1H-pyrrole-3-carboxylic acid amide,
5-(2-Imidazol-1-yl-pyridin-4-yl)-2-piperazin-1-yl-1H-pyrrole-3-carbonitrile,
5-[2-(4-Phenyl-imidazol-1-yl)-pyridin-4-yl]-2-piperazin-1-yl-1H-pyrrole-3-carbonitrile,
5-{2-[(E)-2-(4-Fluoro-phenyl)-vinyl]-pyridin-4-yl}-1-methyl-2-piperazin-1-yl-1H-pyrrole-3-carbonitrile trifluoroacetate,
5-{2-[(E)-2-(4-Fluoro-phenyl)-vinyl]-pyridin-4-yl}-2-(4-methanesulfonyl-piperazin-1-yl)-1H-pyrrole-3-carbonitrile,
5-{2-[(E)-2-(4-Fluoro-phenyl)-vinyl]-pyridin-4-yl}-2-(4-methanesulfonyl-piperazin-1-yl)-1H-pyrrole-3-carboxylic acid amide,
4-(3-Carbamoyl-5-{2-[(E)-2-(4-morpholin-4-ylmethyl-phenyl)-vinyl]-pyridin-4-yl}-1H-pyrrol-2-yl)-piperazine-1-carboxylic acid benzyl ester,
2-Piperazin-1-yl-5-(6′-pyrrolidin-1-yl-[2,3′]bipyridinyl-4-yl)-1H-pyrrole-3-carbonitrile trifluoroacetate,
2-Piperazin-1-yl-5-(6′-pyrrolidin-1-yl-[2,3′]bipyridinyl-4-yl)-1H-pyrrole-3-carboxylic acid amide,
5-(2-{2-[(2-Methoxy-ethyl)-methyl-amino]-pyrimidin-5-yl}-pyridin-4-yl)-2-piperazin-1-yl-1H-pyrrole-3-carbonitrile trifluoroacetate,
5-[6′-(1-Methyl-piperidin-4-yloxy)-[2,3′]bipyridinyl-4-yl]-2-piperazin-1-yl-1H-pyrrole-3-carbonitrile trifluoroacetate,
5-(6′-Morpholin-4-yl-[2,3′]bipyridinyl-4-yl)-2-piperazin-1-yl-1H-pyrrole-3-carbonitrile trifluoroacetate,
5-[2-(2-Morpholin-4-yl-pyrimidin-5-yl)-pyridin-4-yl]-2-piperazin-1-yl-1H-pyrrole-3-carbonitrile trifluoroacetate,
5-(6′-Morpholin-4-yl-[2,3′]bipyridinyl-4-yl)-2-piperazin-1-yl-1H-pyrrole-3-carboxylic amide,
2-Piperazin-1-yl-5-(3,4,5,6-tetrahydro-2H-[1,2′;5′,2″]terpyridin-4″-yl)-1H-pyrrole-3-carboxylic acid amide,
5-[6′-(4-Methyl-piperazin-1-yl)-[2,3′]bipyridinyl-4-yl]-2-piperazin-1-yl-1H-pyrrole-3-carboxylic acid amide,
5-{2-[2-(4-Methyl-piperazin-1-yl)-pyrimidin-5-yl]-pyridin-4-yl}-2-piperazin-1-yl-1H-pyrrole-3-carbonitrile bis trifluoroacetate,
5-{2-[2-(4-Methyl-piperazin-1-yl)-pyrimidin-5-yl]-pyridin-4-yl}-2-piperazin-1-yl-1H-pyrrole-3-carboxylic acid amide,
5-[6′-(4-Cyclopentyl-piperazin-1-yl)-[2,3′]bipyridinyl-4-yl]-2-piperazin-1-yl-1H-pyrrole-3-carbonitrile trifluoroacetate,
5-[6′-(4-Methyl-[1,4]diazepan-1-yl)-[2,3′]bipyridinyl-4-yl]-2-piperazin-1-yl-1H-pyrrole-3-carbonitrile trifluoroacetate,
5-[6′-(4-Benzyl-piperazin-1-yl)-[2,3′]bipyridinyl-4-yl]-2-piperazin-1-yl-1H-pyrrole-3-carbonitrile hydrochloride,
5-[6′-(4-Benzyl-piperazin-1-yl)-[2,3′]bipyridinyl-4-yl]-2-piperazin-1-yl-1H-pyrrole-3-carboxylic acid amide,
5-[6′-(4-Cyclopentyl-piperazin-1-yl)-[2,3′]bipipyridinyl-4-yl]-2-piperazin-1-yl-1H-pyrrole-3-carboxylic acid amide,
5-[2-(2-[1,4]Oxazepan-4-yl-pyrimidin-5-yl)-pyridin-4-yl]-2-piperazin-1-yl-1H-pyrrole-3-carbonitrile trifluoroacetate,
5-[2-(2-Azepan-1-yl-pyrimidin-5-yl)-pyridin-4-yl]-2-piperazin-1-yl-1H-pyrrole-3-carbonitrile trifluoroacetate,
5-{2-[2-(Isobutyl-methyl-amino)-pyrimidin-5-yl]-pyridin-4-yl}-2-piperazin-1-yl-1H-pyrrole-3-carbonitrile trifluoroacetate,
2-Piperazin-1-yl-5-[2-(2-pyrrolidin-1-yl-pyrimidin-5-yl)-pyridin-4-yl]-1H-pyrrole-3-carbonitrile trifluoroacetate,
2-Piperazin-1-yl-5-[2-(2-piperidin-1-yl-pyrimidin-5-yl)-pyridin-4-yl]-1H-pyrrole-3-carbonitrile trifluoroacetate,
5-[2-(2-Methylamino-pyrimidin-5-yl)-pyridin-4-yl]-2-piperazin-1-yl-1H-pyrrole-3-carbonitrile trifluoroacetate,
2-Piperazin-1-yl-5-[2-(2-pyrrolidin-1-yl-pyrimidin-5-yl)-pyridin-4-yl]-1H-pyrrole-3-carboxylic acid amide,
2-Piperazin-1-yl-5-[2-(2-piperidin-1-yl-pyrimidin-5-yl)-pyridin-4-yl]-1H-pyrrole-3-carboxylic acid amide,
5-{2-[2-((2R,6S)-2,6-Dimethyl-morpholin-4-yl)-pyrimidin-5-yl]-pyridin-4-yl}-2-piperazin-1-yl-1H-pyrrole-3-carbonitrile trifluoroacetate,
2-Piperazin-1-yl-5-{2-[2-(3-trifluoromethyl-5,6-dihydro-8H-[1,2,4]triazolo[4,3-a]pyrazin-7-yl)-pyrimidin-5-yl]-pyridin-4-yl}-1H-pyrrole-3-carbonitrile trifluoroacetate,
5-{2-[(E)-2-(4-Fluoro-phenyl)-vinyl]-pyridin-4-yl}-2-methyl-1H-pyrrole-3-carbonitrile,
5-{2-[(E)-2-(4-Fluoro-phenyl)-vinyl]-pyridin-4-yl}-2-methyl-1H-pyrrole-3-carboxylic acid amide,
2-Methyl-5-[6′-(4-methyl-piperazin-1-yl)-[2,3′]bipyridinyl-4-yl]-1H-pyrrole-3-carbonitrile,
2-Methyl-5-{2-[(E)-2-(4-morpholin-4-ylmethyl-phenyl)-vinyl]-pyridin-4-yl}-1H-pyrrole-3-carbonitrile,
5-[6′-(4-Benzyl-piperazin-1-yl)-[2,3′]bipyridinyl-4-yl]-2-methyl-1H-pyrrole-3-carbonitrile,
2-Methyl-5-(2-{(E)-2-[4-(morpholine-4-carbonyl)-phenyl]-vinyl}-pyridin-4-yl)-1H-pyrrole-3-carbonitrile,
2-Methyl-5-[6′-(1-methyl-piperidin-4-yloxy)-[2,3′]bipyridinyl-4-yl)-1H-pyrrole-3-carbonitrile,
5-(2-{2-[(2-Methoxy-ethyl)-methyl-amino]-pyrimidin-5-yl}-pyridin-4-yl)-2-methyl-1H-pyrrole-3-carbonitrile,
5-{2-[4-Methoxy-3-(3-methoxy-propoxy)-phenyl]-pyridin-4-yl}-2-methyl-1H-pyrrole-3-carbonitrile,
5-{2-[4-Methoxy-phenyl]-pyridin-4-yl}-2-methyl-1H-pyrrole-3-carbonitrile,
4-Methyl-5-[2-(2-[1,4]oxazepan-4-yl-pyrimidin-5-yl)-pyridin-4-yl]-2-piperazin-1-yl-1H-pyrrole-3-carboxylic acid amide,
5-[6′-(4-Cyclopentyl-piperazin-1-yl)-[2,3′]bipyridinyl-4-yl]-4-methyl-2-piperazin-1-yl-1H-pyrrole-3-carbonitrile,
4-Methyl-5-{2-[(E)-2-(4-morpholin-4-ylmethyl-phenyl)-vinyl]-pyridin-4-yl}-2-piperazin-1-yl-1H-pyrrole-3-carbonitrile,
2-Isopropyl-5-[2-((E)-styryl)-pyridin-4-yl]-1H-pyrrole-3-carboxylic acid ethyl ester,
2-Isopropyl-5-[2-((E)-styryl)-pyridin-4-yl]-1H-pyrrole-3-carboxylic acid amide,
2-Isopropyl-5-{2-[(E)-2-(4-morpholin-4-ylmethyl-phenyl)-vinyl]-pyridin-4-yl}-1H-pyrrole-3-carbonitrile,
2-Piperidin-4-yl-5-[2-((E)-styryl)-pyridin-4-yl]-1H-pyrrole-3-carbonitrile,
2-Piperidin-4-yl-5-[2-((E)-styryl)-pyridin-4-yl]-1H-pyrrole-3-carboxylic acid,
2-Piperidin-4-yl-5-[2-((E)-styryl)-pyridin-4-yl]-1H-pyrrole-3-carboxylic acid amide,
2-Piperidin-4-yl-5-[2-((E)-styryl)-pyridin-4-yl]-1H-pyrrole-3-carboxylic acid benzylamide,
2-(2-Amino-ethyl)-5-[2-((E)-styryl)-pyridin-4-yl]-1H-pyrrole-3-carbonitrile,
5-[2-((E)-Styryl)-pyridin-4-yl]-2-(1,2,3,6-tetrahydro-pyridin-4-yl)-1H-pyrrole-3-carbonitrile,
5-[2-(2-Morpholin-4-yl-pyrimidin-5-yl)-pyridin-4-yl]-2-piperidin-4-yl-1H-pyrrole-3-carbonitrile,
2-(2-Amino-ethyl)-5-(2-quinolin-3-yl-pyridin-4-yl)-1H-pyrrole-3-carbonitrile,
2-Aminomethyl-5-[2-((E)-styryl)-pyridin-4-yl]-1H-pyrrole-3-carbonitrile,
2-Aminomethyl-5-(2-quinolin-3-yl-pyridin-4-yl)-1H-pyrrole-3-carbonitrile,
5-(2-Quinolin-3-yl-pyridin-4-yl)-2-(1,2,3,6-tetrahydro-pyridin-4-yl)-1H-pyrrole-3-carbonitrile,
2-(2-Amino-ethyl)-5-[2-((E)-styryl)-pyridin-4-yl]-1H-pyrrole-3-carboxylic acid,
2-(2-Amino-ethyl)-5-(2-quinolin-3-yl-pyridin-4-yl)-1H-pyrrole-3-carboxylic acid,
2-(2-Amino-ethyl)-5-(2-phenyl-pyridin-4-yl)-1H-pyrrole-3-carboxylic acid,
2-(2-Hydroxy-ethyl)-5-[2-((E)-styryl)-pyridin-4-yl]-1H-pyrrole-3-carboxylic acid,
2-Aminomethyl-5-[2-((E)-styryl)-pyridin-4-yl]-1H-pyrrole-3-carboxylic acid,
2-Aminomethyl-5-(2-quinolin-3-yl-pyridin-4-yl)-1H-pyrrole-3-carboxylic acid,
2-(2-Amino-ethyl)-5-[2-(2-[1,4]oxazepan-4-yl-pyrimidin-5-yl)-pyridin-4-yl-1H-pyrrole-3-carboxylic acid,
2-(2-Amino-ethyl)-5-[2-(4-methoxy-phenyl)-pyridin-4-yl]-1H-pyrrole-3-carboxylic acid,
2-(2-Amino-ethyl)-5-(5′-methoxy-[2,3′]bipyridinyl-4-yl)-1H-pyrrole-3-carboxylic acid,
2-(2-Amino-ethyl)-5-[2-(3-methanesulfonyl-phenyl)-pyridin-4-yl]-1H-pyrrole-3-carboxylic acid,
2-(2-Amino-ethyl)-5-(6′-methoxy-[2,3′]bipyridinyl-4-yl)-1H-pyrrole-3-carboxylic acid,
2-(2-Amino-ethyl)-5-[2-(2,3-dihydro-benzo[1,4]dioxin-6-yl)-pyridin-4-yl]-1H-pyrrole-3-carboxylic acid,
2-(2-Amino-ethyl)-5-(2-quinolin-6-yl-pyridin-4-yl)-1H-pyrrole-3-carboxylic acid,
2-(2-Amino-ethyl)-5-{2-[(E)-2-(4-morpholin-4-ylmethyl-phenyl)-vinyl]-pyridin-4-yl}-1H-pyrrole-3-carboxylic acid,
2-(2-Amino-ethyl)-5-[2-((E)-styryl)-pyridin-4-yl]-1H-pyrrole-3-carboxylic acid amide,
5-[2-((E)-Styryl)-pyridin-4-yl)-2-(1,2,3,6-tetrahydro-pyridin-4-yl)-1H-pyrrole-3-carboxylic acid amide,
2-Aminomethyl-5-[2-((E)-styryl)-pyridin-4-yl]-1H-pyrrole-3-carboxylic acid amide, or
2-(2-Dimethylamino-ethyl)-5-[2-((E)-styryl)-pyridin-4-yl]-1H-pyrrole-3-carbonitrile.

6. (canceled)

7. (canceled)

8. (canceled)

9. A method of treatment of cytokine mediated conditions, comprising:
administering to a subject in need thereof, an effective amount of the compound according to claim 1 or a pharmaceutically-acceptable salt thereof.

10. A pharmaceutical composition, comprising:
the compound according to claim 1, or a pharmaceutically-acceptable salt thereof, and
a pharmaceutically acceptable excipient, diluent or carrier.

11. (canceled)

12. A combination, comprising:
the compound according to claim 1 and
an active agent selected from the group consisting of: an anti IL-1 agent, anti cytokine and anti-cytokine receptor agent, B-cell and T-cell modulating drug, disease-modifying anti-rheumatic agents, gold salts, penicillamine, hydroxychloroquine, chloroquine, azathioprine, glucocorticoids and non-steroidal anti-inflammatories, cyclooxygenase inhibitors, selective COX-2 inhibitors, agents which modulate migration of immune cells, chemokine receptor antagonists, modulators of adhesion molecules; for simultaneous, sequential or separate administration.

13. The method of claim 9, wherein the cytokine mediated condition is a TNF alpha-mediated disease or condition or a MK2 related disease or condition.

14. The method of claim 9, wherein the cytokine mediated condition is an autoimmune or inflammatory disorder.
